(19) United States
(12) Patent Application Publication
(10) Pub. No.: US 2024/0299596 A1
(43) Pub. Date: Sep. 12, 2024

(54) METHODS AND MATERIALS FOR COMBINING BIOLOGICS WITH MULTIPLE CHELATORS

(71) Applicant: Mayo Foundation for Medical Education and Research, Rochester, MN (US)

(72) Inventors: Mukesh K. Pandey, Rochester, MN (US); Geoffrey B. Johnson, Rochester, MN (US); David J. Bartlett, Rochester, MN (US)

(21) Appl. No.: 18/569,359

(22) PCT Filed: Jun. 17, 2022
(86) PCT No.: PCT/US22/34086

§ 371 Date: Dec. 12, 2023

Related U.S. Application Data
(60) Provisional application No. 63/211,919, filed on Jun. 17, 2021.

(51) Int. Cl. A61K 51/04; A61K 51/08; A61K 51/10; A61P 35/00

(52) U.S. Cl. CPC A61K 51/0482; A61K 51/0455; A61K 51/083; A61K 51/088; A61K 51/1096; A61P 35/00

(57) ABSTRACT

Conjugates that include two or more chelators (e.g., a chelator of a radiotherapy isotope and a chelator of an imaging isotope) covalently attached to one or more binding moieties are provided herein. The conjugates can be used for treating cancer or non-cancer conditions, and can serve as both an imaging and a radiotherapy molecule when the imaging isotope is complexed to the chelator of the imaging isotope and the radiotherapy isotope is complexed to the chelator of the radiotherapy isotope.

### CROSS-REFERENCE TO RELATED APPLICATIONS

This application claims the benefit of U.S. Patent Application Ser. No. 63/211,919, filed on Jun. 17, 2021. The disclosure of the prior application is considered part of (and is incorporated by reference in) the disclosure of this application.

### BACKGROUND

### 1. Technical Field

This document relates to conjugates of two or more chelators (e.g., a conjugate of a chelator of an isotope for imaging and a chelator of an isotope for radiotherapy) and one or more binding moieties, and using such conjugates for treating diseases such as cancer. For example, this document provides methods and materials for combining a binding moiety with two or more chelators, wherein one of the chelators is a chelator of an isotope used for imaging and one of the chelators is a chelator of an isotope used for radiotherapy. A conjugate in which the imaging isotope and the radiotherapy isotope are complexed to the chelators can be administered to a mammal in need of treatment, and can serve as both an imaging and a radiotherapy molecule.

### 2. Background Information

In the field of targeted radionuclide therapy, the ability to accurately calculate dosimetry (how much therapy drug has gone to tumors and tissues in the body) through imaging of a patient is a powerful way to understand the disease pathology, disease progression, and response to radionuclide therapy, and also helps to enhance drug development via a better understanding of pharmacokinetic and pharmacodynamics, expediting regulatory (e.g., FDA) approvals and personalize care for patients (e.g., cancer patients). The field of targeted radionuclide therapy is moving toward more effective and often more expensive alpha-emitters, and away from beta-emitters. However, alpha-emitters are typically not suited for imaging due to the unavailability or low abundance of the appropriate positron or photon-energy emissions (511 KeV for PET and 100-200 KeV for SPECT). The high linear energy transfer (LET) of alpha-emission, and the γ-photons, characteristic x-rays, or bremsstrahlung radiation that accompany decay of the parent alpha emitting radionuclide are poorly suited for quantifying target uptake, dosimetry, and therapy response compared to beta-emitters. Furthermore, even when performing therapy with beta-emitters that can be imaged, the beta-emitters are often imaged poorly with SPECT technology. If imaging of radionuclide therapies could be performed with PET technology, the resolution, accuracy, and quality of the images would be superior. As a result, most research and development, FDA submissions, and clinical programs have to depend on estimated biodistribution/dosimetry based on poor quality images or by using a surrogate imaging probe (a modified drug that can be imaged). These surrogate imaging probes differ significantly from the alpha-emitter therapy drug in multiple ways, making them less optimal for predicting the biodistribution/dosimetry of the alpha-emitting therapy drug. Therefore, there is a need for improved radiotherapies that can be imaged directly and accurately.

### SUMMARY

This document is based, at least in part, on the discovery of a method of combining (e.g., covalently attaching) a binding moiety or motif, e.g., a biologic or drug that binds to a target molecule in a mammal, with multiple chelators such that the resulting conjugate or mixtures of conjugates can serve simultaneously as both an imaging and radiotherapy molecule when suitable isotopes are complexed with the chelators. The resulting conjugates include two or more chelators and a binding moiety (e.g., two or more chelators covalently attached to a binding moiety via one or more linkers), wherein one of the chelators is a chelator of an isotope used for imaging (referred to herein as a “chelator of an imaging isotope”) and one of the chelators is a chelator of an isotope used for radiotherapy (referred to herein as a “chelator of a radiotherapy isotope”). As described herein, the conjugates can be selectively used for imaging or radionuclide therapy as needed by choosing radionuclides for imaging or therapy and filling the other chelator with a non-radioactive version of the imaging or therapy metal ion to maintain the same chemical nature of the molecule. Using the same chemical entity preserves the same biodistribution, and avoids using surrogate imaging probes that differ in structure and can have a different biodistribution. In addition, the same conjugate can be used for both imaging and radionuclide therapy by complexing both the chelators with appropriate imaging and therapy radionuclides, without being forced to choose only a single isotope that is suboptimal at one or both tasks.

The conjugates and methods described herein can allow the biodistribution and dosimetry of alpha-emitting therapy drugs to be evaluated prior to therapy and also evaluated with each cycle of radiotherapy, helping to speedup research and development, speedup FDA approvals, and guide clinical care. In addition, the methods described herein can be used to streamline the ongoing evaluation of patients who are receiving these expensive radiotherapies with more accurate therapy monitoring (e.g., by imaging of the therapy right after it is administered) and can do so with a straightforward clinical workflow. This can result in informed changes in the care-plan mid therapy, saving money by stopping futile therapy early, improving outcomes by adjusting or augmenting therapy when needed, or switching to a more effective therapy sooner.

The conjugates described herein can be designed so the half-life of the imaging isotope (e.g., an isotope for positron emission tomography (PET) or an isotope for single photon emission computed tomography (SPECT)) and the physical half-life of the radiotherapy isotope (e.g., an alpha or beta emitting radionuclide) are matched to ensure that the biodistribution of the therapy over the time it is radioactive can be imaged and therefore dosimetry can be accurately calculated. For example, the half-life of the imaging isotope (e.g., an isotope for PET or an isotope for SPECT), the physical half-life of the radiotherapy isotope (e.g., an alpha or beta emitting radionuclide), and the plasma half-life of a targeting vector (e.g., peptide, antibody, or small molecule) can be matched to ensure that the biodistribution of the therapy over the time it is radioactive can be imaged and dosimetry can be accurately calculated. In some embodiments, an optical imaging (near infra-red) probe can be added to the conjugate. The conjugates and methods described herein provide a robust platform to stage the disease, treat the disease, monitor the response to therapy or progression, and/or minimize side effects to healthy organs and tissues, all with versions of the same molecule (chemically and biologically identical). This can be achieved by simply choosing whether a conjugate described herein is complexed with an isotope for imaging and/or complexed with an isotope for radiotherapy or non-radioactive versions of these same isotopes (i.e., radionuclides can be swapped with non-radioactive isotopes that have different nuclear structures but are chemically identical) for the desired use of the conjugate. In some embodiments, two or more conjugates can be used. For example, in some embodiments, one conjugate described herein is complexed with an alpha-emitting isotope for therapy and one conjugate described herein is complexed with a positron-emitting isotope for imaging. Additionally, the conjugates described herein can include more than one binding moiety or motif to enhance the uptake in the targeted tissues/organs.

In one general aspect, this document provides a conjugate comprising two or more chelators and a binding moiety, wherein one of said chelators is a chelator of an imaging isotope and one of said chelators is a chelator of a radiotherapy isotope.

In some embodiments, said isotope used for radiotherapy is an α-emitter. In some embodiments, said isotope used for radiotherapy is both an α-emitter and a β-emitter.

In some embodiments, said radiotherapy isotope is ²²⁵Ac, ²¹²Pb, ²¹¹At, ²¹³Bi, ²¹²Bi, ²¹¹Bi, ²²⁷Th, ²²³Ra, ²¹¹Po, ²²¹Fr, ²¹⁷At, ²¹³Po, ²¹²Po, ²¹⁵Po, or ¹⁷⁷Lu. In some embodiments, said radiotherapy isotope is ²²⁵Ac, ²¹²Pb, ²¹¹At, ²¹³Bi, ²¹²Bi, ²¹¹Bi, ^{152/160/161}Tb, ²²⁷Th, ²²³Ra, ²¹¹Po ²²Fr, ²¹⁷At, ²¹³Po, ²¹²Po ²¹⁵Po, or ¹⁷⁷Lu.

In some embodiments, said imaging isotope is ⁶⁸Ga, ⁴⁴Sc, ^{60/61/62/64}Cu, ^{84/86/87/89}Zr, ⁶³Zn, ^{43/44}Sc, ^{192/193/194/196}Au, ^{52m}Mn, ^{90/92ml}Nb, ^{51/52}Mn ⁴⁵Ti, ^{65/66}Ga, ^{94m}Tc, ⁵⁵Co, ^{80/81/83}Sr, ³⁸K, ^{70/71/72/74}As, ^{81/82m}Rb, ⁵²Fe, or ⁸⁶Y. In some embodiments, said imaging isotope is ⁶⁸Ga ⁴⁴Sc, ^{60/61/62/64}Cu, ^{84/86/87/89}Zr ⁶³Zn, ^{43/44}Sc, ^{192/193/194/196}Au, ^{52m}Mn, ^{90/92ml}Nb, ^{51/52}Mn, ^{148/151/151m/152}Tb, ⁴⁵Ti, ^{65/66/67}Ga, ^{94m}Tc ⁵⁵Co, ^{80/81/83}Sr, ³⁸K, ^{70/71/72/74}As, ^{81/82m}Rb, ⁵²Fe, or ⁸⁶Y.

In some embodiments, said imaging isotope is ⁶⁴Cu and wherein said radiotherapy isotope is ²¹²Pb.

In some embodiments, said imaging isotope is complexed to said chelator of said imaging isotope.

In some embodiments, said radiotherapy isotope is complexed to said chelator of said radiotherapy isotope.

In some embodiments, each of said chelators independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), and diethylenetraminepentacetic acid (DTPA), and N,N′-bis[(6-carboxy-2-pyridil)methyl]-4,13-diaza-18crown-6 (MACROPA). In some embodiments, each of said chelators independently comprises a compound selected from the group consisting ofNOTA, DOTA, TCMC, DiAmSar, HBED, DFO, DTPA, 2,2′,2″-nitrilotriacetic acid; (NTA), 2,2-bis(hydroxymethyl)-2,2′,2″-nitrilotriethanol (BisTris), ethylene glycol-bis(2-aminoethyl ether)-N,N,N′,N′-tetraacetic acid (EGTA), ethylenediamine-N,N,N′,N′-tetraacetic acid (EDTA), 1,2-bis(2-aminophenoxy)ethane-N,N,N′,N′-tetraacetic acid (BAPTA), 1,4,7,10-tetraazacyclododecane-1,7-diacetic acid (DO2A), 1,4,7,10-tetraazacyclododecane-1,4,7-triacetic acid (DO3A) and MACROPA.

In some embodiments, said binding moiety is a polypeptide.

In some embodiments, said polypeptide binds prostate specific membrane antigen, a somatostatin receptor, a fibroblast activation protein, or a melanocortin-1 receptor.

In some embodiments, said polypeptide is an antibody.

In some embodiments, said binding moiety is a small molecule.

In some embodiments, said small molecule is a glutamate carboxypeptidase II inhibitor.

In some embodiments, said chelators are covalently attached to said binding moiety.

In some embodiments, said chelators and said binding moiety are covalently attached via a linker.

In some embodiments, said chelators and said binding moiety are linked via a moiety of Formula (I):

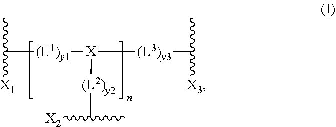

wherein:
each X is independently selected from N, P, P(═O), CR^{N}, and a moiety of formula (i):

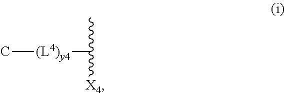

each of x₁, x₂, x₃, and x₄ independently indicates a point of attachment of the moiety of Formula (I) to a chelator or a binding moiety;
each of L¹, L², L³, and L⁴ is independently selected from C(═O), C(═S), N(R^{N}), O, S, S(═O), S(═O)₂, —CR^{N}═NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

In some embodiments, the moiety of Formula (I) has any one of the following formulae:

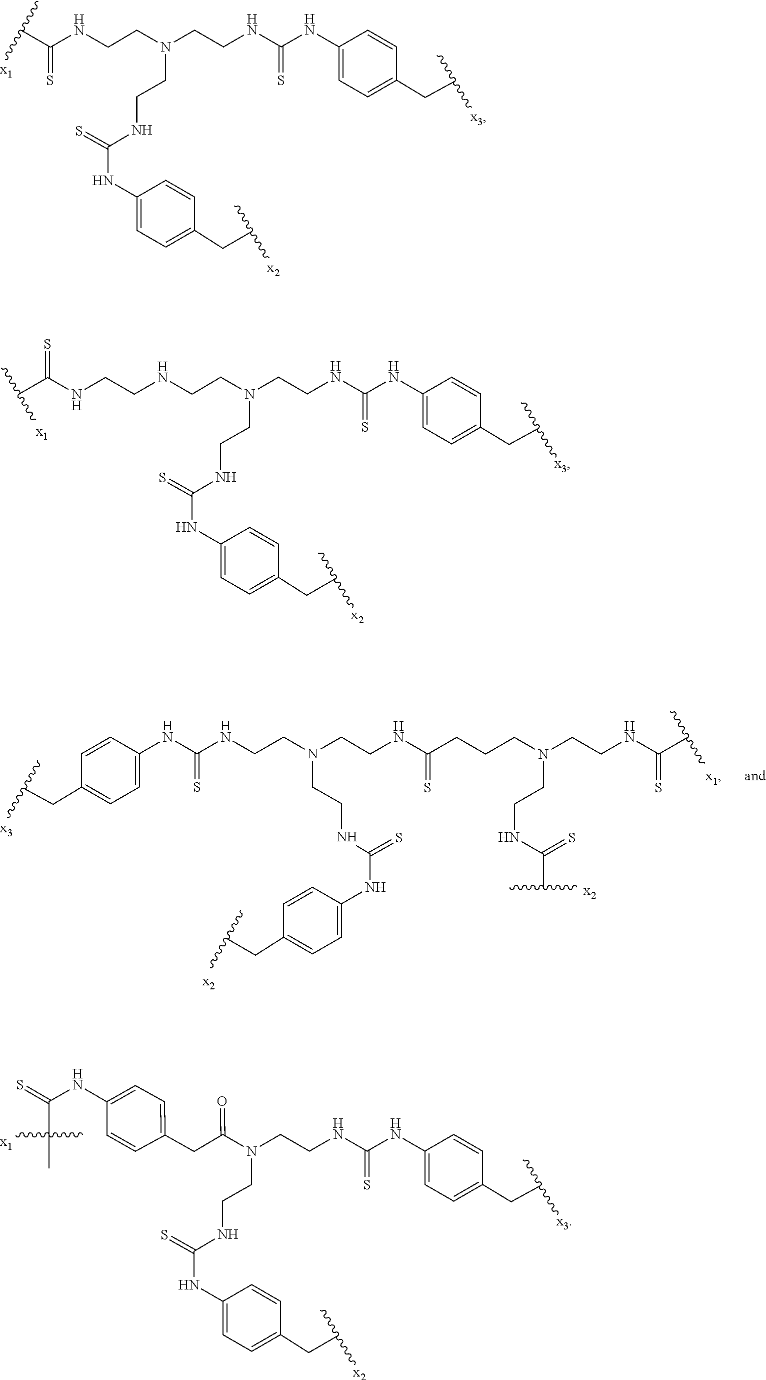

In some embodiments, said chelators and said binding moiety are linked via a moiety of Formula (II):

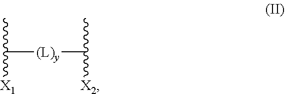

wherein:
x₁ indicates a point of attachment of the Formula (II) to the chelator;
x₂ indicates a point of attachment of the Formula (II) to the chelator or the binding moiety;
each L is independently selected from C(═O), C(═S), N(R^{N}), O, S, S(═O), S(═O)₂, —CR^{N}═NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
y is an integer from 1 to 30; and
each R^{N} is independently selected from H, C₁₋₃ alkyl, and C₁₋₃ haloalkyl.

In some embodiments, the moiety of Formula (II) has any one of the following formulae:

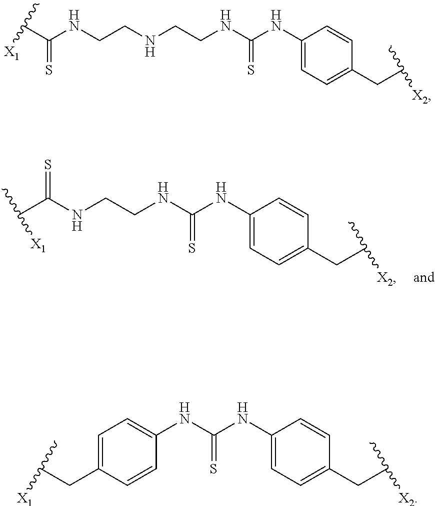

In another general aspect, this document provides a method of treating cancer in a mammal in need thereof, wherein said method comprises administering a conjugate as described herein to said mammal, wherein said conjugate comprises said imaging isotope complexed to said chelator of said imaging isotope and wherein said conjugate comprises said radiotherapy isotope complexed to said chelator of said radiotherapy isotope.

In another general aspect, this document provides a method of treating cancer in a mammal, wherein said method comprises:
a) administering, to said mammal, a first conjugate comprising two or more chelators and a binding moiety, wherein one of said chelators is a chelator of an imaging isotope and one of said chelators is a chelator of a radiotherapy isotope, wherein said first conjugate comprises said imaging isotope complexed to said chelator of said imaging isotope;
b) determining, in said mammal, the biodistribution of said first conjugate; and
c) administering, to said mammal, an amount of a second conjugate that is identical to said first conjugate except that said second conjugate comprises said radiotherapy isotope complexed to said chelator of said radiotherapy isotope.

In some embodiments, said method further comprises determining, in said mammal, the biodistribution of said second conjugate comprising said imaging isotope complexed to said chelator of said imaging isotope and said radiotherapy isotope complexed to said chelator of said radiotherapy isotope.

In some embodiments, said cancer is selected from the group consisting of prostate cancer, a neuroendocrine cancer, colon cancer, lung cancer, pancreatic cancer, melanoma, and a lymphoid cancer.

In another general aspect, this document provides a method of treating cancer in a mammal in need thereof, wherein said method comprises administering, to said mammal, two or more conjugates,
wherein each conjugate comprises two or more chelators and a binding moiety, wherein one of said chelators is a chelator of an imaging isotope and one of said chelators is a chelator of a radiotherapy isotope,
wherein one of said conjugates administered to said mammal comprises an imaging isotope complexed to said chelator of said imaging isotope, and
wherein one of said conjugates administered to said mammal comprises a radiotherapy isotope complexed to said chelator of said radiotherapy isotype.

In some embodiments, said conjugate comprises two or more binding moieties. In some embodiments, said binding moiety can be a polypeptide. In some embodiments, each of said polypeptides can independently bind prostate specific membrane antigen, a somatostatin receptor, a fibroblast activation protein, or a melanocortin-1 receptor.

In some embodiments, said conjugate comprises three or more chelators. In some embodiments, each of said chelators can independently comprise a compound selected from the group consisting ofNOTA, DOTA, TCMC, DiAmSar, HBED, DFO, DTPA, DFO, NTA, BisTris, EGTA, EDTA, BAPTA, DO2A, DTPA, DO3A, and MACROPA.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a decay scheme of ²¹²Pb.

FIGS. 2A and 2B are examples of conjugates of two or more chelators linked to a binding moiety.

FIG. 3 is a scheme for diamsar (Cu) and TCMC (Pb) platform for peptide conjugation.

FIG. 4 is a scheme for NOTA (Cu) and TCMC (Pb) platform for peptide conjugation.

FIG. 5 is a scheme for diamsar (Cu) and TCMC (Pb) platform for dual peptide conjugation.

FIG. 6 is a scheme for NOTA (Cu) and TCMC (Pb) platform for dual peptide conjugation.

FIG. 7 is a representative example of the synthesis of a NOTA(Cu), TCMC (Pb) and peptide (PSMA) conjugate with a different linker molecule.

FIG. 8 is a representative example of the synthesis of a diamsar (Cu), TCMC (Pb) and peptide (PSMA) conjugate with a different linker molecule.

FIG. 9 is an example of conjugates having linear configuration of chelators and a binding moiety using the diamsar (Cu) and TCMC (Pb) platform for peptide conjugation.

FIG. 10 is a high performance liquid-chromatography (HPLC) trace of a conjugate including NOTA (Cu) and TCMC (Pb) with an aniline linker (e.g., Conjugate 1).

FIG. 11 is a graph of the HPLC calibration curve of Conjugate 1.

FIG. 12 is a HPLC trace of unlabeled ⁶⁴Cu.

FIG. 13 is a thin-layer chromatography (TLC) trace of unlabeled ⁶⁴Cu.

FIG. 14 is an example of labeling Conjugate 1 with ⁶⁴Cu to form a 64Cu-Conjugate 1.

FIG. 15 is a TLC trace of the ⁶⁴Cu-Conjugate 1.

FIG. 16 is an HPLC trace of the ⁶⁴Cu-Conjugate 1.

FIG. 17 is an HPLC trace of a conjugate including NOTA (Cu) and TCMC (Pb) with an amino acid linker (e.g., Conjugate 2).

FIG. 18 is an example of labeling Conjugate 2 with ⁶⁴Cu to form a 64Cu-Conjugate 2.

FIG. 19 is a graph of the HPLC calibration curve of Conjugate 2.

FIG. 20 is a TLC trace of ⁶⁴Cu-Conjugate 2.

FIG. 21 is an HPLC trace of ⁶⁴Cu-Conjugate 2.

FIG. 22 is an HPLC trace of ⁶⁴Cu-Conjugate 2 after 40 minutes.

FIG. 23 is an HPLC trace of ⁶⁴Cu-Conjugate 2 after 2 hours.

FIG. 24 is an HPLC trace of ⁶⁴Cu-Conjugate 2 after 4 hours.

FIG. 25 is an HPLC trace of ⁶⁴Cu-Conjugate 2 after 8 hours.

FIG. 26 is a TLC trace of ⁶⁴Cu-Conjugate 2 after 40 minutes.

FIG. 27 is a TLC trace of ⁶⁴Cu-Conjugate 2 after 2 hours.

FIG. 28 is a TLC trace of ⁶⁴Cu-Conjugate 2 after 4 hours.

FIG. 29 is a TLC trace of ⁶⁴Cu-Conjugate 2 after 8 hours.

FIG. 30 is a graph of the percent of cellular uptake of ⁶⁴Cu-Conjugate 2 with and without an inhibitor.

FIG. 31 is a graph of the standardized uptake value (SUV) of ⁶⁴Cu-Conjugate 2 in the organs of nude mice.

FIG. 32 is a blow-up of the graph of the SUV of ⁶⁴Cu-Conjugate 2 in the organs of nude mice.

FIG. 33 contains micro PET images of normal mice injected with the ⁶⁴Cu-Conjugate 2 at different time intervals.

FIG. 34 is an in vivo PET image of the proximal tubules in the kidney of a nude mouse injected with the ⁶⁴Cu-Conjugate 2.

FIG. 35 is an HPLC trace of unlabeled ²⁰³Pb.

FIG. 36 is a TLC trace of unlabeled ²⁰³Pb.

FIG. 37 is an example of labeling Conjugate 1 with ²⁰³Pb to form ²⁰³Pb-Conjugate 1.

FIG. 38 is an HPLC trace of ²⁰³Pb-Conjugate 1.

FIG. 39 is an example of labeling Conjugate 2 with ²⁰³Pb to form ²⁰³Pb-Conjugate 2.

FIG. 40 is a TLC trace of ²⁰³Pb-Conjugate 2.

FIG. 41 is an HPLC trace of ²⁰³Pb-Conjugate 2.

FIG. 42 is a TLC trace of ²⁰³Pb-Conjugate 2 after 40 minutes.

FIG. 43 is a TLC trace of ²⁰³Pb-Conjugate 2 after 2 hours.

FIG. 44 is a TLC trace of ²⁰³Pb-Conjugate 2 after 4 hours.

FIG. 45 is a TLC trace of ²⁰³Pb-Conjugate 2 after 21 hours.

FIG. 46 is an example of mixed labeling Conjugate 2 with ⁶⁴Cu and ²⁰³Pb to form ⁶⁴Cu/²⁰³Pb-Conjugate 2.

FIG. 47 is a TLC trace of ⁶⁴Cu/²⁰³Pb-Conjugate 2 using a 0.15M NH₄Ac mobile phase.

FIG. 48 is a second TLC trace of ⁶⁴Cu/²⁰³Pb-Conjugate 2 using a 0.1M sodium citrate mobile phase.

FIG. 49 is a TLC trace of ⁶⁴Cu/²⁰³Pb-Conjugate 2 after 1 hour using two separate solvent systems. The first solvent system is 0.1 M sodium citrate. The second solvent system is 0.15M NH₄Ac.

FIG. 50 is a TLC trace of ⁶⁴Cu/²⁰³Pb-Conjugate 2 after 4 hours using two separate solvent systems. The first solvent system is 0.1 M sodium citrate. The second solvent system is 0.15M NH₄Ac.

FIG. 51 is a TLC trace of ⁶⁴Cu/²⁰³Pb-Conjugate 2 after 21 hours using two separate solvent systems. The first solvent system is 0.1 M sodium citrate. The second solvent system is 0.15M NH₄Ac.

FIG. 52 is an example of mixed labeling Conjugate 2 with ⁶⁴Cu and non-radioactive Pb to form ⁶⁴Cu/Pb-Conjugate 2.

FIG. 53 is a TLC trace of ⁶⁴Cu/Pb-Conjugate 2.

FIG. 54 is an HPLC trace of ⁶⁴Cu/Pb-Conjugate 2.

FIG. 55 is a graph of the in vitro cellular uptake of ⁶⁴Cu/Pb-Conjugate 2 with and without Pb.

FIG. 56 contains various PET images of the in vivo cellular uptake of ⁶⁴Cu/Pb-Conjugate 2 in mice at various time points post injection.

FIG. 57 is a graph of the SUV of ⁶⁴Cu/Pb-Conjugate 2 in the organs of both normal and tumor bearing mice.

FIG. 58 is a graph of the SUV of ⁶⁴Cu/Pb-Conjugate 2 having a molar specific activity of 0.325 GBq/μmol in the organs of mice.

FIG. 59 is a graph of the SUV of ⁶⁴Cu/Pb-Conjugate 2 having a molar specific activity of 52 GBq/μmol in the organs of mice.

FIG. 60 contains various PET images of the in vivo uptake of ⁶⁴Cu/Pb-Conjugate 2 in mice at various time points post injection.

FIG. 61 contains various PET images of the in vivo uptake of ⁶⁴Cu/Pb-Conjugate 2 in mice at various time points post injection.

FIG. 62 contains various graphs of the SUV of ⁶⁴Cu/Pb-Conjugate 2 in the tumors and kidneys of mice.

FIG. 63 contains various PET images of the in vivo uptake of ⁶⁴Cu/Pb-Conjugate 2 in mice at various time points post injection.

FIG. 64 contains various PET images of the in vivo uptake of ⁶⁴Cu/Pb-Conjugate 2 in mice at various time points post injection.

FIG. 65 contains various graphs of the SUV of ⁶⁴Cu/Pb-Conjugate 2 in the tumors and kidneys of mice.

FIG. 66 is a graph of the in vitro cellular uptake of ⁶⁴Cu-Conjugate 2 with and without an inhibitor.

FIG. 67 is a graph of the SUV of ⁶⁴Cu-Conjugate 2 in the kidney, tumor and salivary gland of tumor bearing mice 120 minutes post injection.

FIG. 68 is a graph of the SUV ratio of ⁶⁴Cu-Conjugate 2 in the kidney over muscle, blood over muscle, tumor over muscle, and salivary gland over muscle of normal and tumor bearing mice.

FIG. 69 contains various micro PET images of the in vivo uptake of ⁶⁴Cu-Conjugate 2 in mice at various time points post injection.

FIG. 70 is a representative example of the synthesis of a dual PSMA targeting conjugate with a different linker molecule as well as NOTA and TCMC chelators.

FIG. 71 is a representative example of the synthesis of a dual PSMA targeting conjugate with a different linker molecule as well as NOTA and TCMC chelators.

FIG. 72 is a representative example of the synthesis of a dual PSMA targeting conjugate with a different linker molecule as well as NOTA and MACROPA chelators.

FIG. 73 is a representative example of the synthesis of a dual PSMA targeting conjugate with a different linker molecule as well as NOTA and MACROPA chelators.

FIG. 74 is a representative example of the synthesis of a dual PSMA targeting conjugate with a different linker molecule as well as DFO and MACROPA chelators.

FIG. 75 is a representative example of the synthesis of a dual PSMA targeting conjugate with a different linker molecule as well as DFO and MACROPA chelators.

FIG. 76 is a representative example of the synthesis of a single PSMA targeting conjugate with a NOTA chelator and a MACROPA chelator.

FIG. 77 is a representative example of the synthesis of a single PSMA targeting conjugate with a DFO chelator and a MACROPA chelator.

FIG. 78 is a representative example of the synthesis of a single FAP targeting conjugate with a NOTA chelator and a MACROPA chelator.

FIG. 79 is a representative example of the synthesis of a single FAP targeting conjugate with a DFO chelator and a MACROPA chelator.

FIG. 80 is a representative example of the synthesis of a single octreotide targeting conjugate with a NOTA chelator and a MACROPA chelator.

FIG. 81 is a representative example of the synthesis of a single octreotide targeting conjugate with a DFO chelator and a MACROPA chelator.

FIG. 82 is an example of labeling of a NOTA(Cu), TCMC(Pb) and FAPI conjugate (Conjugate 3) with ⁶⁴Cu to form ⁶⁴Cu-Conjugate 3.

FIG. 83 is an example of dual labeling of a NOTA(Cu), TCMC(Pb) and FAPI conjugate (Conjugate 3) with ⁶⁴Cu and nonradioactive Pb to form ⁶⁴Cu/Pb-Conjugate 3.

FIG. 84 is a UV HPLC trace of the ⁶⁴Cu-Conjugate 3.

FIG. 85 is a rad-TLC trace of free [⁶⁴Cu]CuCl₂.

FIG. 86 is a rad-TLC trace of ⁶⁴Cu-Conjugate 3.

FIG. 87 is a rad-TLC trace of ⁶⁴Cu/Pb-Conjugate 3.

FIG. 88 is a UV HPLC trace of ⁶⁴Cu/Pb-Conjugate 3.

FIG. 89 is a radiation HPLC trace of ⁶⁴Cu/Pb-Conjugate 3.

FIG. 90 is an example of dual labeling of a NOTA(Cu), TCMC(Pb) and octreotide conjugate (Conjugate 4) with ⁶⁴Cu and nonradioactive Pb to form ⁶⁴Cu/Pb-Conjugate 4.

FIG. 91 is a UV HPLC trace of ⁶⁴Cu/Pb-Conjugate 4.

FIG. 92 is a radiation HPLC trace of ⁶⁴Cu/Pb-Conjugate 4.

FIG. 93 is a rad-TLC trace of free [⁶⁴Cu]CuCl₂.

FIG. 94 is a rad-TLC trace of ⁶⁴Cu/Pb-Conjugate 4.

FIG. 95 is an example of labeling of Conjugate 2 with ²¹²Pb to form ²¹²Pb-Conjugate 2.

FIG. 96 is a rad-TLC trace of [²¹²Pb]PbCl₂.

FIG. 97 is a rad-TLC trace of ²¹²Pb-Conjugate 2.

FIG. 98 is a rad-TLC trace of ²¹²Pb-Conjugate 2 two hours post synthesis.

FIG. 99 is a rad-TLC trace of ²¹²Pb-Conjugate 2 twenty-two hours post synthesis.

FIG. 100 is a series of images of a nude mouse with LNCaP tumors prior to injection with the ²¹²Pb-Conjugate 2 and images of the nude mouse post-injection with ²¹²Pb-Conjugate 2.

FIG. 101 is a series of PET images of a nude mouse with LNCaP tumors pre-therapy with ²¹²Pb-Conjugate 2 and post-therapy with ²¹²Pb-Conjugate 2.

FIG. 102 is a representative example of a conjugate as described herein with a cleavable linker.

### DETAILED DESCRIPTION

This document provides conjugates that include two or more chelators and one or more binding moieties or motifs, wherein one of the chelators is a chelator of an imaging isotope and one of the chelators is a chelator of a radiotherapy isotope. A trifunctional compound (e.g., such as N′,N′-bis(2-aminoethyl)ethane-1,2-diamine), which can act as a linker, can be selectively reacted with two different chelators, one for an imaging isotope and one for a radiotherapy isotope, to produce a dual chelator compound. The dual chelator compound can be modified to make it suitable to react with the binding moiety (e.g., modified at room temperature under mild reaction condition (such as an aqueous medium) to protect the nature and functionality of the binding moieties, to produce a conjugate in which the two or more chelators are covalently attached to the one or more binding moieties or motifs. Only one functional group on the targeted binding moiety (e.g., a primary NH₂) is needed to produce the conjugate. As described below, the combination of chelators and isotopes can be varied as needed for the method of treatment or imaging.

In some embodiments, the chelators can be linked to the binding moiety with a moiety of Formula (I):

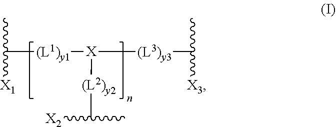

wherein:
each X is independently selected from N, P, P(═O), CR^{N}, and a moiety of formula (i):

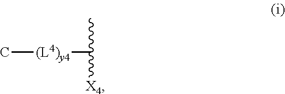

each of x₁, x₂, x₃, and x₄ independently indicates a point of attachment of the moiety of Formula (I) to a chelator or a binding moiety;
each of L¹, L², L³, and L⁴ is independently selected from C(═O), C(═S), N(R^{N}), O, S, S(═O), S(═O)₂, —CR^{N}═NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

In some embodiments, X is N.

In some embodiments, X is P.

In some embodiments, X is P(═O).

In some embodiments, X is CR^{N}.

In some embodiments, X is the moiety of formula (i).

In some embodiments, X is selected from N and CR^{N}.

In some embodiments, X is selected from N, CR^{N}, and the moiety of formula (i).

In some embodiments, each L¹ independently selected from C(═O), C(═S), NH, O, —C₁₋₃ alkylene-, and C₆₋₁₀ arylene. In some embodiments, moiety (L¹)_{y1} comprises at least one moiety of formula NHC(═S)NH or C₆₋₁₀ arylene-C₁₋₃ alkylene-.

In some embodiments, each L² independently selected from C(═O), C(═S), NH, O, —C₁₋₃ alkylene-, and C₆₋₁₀ arylene. In some embodiments, moiety (L²)_{y2} comprises at least one moiety of formula NHC(═S)NH or C₆₋₁₀ arylene-C₁₋₃ alkylene-.

In some embodiments, each L³ independently selected from C(═O), C(═S), NH, O, —C₁₋₃ alkylene-, and C₆₋₁₀ arylene. In some embodiments, moiety (L³)_{y3} comprises at least one moiety of formula NHC(═S)NH or C₆₋₁₀ arylene-C₁₋₃ alkylene-.

In some embodiments, each L⁴ independently selected from C(═O), C(═S), NH, O, —C₁₋₃ alkylene-, and C₆₋₁₀ arylene. In some embodiments, moiety (L⁴)_{y4} comprises at least one moiety of formula NHC(═S)NH or C₆₋₁₀ arylene-C₁₋₃ alkylene-.

In some embodiments, y₁ is an integer selected from 1, 2, 3, 4, 5, and 6. In some embodiments, y₂ is an integer selected from 1, 2, 3, 4, 5, and 6. In some embodiments, y₃ is an integer selected from 1, 2, 3, 4, 5, and 6. In some embodiments, y₄ is an integer selected from 1, 2, 3, 4, 5, and 6.

In some embodiments, R^{N} is H. In some embodiments, R^{N} is C₁₋₃ alkyl. In some embodiments, R^{N} is selected from H and C₁₋₃ alkyl.

In some embodiments, n is 1. In some embodiments, n is 2. In some embodiments, n is 3. In some embodiments, n is 4.

In some embodiments, the compound of Formula (I) has formula:

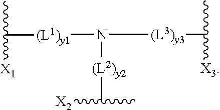

In some embodiments, the compound of Formula (I) has formula:

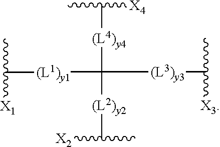

In some embodiments, the compound of Formula (I) has formula:

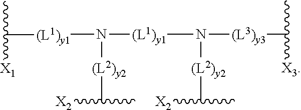

In some embodiments, the moiety of Formula (I) can have any one of the following formulae:

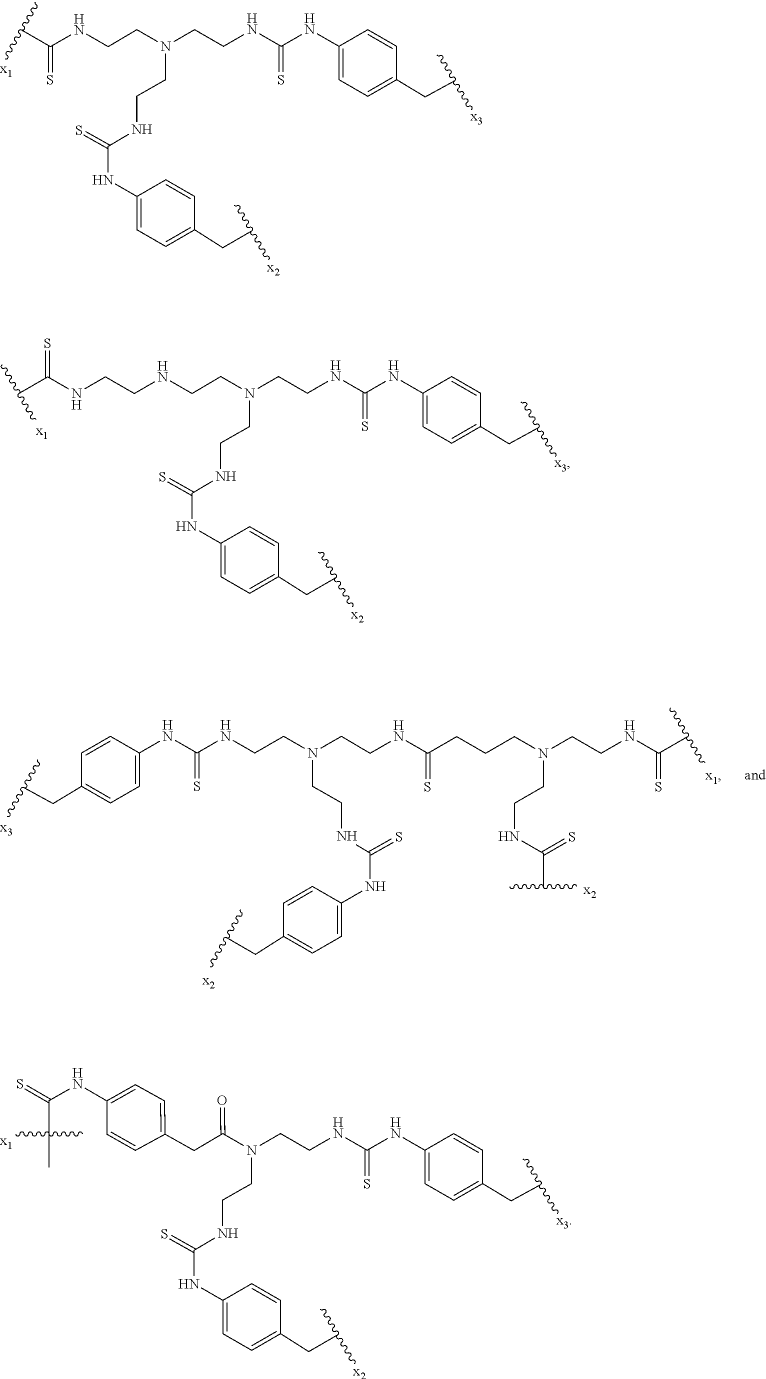

In some embodiments, the chelators are linked and/or the chelator and the binding moiety are linked with a moiety of Formula (II):

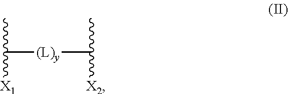

wherein:
x₁ indicates a point of attachment of the Formula (II) to the chelator;
x₂ indicates a point of attachment of the Formula (II) to the chelator or the binding moiety;
each L is independently selected from C(═O), C(═S), N(R^{N}), O, S, S(═O), S(═O)₂, —CR^{N}═NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
y is an integer from 1 to 30; and
each R^{N} is independently selected from H, C₁₋₃ alkyl, and C₁₋₃ haloalkyl.

In some embodiments, x₂ indicates a point of attachment of the Formula (II) to the chelator. In some embodiments, x₂ indicates a point of attachment of the Formula (II) to or the binding moiety.

In some embodiments, each L independently selected from C(═O), C(═S), NH, O, —C₁₋₃ alkylene-, and C₆₋₁₀ arylene. In some embodiments, moiety (L)_{y} comprises at least one moiety of formula NHC(═S)NH or C₆₋₁₀ arylene-C₁₋₃ alkylene-.

In some embodiments, y is an integer from 1 to 10. In some embodiments, y is 1, 2, 3, 4, 5, 6, 7, 8, 9, or 10. In some embodiments, R^{N} is H. In some embodiments, R^{N} is C₁₋₃ alkyl. In some embodiments, R^{N} is selected from H and C₁₋₃ alkyl.

In some embodiments, the moiety of Formula (II) has any one of the following formulae:

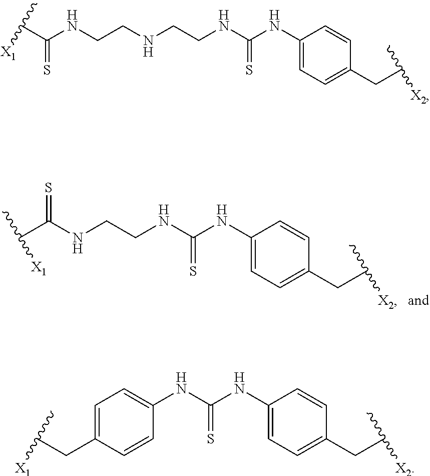

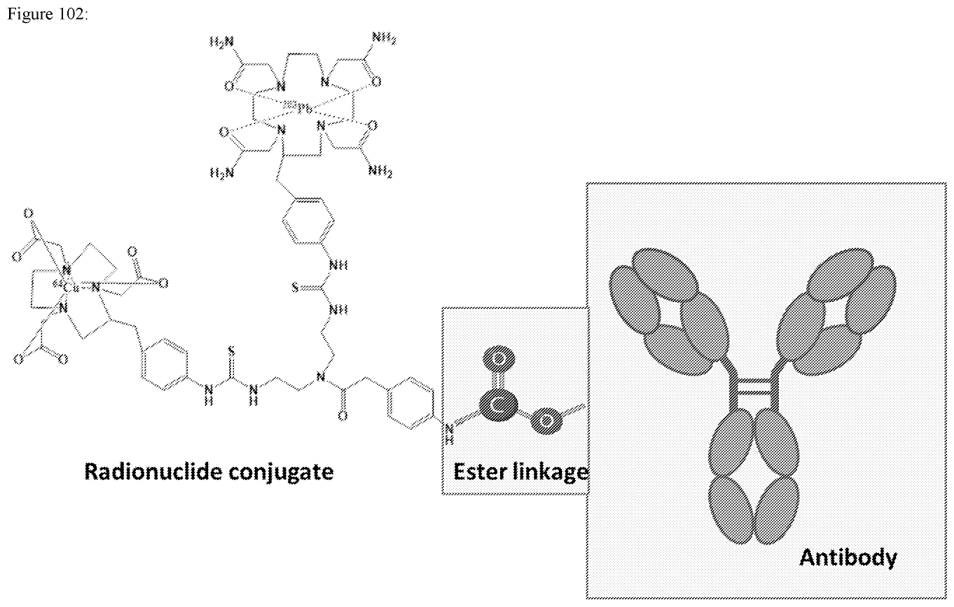

In some embodiments, the chelator can be linked to the binding moiety with a cleavable linker. As used herein, the term “cleavable linker” refers to a linker that is readily catabolized or metabolized under specific conditions. In some cases, a cleavable linker can remain intact under most conditions (e.g., while in storage) but can be cleaved when exposed to a particular compound (e.g., a compound present in the body such as a particular protease) such that the linker is cleaved when in the presence of that compound. In some cases, a cleavable linker can remain intact under most conditions (e.g., while in storage) but can be cleaved under physiological conditions (e.g., at a human's natural blood pH) such that the linker is cleaved when administered to a mammal (e.g., a human). For example, in some embodiments, the cleavable linker can be acid cleavable, GSH cleavable, Fe(II) cleavable, cathepsin cleavable, glycosidase cleavable, phosphatase cleavable, sulfatase cleavable, photo-responsive cleavable, or biorthogonal cleavable. See, for example, Zheng et al., Acta Pharm Sin B. 2021 Dec.; 11(12):3889-3907 and Tsuchikama et al., Protein Cell. 2018 Jan.; 9(1):33-46. In some cases, the cleavable moiety can be as described in U.S. Pat. No. 11,191,854 or 10,093,741. For example, in some embodiments, the cleavable moiety can comprise an ester bond, a phosphate bond, or a disulfide bond. An ester linkage can be cleavable by an esterase native to the cellular environment or hydrolyzable by a neutral or acidic buffered environment. A phosphate linkage can be cleavable by a phosphatase or hydrolyzable by a neutral or acidic buffered environment. A disulfide linkage can be cleavable by the reducing environment of the microenvironment, soluble GSH, thioredoxin, or glutaredoxin. Once cleavage occurs, the binding moiety can maintain its extended retention within the body while the chelators and associated radionuclei can be rapidly excreted. FIG. 102 represents one such schematic for a conjugate as described herein with a cleavable ester linkage connecting an antibody to chelators for both Cu and Pb. In FIG. 102, the ester linkage can be replaced with a phosphate or disulfide linkage.

In some embodiments, the cleavable linker can connect the binding moiety to one or more chelators. For example, the cleavable linker can connect the binding moiety to two chelators. In some embodiments, cleavage of the linker can separate one or more chelators from the binding moiety.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range that includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms. In a similar manner, the term “Cₙ₋ₘ alkenylene” refers to, employed alone or in combination with other terms, refers to a divalent alkenyl linking group having n to m carbons, and the term Cₙ₋ₘ alkynyl,” employed alone or in combination with other terms, refers to a divalent alkynyl linking group having n to m carbons.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “carboxy” refers to a —C(O)OH group. As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, each of the chelators independently can be, for example, NOTA, DOTA, TCMC, DiAmSar, HBED, DFO, DTPA, NTA, BisTris, EGTA, EDTA, BAPTA, DO2A, DO3A and MACROPA. In general, a combination of chelators for imaging and therapy isotopes can be selected for a particular application. For example, in some embodiments, one chelator can be DiAmSar and one chelator can be TCMC. In some embodiments, one chelator can be NOTA and one chelator can be TCMC. In some embodiments, each of the chelators independently can be a supermagnetic iron oxide nanoparticle (SPION). In some embodiments, the SPION can be ferumoxytol. Certains aspects of these embodiments are described, for example, in Advanced Drug Delivery Reviews, Volume 63, Issues 1-2, January-February 2011, Pages 24-46; and Kidney Int. 2017 Jul.; 92(1): 47-66, which are incorporated herein by reference in their entirety.

In some embodiments, the conjugate can include three or more chelators. For example, in some embodiments, the conjugate can include three chelators, or four chelators, or five chelators. For example, in some embodiments, one chelator can be DiAmSar, one chelator can be TCMC, and one chelator can be NOTA. In some embodiments, each of the three chelators can be NOTA or each of the chelators can be SPION. In some embodiments, one chelator can be MACROPA, one chelator can be DFO, and one chelator can be DOTA. For example, in some embodiments, the conjugate can include three or more of DOTA, NOTA, TCMC, MACROPA, DiAmSar, and HBED. In some cases, one chelator can be DOTA, one chelator can be NOTA, one chelator can be TCMC, one chelator can be MACROPA, one chelator can be DiAmSar, and one chelator can be HBED.

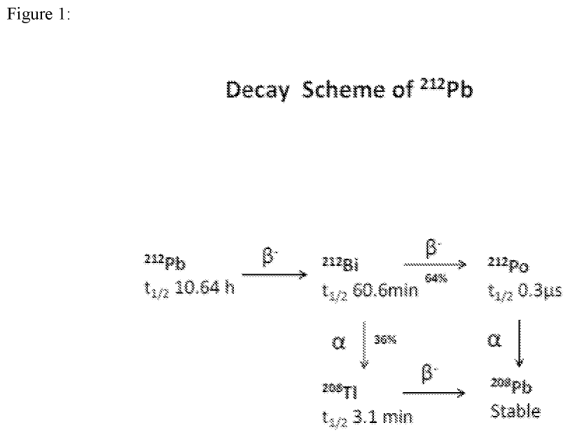

The imaging isotope and the radiotherapy isotope of a conjugate described herein can be selected such that the half-lives are similar. For example, the radiotherapy isotope can be an α-emitter such as ²²⁵Ac, ²¹²Pb, ²¹¹At, ²¹³Bi, ²¹²Bi, ²¹¹Bi, ^{152/160/161}Tb, ²²⁷Th, ²²³Ra, ²¹¹Po, ²²¹Fr, ²¹⁷At, ²¹³Po, ²¹²Po, ²¹⁵Po, or ¹⁷⁷Lu and the imaging isotope can be ⁶⁸Ga, ⁴⁴Sc, ^{60/61/62/64}Cu, ^{84/86/87/89}Zr, ⁶³Zn, ^{43/44}Sc, ^{192/193/194/196}Au, ^{52m}Mn, ^{90/92ml}Nb, ^{51/52}Mn, ^{148/151/151m/152}Tb, ⁴⁵Ti, ^{65/66/67}Ga, ^{94m}Tc, ⁵⁵Co, ^{80/81/83}Sr, ³⁸K, ^{70/71/72/74}As, ^{81/82m}Rb, ⁵²Fe, or ⁸⁶Y. In some embodiments, the imaging isotope is ⁶⁴Cu and the radiotherapy isotope is ²¹²Pb. ⁶⁴Cu is a positron-emitting PET imaging radionuclide, which decays to stable non-radioactive daughter nuclides ⁶⁴Ni and ⁶⁴Zn. ²¹²Pb is a parent isotope of ²¹²Bi, which is an alpha-emitting therapeutic radionuclide, which eventually decays to a stable non-radioactive daughter nuclide ²⁰Pb. See, e.g., FIG. 1. ⁶⁴Cu has a physical half-life of 12.7 hours and ²¹²Pb has a physical half-life of 10.6 hours (or an effective physical half-life for alpha-emission of 11.65 hours, as described below), making them an ideal pair for evaluating the relevant radioactive biodistribution and dosimetry of ²¹²Pb using ⁶⁴Cu as the imaging readout. The longer half-lives (as compared to ⁶⁸Ga or ¹⁸F) also allow for a central location for production to cover large parts of the USA and long-distance distribution of the resulting compounds. Strictly speaking, in terms of the radioactive decay, ²¹²Pb is a beta-emitter that decays into an alpha-emitter, ²¹²Bi. ²¹²Pb is commonly referred to as an alpha-emitter among physicians because the beta-emissions that result from decay of ²¹²Pb are of little consequence physiologically relative to the alpha-emissions. Specifically, after a ²¹²Pb radionuclide gives off a beta-emission, the ²¹²Pb becomes ²¹²Bi (a daughter product) and remains in the chelator and part of the therapy drug. The ²¹²Bi then further decays by one of two equivalent pathways (see FIG. 1); (1) ²¹²Bi gives off an alpha-emission and becomes ²⁰⁸Tl, then gives off a beta-emission, or (2)²¹²Bi gives off a beta-emission, becomes ²¹²Po and stays in the chelator, then immediately gives off an alpha-emission. Thus, ²¹²Pb and drugs containing ²¹²Pb (including those described herein) can be thought of as alpha-emitters with a physical half-life of 11.65 hours prior to alpha-emission (10.64 hours for ²¹²Pb plus 60.6 minutes for ²¹²Bi). As described herein, the decay scheme of ²¹²Pb (FIG. 1) results in 1 alpha-emission also happens to give off 2 beta-emissions as it decays to stable ²⁰⁸Pb. The beta-emissions are of no significant consequence because a beta-emission has ˜10,000 times less mass than an alpha-emission and therefore the 2 beta-emission are inconsequential by comparison to the alpha-emission in terms of the effects within the body. When beta-emitters are used for therapy, the total amount of radioactive drug that needs to be injected to see an effect is orders of magnitude higher than the dose of a comparable alpha-emitting drug.

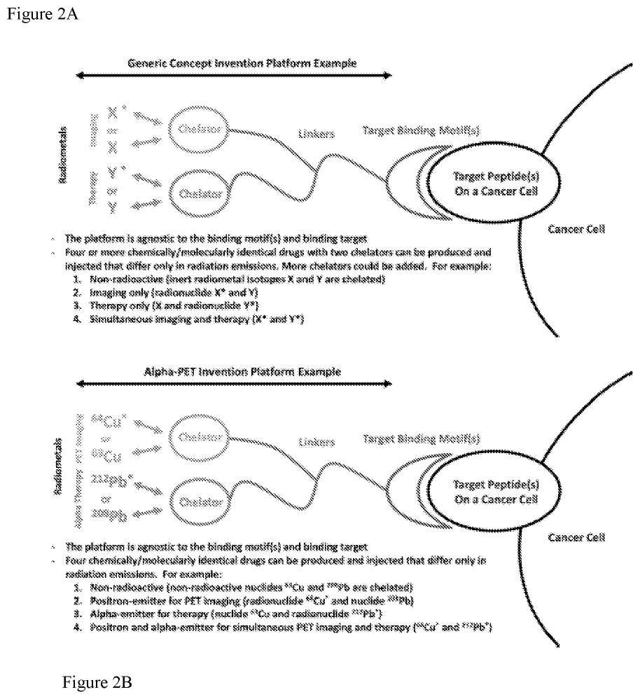

As shown in FIGS. 2A and 2B, depending on the desired use of the conjugate, different combinations of imaging isotopes and radiotherapy isotopes can be selected, resulting in conjugates that differ only in emissions of radiation, but are identical in chemical structure, and therefore identical in binding affinity and biodistribution. For example, for a non-radioactive conjugate, inert radiometal isotopes (e.g., ⁶³Cu and ²⁰⁸Pb) can be selected for chelation with the two or more chelators. For an imaging only conjugate, an imaging isotope (e.g., ⁶⁴Cu) and an inert radiotherapy isotope (e.g., ²⁰⁸Pb) can be selected for chelation with the two or more chelators. For a therapy only conjugate, a radiotherapy isotope (e.g., ²¹²Pb) and an inert imaging isotope (e.g., ⁶³Cu) can be selected for chelation with the two or more chelators. In some embodiments, an imaging only conjugate and a therapy only conjugate can be prepared such that the desired dose (radioactively speaking) of each radioisotope is administered at the time of injection. For a conjugate that can be used for simultaneous imaging and therapy, an imaging isotope (e.g., ⁶⁴Cu) and a radiotherapy isotope (e.g., ²¹²Pb) can be selected for chelation with the two or more chelators.

In some embodiments, a fluorescent dye is used instead of an imaging isotope. Non-limiting examples of fluorescent dyes such as coumarin, cyanine, carboxyfluorescein, quantum dots, green fluorescent protein (GFP), yellow fluorescent protein, red fluorescent protein, phycobiliproteins (e.g., phycoerythrin, phycocyanin, or allophycocyanin), a xanthene derivative such as fluorescein or fluorescein isthiocyanate (FITC), rhodamine, Oregon green, eosin, and Texas red, a cyanine derivative such as cyanine, indocarbocyanine, oxacarbocyanine, thiacarbocyanine, and merocyanine; a squaraine derivative and ring-substituted squaraines, including Seta and Square dyes; squaraine rotaxane derivatives (e.g., Tau dyes), naphthalene derivatives (e.g., dansyl and prodan derivatives); a coumarin derivative, an oxadiazole derivatives (e.g., pyridyloxazole, nitrobenzoxadiazole and benzoxadiazole); an anthracene derivative (e.g., an anthraquinone, including DRAQ5, DRAQ7 and CyTRAK Orange); a pyrene derivative (e.g., cascade blue); an oxazine derivatives (e.g., Nile red, Nile blue, cresyl violet, oxazine 170); an acridine derivative (e.g., proflavin, acridine orange, acridine yellow); an arylmethine derivatives (e.g., auramine, crystal violet, malachite green); a tetrapyrrole derivative (e.g., porphin, phthalocyanine, bilirubin); a dipyrromethene derivative (e.g., BODIPY, aza-BODIPY); an amino group (active ester, carboxylate, isothiocyanate, hydrazine), carboxyl groups (carbodiimide), thiol (maleimide, acetyl bromide), or azide (via click chemistry or non-specifically (glutaraldehyde)).

For any of the conjugates, the binding moiety can be one or more small molecules, nanoparticles, liposomes, exosomes, polypeptides (e.g., an antibody or peptide), or any other targeted biologic that binds to a target molecule on a cell (e.g., a cancer cell). In some cases, the binding moiety can target a molecule on the surface of a cell (e.g., a cell surface receptor). For example, a small molecule such as a Glu-ureido based prostate specific membrane antigen (PSMA) inhibitor (also referred to as glutamate carboxypeptidase II inhibitors) can be used as a binding moiety. See, e.g., Kopka, et al., J. Nucl. Med., 58(Supplement 2):17S-26S (2017). PSMA (also is referred to as folate hydrolase 1 (FOLH1), FGCP, FOLH, GCP2, PSM, mGCP, GCPII, NAALAD1, or NAALAdase) is a cell membrane peptidase that belongs in the M28B subfamily of the M28 peptidase family. For example, nanoparticles containing a glutamate carboxypeptidase II inhibitor can be used a binding moiety. In some embodiments, a nanoparticle can be a hydrophilic polyethylene glycol corona with small-molecule PSMA targeting ligands, See, for example, Autio, et al., JAMA Oncology, 4(10):1344-1351 (2018). An exosome such as a dendritic cell derived exosome (see, e.g., Xu, et al., Molecular Cancer, 19, 160 (2020)) can be used a binding moiety.

For example, in some embodiments, the binding moiety can be a polypeptide that binds PSMA, a somatostatin receptor, a fibroblast activating protein (FAP) polypeptide, a melanocortin-1 receptor, a B7-H3 protein, a CA19-9 expressing tumor, a cluster of differentiation 37 (CD37), a cluster of differentiation 3 (CD3), a cluster of differentiation 20 (CD20), a c-x-c-motifchemokine receptor 4 (CXCR4), a gastrin releasing peptide receptor (GRPR), a human epidermal growth factor receptor 2 (HER2), a melanocortin 1 receptor (MCIR), a somatostatin receptor 2 (SSTR2), a vascular endothelial growth factor (VEGF), a programmed death-ligand 1 (PD-L1) polypeptide, a tumor associated calcium signal transducer 2 (TROP2) polypeptide, a protein tyrosine kinase 2 (PTK2) polypeptide, an integrin beta 6 (ITGB6) polypeptide, a neurotensin receptor ligand, CD8, or vitamin B-12. See, e.g., Langbein et al., J. Nucl. Med., 60(Supplement 2):13S-19S (2019). For example, the polypeptide can be a somatostatin analog such as Phe1-Tyr3-octreotate (TATE) or Phe1-Tyr3-octreotide (TOC). See, e.g., Stueven et al., Int. J Mol. Sci., 20(12):3049 (2019). In some embodiments, the conjugate includes two different polypeptides. In some embodiments, the polypeptide can be an antibody or an antibody fragment having the ability to bind an antigen. The term “antibody” as used herein includes monoclonal antibodies, polyclonal antibodies, recombinant antibodies, humanized antibodies, chimeric antibodies, nanobodies, or multispecific antibodies (e.g., bispecific antibodies) formed from at least two antibodies. The term “antibody fragment” comprises any portion of the afore-mentioned antibodies, such as their antigen binding or variable regions (e.g., single VH domains). The term “epitope” refers to an antigenic determinant on an antigen to which the paratope of an antibody binds. Epitopic determinants usually consist of chemically active surface groupings of molecules (e.g., amino acid or sugar residues) and usually have specific three-dimensional structural characteristics as well as specific charge characteristics.

Examples of antibody fragments include Fab fragments, Fab′ fragments, F(ab′)₂ fragments, Fv fragments, diabodies, single chain antibody molecules, single VH domains, and other fragments as long as they exhibit the desired capability of binding to the target molecule. An “Fv fragment” is the minimum antibody fragment that contains a complete antigen-recognition and binding site. This region consists of a dimer of one heavy chain variable domain and one light chain variable domain in tight, non-covalent association. It is in this configuration that the three complementarity determining regions (CDRs) of each variable domain interact to define an antigen-binding site on the surface of the VH-VL dimer. Collectively, the six CDR's confer antigen-binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (C_{H1}) of the heavy chain. The “Fab fragment” differs from the “Fab′ fragment” by the addition of a few residues at the carboxy terminus of the heavy chain C_{H1} domain, including one or more cysteines from the antibody hinge region. The “F(ab′)₂ fragment” originally is produced as a pair of “Fab′ fragments” which have hinge cysteines between them. Methods of preparing such antibody fragments, such as papain or pepsin digestion, can be performed using any appropriate method.

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat′l. Acad. Sci. USA 86:3833 (1989). Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al., Proc. Nat′l. Acad. Sci. USA 89:4285 (1992); and Sandhu, Crit. Rev. Biotech. 12:437 (1992); Singer et al., J. Immunol. 150:2844 (1993). In some cases, humanization such as super humanization can be used as described by Hwang et al., Methods, 36:35-42 (2005). In some cases, CDR grafting (Kashmiri et al., Methods, 36:25-34 (2005)), human string content optimization (Lazar et al., Mol. Immunol., 44:1986-1998 (2007)), framework shuffling (Dall′Acqua et al., Methods, 36:43-60 (2005); and Damschroder et al., Mol. Immunol., 44:3049-3060 (2007)), and phage display approaches (Rosok et al., J. Biol. Chem., 271:22611-22618 (1996); Radar et al., Proc. Natl Acad. Sci. USA, 95:8910-8915 (1998); and Huse et al., Science, 246:1275-1281 (1989)) can be used to obtain antibody preparations that bind to a target molecule. In some cases, fully human antibodies can be generated from recombinant human antibody library screening techniques as described, for example, by Griffiths et al., EMBO J., 13:3245-3260 (1994); and Knappik et al., J. Mol. Biol., 296:57-86 (2000).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, Fab fragments can be produced by enzymatic cleavage of antibodies with papain. In some cases, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

An antibody can be of the IgA-, IgD-, IgE-, IgG- or IgM-type, including IgG- or IgM-types such as, without limitation, IgG1-, IgG2-, IgG3-, IgG4-, IgM1- and IgM2-types. For example, in some cases, an antibody is of the IgG1-, IgG2- or IgG4-type.

In some embodiments, the antibody can be an antibody that binds PSMA. For example, an antibody that binds PSMA can include CDRs that comprise, consist essentially of, or consist of the CDR amino acid sequences set forth in SEQ ID NOs: 1-6. In some cases, an antibody that binds PSMA can have one or more CDRs that are a variant of (e.g., are not 100% identical to) a CDR set forth in any one of SEQ ID NOs:1-6, provided that the antigen binding domain retains the ability to bind to PSMA. For example, one or more CDRs of an antibody that binds PSMA can consist of an amino acid sequence set forth in any one of SEQ ID NOs:1-6, except that the variant polypeptide includes one, two, three, four, or five amino acid substitutions within the articulated sequence of the sequence identifier (e.g., any one of SEQ ID NOs:1-6), has one, two, three, four, or five amino acid residues preceding the articulated sequence of the sequence identifier (e.g., any one of SEQ ID NOs:1-6), and/or has one, two, three, four, or five amino acid residues following the articulated sequence of the sequence identifier (e.g., any one of SEQ ID NOs:1-6), provided that the antibody retains the ability to bind PSMA. Examples of CDR amino acid sequences that comprise, consist essentially of, or consist of the CDR amino acid sequences set forth in SEQ ID NOs: 1-6 and can be used in an antibody that binds PSMA include, without limitation, those amino acid sequences shown in Table 1 (see, also, Example 17).

TABLE 1
Exemplary CDR sequences for anti-PSMA antibodies.
VL refers to variable light chain, and VH refers
to variable heavy chain.

| CDR | Amino Acid Sequence | SEQ ID NO: |
|---|---|---|
| VL CDR1 | QSINNY | 1 |
| | QGIRND | 49 |
| | SASSSISSNYLH | 50 |
| | RASQGISSALA | 51 |
| | RASQDISSALA | 52 |
| | RASQSVSSYLA | 53 |
| | KASQDVGTAVD | 54 |
| | KASENVGTYVS | 55 |
| | RASESIDSYDNTFMH | 56 |
| | KASQNVGSDVA | 57 |
| | KSISKY | 58 |
| VL CDR2 | TAS | 2 |
| | GAS | 59 |
| | RTSNLAS | 60 |
| | DASSLES | 61 |
| | DASNRAT | 62 |
| | WASTRHT | 63 |
| | GASNRFT | 64 |
| | RASILES | 65 |
| | STSYRYS | 66 |
| | SGS | 67 |
| VL CDR3 | QQSFSTPPIT | 3 |
| | LQHNSHPYT | 68 |
| | QQGSYIPFT | 69 |
| | QQNSYPLT | 70 |
| | QQFNSYPLT | 71 |
| | QQRSNWPLFT | 72 |
| | GQSYTFPYT | 73 |
| | HQSIEDPYT | 74 |
| | QQYNSYPLT | 75 |
| | QQHIEYPWT | 76 |
| VH CDR1 | GFTFADFT | 4 |
| | GFTFITYG | 77 |
| | GFTFSNYN | 78 |
| | GFSFSGYG | 79 |
| | GFTFSSYG | 80 |
| | GFTFSDFYMY | 81 |
| | SYAMH | 82 |
| | SNWIG | 83 |
| | SNWIG | 84 |
| | NYWIG | 85 |
| | GYTFTEYTIH | 86 |
| | GFTFSNYWMN | 87 |
| | GYTFGTYVMH | 88 |
| | GFSLTAYGIN | 89 |
| | SGYTFTDYYMH | 90 |
| VH CDR2 | ISWNSNSI | 5 |
| | IYYDESNK | 91 |
| | ISTGSSDI | 92 |
| | MSYDGSNK | 93 |
| | TWYDGSNK | 94 |
| | ISYAGNNK | 95 |
| | TISDGGGYTSYPDSVKG | 96 |
| | VISYDQNNKYYADSVK | 97 |
| | IIYPQDSDTRYSPSFQ | 98 |
| | IIYPGDSDTRYSPSFQ | 99 |
| | IIYPGDSDTRYSPSFQQ | 100 |
| | NINPNNGGTTYNQKFED | 101 |
| | EIRSQSNNFATHYAESVKG | 102 |
| | YINPYNDVTRYNGKFKG | 103 |
| | VIWPCGNTDYNSTLKS | 104 |
| | YFNPYNDYTR | 105 |
| VH CDR3 | VKDRSGYSRF | 6 |
| | YYGMDV | 106 |
| | ARAPRVAVEE | 107 |
| | DYSYYYGMDV | 108 |
| | ARDIIGTTRD | 109 |
| | AKDGYYDFLTFDYTLDY | 110 |
| | ARDGNWGGPYWYFDL | 111 |
| | AKDPYYDFLTGSDYFDY | 112 |
| | GLWLRDALDY | 113 |
| | AVPWQSRYYYYQMDV | 114 |
| | QTGFLWSSDL | 115 |
| | QTGFLWSFDL | 116 |
| | PGYTSSWTSFDY | 117 |
| | GWNFDY | 118 |
| | RWNNF | 119 |
| | GENWYYFDS | 120 |
| | DSYGNFKRGWFDF | 121 |
| | CARSDGYYDAMDYW | 122 |

In some embodiments, an antibody that binds PSMA can be as described elsewhere. See, e.g., U.S. Pat. No. 10,179,819, International Patent Application Publication No. WO 2018/129284, International Patent Application Publication No. WO 2002/098897, U.S. Patent Application Publication No. 2014/0273078, EP Patent Application Publication No. 3192810 A1, CN 108699157, EP Patent No. 2,363,404, U.S. Patent Application Publication No. 2014/0234215, International Patent Application Publication No. WO 2005/094882, U.S. Pat. Nos. 7,666,414, 8,114,965, 8,470,330, International Patent Application Publication No. WO 2014/4057113, U.S. Pat. Nos. 9,242,012, 10,179,819, and 9,782,478.

In some embodiments, the antibody that binds PSMA can be the J591 monoclonal antibody or a humanized J591 monoclonal antibody. See, e.g., Milowsky et al., J. Nucl. Med., 50:606-11 (2009). A fully human monoclonal antibody that binds PSMA also can be used. See, e.g., Ma et al., Clin. Cancer Res., 12(8):2591-6 (2006).

In some embodiments, the antibody can be an antibody that binds a somatostatin receptor polypeptide. Examples of somatostatin receptor polypeptides include, without limitation, sstr1 receptor polypeptides, sstr2a receptor polypeptides, sstr2b receptor polypeptides, sstr3 receptor polypeptides, sstr4 receptor polypeptides, and sstr5 receptor polypeptides. For example, an antibody that binds a somatostatin receptor can include CDRs that comprise, consist essentially of, or consist of the CDR amino acid sequences set forth in SEQ ID NOs: 7-12. In some cases, an antibody that binds a somatostatin receptor provided herein can have one or more CDRs that are a variant of (e.g., are not 100% identical to) a CDR set forth in any one of SEQ ID NOs: 7-12, provided that the antigen binding domain retains the ability to bind to a somatostatin receptor. For example, one or more CDRs of an antibody that binds a somatostatin receptor provided herein can consist of an amino acid sequence set forth in any one of SEQ ID NOs: 7-12, except that the variant polypeptide includes one, two, three, four, or five amino acid substitutions within the articulated sequence of the sequence identifier (e.g., any one of SEQ ID NOs:7-12), has one, two, three, four, or five amino acid residues preceding the articulated sequence of the sequence identifier (e.g., any one of SEQ ID NOs: 7-12), and/or has one, two, three, four, or five amino acid residues following the articulated sequence of the sequence identifier (e.g., any one of SEQ ID NOs:7-12), provided that the antigen binding domain retains the ability to bind to a somatostatin receptor. Examples of CDR amino acid sequences that comprise, consist essentially of, or consist of the CDR amino acid sequences set forth in SEQ ID NOs: 7-12 and can be used in an antibody that binds a somatostatin receptor include, without limitation, those amino acid sequences shown in Table 2 (see, also, Example 17).

TABLE 2
Exemplary CDR sequences for anti-somatostatin
antibodies.

| CDR | Amino Acid Sequence | SEQ ID NO: |
|---|---|---|
| VL CDR1 | RSSQSLVHSNGNTYLH | 7 |
| | KSSQSLLNSRNRKNYLA | 123 |
| | KSSQSLLNSRTRKNYLA | 124 |
| VL CDR2 | KVSNRFS | 8 |
| | WASTRES | 125 |
| | WASTRAS | 126 |
| VL CDR3 | SQSTHVPFT | 9 |
| | SQSTRVPFT | 127 |
| | KQSYYLWT | 128 |
| VH CDR1 | DYHLN | 10 |
| | DYHMN | 129 |
| | DYGMA | 130 |
| VH CDR2 | IRNKRYGYRTEYSASVKG | 11 |
| | LIRNKANGYRTEYSASVKG | 131 |
| | FISNLGYSIYYADSVKG | 132 |
| | FISNLAYSIYYADSVKG | 133 |
| VH CDR3 | DFYDPFAY | 12 |
| | APYDYDSFYPMDY | 134 |
| | APYDYDSFDPMDY | 135 |

In some embodiments, an antibody that binds a somatostatin receptor can be UMB1, UMB4, UMB5, or UMB7.

In some embodiments, an antibody that binds a somatostatin receptor can be as described elsewhere. See, e.g., International Patent Application Publication No. WO 2018/005706, U.S. Patent Application Publication No. 2009/0016989, U.S. Patent Application Publication No. 2021/0340264, U.S. Pat. No. 11,225,521, NZ749841A, AU2017290086A, CN 201780041351.9A, and Korner et al., Am J Surg Pathol. 2012 Feb.; 36(2):242-52.

In some embodiments, the antibody can be an antibody that binds a FAP polypeptide. For example, an antibody that binds a FAP polypeptide can include CDRs that comprise, consist essentially of, or consist of the CDR amino acid sequences set forth in SEQ ID NOs: 13-18. In some cases, an antibody that binds a FAP polypeptide can have one or more CDRs that are a variant of (e.g., are not 100% identical to) a CDR set forth in any one of SEQ ID NOs: 13-18, provided that the antigen binding domain retains the ability to bind to a FAP polypeptide. For example, one or more CDRs of an antibody that binds a FAP polypeptide can consist of an amino acid sequence set forth in any one of SEQ ID NOs: 13-18, except that the variant polypeptide includes one, two, three, four, or five amino acid substitutions within the articulated sequence of the sequence identifier (e.g., any one of SEQ ID NOs:13-18), has one, two, three, four, or five amino acid residues preceding the articulated sequence of the sequence identifier (e.g., any one of SEQ ID NOs: 13-18), and/or has one, two, three, four, or five amino acid residues following the articulated sequence of the sequence identifier (e.g., any one of SEQ ID NOs: 13-18), provided that the antigen binding domain retains the ability to bind to a FAP polypeptide. Examples of CDR amino acid sequences that comprise, consist essentially of, or consist of the CDR amino acid sequences set forth in SEQ ID NOs: 13-18 and can be used in an antibody that binds a FAP polypeptide include, without limitation, those amino acid sequences shown in Table 3 (see, also, Example 17).

TABLE 3
Exemplary CDR sequences for anti-FAP antibodies.

| CDR | Amino Acid Sequence | SEQ ID NO: |
|---|---|---|
| VL CDR1 | RASQSISSYLN | 13 |
| | KSSQSLLYSRNQKNYLA | 136 |
| | RASQSVSRNYLA | 137 |
| | RASQSVTSSYLA | 138 |
| VL CDR2 | AASSLQS | 14 |
| | WASTRES | 139 |
| | GASSRAT | 140 |
| | GASTRAT | 141 |
| | VGSRRAT | 142 |
| VL CDR3 | QQSYKPYT | 15 |
| | QQSYSTPRT | 143 |
| | QQFSYPLT | 144 |
| | QQSLGYPPT | 145 |
| | QQGQVIPPT | 146 |
| VH CDR1 | SYAMS | 16 |
| | SYAMS | 147 |
| | SYAMS | 148 |
| | SYAMN | 149 |
| | SYAMS | 150 |
| | RYAM | 151 |
| VH CDR2 | AISGSGGTTYYADSVKG | 17 |
| | AISGSGGGTRYADSVKG | 152 |
| | GISGSGGTYYADSVKG | 153 |
| | TISSSGRTYYADSVKG | 154 |
| | GISGGGGSTYYADSVKG | 155 |
| | SISAGGTYYADSVKG | 156 |
| VH CDR3 | QQSYKPYT | 18 |
| | HSSGFHWYFDY | 157 |
| | ISFYPGGTYFDY | 158 |
| | GLVASAPFD | 159 |
| | IAHSRIGWHFDY | 160 |
| | TFSGYAHYDFDY | 161 |

In some embodiments, an antibody that binds a FAP polypeptide can be sibrotuzumab or BMS168.

In some embodiments, an antibody that binds a FAP polypeptide can be as described elsewhere. See, e.g., JP7017599 B2, JP 2009522329 A, U.S. Patent Application Publication No. 2021/0253736, EP 3269740 A1, U.S. Pat. No. 8,999,342, U.S. Patent Application Publication No. 2017/0369592, IL 281739 DO, U.S. Pat. No. 9,481,730, and ES 2348556 T3.

In some embodiments, the antibody can be an antibody that binds a CD3 polypeptide. For example, an antibody that binds a CD3 polypeptide can include CDRs that comprise, consist essentially of, or consist of the CDR amino acid sequences set forth in SEQ ID NOs: 19-24. In some cases, an antibody that binds a CD3 polypeptide can have one or more CDRs that are a variant of (e.g., are not 100% identical to) a CDR set forth in any one of SEQ ID NOs: 19-24, provided that the antigen binding domain retains the ability to bind to a CD3 polypeptide. For example, one or more CDRs of an antibody that binds a CD3 polypeptide can consist of an amino acid sequence set forth in any one of SEQ ID NOs: 19-24, except that the variant polypeptide includes one, two, three, four, or five amino acid substitutions within the articulated sequence of the sequence identifier (e.g., any one of SEQ ID NOs: 19-24), has one, two, three, four, or five amino acid residues preceding the articulated sequence of the sequence identifier (e.g., any one of SEQ ID NOs: 19-24), and/or has one, two, three, four, or five amino acid residues following the articulated sequence of the sequence identifier (e.g., any one of SEQ ID NOs: 19-24), provided that the antigen binding domain retains the ability to bind to a CD3 polypeptide. Examples of CDR amino acid sequences that comprise, consist essentially of, or consist of the CDR amino acid sequences set forth in SEQ ID NOs: 19-24 and can be used in an antibody that binds a CD3 polypeptide include, without limitation, those amino acid sequences shown in Table 4 (see, also, Example 17).

TABLE 4
Exemplary CDR sequences for anti-CD3 antibodies.

| CDR | Amino Acid Sequence | SEQ ID NO: |
|---|---|---|
| VL CDR1 | RASQSVSSYLA | 19 |
| | WASQGISSYLA | 162 |
| | RASQGISSALA | 163 |
| VL CDR2 | DASNRAT | 20 |
| | GASSRAT | 164 |
| | YASSLQS | 165 |
| | DASSLGS | 166 |
| | DASSLES | 167 |
| VL CDR3 | QQRSNWPPLT | 21 |
| | QQYGSSPIT | 168 |
| | QQYYSTLT | 169 |
| | QQRSNWPWT | 170 |
| | QQFNSYPIT | 171 |
| VH CDR1 | GYGMH | 22 |
| | SYGMH | 172 |
| VH CDR2 | VIWYDGSKKYYVDSVKG | 23 |
| | IIWYDGSKKNYADSVKG | 173 |
| | AIWYNGRKQDYADSVKG | 174 |
| VH CDR3 | QMGYWHFDL | 24 |
| | GTGYNWFDP | 175 |

In some embodiments, an antibody that binds a CD3 polypeptide can be muromonab or blinatumomab.

In some embodiments, an antibody that binds a CD3 polypeptide can be as described elsewhere. See, e.g., CN 1984931 A, EP 1753783 B1, AU 2009/299792 B2, CN 102796199 A, and JP 6817211 B2.

In some embodiments, the antibody can be an antibody that binds a CD20 polypeptide. For example, an antibody that binds a CD20 polypeptide can include CDRs that comprise, consist essentially of, or consist of the CDR amino acid sequences set forth in SEQ ID NOs: 25-30. In some cases, an antibody that binds a CD20 polypeptide can have one or more CDRs that are a variant of (e.g., are not 100% identical to) a CDR set forth in any one of SEQ ID NOs: 25-30, provided that the antigen binding domain retains the ability to bind to a CD20 polypeptide. For example, one or more CDRs of an antibody that binds a CD20 polypeptide can consist of an amino acid sequence set forth in any one of SEQ ID NOs: 25-30, except that the variant polypeptide includes one, two, three, four, or five amino acid substitutions within the articulated sequence of the sequence identifier (e.g., any one of SEQ ID NOs: 25-30), has one, two, three, four, or five amino acid residues preceding the articulated sequence of the sequence identifier (e.g., any one of SEQ ID NOs: 25-30), and/or has one, two, three, four, or five amino acid residues following the articulated sequence of the sequence identifier (e.g., any one of SEQ ID NOs: 25-30), provided that the antigen binding domain retains the ability to bind to a CD20 polypeptide. Examples of CDR amino acid sequences that comprise, consist essentially of, or consist of the CDR amino acid sequences set forth in SEQ ID NOs: 25-30 and can be used in an antibody that binds a CD20 polypeptide include, without limitation, those amino acid sequences shown in Table 5 (see, also, Example 17).

TABLE 5
Exemplary CDR sequences for anti-CD20 antibodies.

| CDR | Amino Acid Sequence | SEQ ID NO: |
|---|---|---|
| VL CDR1 | RASSSVSYIH | 25 |
| | RASSSLSFMH | 176 |
| | RASSSVSYMH | 177 |
| | RASGSVDSFGNSFMH | 178 |
| | RASESVDNFGNSFMH | 179 |
| VL CDR2 | ATSNLAS | 26 |
| | RASNLES | 180 |
| VL CDR3 | QQWTSNPPT | 27 |
| | HQWSSNPLT | 181 |
| | QQSFSNPPT | 182 |
| | QQSYEDPFT | 183 |
| VH CDR1 | SYNMH | 28 |
| | NYGMN | 184 |
| | NVGMN | 185 |
| VH CDR2 | AIYPGNGDTSYNQKFKG | 29 |
| | WINTYTGEPS | 186 |
| | WINTYTGEPA | 187 |
| | AIYPGNGDTSYNQKFK | 188 |
| VH CDR3 | STYYGGDWYFDV | 30 |
| | SHYGSNYVDYFDV | 189 |
| | WYYSNSYWYFDV | 190 |
| | GAYYRYDLGMDY | 191 |
| | STYYGGDWYFNV | 192 |

In some embodiments, an antibody that binds a CD20 polypeptide can be tositumomab, tituximab, ofatumumab, obinutuzumab, ocrelizumab, or ublituximab.

In some embodiments, an antibody that binds a CD20 polypeptide can be as described elsewhere. See, e.g., EP 1740946 B1, U.S. Pat. No. 8,147,832, EP 1692182 B1, EP 2295468 B1, U.S. Patent Application Publication No. 2004/0093621 A1, U.S. Pat. No. 7,744,877, CN 1210307 C, and CN 104558191 A.

In some embodiments, the antibody can be an antibody that binds a CXCR4 polypeptide. For example, an antibody that binds a CXCR4 polypeptide can include CDRs that comprise, consist essentially of, or consist of the CDR amino acid sequences set forth in SEQ ID NOs: 31-36. In some cases, an antibody that binds a CXCR4 polypeptide can have one or more CDRs that are a variant of (e.g., are not 100% identical to) a CDR set forth in any one of SEQ ID NOs: 31-36, provided that the antigen binding domain retains the ability to bind to a CXCR4 polypeptide. For example, one or more CDRs of an antibody that binds a CXCR4 polypeptide can consist of an amino acid sequence set forth in any one of SEQ ID NOs: 31-36, except that the variant polypeptide includes one, two, three, four, or five amino acid substitutions within the articulated sequence of the sequence identifier (e.g., any one of SEQ ID NOs: 31-36), has one, two, three, four, or five amino acid residues preceding the articulated sequence of the sequence identifier (e.g., any one of SEQ ID NOs: 31-36), and/or has one, two, three, four, or five amino acid residues following the articulated sequence of the sequence identifier (e.g., any one of SEQ ID NOs: 31-36), provided that the antigen binding domain retains the ability to bind to a CXCR4 polypeptide. Examples of CDR amino acid sequences that comprise, consist essentially of, or consist of the CDR amino acid sequences set forth in SEQ ID NOs: 31-36 and can be used in an antibody that binds a CXCR4 polypeptide include, without limitation, those amino acid sequences shown in Table 6 (see, also, Example 17).

TABLE 6
Exemplary CDR sequences for anti- CXCR4 antibodies.

| CDR | Amino Acid Sequence | SEQ ID NO: |
|---|---|---|
| VL CDR1 | QSLYNSRTRKNY | 31 |
| | QSFNSRTRKNY | 193 |
| | KSSQSLFNSRTRKNSL | 194 |
| | TGTISDVGGHNFVS | 195 |
| | TGNSNNVGNQGAA | 196 |
| | QGDSLRKFFAS | 197 |
| | RASQSVNTNLA | 198 |
| | QGDSLRSYYAS | 199 |
| | SGSRSNIGSNTVN | 200 |
| | SGSRSNIGGNTVN | 20 |
| VL CDR2 | WAS | 32 |
| | WASARDS | 202 |
| | WASIRES | 203 |
| | EVTKRPA | 204 |
| | RNNNRPS | 205 |
| | GKNSRPS | 206 |
| | GASSRAT | 207 |
| | GKNNRPS | 208 |
| | TNNQRPS | 209 |
| | ANNQRPS | 210 |
| VL CDR3 | KQSYNLRT | 33 |
| | MQSFNLRT | 21 |
| | KQSTNLRT | 212 |
| | SSYGGSNDVI | 213 |
| | SAWDNRLKTYV | 214 |
| | NSRDSRDNHQV | 215 |
| | QHYGSSPLT | 216 |
| | NSRSGSQRV | 217 |
| | LSFDSSLTSYV | 218 |
| | AAWDDNLSGHW | 219 |
| VH CDR1 | TDYY | 34 |
| | DNY | 220 |
| | GFTFTDNY | 221 |
| | GFSLTDYG | 222 |
| | DNTMS | 223 |
| | DTQVY | 224 |
| | SYGMH | 225 |
| | SNFVAWN | 226 |
| | SYGIS | 227 |
| | SYPMH | 228 |
| | NYGLH | 229 |
| | RYGMH | 230 |
| | SYAMS | 231 |
| | IHTMS | 232 |
| | DYAIG | 233 |
| | GYTIA | 234 |
| | PSAMA | 235 |
| | DYAMS | 236 |
| | MG | 237 |
| | LNAMG | 238 |
| | VNDMG | 239 |
| | YYTIG | 240 |
| | YHAIV | 241 |
| | NYAMG | 242 |
| | INAMG | 243 |
| VH CDR2 | IRNKANGYTT | 35 |
| | IWGDGTT | 244 |
| | FIRNKANGYTTDYSASVRG | 245 |
| | MIWGDGTTDYNSALKS | 246 |
| | VISYDGSNKYYADSVKG | 247 |
| | RTYYRSRWYNDYAVSVQS | 248 |
| | WISAYNGNTNYAQKLQG | 249 |
| | VISSDGRNKYYPDSVKG | 250 |
| | NIKQDGSEKYYVDSVKG | 251 |
| | VISHDGTKKYYADSVKG | 252 |
| | LISYDGSKTFYGESVKG | 253 |
| | GIKSSGDSTRYAGSVKG | 254 |
| | TIKPSGDTTNYANAVKG | 255 |
| | AISWNGGSTDYADSVKG | 256 |
| | CISGSDGSTTYADSVKG | 257 |
| | YHRWSDGANLYADSVKG | 258 |
| | STIWSRGDTYFADSVKG | 259 |
| | AISWNGGSADYADSVKG | 260 |
| | TSRLITDNIIYADSVKG | 261 |
| | GITSSTSTYYADSVKG | 262 |
| | VITSGGGTNYVDSVKG | 263 |
| | CISSSDGSTAYLGSVQG | 264 |
| | CITSRDSITYYASFVKG | 265 |
| | AITRSGVRSGVSAIYGDSVKD | 266 |
| | SITSGGSTVYADSVKG | 267 |
| VH CDR3 | DIPGFAY | 36 |
| | ARDVGSNYFDY | 268 |
| | ARGRQFGFDY | 269 |
| | DVGSNYFDY | 270 |
| | GRQFGFDY | 271 |
| | DLVAAAGTAFDI | 272 |
| | GQHSGFDF | 273 |
| | DTPGIAARRYYYYGMDV | 274 |
| | GGYHDFWSGPDY | 275 |
| | DQVSGITIFGGKWRSPDV | 276 |
| | DGGYCSGGRCYSYGMDV | 277 |
| | ATVTTDGYYYMDV | 278 |
| | SRVSRTGLYTYDN | 279 |
| | DYFGTGV | 280 |
| | DQGPFYSGTYYYTRQYGY | 281 |
| | ARMTTSNDKEYLY | 282 |
| | QYGVGGRVVCPGPYEYDV | 283 |
| | AWMTTSNDKEYLY | 284 |
| | RVRPYGQYDY | 285 |
| | DQGPFYSGTYYYTKGYAY | 286 |
| | RQNYSRSVFGAKDYDY | 287 |
| | DCPDYYSDYECPLED | 288 |
| | YYSSGISTLRS | 289 |
| | BSADSRCSIGSIGFTWLYNN | 290 |
| | BTSMTCPTLIVRENY | 291 |
| | SAIGSGALRRFEYDY | 292 |
| | DGVPEWGKVQYPDTY | 293 |

In some embodiments, an antibody that binds a CXCR4 polypeptide can be ibalizumab, MAB172-100, PA3-305, or hz515H7.

In some embodiments, an antibody that binds a CXCR4 polypeptide can be as described elsewhere. See, e.g., EP 2285833 B1, JP 5749330 B2, U.S. Pat. No. 7,138,496, U.S. Patent Application Publication No. 2005/0002939, EP 2246364 A1, CA 2724409 A1, International Patent Application Publication No. WO 2006/089141, Broussas et al., Mol. Cancer Ther., 2016 Aug.; 15(8):1890-9, International Patent Application Publication No. WO 2000/042074, International Patent Application Publication No. WO 2004/059285, EP 1449850 A1, TW 1469792 B, U.S. Pat. Nos. 8,329,178, 7,892,546, International Patent Application Publication No. WO 2009/138519, International Patent Application Publication No. WO 2009/140124, International Patent Application Publication No. WO 2008/142303, International Patent Application Publication No. WO 2008/060367, U.S. Pat. No. 8,748,107, TW 1469792 B, RU 2636032 C2, U.S. Pat. No. 10,428,151, CN 106211774 B, EP 1871807 B1, U.S. Patent Application Publication No. 2019/0276544, EP 06748215 A, U.S. Pat. No. 8,329,178, and CA 2597717 A.

In some embodiments, the antibody can be an antibody that binds a GRPR polypeptide.

In some embodiments, an antibody that binds a GRPR polypeptide can be ABR-002, sc-398549, A30653.

In some embodiments, an antibody that binds GRPR polypeptide can be as described elsewhere. See, e.g., CA 2089212 C, DE 69637411 T2, EP 0981369 B1, CN 109422810 A, CN 106132993 A, and International Patent Application Publication No. WO 2015/143525.

In some embodiments, the antibody can be an antibody that binds a HER2 polypeptide. For example, an antibody that binds a HER2 polypeptide can include CDRs that comprise, consist essentially of, or consist of the CDR amino acid sequences set forth in SEQ ID NOs: 37-42. In some cases, an antibody that binds a HER2 polypeptide can have one or more CDRs that are a variant of (e.g., are not 100% identical to) a CDR set forth in any one of SEQ ID NOs: 37-42, provided that the antigen binding domain retains the ability to bind to a HER2 polypeptide. For example, one or more CDRs of an antibody that binds a HER2 polypeptide can consist of an amino acid sequence set forth in any one of SEQ ID NOs: 37-42, except that the variant polypeptide includes one, two, three, four, or five amino acid substitutions within the articulated sequence of the sequence identifier (e.g., any one of SEQ ID NOs: 37-42), has one, two, three, four, or five amino acid residues preceding the articulated sequence of the sequence identifier (e.g., any one of SEQ ID NOs: 37-42), and/or has one, two, three, four, or five amino acid residues following the articulated sequence of the sequence identifier (e.g., any one of SEQ ID NOs:37-42), provided that the antigen binding domain retains the ability to bind to a HER2 polypeptide. Examples of CDR amino acid sequences that comprise, consist essentially of, or consist of the CDR amino acid sequences set forth in SEQ ID NOs: 37-42 and can be used in an antibody that binds a HER2 polypeptide include, without limitation, those amino acid sequences shown in Table 7 (see, also, Example 17).

TABLE 7
Exemplary CDR sequences for anti-HER2 antibodies.

| CDR | Amino Acid Sequence | SEQ ID NO: |
|---|---|---|
| VL CDR1 | KASQDVSIGVA | 37 |
| | RASQSVSGSRFTYMH | 294 |
| | RASQDVNTAVA | 295 |
| | KASQDVSTAVA | 295 |
| VL CDR2 | SASYRYS | 38 |
| | SASYRES | 296 |
| | SASYLYS | 297 |
| | SASYLES | 298 |
| | SASYRET | 299 |
| | SASYLYT | 300 |
| | SASYLET | 301 |
| | YASILES | 302 |
| | SASYRYT | 303 |
| | SASFLYS | 304 |
| | SASFRYT | 305 |
| VL CDR3 | QQYYIYPYT | 39 |
| | QHSWEIPPWT | 306 |
| VH CDR1 | GFTFTDYTMD | 40 |
| | GFTFTDYTMS | 307 |
| | GYWMN | 308 |
| | GFNIKDTYIH | 309 |
| | GFTFNDYTMD | 310 |
| VH CDR2 | DVNPNSGGSIYNQRFKG | 41 |
| | MIHPLDAEIRANQKFR | 311 |
| | DVNPNSGGSIYNQRFK | 312 |
| | RIYPTNGYTRYADSVK | 313 |
| | DVNPNSGGSIVNRRFK | 314 |
| VH CDR3 | NLGPSFYFDY | 42 |
| | GTYDGGFEY | 315 |
| | WGGDGFYAMDY | 316 |
| | NLGPFFYFDY | 317 |

In some embodiments, an antibody that binds a BTER2 polypeptide can be trastuzumab, pertuzumab, margetuximab, ZW25, or zumuzumab.

In some embodiment, the antibody that binds a BTER2 polypeptide can be described elsewhere. See, e.g., Jones et al., Nature, 321, 522-525 (1986), CN 105829346 B, CN 107001479 B, KR 2014/0032004 A, AU 2005/32520, TW 1472339 B, CN 102167742 B, ES 2640449 T3, KR 20170055521 A, CN 111741979 A, International Patent Application Publication No. WO 2021/097220, and CN 107001479 B.

In some embodiments, the antibody can be an antibody that binds a MCR1 polypeptide.

In some embodiments, an antibody that binds a MCR1 polypeptide can be ARC0638 or EPR6530.

In some embodiments, the antibody can be an antibody that binds a VEGF polypeptide. Examples of VEGF polypeptides include VEGF1, VEGFB, VEGFC, and VEGFD. For example, an antibody that binds a VEGF polypeptide can include CDRs that comprise, consist essentially of, or consist of the CDR amino acid sequences set forth in SEQ ID NOs: 43-48. In some cases, an antibody that binds a VEGF polypeptide can have one or more CDRs that are a variant of (e.g., are not 100% identical to) a CDR set forth in any one of SEQ ID NOs: 43-48, provided that the antigen binding domain retains the ability to bind to a VEGF polypeptide. For example, one or more CDRs of an antibody that binds a VEGF polypeptide can consist of an amino acid sequence set forth in any one of SEQ ID NOs: 43-48, except that the variant polypeptide includes one, two, three, four, or five amino acid substitutions within the articulated sequence of the sequence identifier (e.g., any one of SEQ ID NOs: 43-48), has one, two, three, four, or five amino acid residues preceding the articulated sequence of the sequence identifier (e.g., any one of SEQ ID NOs: 43-48), and/or has one, two, three, four, or five amino acid residues following the articulated sequence of the sequence identifier (e.g., any one of SEQ ID NOs: 43-48), provided that the antigen binding domain retains the ability to bind to a MCR1 polypeptide. Examples of CDR amino acid sequences that comprise, consist essentially of, or consist of the CDR amino acid sequences set forth in SEQ ID NOs: 43-48 and can be used in an antibody that binds a VEGF polypeptide include, without limitation, those amino acid sequences shown in Table 8 (see, also, Example 17).

TABLE 8
Exemplary CDR sequences for anti-VEGF antibodies.

| CDR | Amino Acid Sequence | SEQ ID NO: |
|---|---|---|
| VL CDR1 | SASQDISNYLN | 43 |
| | RASQDVSTAVA | 318 |
| | RASQVIRRSLA | 319 |
| | RASQSYAYAVA | 320 |
| | CRASQASYYDVA | 321 |
| VL CDR2 | FTSSLHS | 44 |
| | SASFLYS | 322 |
| | AASNLAS | 323 |
| | DASYLYS | 324 |
| | AASYLYS | 325 |
| VL CDR3 | QQYSTVPWT | 45 |
| | QQSYTTPPT | 326 |
| | QQSNTSPLT | 327 |
| | QQAYSSPDT | 328 |
| | CQQYYYAPAT | 329 |
| VH CDR1 | GYTFTNYGMN | 46 |
| | GYDFTNYGMN | 330 |
| | GYTFTHYGMN | 331 |
| | GYDFTHYGMN | 332 |
| | ASWIH | 333 |
| | GSWIF | 334 |
| | GFAISDYDIH | 335 |
| | DYDIH | 336 |
| VH CDR2 | WLNTYTGEPTYAADFKR | 47 |
| | WINTYTGEPTYAADFKR | 337 |
| | AIYPYSGYTNYADSVKG | 338 |
| | AIWPFGGYTHYADSVKG | 339 |
| | DIAPYAGATAYADSVKG | 340 |
| | AIAPYSGSTYYADSVK | 341 |
| VH CDR3 | YPYYYGSSHWYFDV | 48 |
| | WGHSTSPWAMDY | 342 |
| | SSYAYYAAMDY | 343 |
| | SYAYYSAMDY | 344 |

In some embodiments, an antibody that binds a VEGF polypeptide can be bevacizumab, ranibizumab, brolucizumab, or faricimab.

In some embodiments, the antibody can be an antibody that binds a PD-L1 polypeptide. For example, an antibody that binds a PD-L1 polypeptide can include CDRs that comprise, consist essentially of, or consist of the CDR amino acid sequences set forth in SEQ ID Nos: 345-350. In some cases, an antibody that binds a PD-L1 polypeptide can have one or more CDRs that are a variant of (e.g., are not 100% o identical to) a CDR set forth in any one of SEQ ID NOs: 345-350, provided that the antigen binding domain retains the ability to bind to a PD-L1 polypeptide. For example, one or more CDRs of an antibody that binds a PD-L1 polypeptide can consist of an amino acid sequence set forth in any one of SEQ ID NOs: 345-350, except that the variant polypeptide includes one, two, three, four, or five amino acid substitutions within the articulated sequence of the sequence identifier (e.g., any one of SEQ TD NOs: 345-350), has one, two, three, four, or five amino acid residues preceding the articulated sequence of the sequence identifier (e.g., any one of SEQ ID NOs: 345-350), and/or has one, two, three, four, or five amino acid residues following the articulated sequence of the sequence identifier (e.g., any one of SEQ ID NOs: 345-350), provided that the antigen binding domain retains the ability to bind to a PD-L1 polypeptide. Examples of CDR amino acid sequences that comprise, consist essentially of, or consist of the CDR amino acid sequences set forth in SEQ ID NOs: 345-350 and can be used in an antibody that binds a PD-L1 polypeptide include, without limitation, those amino acid sequences shown in Table 9 (see, also, Example 17).

TABLE 9
Exemplary CDR sequences for anti-PD-L1 antibodies.

| CDR | Amino Acid Sequence | SEQ ID NO: |
|---|---|---|
| VL CDR1 | DVSTAVA | 345 |
| | RVSSSYLA | 357 |
| | SVSSYLA | 358 |
| | VGGYNYVS | 359 |
| | TRSSGSIDSNYVQ | 360 |
| VL CDR2 | SASFLY | 346 |
| | DASSRA | 361 |
| | DASNRA | 362 |
| | DVSNRP | 363 |
| | EDNQRPS | 364 |
| VL CDR3 | QQYLYHPAT | 347 |
| | QQYGSLPWT | 365 |
| | QQRSNWPT | 366 |
| | SSYTSSSTRV | 367 |
| | QSYDSNNRHVI | 368 |
| VH CDR1 | DSWIH | 348 |
| | RYWMS | 369 |
| | TYAIS | 370 |
| | SYIMM | 371 |
| | GTFSRSAIS | 372 |
| VH CDR2 | WISPYGGSTY | 349 |
| | NIKQDGSEKY | 373 |
| | GIIPIFGKAH | 374 |
| | SIYPSGGITF | 375 |
| | VIIPAFGEANYAQKFQG | 376 |
| VH CDR3 | RHWPGGF | 350 |
| | EGGWFGELAF | 377 |
| | KFHFVSGSPFGM | 378 |
| | IKLGTVTTV | 379 |
| | ARGRQMFGAGIDF | 380 |

In some embodiments, an antibody that binds a PD-L1 polypeptide can be atezolizumab, avelumab, durvalumab, BMS 936559, or cosibelimab.

In some embodiments, the antibody can be an antibody that binds a TROP2 polypeptide. For example, an antibody that binds a VEGF polypeptide can include CDRs that comprise, consist essentially of, or consist of the CDR amino acid sequences set forth in SEQ ID Nos: 351-356. In some cases, an antibody that binds a TROP2 polypeptide can have one or more CDRs that are a variant of (e.g., are not 100% identical to) a CDR set forth in any one of SEQ ID NOs: 351-356, provided that the antigen binding domain retains the ability to bind to a TROP2 polypeptide. For example, one or more CDRs of an antibody that binds a TROP2 polypeptide can consist of an amino acid sequence set forth in any one of SEQ ID NOs: 351-356, except that the variant polypeptide includes one, two, three, four, or five amino acid substitutions within the articulated sequence of the sequence identifier (e.g., any one of SEQ ID NOs: 351-356), has one, two, three, four, or five amino acid residues preceding the articulated sequence of the sequence identifier (e.g., any one of SEQ ID NOs: 351-356), and/or has one, two, three, four, or five amino acid residues following the articulated sequence of the sequence identifier (e.g., any one of SEQ ID NOs: 351-356), provided that the antigen binding domain retains the ability to bind to a TROP2 polypeptide. Examples of CDR amino acid sequences that comprise, consist essentially of, or consist of the CDR amino acid sequences set forth in SEQ ID NOs: 351-356 and can be used in an antibody that binds a TROP2 polypeptide include, without limitation, those amino acid sequences shown in Table 10 (see, also, Example 17).

TABLE 10
Exemplary CDR sequences for anti-TROP2 antibodies.

| CDR | Amino Acid Sequence | SEQ ID NO: |
|---|---|---|
| VL CDR1 | KASQDVSIAVA | 351 |
| | RASQSISSYLN | 381 |
| | KASQDVSIAV | 382 |
| VL CDR2 | SASYRTT | 352 |
| | AASSLQS | 383 |
| | SASYRYT | 384 |
| VL CDR3 | QQHYITPLT | 353 |
| | QQSYSTPLT | 385 |
| | QQHYITPL | 386 |
| VH CDR1 | YNYGM | 354 |
| | TSYAM | 387 |
| | TNYGM | 388 |
| VH CDR2 | WINTYTGEPTYTDDFKG | 355 |
| | WINTNTGNPTYAQGFTG | 389 |
| | WINTYTGEPTYTDDFK | 390 |
| VH CDR3 | GGFGSSYWYFDV | 356 |
| | EDSNGYKIFDY | 391 |
| | GGFGSSYWYFD | 392 |

In some embodiments, an antibody that binds a TROP2 polypeptide can be sacituzumab or datopotamab.

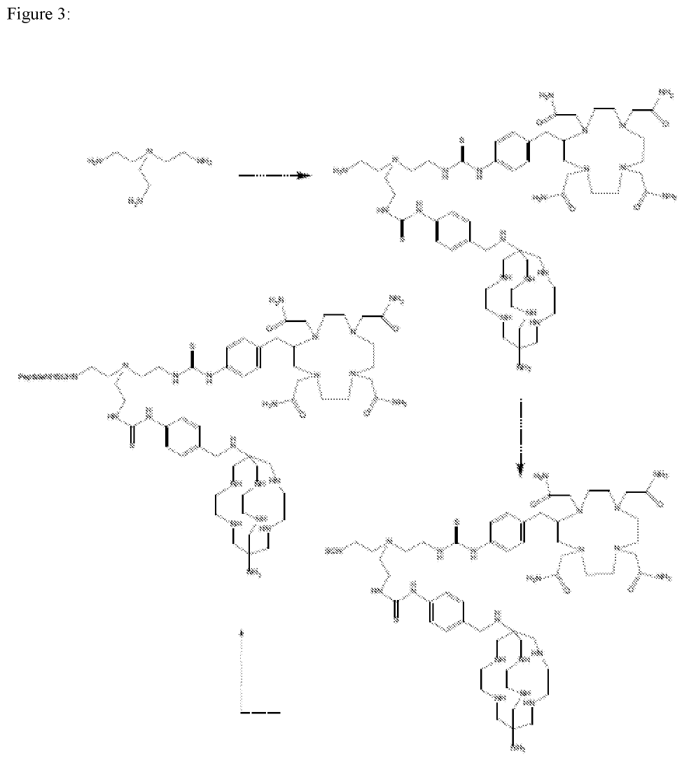

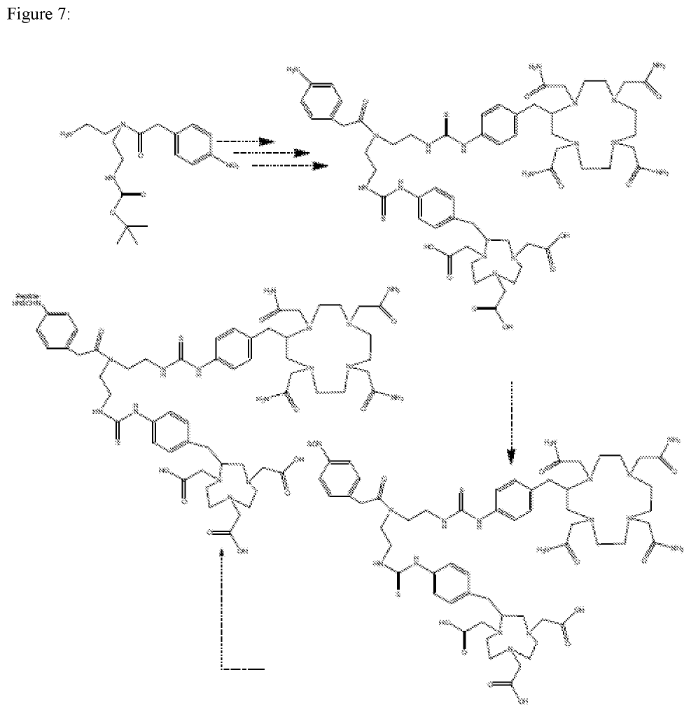

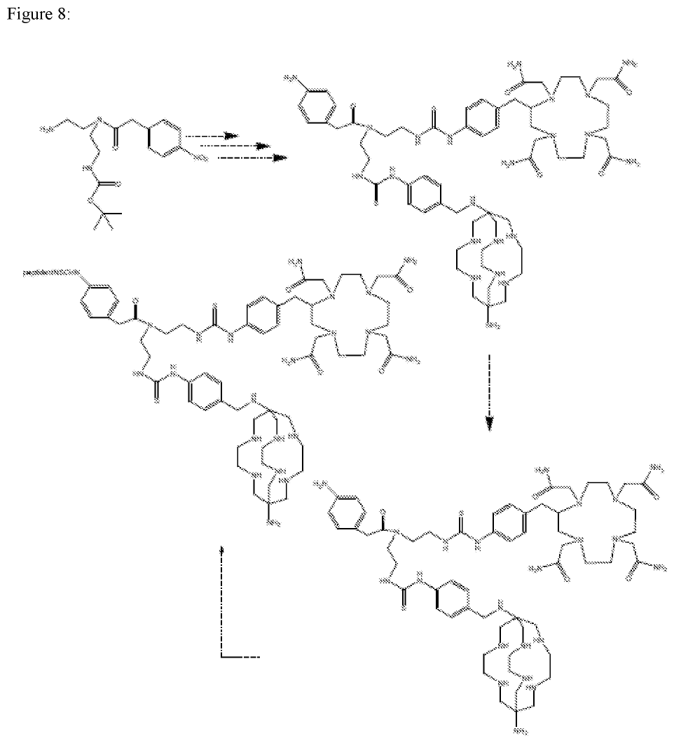

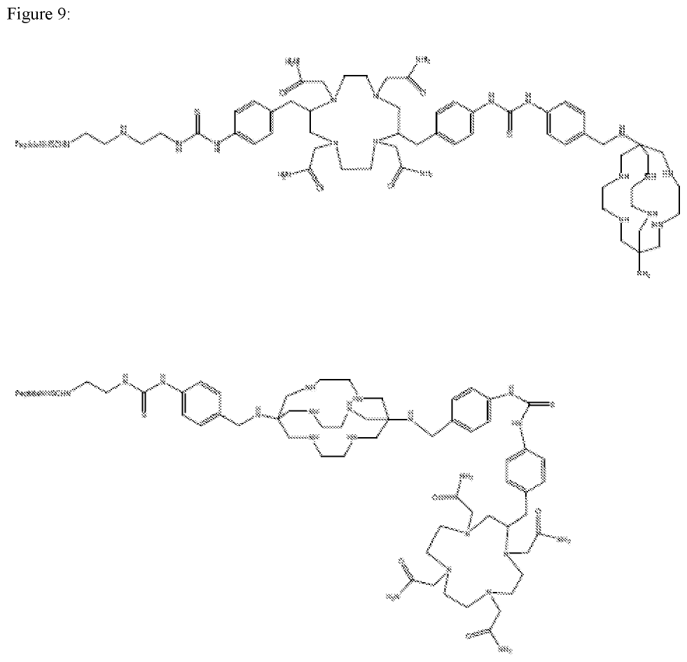

In some embodiments, a conjugate can be prepared as shown in any one or more of FIGS. 3-9 and 70-81. For example, in some embodiments, one chelator can be DiAmSar and one chelator can be TCMC. In some embodiments, one chelator can be NOTA and one chelator can be TCMC. In some embodiments, the binding moiety is a PSMA peptide. In some embodiments, the PSMA peptide is piflufostat. In some embodiments, the binding moiety is a fibroblast activating protein inhibitor (FAPI). In some embodiments, the FAPI is N-[2-[(2S)-2-cyano-4,4-difluoropyrrolidin-1-yl]-2-oxoethyl]-6-hydroxyquinoline-4-carboxamide. In some embodiments, the binding moiety is a ligand for a somatostatin receptor. In some embodiments, the ligand for the somatostatin receptor is octreotide, pasireotide, vapreotide, lanreotide, somatostatin, edotreotide, or oxodotreotide. In some embodiments, the binding moiety is a CD3 inhibitor. In some embodiments, the binding moiety is a CD20 inhibitor. In some embodiments, the binding moiety is a CXCR4 inhibitor. In some embodiments, the CXCR4 inhibitor is framycetin, plerixafor, baclofen, mavorixafor, or MSX-122. In some embodiments, the binding moiety is a GRPR inhibitor. In some embodiments, the GRPR inhibitor is bombesin, RC-3095, PD 168368, GRPR antagonist 1, GRPR antagonist 2, or PD 176252. Some examples of GRPR antagonists that can be used as described herein are as set forth in Yu et al., Med Chem Res 30, 2069-2089 (2021), which is hereby incorporated by reference. In some embodiments, the binding moiety is a HER2 inhibitor. In some embodiments, the HER2 inhibitor is lapatinib, tesevatinib, varlitinib, tucatinib, afatinib, brigatinib, fostamatinib, zanubrutinib, tucatinib, or neratinib. In some embodiments, the binding moiety is a MC1R ligand. In some embodiments, the MC1R ligand is 4-phenylbutyryl-Hi s-DPhe-Arg-Trp-Gly-Lys(hex-5-ynoyl)-NH2, H-Lys(hex-5-ynoyl)-Tyr-Val-Nle-Gly-His-DNal(2′)-Arg-DTrp-Asp-Arg-Phe-Gly-NH2, H-Lys(hex-5-ynoyl)Tyr-Val-Nle-Gly-His-DNal(2′)-Arg-DPhe-Asp-Arg-Phe-Gly-NH2, adrenocorticotropic hormone, alpha melanocyte-stimulating hormone, beta melanocyte-stimulating hormone, gamma melanocyte-stimulating hormone, or MC1RL. Some further examples of MC1R ligands that can be used as described herein are as set forth in one or more of the following: Tafreshi et al., J. Nucl. Med. 60(8), 1124-1133 (2019); and U.S. Pat. Nos. 8,492,517, 8,933,194, and 11,286,280, which are hereby incorporated by reference. In some embodiments, the binding moiety is a VEGF inhibitor. In some embodiments, the VEGF inhibitor is sunitinib, vatalanib, linifanib, denibulin, pazopanib, axitinib, regorafenib, sorafenib, lenvatinib, nintedanib, polaprezinc, fostamatinib, selpercatinib, or tivozanib. In some embodiments, the binding moiety is a PD-L1 inhibitor. In some embodiments, the PD-L1 inhibitor is AUNP-12, CA-170, (3S,3aR,6S,6aR)-N6-[4-(3-fluorophenyl)-pyrimidin-2-yl]-N3-(2-pyridylmethyl)-2,3,3a,5,6,6a-hexahydrofu, or 1-isopropyl-3-[(3S,5S)-1-methyl-5-[3-(2-naphthyl)-1,2,4-oxadiazol-5-yl]pyrrolidin-3-yl]urea. In some embodiments, the binding moiety is a PTK2 inhibitor. In some embodiments, the PTK2 inhibitor is endostatin, fostamatinib, 7-Pyridin-2-Yl-N-(3,4,5-Trimethoxyphenyl)-7h-Pyrrolo[2,3-D]pyrimidin-2-Amine, 2-({5-Chloro-2-[(2-Methoxy-4-Morpholin-4-Ylphenyl)amino]pyrimidin-4-Yl}amino)-N-Methylbenzamide, GSK2256098, defactinib, or VS-4718. In some embodiments, the binding moiety is an ITGB6 binder. In some embodiments, the ITGB6 binder is the cyclic peptide cyclo(FRGDLAFp(NMe)K) or trivehexin, as described in Quigley et al., Eur J. Nucl. Med Mol. Imaging. 49(4), 1136-1147 (2022). Sometimes the binding moiety is 3-fluoro-2,2-dimethylpropionic acid or 2,2-dimethylpropionic acid.

As described herein, a conjugate provided herein can include one or more binding moieties (e.g., one, two, three, four, five, or more binding moieties). In some cases, a binding moiety of a conjugate described herein can have the ability to bind to one or more target molecules. For example, a binding moiety of a conjugate described herein can have the ability to bind to one, two, three, four, five, or more target molecules such as one, two, three, four, five, or more target molecules present on a cell (e.g., a cancer cell).

In some embodiments, a conjugate provided herein having two or more binding moieties can advantageously, for example, bind to antigens present on two different cells (e.g., two different cancer cells), or bind to two different antigens on the same cell (e.g., the same cancer cell). In some embodiments, having more than one binding moiety provides one or more advantages, such as, the conjugate having enhanced uptake and/or increased in vivo stability.

In some embodiments, one or more conjugates described herein can be used to treat a cancer (e.g., prostate cancer, a neuroendocrine cancer, colon cancer, lung cancer, pancreatic cancer, melanoma, or a lymphoid cancer) in a mammal (e.g., a human patient). For example, for treating prostate cancer, a conjugate that includes a binding moiety that targets PSMA or its activity can be used. For treating a neuroendocrine cancer, a conjugate that includes a binding moiety that targets a somatostatin receptor (e.g., a somatostatin analog) can be used. For treating lung cancer, a conjugate that includes a binding moiety that targets the B7-H3 protein can be used. For treating pancreatic cancer, a conjugate that includes a binding moiety that targets C9-19 can be used. For treating melanoma, a conjugate that includes a binding moiety that targets the melanocortin 1 receptor can be used.

In some embodiments, one or more conjugates described herein can be used to treat a non-cancer condition (e.g., a benign tumor, an inflammatory condition, a hematologic process, a histiocytic process, a cystic disease or infection) in a mammal (e.g., a human patient).

In some embodiments, one or more conjugates described herein can be administered to a mammal (e.g., a human patient) once or multiple times over a period of time ranging from days to months to treat a cancer or non-cancer condition in a mammal (e.g., a human patient). In some embodiments, one or more conjugates described herein (e.g., a conjugate that includes two or more chelators covalently attached to a binding moiety via a linker, wherein one of the chelators is a chelator of an isotope used for imaging and one of the chelators is a chelator of an isotope used for radiotherapy, wherein the isotope used for imaging and the isotope used for radiotherapy are each complexed (chelated) to the chelator, and wherein the binding moiety binds to a tumor in the patient) can be formulated into a pharmaceutically acceptable composition for administration to a patient (e.g., a patient identified as having cancer) to treat a cancer within that patient. In some embodiments, a mixture of two conjugates can be administered to, for example, provide a suitable dose (radioactively speaking) of each radioisotope at the time of injection. In such embodiments, the appropriate isotopes can be complexed with the chelators of the two conjugates and mixed at the time of injection to account for the decay at different rates.

In some embodiments, the biodistribution of the conjugate (i.e., location of the conjugate within the mammal) can be determined in the patient (e.g., by PET) by administering a conjugate that includes two or more chelators covalently attached to a binding moiety via a linker, wherein one of the chelators is a chelator of an imaging isotope and one of the chelators is a chelator of a radiotherapy isotope, wherein the imaging isotope is chelated to the chelator, and wherein the binding moiety binds to a tumor in the patient. After the biodistribution is determined, the same conjugate, except having both the imaging isotope and radiotherapy isotope chelated to the chelator, can be administered to the patient. Determining the biodistribution allows the dose of the therapy to be tailored to the patient, reducing side effects. Imaging can be performed after each administration of conjugate to monitor therapy.

A therapeutically effective amount of a conjugate described herein can be formulated together with one or more pharmaceutically acceptable carriers (additives or excipients) and/or diluents. In some embodiments, the additives stabilize against radiolysis. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more conjugates can be designed for oral or parenteral (including subcutaneous, intramuscular, intravenous, intradermal, inhaled/aerosolized, intraarterial, intrathecal, intratumoral, intracystic, peritumoral, intraperitomeal, intraluminal, intrapleural) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, or solutes that render the formulation isotonic with the blood of the intended recipient. The formulations can be presented in unit-dose or multi-dose containers, for example, sealed ampules and vials, and may be stored (e.g., in a freeze dried (lyophilized) condition) requiring only the addition of the sterile liquid carrier, for example, water or saline for injections immediately prior to use. In some embodiments, the formulations can be presented in a form that only requires the addition of a sterile carrier (e.g., water or saline) and the desired radionuclide(s). Extemporaneous injection solutions and suspensions may be prepared from sterile powders, granules, and tablets.

In some cases, a pharmaceutically acceptable composition including one or more conjugates described herein can be administered locally or systemically. For example, a composition provided herein can be administered systemically by intravenous injection or blood infusion. For example, a composition provided herein can be administered locally, e.g., intratumoral, intramuscular, intradermal or subcutaneous). For example, an intraarterial injection can be used to locally direct the composition, e.g., injection into the hepatic artery to target cancer in the liver). In some cases, a composition provided herein can be administered systemically, orally, or by injection to a mammal (e.g., a human patient).

An effective amount of a composition containing one or more conjugates can be any amount that provides an anti-tumor response (e.g., slowing, stopping, or reversing tumor growth by stopping tumor cell multiplication and/or killing tumor cells) without producing significant toxicity to the patient. For example, an effective amount of a conjugate that includes a positron-emitting PET isotope can range from 1 mCi to 20 mCi (e.g., about 1 mCi to about 15 mCi, about 1 mCi to about 10 mCi, about 2 mCi to about 18 mCi, about 3 mCi to about 17 mCi, about 4 mCi to about 18 mCi, about 4 mCi to about 15 mCi, about 5 mCi to about 20 mCi, about 5 mCi to about 15 mCi, about 10 mCi to about 20 mCi, about 15 mCi to about 20 mCi). In some embodiments, an effective amount of a conjugate that includes a beta-emitting isotope can range, for example, from about 10 mCi to 1.5 Ci (1,500 mCi) per cycle (e.g., about 15 mCi to about 1,400 mCi, about 25 mCi to about 1,500 mCi, about 50 mCi to about 1,250 mCi, about 75 mCi to about 1,500 mCi, about 100 mCi to about 1,000 mCi, about 100 mCi to about 1,400 mCi, about 150 mCi to about 1,250 mCi, about 200 mCi to about 1,200 mCi, about 300 mCi to about 1,100 mCi, about 400 mCi to about 1,000 mCi, about 500 mCi to about 1,500 mCi, about 600 mCi to about 1,400 mCi, about 700 mCi to about 1,300 mCi, about 800 mCi to about 1,200 mCi, or about 1,000 mCi to about 1,500 mCi per cycle). In some embodiments, an effective amount of a conjugate that includes a gamma-emitting isotope (e.g., a SPECT agent) can range, for example, from about 0.1 mCi to about 40 mCi (e.g., about 0.2 mCi to about 40 mCi, about 0.5 mCi to about 35 mCi, about 0.5 mCi to about 25 mCi, about 1 mCi to about 35 mCi, about 1 mCi to about 30 mCi, about 2 mCi to about 38 mCi, about 3 mCi to about 30 mCi, about 4 mCi to about 35 mCi, about 4 mCi to about 35 mCi, about 5 mCi to about 40 mCi, about 5 mCi to about 35 mCi, about 5 mCi to about 30 mCi, about 5 mCi to about 25 mCi, about 5 mCi to about 20 mCi, about 10 mCi to about 30 mCi, about 15 mCi to about 40 mCi, about 20 mCi to about 40 mCi, or about 25 mCi to about 40 mCi). In some embodiments, an effective amount of a conjugate that includes an alpha-emitting isotope can range, for example, from about 0.05 mCi to 100 mCi per cycle (e.g., about 0.05 to about 90 mCi, about 0.1 mCi to about 100 mCi, about 0.2 mCi to about 90 mCi, about 0.5 mCi to about 95 mCi, about 0.5 mCi to about 85 mCi, about 1 mCi to about 95 mCi, about 1 mCi to about 85 mCi, about 2 mCi to about 95 mCi, about 3 mCi to about 90 mCi, about 4 mCi to about 85 mCi, about 4 mCi to about 80 mCi, about 5 mCi to about 100 mCi, about 5 mCi to about 85 mCi, about 5 mCi to about 70 mCi, about 5 mCi to about 60 mCi, about 5 mCi to about 50 mCi, about 10 mCi to about 100 mCi, about 15 mCi to about 60 mCi, about 20 mCi to about 80 mCi, or about 25 mCi to about 100 mCi per cycle).

In some embodiments, in which two or more conjugates are to be administered, the effective amount of each conjugate may be different. For example, when it is desired to use an alpha-emitting isotope for therapy and a positon-emitting isotope for imaging, different amounts of the conjugates can be administered.

For example, an effective amount of one or more conjugates described herein can be administered to an average sized human (e.g., about 75-85 kg human) per administration (e.g., per daily, weekly, monthly, bimonthly, or quarterly administration). In some cases, a conjugate can be administered once followed by a rest period of between two and sixteen weeks (e.g., two, three, four, five, six, seven, eight, nine, 10, 11, 12, 13, 14, 15, or 16 weeks) to monitor the patient for adverse effects (e.g., by monitoring complete blood counts, white blood cell count, platelet count, hemoglobin levels, or bone marrow injury) before repeating the administration. Each administration and rest period is referred to as a cycle of therapy.

If a particular mammal fails to respond to a particular amount of therapy drug conjugate, or the calculated amount of drug arriving at a target tumor is too low, then the amount of a conjugate injected in the next cycle can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of a conjugate described herein can be any frequency that provides an anti-tumor response (e.g., stopping tumor growth or killing tumor cells) without producing significant toxicity to the mammal. For example, the frequency of administration of a conjugate can be from about once a day, once a month, once every six weeks, once every two months, or about once every three months, or about once every 16 weeks. The frequency of administration of a conjugate described herein can remain constant or can be variable during the duration of treatment (e.g., more frequent administration with less toxicity). As described above, a course of treatment with a composition containing a conjugate can include rest periods. For example, a composition containing one or more conjugates can be administered once followed by a rest period of between two and sixteen weeks (e.g., two, three, four, five, six, seven, eight, nine, 10, 11, 12, 13, 14, 15, or 16 weeks), and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more conjugates can be any duration that provides an anti-tumor response (e.g., stopping tumor growth or killing tumor cells) within a mammal identified as having cancer without producing significant toxicity to the mammal. In some cases, the effective duration can vary from several days to several months. In general, the effective duration for providing an anti-tumor response (e.g., stopping tumor growth or killing tumor cells) within a mammal identified as having cancer can range in duration from about six weeks to about ten months. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### EXAMPLES

### Example 1—Diamsar and TCMC Platform for Polypeptide Conjugation

As shown in FIG. 3, diamsar (1-N-(4-Aminobenzyl)-3,6,10,13,16,19-hexaazabicyclo[6.6.6]-eicosane-1,8-diamine (SarAr) with CN=6)-TCMC (,4,7,10-tetrakis(carbamoylmethyl)-1,4,7,10tetraazacyclododecane, N₄O₄, with coordination number (CN) 8) platform can be easily conjugated to any peptide or antibody at room temperature (may be needed to heat up to 37° C. max). TCMC can conjugate with Pb and Diamsar can conjugate with Cu and both complexation reactions are feasible between room temperature and 37° C. If needed, the chain length of the third arm containing NCS group can be adjusted/enlarged to mitigate any potential steric hindrance. The competitive conjugation of Pb and Cu can be tested in presence of both of the chelators at any given pH, buffer and temperature to confirm the conjugation. Due to the lipophilic nature of the Diamsar, overall lipophilic character and related properties of the peptide are expected to be increased with addition of Diamsar-TCMC conjugation.

### Example 2—NOTA and TCMC Platform for Polypeptide Conjugation

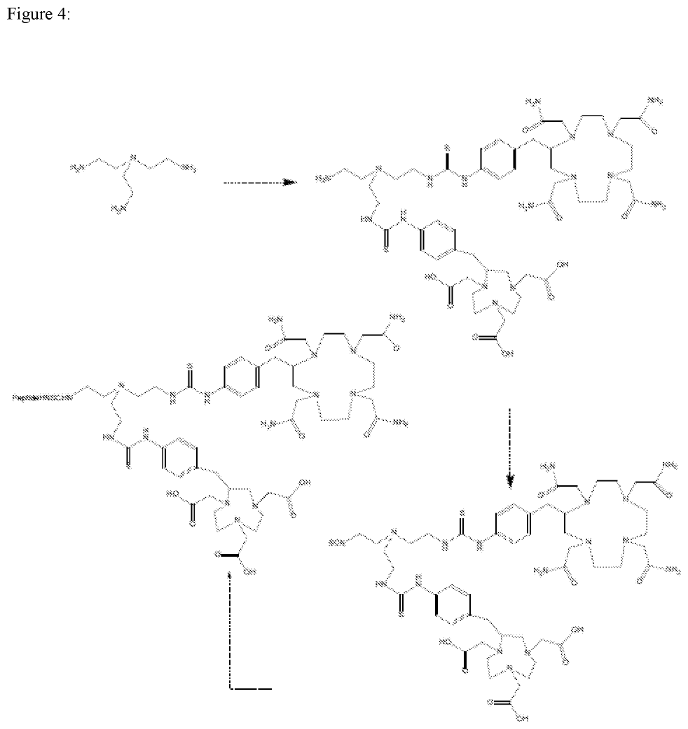

As shown in FIG. 4, NOTA (p-SCN-Bn-NOTA, is chemically 1,4,7-triazacyclononane-1,4,7-triacetic acid, CN=6, N₃O₃ with CN=6)-TCMC platform can be easily conjugated to any peptide or antibody at room temperature (may be needed to heat up to 37° C. max). TCMC can conjugate with Pb and NOTA can conjugate with Cu and both complexation reactions are feasible between room temperature and 37° C. If needed, the chain length of the third arm containing NCS group can be adjusted/enlarged to mitigate any potential steric hindrance. The competitive conjugation of Pb and Cu can be tested in presence of both of the chelators at any given pH, buffer and temperature to confirm the conjugation.

### Example 3—Diamsar and TCMC Platform for Dual Polypeptide Conjugation

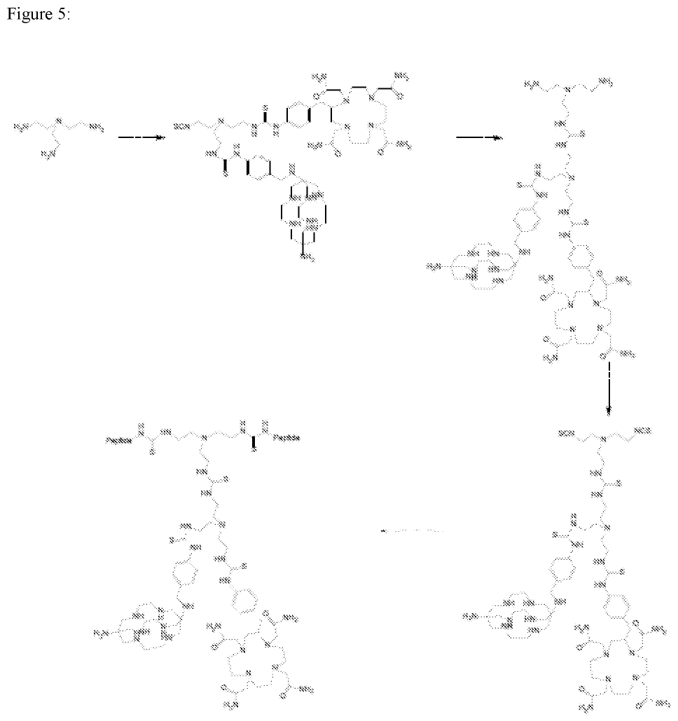

As shown in FIG. 5, diamsar-TCMC platform can be easily conjugated to any peptide or antibody at room temperature (may be needed to heat up to 37° C. max). TCMC can conjugate with Pb and Diamsar can conjugate to Cu, and both complexation reactions are feasible between room temperature and 37° C. If needed, the chain length of the third arm containing NCS group can be adjusted/enlarged to mitigate any potential steric hindrance. The competitive conjugation of Pb and Cu can be tested in presence of both of the chelators at any given pH, buffer and temperature to confirm the conjugation. Due to the lipophilic nature of the Diamsar, overall lipophilic character and related properties of the peptide are expected to be increased with addition of Diamsar-TCMC conjugation. This approach allows dual conjugation of peptides and antibodies to facilitate enhanced binding to the targeted receptors.

### Example 4—NOTA and TCMC Platform for Dual Polypeptide Conjugation

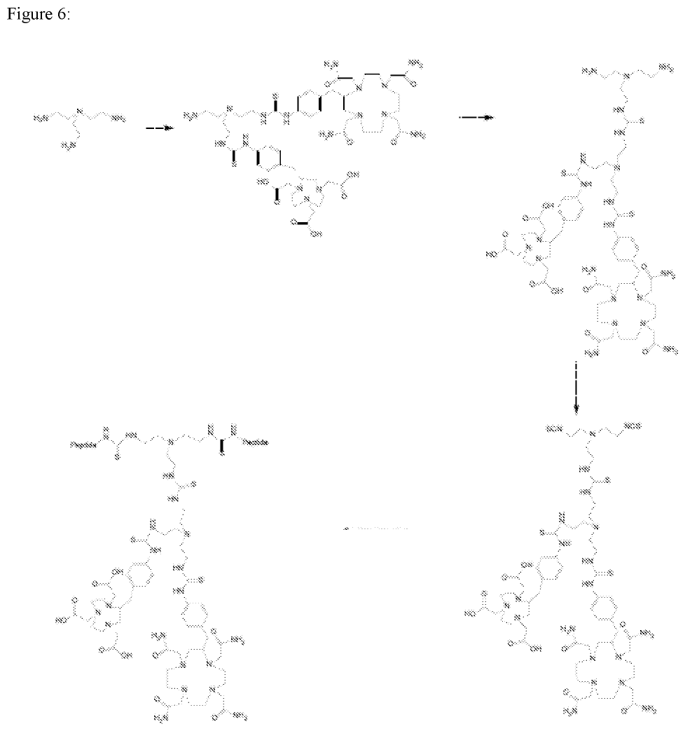

As shown in FIG. 6, the NOTA-TCMC platform can be easily conjugated to any peptide or antibody at room temperature (may be needed to heat up to 37° C. max). TCMC can conjugate with Pb and NOTA can conjugate to Cu, and both complexation reactions are feasible between room temperature and 37° C. If needed, the chain length of the third arm containing NCS group can be adjusted/enlarged to mitigate any potential steric hindrance. The competitive conjugation of Pb and Cu can be tested in presence of both of the chelators at any given pH, buffer and temperature to confirm the conjugation. This approach allows dual conjugation of peptides and antibodies to facilitate enhanced binding to the targeted receptors.

### Example 5—⁶⁴Cu-Labeling of Conjugate 1

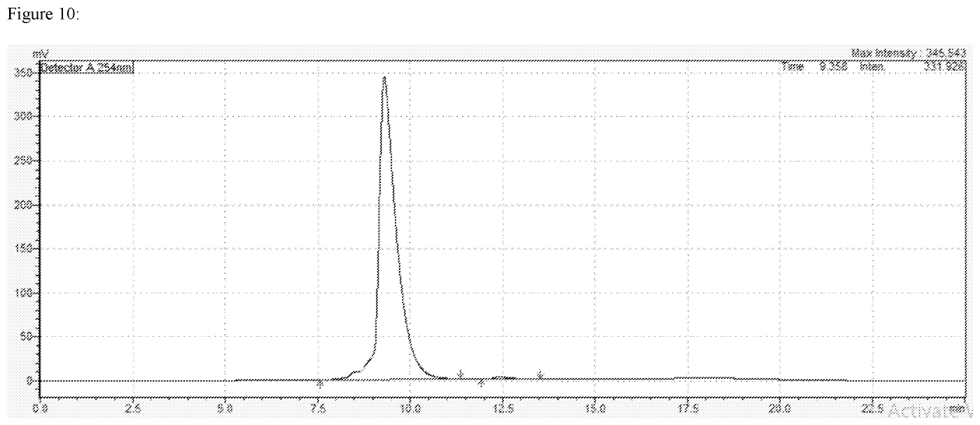

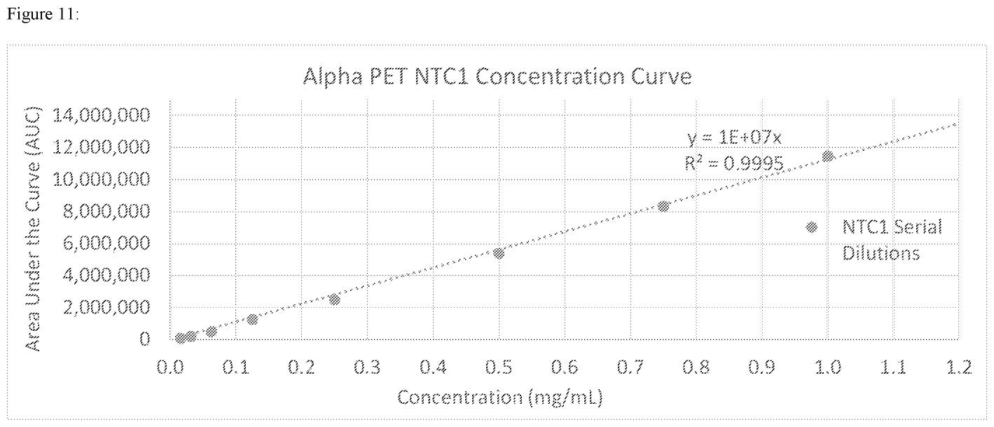

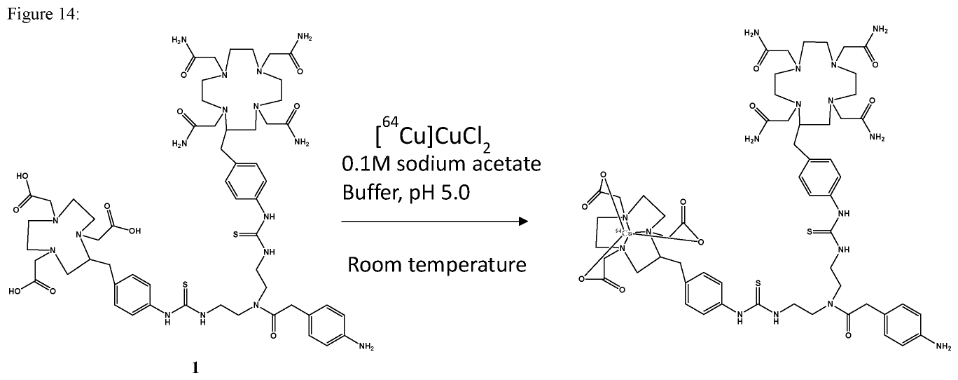

As shown in FIG. 10, a high performance liquid-chromatography (HPLC) method was developed for Conjugate 1 and ⁶⁴Cu-Conjugate 1 (FIG. 14). The method used a Schmadzu HPLC system, which was equipped with dual UV and radioactivity detectors. The method was developed and optimized using a reverse phase HPLC column (C-18) from Phenomenex (Luna 5 μm C18(2) 100 Å LC Column 250×4.6 mm, (00G-4252-e0) using a UV wavelength of 254 nm. For analyte analysis, a 20 μL injection loop was installed and used for all analysis at room temperature. For mobile phase, a dual solvent system was used composed of solvent A as 0.1% trifluoroacetic acid (TFA) in acetonitrile and solvent B as 0.1% TFA in water. For peak separation, a gradient method as described in Tables 12A and 12B was used with 1.1 mL/minute flow rate of the mobile phase. Table 11 shows the results of the HPLC method. FIG. 11 shows the HPLC calibration curve of varying concentrations shown in Table 12A. For analysis, the compound was dissolved in water and a calibration curve was prepared to estimate the specific activity of the synthesized compound. Depending upon the chemical nature of the compound a different retention time was observed.

TABLE 11

| | Retention Time | Area under the curve | |
|---|---|---|---|
| Peak | (minute) | (AUC) | % AUC |
| 1 | 9.31 | 11573801 | 99.3 |
| 2 | 12.42 | 81067 | 0.7 |

TABLE 12A

| Number of | | | |
|---|---|---|---|
| samples | Concentration | | Retention Time |
| injected | (μg/mL) | AUC | (minute) |
| 1 | 15.6 | 71494 | 8.14 |
| 2 | 31.25 | 189275 | 8.24 |
| 3 | 62.5 | 478435 | 8.14 |
| 4 | 125 | 1245187 | 8.22 |
| 5 | 250 | 2486055 | 8.10 |
| 6 | 500 | 5383117 | 8.01 |
| 7 | 750 | 8316302 | 8.05 |
| 8 | 1000 | 11624995 | 8.01 |

TABLE 12B

| | % of solvent A | % of solvent B |
|---|---|---|
| Time | (0.1% TFA in acetonitrile) | (0.1% TFA in water) |
| 0.01 minutes | 5 | 95 |
| 12.0 minutes | 50 | 50 |
| 23.0 minutes | 5 | 95 |
| 25.0 minutes | 5 | 95 |

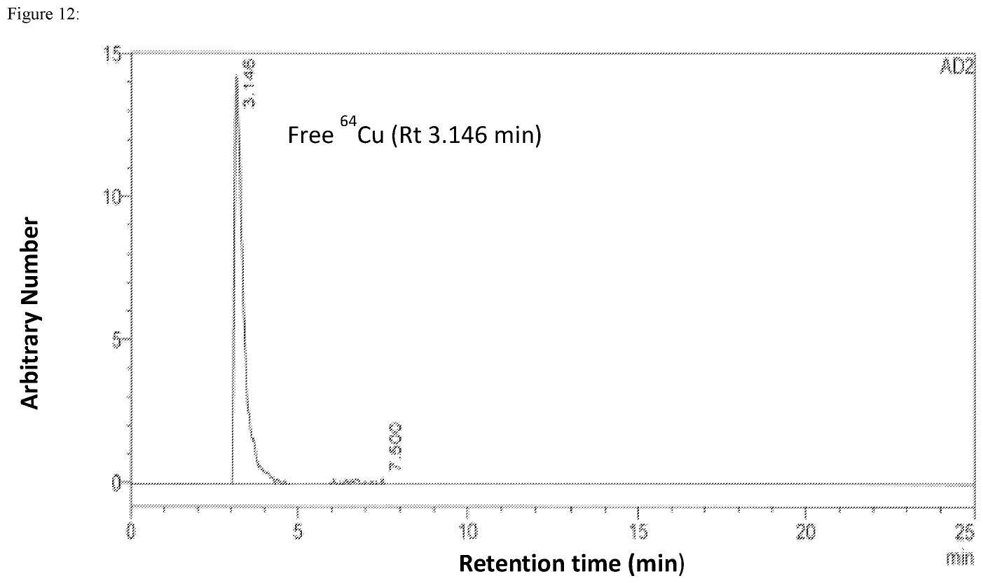

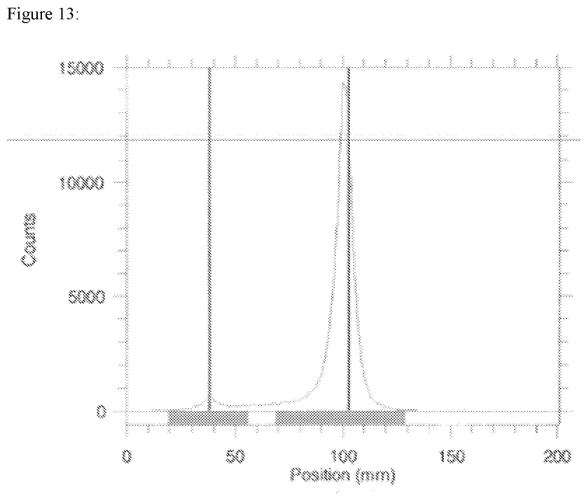

Further, an HPLC method was developed for unlabeled ⁶⁴Cu. FIG. 12 shows the HPLC trace of unlabeled ⁶⁴Cu. The method used a Schmadzu HPLC system was used, which was equipped with dual UV and radioactivity detectors. The method was developed and optimized using a reverse phase HPLC column (C-18) from Phenomenex (Luna 5 μm C18(2) 100 Å LC Column 250×4.6 mm, (00G-4252-e0) using a UV wavelength of 254 nm. For analyte analysis, a 20 μL injection loop was installed and used for all analysis at room temperature. For mobile phase, a dual solvent system was used composed of solvent A as 0.1% trifluoroacetic acid (TFA) in acetonitrile and solvent B as 0.1% TFA in water. For peak separation, a gradient method was used as described in Table 14B below using 1.0 mL/minute flow rate of the mobile phase. Table 13 shows the results of the HPLC method of unlabeled ⁶⁴Cu. A thin-layer chromatography (TLC) method was developed for unlabeled ⁶⁴Cu, using silica gel as the solid phase and 0.1 M sodium citrate as the mobile phase. FIG. 13 shows the TLC trace of unlabeled ⁶⁴Cu and Table 14A shows the results.

TABLE 13

| | Ret. Time | | | |
|---|---|---|---|---|
| Peak | (minute) | Area | Height | % Concentration |
| 1 | 3.146 | 270310 | 14291 | 98.093 |
| 2 | 7.500 | 5255 | 228 | 1.907 |
| | | 275565 | 14518 | 100.00 |

TABLE 14A

| | Start | Stop | Centroid | | Region | Region | % of | % of |
|---|---|---|---|---|---|---|---|---|
| Peak | (mm) | (mm) | (mm) | RF | Counts | CPM | Total | ROI |
| 1 | 19.4 | 56.0 | 40.3 | 0.036 | 10042.0 | 60252.0 | 5.10 | 5.22 |
| 2 | 68.8 | 128.4 | 99.5 | 0.947 | 182232.0 | 1093392.0 | 92.53 | 94.78 |
| | | | | | 192274.0 | 1153644.0 | 97.63 | 100.00 |

TABLE 14B

| | % of solvent A | % of solvent B |
|---|---|---|
| Time | (0.1% TFA in acetonitrile) | (0.1% TFA in water) |
| 0.01 minutes | 5 | 95 |
| 12.0 minutes | 55 | 45 |
| 23.0 minutes | 5 | 95 |
| 25.0 minutes | 5 | 95 |

As shown in FIG. 14, Conjugate 1 was labeled with ⁶⁴Cu to form the ⁶⁴Cu-Conjugate 1. The Conjugate-1 was radiolabeled with Cu-64 using [⁶⁴Cu]CuCl₂ produced from cyclotron and formulated in 0.1M hydrochloric acid. Different amounts (50 μg, 100 μg) of Conjugate-1 were used and the pH was adjusted to 5.0 using 0.1M sodium acetate after addition of [⁶⁴Cu]CuCl₂. The resultant reaction mixture was stirred at room temperature for different durations as 10 minutes, 20 minutes, 30 minutes, and 40 minutes to optimize radiolabeling yield with reaction time. The progress and yield of the reactions were monitored by radioactive thin layer chromatography (r-TLC) using iTLC (silica gel coated on paper, Agilent Technologies Inc., Santa Clara, CA) and 0.1M sodium citrate as a mobile phase. In this r-TLC condition, unconjugated (Free)⁶⁴Cu moves to the solvent front of the r-TLC and radiolabeled ⁶⁴Cu-Conjugate-1 stays at the origin of the r-TLC plate. Based on our tested radiolabeling condition, the reaction achieved >99% yield radiolabeling in 10 minutes and at all other time points at pH 5.0 using sodium acetate as a reaction buffer via stirring at room temperature. Our radiolabeling yields, as function of reaction time, temperature, and mass of the starting conjugate-1 are summarized in Table 16. Formation of radiolabeled ⁶⁴Cu-Conjugate-1 was also confirmed by radio HPLC.

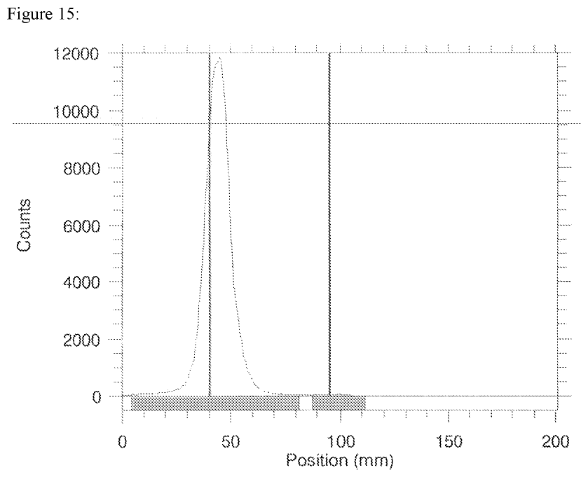

The TLC trace of the product, ⁶⁴Cu-Conjugate 1, is shown in FIG. 15. The TLC was performed with a silica gel solid phase and 0.1M sodium citrate mobile phase. Table 15 shows the TLC results.

TABLE 15

| | Start | Stop | Centroid | | Region | Region | % of | % of |
|---|---|---|---|---|---|---|---|---|
| Peak | (mm) | (mm) | (mm) | RF | Counts | CPM | Total | ROI |
| 1 | 4.0 | 81.6 | 44.2 | 0.075 | 197700.0 | 1078363.6 | 99.31 | 99.58 |
| 2 | 87.5 | 111.4 | 98.2 | 1.058 | 829.0 | 4521.8 | 0.42 | 0.42 |
| | | | | | 198529.0 | 1082885.5 | 99.72 | 100.00 |

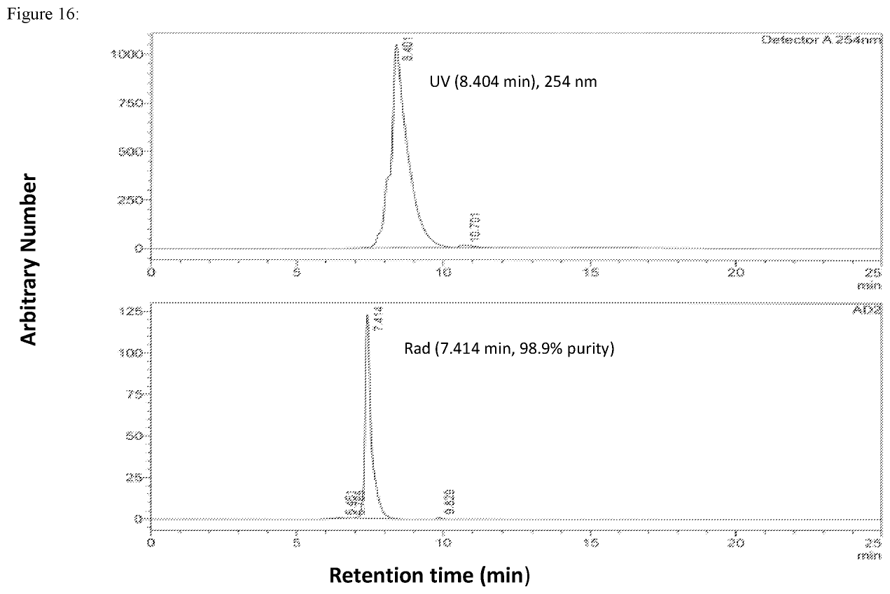

The same HPLC method used for unlabeled ⁶⁴Cu was also used for the ⁶⁴Cu-Conjugate 1. FIG. 16 shows the HPLC traces of the ⁶⁴Cu-Conjugate 1.

The radiolabeling yields for the ⁶⁴Cu-Conjugate 1 using varying reaction conditions are shown in Table 16 below. The molar activity (Aₘ) of ⁶⁴Cu-Conjugate 1 was 0.325 GBq/μmol.

TABLE 16

| | Volume of | | | | | Reaction | % |
|---|---|---|---|---|---|---|---|
| | Solution | Mass | Buffer | Temp | | time | Radiolabeling |
| Rxn | (μL) | (μg) | and pH | (C.) | pH | (minute) | Yield |
| 1 | 100 | 50 | 0.1M Sodium | 23 | 5 | 10 | 99.58 |
| | | | Acetate pH | | | | |
| | | | 5 in water | | | | |
| | | | | | 5 | 20 | 99.83 |
| | | | | | 5 | 30 | 99.40 |
| | | | | | 5 | 40 | 99.44 |
| 2 | 100 | 100 | 0.1M Sodium | 23 | 5 | 10 | 99.51 |
| | | | Acetate pH | | | | |
| | | | 5 in water | | | | |
| | | | | | 5 | 20 | 96.45 |
| | | | | | 5 | 30 | 99.63 |
| | | | | | 5 | 40 | 99.33 |
| 3 | 100 | 100 | 0.1M Sodium | 23 | 5 | 20 | 99.77 |
| | | | Acetate pH | | | | |
| | | | 5 in water | | | | |

### Example 6—⁶⁴Cu-Labeling of Conjugate 2

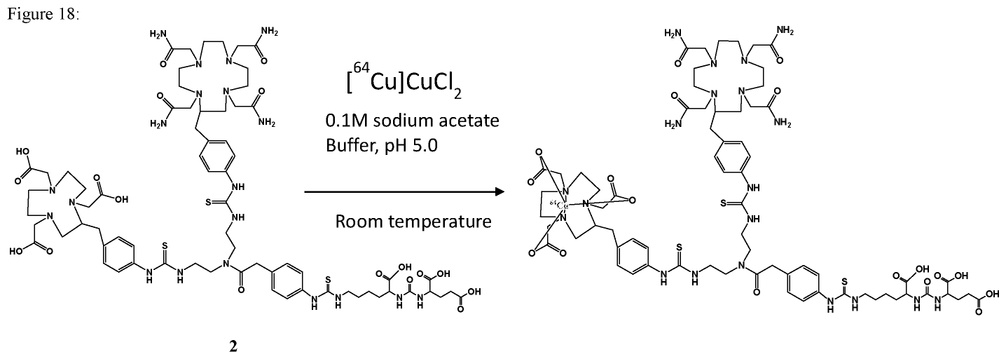

As shown in FIG. 18, Conjugate 2 was labeled with ⁶⁴Cu to form the ⁶⁴Cu-Conjugate 2. Conjugate2 was radiolabeled with Cu-64 using [⁶⁴Cu]CuCl₂ produced from cyclotron, formulated in 0.1M hydrochloric acid. Different amounts (50 μg, 100 μg) of Conjugate2 were used and the pH was adjusted to 5.0 using 0.1M sodium acetate after addition of [⁶⁴Cu]CuCl₂. The resultant reaction mixture was stirred at room temperature for different time points as 10 minutes, 20 minutes and 30 minutes to optimize radiolabeling yield as function of reaction time. The progress and yield of the reactions were monitored by radioactive thin layer chromatography (r-TLC) using iTLC (silica gel coated on paper, Agilent Technologies Inc., Santa Clara, CA) and 0.1 M sodium citrate as a mobile phase. Based on our tested radiolabeling condition, the reaction achieved >99% yield radiolabeling in 10 minutes and at all other timepoints at pH 5.0 using sodium acetate as a reaction buffer via stirring at room temperature. Our radiolabeling yields, as function of reaction time, temperature, and mass of the starting Conjugate2 are summarized in Table 22. Formation of radiolabeled ⁶⁴Cu-Conjugate2 was also confirmed by radio HPLC.

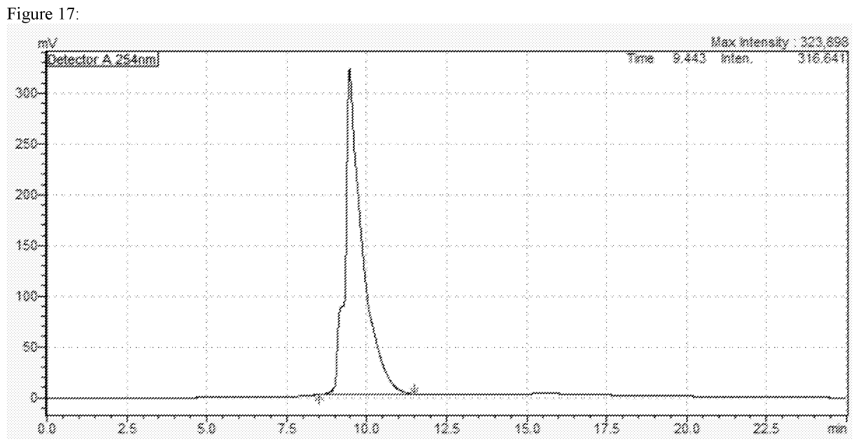

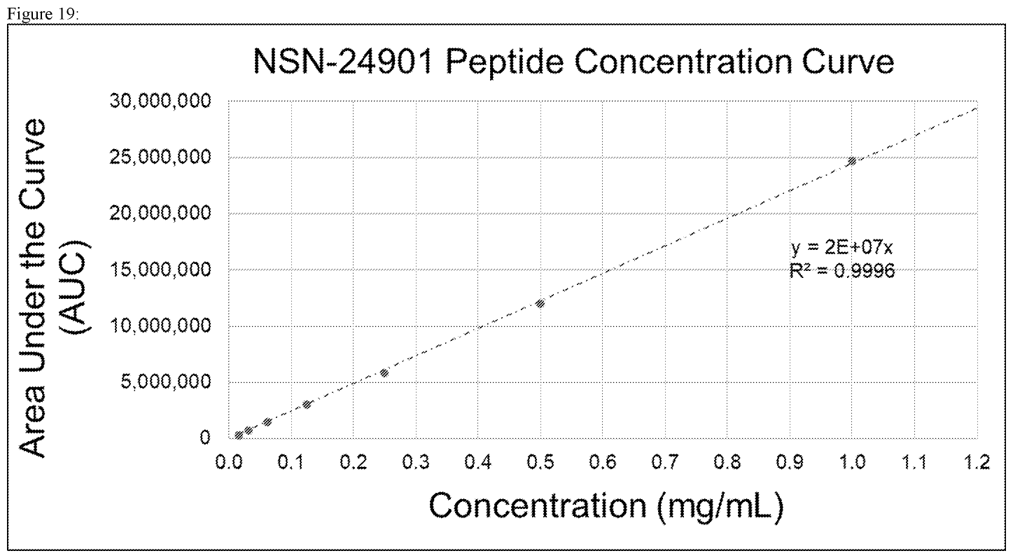

As shown in FIG. 17, an HPLC method was developed for Conjugate 2. The method used a Schmadzu HPLC system, which was equipped with dual UV and radioactivity detectors. The method was developed and optimized using a reverse phase HPLC column (C-18) from Phenomenex (Luna 5 μm C18(2) 100 Å LC Column 250×4.6 mm, (00G-4252-e0) using a UV wavelength of 254 nm. For analyte analysis, a 20 μL injection loop was installed and used for all analysis at room temperature. For mobile phase, a dual solvent system was used composed of solvent A as 0.1% trifluoroacetic acid (TFA) in acetonitrile and solvent B as 0.1% TFA in water. For peak separation, we used a gradient method as described in Table 18B using 1.0 mL/minute flow rate of the mobile phase. Table 17 below shows the results of the HPLC method. Conjugate 2 was tested with varying concentrations (Table 18A below). FIG. 19 shows the HPLC calibration curve of Conjugate 2 at varying concentrations. For analysis, the compound was dissolved in water and a calibration curve was prepared to estimate the specific activity of the synthesized compound. Depending upon the chemical nature of the compound a different retention time was observed.

TABLE 17

| | Retention Time | Area under the curve | |
|---|---|---|---|
| Peak | (minute) | (AUC) | % AUC |
| 1 | 9.47 | 12020852 | 100 |

TABLE 18A

| Number of | | | |
|---|---|---|---|
| samples | Concentration | Area Under the Curve | Retention Time |
| injected | (μg/mL) | (AUC) | (minute) |
| 1 | 1000 | 24702629 | 9.995 |
| 2 | 500 | 12020852 | 9.467 |
| 3 | 250 | 5848783 | 9.441 |
| 4 | 125 | 3012082 | 9.695 |
| 5 | 62.5 | 1489166 | 9.413 |
| 6 | 31.25 | 692468 | 9.708 |
| 7 | 15.6 | 281256 | 9.719 |

TABLE 18B

| | % of solvent A | % of solvent B |
|---|---|---|
| Time | (0.1% TFA in acetonitrile) | (0.1% TFA in water) |
| 0.01 minutes | 5 | 95 |
| 12.0 minutes | 55 | 45 |
| 23.0 minutes | 5 | 95 |
| 25.0 minutes | 5 | 95 |

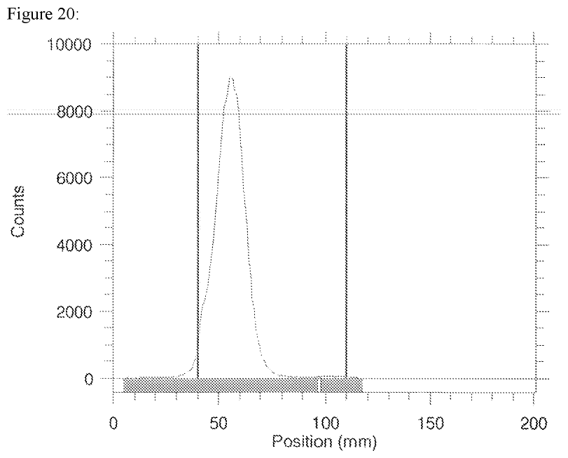

The TLC trace of the product, the ⁶⁴Cu-Conjugate 2, is shown in FIG. 20, using a silica gel solid phase and 0.1M sodium citrate mobile phase. Table 19 shows the TLC results.

TABLE 19

| | Start | Stop | Centroid | | Region | Region | % of | % of |
|---|---|---|---|---|---|---|---|---|
| Peak | (mm) | (mm) | (mm) | RF | Counts | CPM | Total | ROI |
| 1 | 4.9 | 96.9 | 55.4 | 0.220 | 183383.0 | 785927.1 | 99.28 | 99.40 |
| 2 | 97.8 | 117.4 | 105.7 | 0.939 | 1113.0 | 4770.0 | 0.60 | 0.60 |
| | | | | | 184496.0 | 790697.1 | 99.88 | 100.00 |

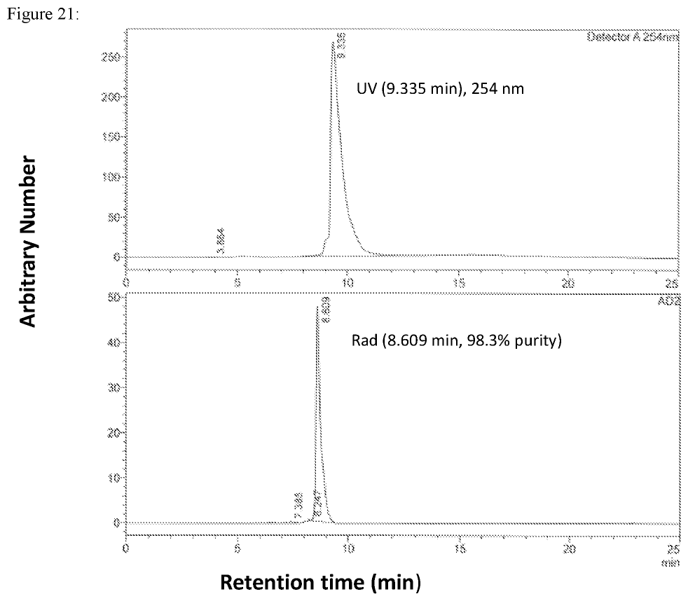

The same HPLC method used for Conjugate 2 was also used for the ⁶⁴Cu-Conjugate 2. FIG. 21 shows the r-HPLC traces of the ⁶⁴Cu-Peptide conjugate. Tables 20 and 21 show the HPLC results for the ⁶⁴Cu-Conjugate 2.

TABLE 20

| Peak # | Ret. Time | Area | % Conc. |
|---|---|---|---|
| 1 | 3.864 | 10451 | 0.106 |
| 2 | 9.335 | 9875720 | 99.894 |
| Total | | 9886171 | 100.00 |

TABLE 21

| Peak # | Ret. Time | Area | % Conc. |
|---|---|---|---|
| 1 | 7.385 | 12214 | 1.712 |
| 2 | 8.247 | −66 | −0.009 |
| 3 | 8.609 | 701191 | 98.297 |
| Total | | 713338 | 100.00 |

The ⁶⁴Cu-Conjugate 2 was tested for radiolabeling yields using varying reaction conditions (Table 22 below). The molar activity (Aₘ) of the ⁶⁴Cu-Conjugate 2 was 0.8-1.35 GBq/μmol.

TABLE 22

| | Volume of | | | | Reaction | % |
|---|---|---|---|---|---|---|
| | Solution | Mass | Buffer | Temp | time | Radiolabeling |
| Rxn | (μL) | (μg) | and pH | (C.) | (minute) | Yield |
| 4 | 100 | 50 | 0.1M Na | 23 | 10 | 99.56 |
| | | | Acetate pH | | | |
| | | | 5 in water | | | |
| | | | | | 20 | 99.63 |
| | | | | | 30 | 99.64 |
| 5 | 100 | 100 | 0.1M Na | 23 | 10 | 98.35 |
| | | | Acetate pH | | | |
| | | | 5 in water | | | |
| | | | | | 20 | 97.90 |
| | | | | | 30 | 99.42 |
| 6 | 300 | 100 | 0.1M Na | 23 | 30 | 99.62 |
| | | | Acetate pH | | | |
| | | | 5 in water | | | |
| 7 | 300 | 100 | 0.1M Na | 23 | 30 | 99.87 |
| (1) | | | Acetate pH | | | |
| | | | 5 in water | | | |

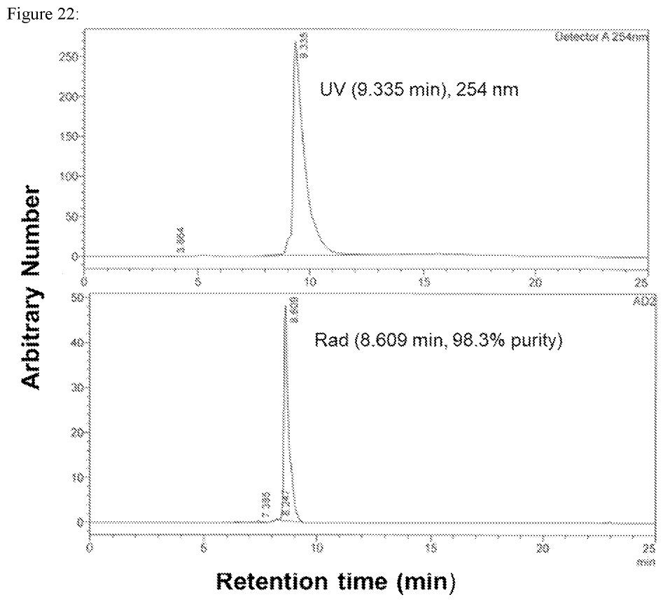

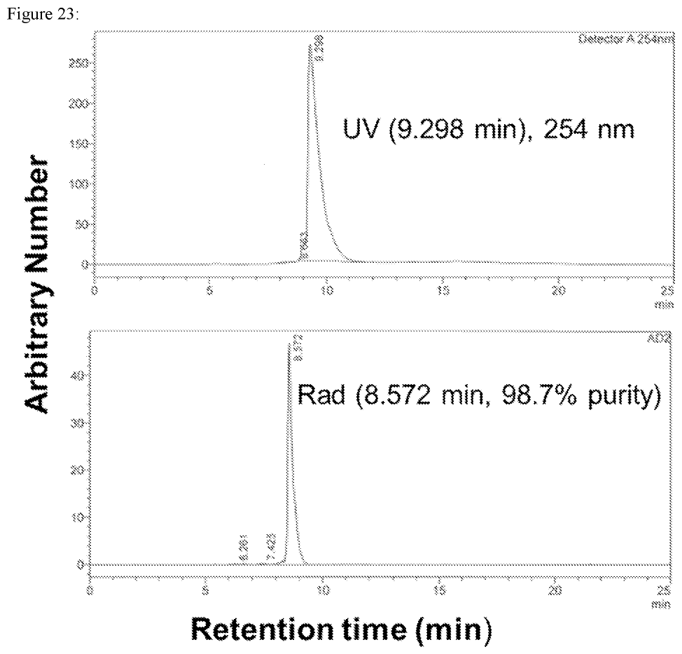

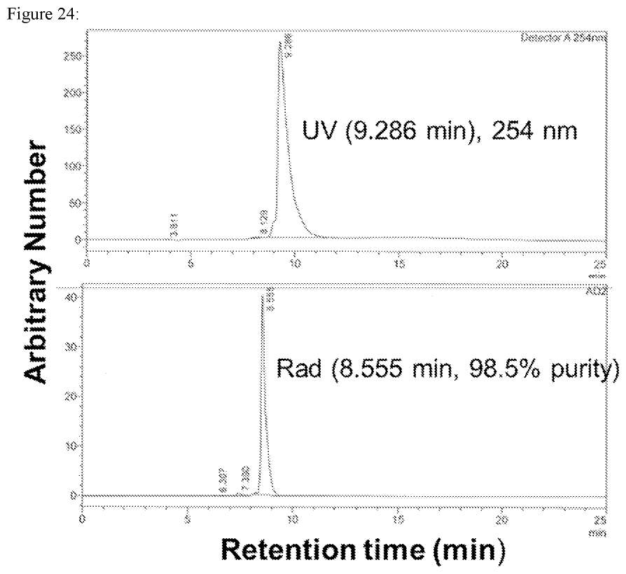

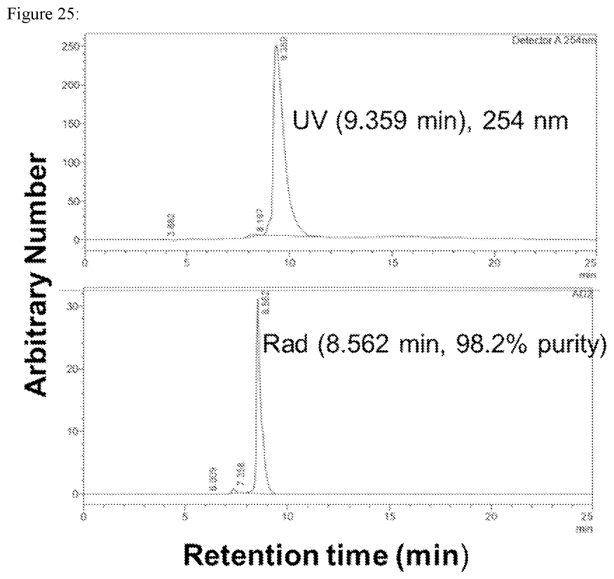

The stability of the ⁶⁴Cu-Conjugate 2 was tested using the same HPLC method as the Conjugate 2 at various time points. The time points included: 40 minutes (Tables 23-24 and FIG. 22), 2 hours (Tables 25-26 and FIG. 23), 4 hours (Tables 27-28 and FIG. 24), and 8 hours (Tables 29-30 and FIG. 25).

TABLE 23

| Peak # | Ret. Time | Area | % Conc. |
|---|---|---|---|
| 1 | 3.864 | 10451 | 0.106 |
| 2 | 9.335 | 9875720 | 99.894 |
| Total | | 9886171 | 100.00 |

TABLE 24

| Peak # | Ret. Time | Area | % Conc. |
|---|---|---|---|
| 1 | 7.385 | 12214 | 1.712 |
| 2 | 8.247 | −66 | −0.009 |
| 3 | 8.609 | 701191 | 98.297 |
| Total | | 713338 | 100.00 |

TABLE 25

| Peak | Ret. Time | Area | % Conc. |
|---|---|---|---|
| 1 | 8.663 | 21833 | 0.230 |
| 2 | 9.298 | 9454504 | 99.770 |
| Total | | 9476338 | |

TABLE 26

| Peak | Ret. Time | Area | % Conc. |
|---|---|---|---|
| 1 | 6.261 | 3827 | 0.532 |
| 2 | 7.425 | 5398 | 0.750 |
| 3 | 8.572 | 710384 | 98.718 |
| Total | | 719608 | |

TABLE 27

| Peak | Ret. Time | Area | % Conc. |
|---|---|---|---|
| 1 | 3.811 | 9223 | 0.097 |
| 2 | 8.129 | 41821 | 0.439 |
| 3 | 9.286 | 9464636 | 99.463 |
| Total | | 9515690 | |

TABLE 28

| Peak | Ret. Time | Area | % Conc. |
|---|---|---|---|
| 1 | 6.307 | 773 | 0.128 |
| 2 | 7.390 | 8595 | 1.42 |
| 3 | 8.555 | 596042 | 98.453 |
| Total | | 605410 | |

TABLE 29

| Peak | Ret. Time | Area | % Conc. |
|---|---|---|---|
| 1 | 3.862 | 10209 | 0.1110 |
| 2 | 8.197 | 72329 | 0.782 |
| 3 | 9.359 | 9164041 | 99.107 |
| Total | | 9246580 | |

TABLE 30

| Peak | Ret. Time | Area | % Conc. |
|---|---|---|---|
| 1 | 6.009 | 59 | 0.012 |
| 2 | 7.358 | 9036 | 1.815 |
| 3 | 8.562 | 488865 | 98.173 |
| Total | | 497960 | |

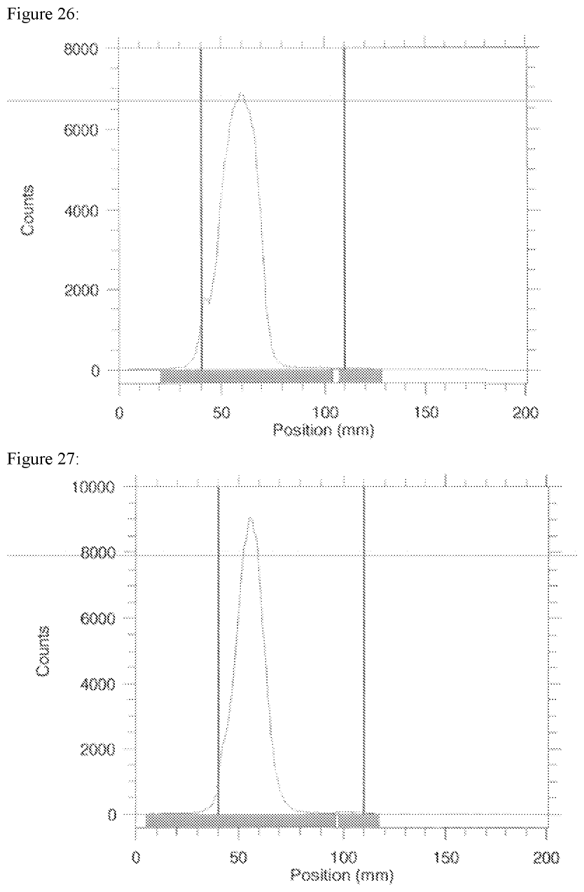

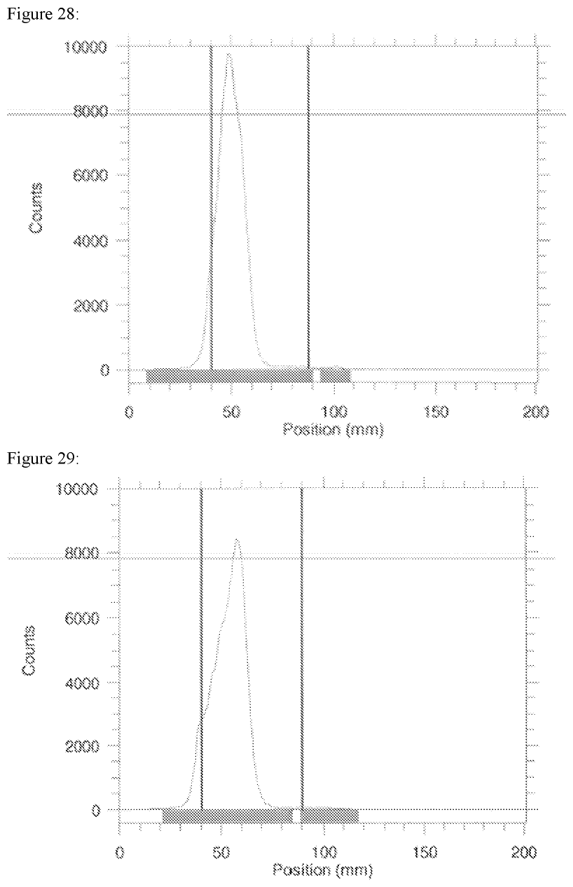

The stability of the ⁶⁴Cu-Conjugate 2 was also analyzed by TLC using the same TLC method as the ⁶⁴Cu-Conjugate 1 at various time points. The time points included: 40 minutes (Table 31 and FIG. 26), 2 hours (Table 32 and FIG. 27), 4 hours (Table 33 and FIG. 28), 8 hours (Table 34 and FIG. 29).

TABLE 31

| | Start | Stop | Centroid | Region | Region | | % of |
|---|---|---|---|---|---|---|---|
| Region | (mm) | (mm) | (mm) | Counts | CPM | RF | ROI |
| Rgn 1 | 20.2 | 103.7 | 58.4 | 183691.0 | 785927.1 | 0.263 | 99.62 |
| Rgn 2 | 107.1 | 128.4 | 115.1 | 698.0 | 2326.7 | 1.072 | 0.38 |

TABLE 32

| | Start | Stop | Centroid | Region | Region | | % of |
|---|---|---|---|---|---|---|---|
| Region | (mm) | (mm) | (mm) | Counts | CPM | RF | ROI |
| Rgn 1 | 4.9 | 96.9 | 55.4 | 183383.0 | 785927.1 | 0.220 | 99.40 |
| Rgn 2 | 97.8 | 117.4 | 105.7 | 1113.0 | 4770.0 | 0.939 | 0.60 |

TABLE 33

| | Start | Stop | Centroid | Region | Region | | % of |
|---|---|---|---|---|---|---|---|
| Region | (mm) | (mm) | (mm) | Counts | CPM | RF | ROI |
| Rgn 1 | 8.3 | 90.1 | 49.7 | 183095.0 | 477639.1 | 0.202 | 99.46 |
| Rgn 2 | 94.4 | 108.0 | 100.1 | 666.0 | 1737.4 | 1.252 | 0.36 |

TABLE 34

| | Start | Stop | Centroid | Region | Region | | % of |
|---|---|---|---|---|---|---|---|
| Region | (mm) | (mm) | (mm) | Counts | CPM | RF | ROI |
| Rgn 1 | 21.1 | 85.0 | 53.4 | 18319.0 | 526625.7 | 0.268 | 99.19 |
| Rgn 2 | 89.3 | 117.4 | 99.7 | 1500.0 | 4285.7 | 1.194 | 0.81 |

The stability of the ⁶⁴Cu-Conjugate 2 was tested in mouse serum and human serum at 37° C. using the Rad-iTLC method. Approximately 1.0 mL of mouse serum was extracted from blood of the mice and ˜1.0 mL of human serum was extracted from blood obtained from the Mayo Clinic's blood bank to measure the stability of radiolabeled ⁶⁴Cu-Conjugate 2. Obtained mouse and human serums were distributed separately in 100 μL aliquots in 1.5 mL microcentrifuge tubes (n=3). To which, 20 μL of ⁶⁴Cu-Conjugate2 was added in each 100 μL serum aliquot and mixed thoroughly. From this mixture, a small amount of reaction mixture was taken out using glass capillary tube and immediately spotted on an iTLC plate for analysis as T=0 timepoint (n=3). The rest of the reaction mixtures were incubated at 37° C. for up to 2 hours and small fractions were taken out at 1 hour and 2 hours post incubation to analyze the stability of ⁶⁴Cu-Conjugate 2 over time using radioactive thin layer chromatography (r-TLC). To do r-TLC analysis, iTLC (silica gel coated on paper, Agilent Technologies Inc., Santa Clara, CA) was used as a solid phase and 0.1M sodium citrate as a mobile phase. In this r-TLC condition, unconjugated (free)⁶⁴Cu moves to the solvent front of the r-TLC and radiolabeled ⁶⁴Cu-Conjugate 2 stays at the origin of the r-TLC plate. Based on the relative % of radioactivity at origin and at the solvent front, the % of intact ⁶⁴Cu-Conjugate2 and free ⁶⁴Cu was measured as presented in Tables 35 and 36. The results of the stability testing are shown in Table 35 below. The ⁶⁴Cu-Conjugate 2 was found to be stable up to 2 hours in mouse serum.

TABLE 35

| | | | Cu-64 labeled | | | | | | |
|---|---|---|---|---|---|---|---|---|---|
| | Mouse | [⁶⁴Cu]Cu- | tracer (μCi) | T = 0 | | T = 1 h | | T = 2 h | |
| Tube | Serum | Conjugate | and time of | Intact | Broken | Intact | Broken | Intact | Broken |
| # | (μL) | 2 (μL) | measurement | (%) | (%) | (%) | (%) | (%) | (%) |
| 1 | 100 | 20 | 30.76 | 98.25 | 1.75 | 96.86 | 3.14 | 97.43 | 2.57 |
| 2 | 100 | 20 | 30.76 | 98.38 | 1.62 | 96.9 | 3.1 | 97.31 | 2.69 |
| 3 | 100 | 20 | 30.76 | 98.14 | 1.86 | 96.72 | 3.28 | 98.08 | 1.92 |
| | | | Average | 98.26 | 1.74 | 96.83 | 3.17 | 97.61 | 2.39 |
| | | | SD | 0.12 | 0.12 | 0.09 | 0.09 | 0.41 | 0.41 |

TABLE 36

| | | Cu-64 | Cu-64 labeled | | | | | | |
|---|---|---|---|---|---|---|---|---|---|
| | Human | labeled | tracer (μCi) | T = 0 | | T = 1 h | | T = 2 h | |
| Tube | Serum | tracer | and time of | Intact | Broken | Intact | Broken | Intact | Broken |
| # | (μL) | (μL) | measurement | (%) | (%) | (%) | (%) | (%) | (%) |
| 1 | 100 | 30 | 53 | 99.68 | 0.32 | 99.79 | 0.21 | 99.63 | 0.37 |
| | | | 13:38 | | | | | | |
| 2 | 100 | 30 | 53 | 99.67 | 0.33 | 99.79 | 0.21 | 99.84 | 0.16 |
| | | | 13:38 | | | | | | |
| 3 | 100 | 30 | 53 | 99.71 | 0.29 | 99.59 | 0.24 | 99.79 | 0.21 |
| | | | 13:38 | | | | | | |

| | | | | | | |
|---|---|---|---|---|---|---|
| Average | 99.69 | 0.31 | 99.72 | 0.22 | 99.75 | 0.25 |
| SD | 0.02 | 0.02 | 0.12 | 0.02 | 0.11 | 0.11 |

The cellular uptake of the ⁶⁴Cu-Conjugate 2 was studied using LNCaP cells. The cellular uptake of the ⁶⁴Cu-Conjugate 2 was studied using LNCaP cells. The LNCaP cells were from American Type Culture Collection, Manassas, VA, and were cultured in Corning® BioCoat™ Poly-Lysine 6 well plate (Corning, Glendale, AZ) in complete Roswell Park Memorial Institute (RPMI) 1640 medium with 10% fetal bovine serum (FBS) (Gibco-ThermoFisher Scientific, Waltham, MA) and 1 time with Penicillin/Streptomycin (Gibco-ThermoFisher Scientific, Waltham, MA) in a CO₂ incubator at 37° C. On the day of the uptake experiment, the cell culture medium of wells culturing the cells was changed to preincubation medium (RPMI 1640 with 5% Bovine Serum Albumin (BSA)), and cells were preincubated for 60 minutes. Following preincubation, the cells were re-incubated in RPMI1640 medium having 5% BSA with ⁶⁴Cu-Conjugate2 (1.4±0.22 MBq/well at the beginning of incubation) for 60 minutes at 37° C. Following incubation with ⁶⁴Cu-Conjugate2, the cells were washed 3 times with chilled phosphate buffered saline (PBS) with or without 10 μM 2-(phosphonomethyl)pentane-1,5-dioic acid (PMPA). The PMPA is a potent PSMA inhibitor. The cells washed with 10 μM PMPA gave the information about uptake contributed by internalization of ⁶⁴Cu-Conjugate 2, whereas the cells washed without PMPA gave the estimation of uptake contributed by both internalization and cell membrane binding of ⁶⁴Cu-Conjugate2. For negative control, the cells were exposed to 100 μM PMPA at the preincubation and incubation steps. Following final washing, the cells were collected from the wells, and radioactivity was counted in gamma counter. The uptake was calculated as per following formula:

% Uptake=(Decay corrected radioactivity in cells after washing/Decay corrected radioactivity in incubation medium)×100.

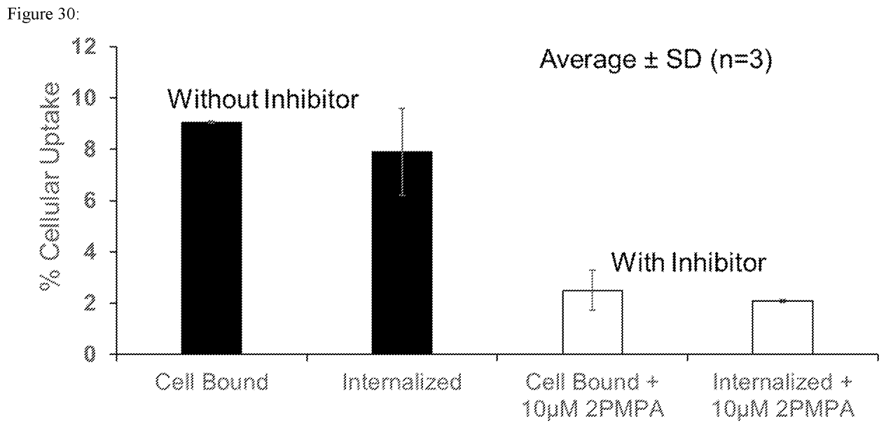

The molar activity the ⁶⁴Cu-Conjugate 2 was 1.35 GBq/μmol. The concentration per well was 1.52 nmols, with a cell number of 6.5×10⁵ per well in a 6 well plate. The % cellular uptake is shown in FIG. 30.

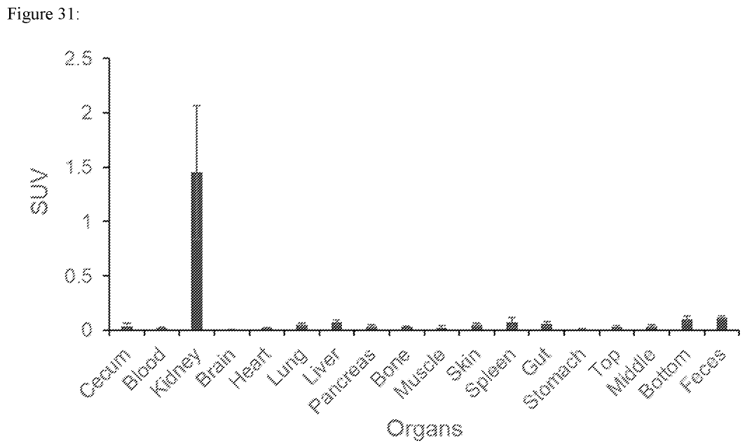

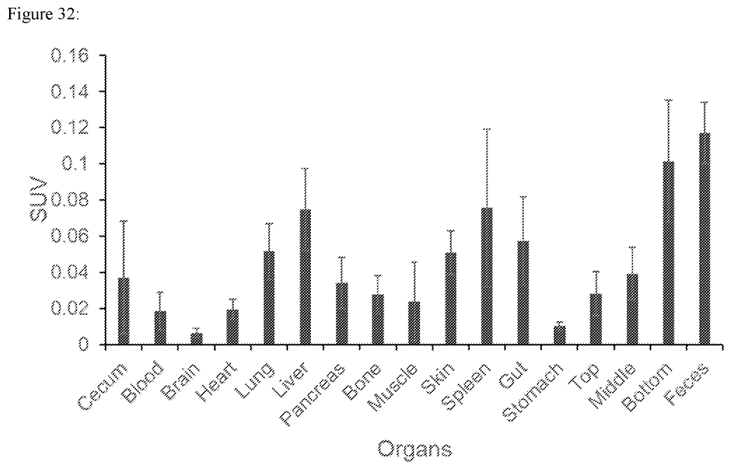

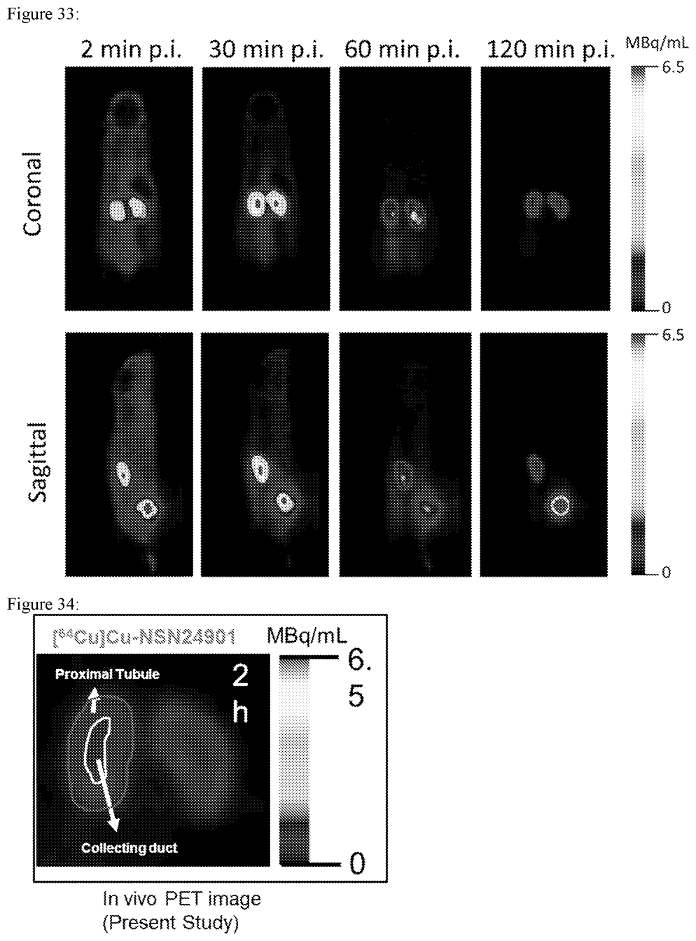

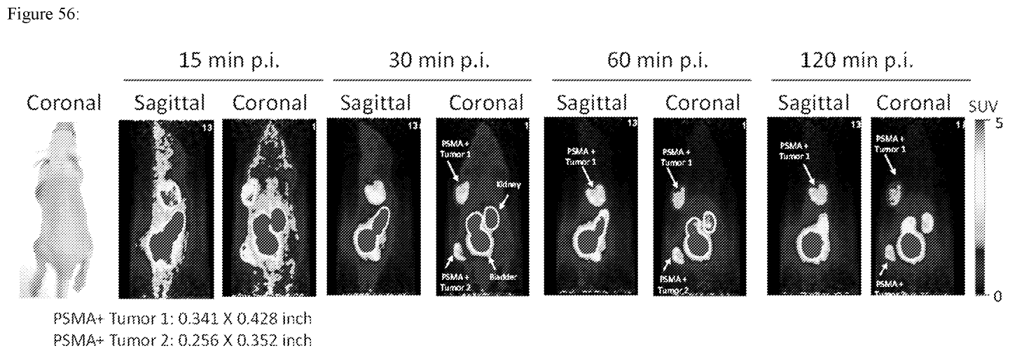

The in vivo evaluation of the ⁶⁴Cu-Conjugate 2 two hours post injection of normal nude mice (strain: 002019, NU/J) is shown in FIGS. 31 and 32. Micro PET imaging was done on normal mice with the ⁶⁴Cu-Conjugate 2 at different time intervals and is shown in FIG. 33. Micro PET imaging was done on normal mice (strain: 002019, NU/J) and athymic nude mice bearing LNCaP tumors with the ⁶⁴Cu-Conjugate 2 at different time intervals and is shown in FIG. 33 and FIG. 56. The ⁶⁴Cu-Conjugate2 (5.64±0.25 MBq, 52 GBq/μmol; n=3) was injected into normal and athymic nude mice bearing LNCaP tumors. PET images (10 minutes static) were acquired at 30 minutes, 60 minutes and 120 minutes post-injection using small animal PET system (Sofie BioSystems Genesys4, Culver City, CA, USA). The acquired PET images were analyzed using image analysis software, AMIDE (Amide's a Medical Imaging Data Examiner) for calculation of uptake as Standardized Uptake Value (SUV), SUVₘₐₓ and SUVₘₑₐₙ by drawing region of interest (ROI). Following final image acquisition, the animals were euthanized, and tumor tissue and major organs of interest like kidney were harvested for gamma counting for ex-vivo biodistribution. The uptake as SUV in tissues of interest were calculated as per following formula:

SUVof tissue of interest=((activity/mL in tissue of interest)/(injected dose))×animal weight

in accordance with Loening AM and Gambhir SS. AMIDE: a free software tool for multimodality medical image analysis. Mol Imaging 2003; 2:131-7. It was found that the ⁶⁴Cu-Conjugate 2 accumulates in proximal tubules in the kidney where high prostate specific membrane antigen (PSMA) expression is known (FIG. 34). The results showed that the ⁶⁴Cu-Conjugate 2 was well tolerated by the animals and reached the expected organs of the body.

### Example 7—²⁰³Pb-Labeling of Conjugate 1

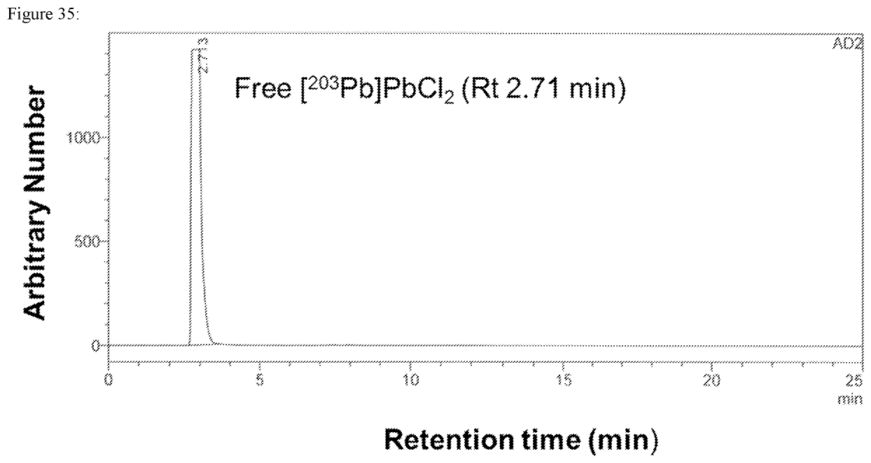

The same HPLC method was used to analyze unlabeled ^{203/212}Pb as was used for ⁶⁴Cu described above. The HPLC trace is shown in FIG. 35 and Table 37 below.

TABLE 37

| | Ret. Time | | | |
|---|---|---|---|---|
| Peak | (minute) | Area | Height | % Concentration |
| 1 | 2.713 | 32630652 | 1418138 | 100.00 |
| | | 32630652 | 1418138 | 100.00 |

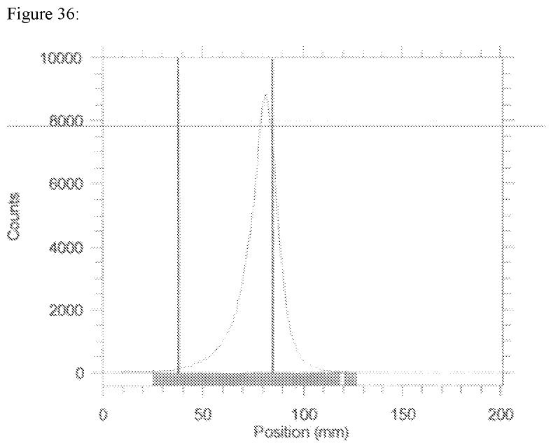

A TLC method was developed for unlabeled ^{203/212}Pb, the method included a silica gel (iTLC) solid phase and a 0.15M NH₄Ac, pH 4.0 mobile phase. The TLC results are shown in FIG. 36 and Table 38.

TABLE 38

| | Start | Stop | Centroid | | Region | Region | % of | % of |
|---|---|---|---|---|---|---|---|---|
| Peak | (mm) | (mm) | (mm) | RF | Counts | CPM | Total | ROI |
| 1 | 25.3 | 118.2 | 78.6 | 0.863 | 192230.0 | 1153380.0 | 99.45 | 99.95 |
| 2 | 120.8 | 126.7 | 99.5 | 0.01 | 97.0 | 582.0 | 0.05 | 0.05 |
| | | | | | 192327.0 | 1153962.0 | 99.50 | 100.00 |

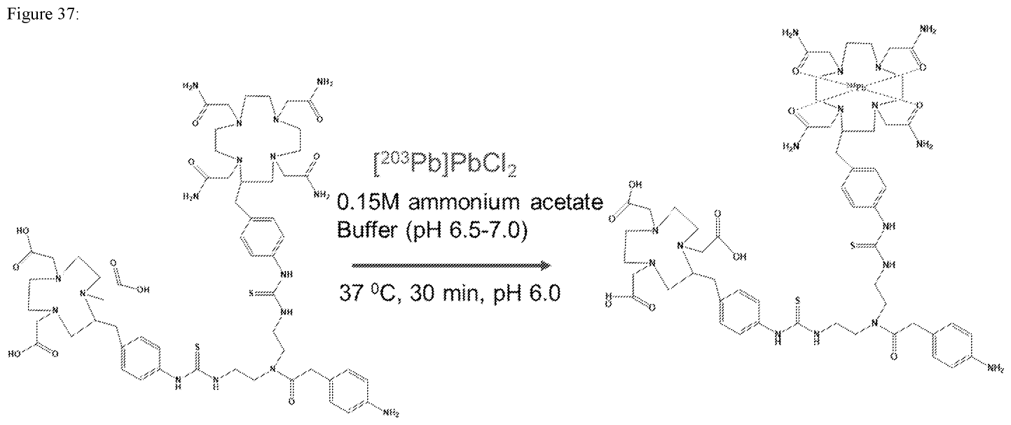

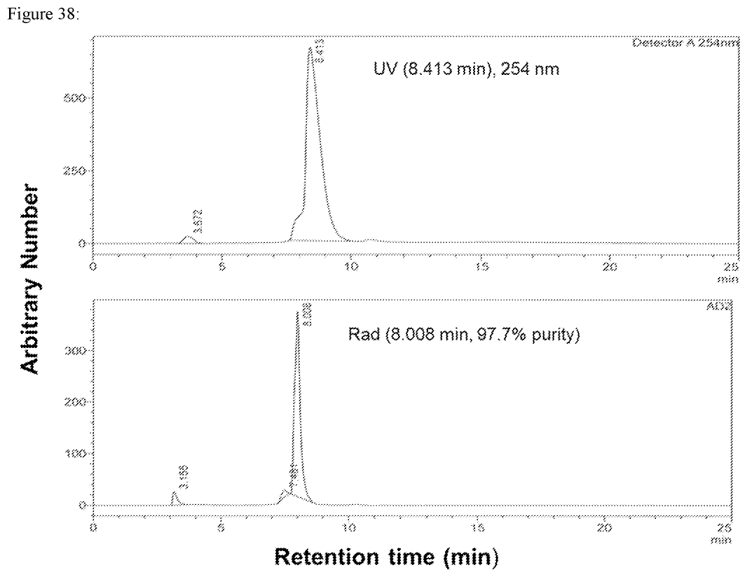

As shown in FIG. 37, Conjugate 1 was labeled with ²⁰³Pb to form ²⁰³Pb-Conjugate 1. To radiolabel Conjugate-1 with ²⁰³Pb, radioactive Pb-203 as [²⁰³Pb]PbCl₂ was used as a surrogate radioisotope for Pb-212 to test the feasibility of radiolabeling. Approximately, 100 μg of Conjugate-1 was used and the pH was adjusted to 6.0 using 0.15M ammonium acetate (pH 6.5-7.0) after addition of [²⁰³Pb]PbCl₂, the resultant reaction mixture was stirred at 37° C. for 10 minutes, and 30 minutes to optimize radiolabeling yield with reaction time. The progress and yield of the reactions were monitored by radioactive thin layer chromatography (r-TLC) using iTLC (silica gel coated on paper, Agilent Technologies Inc., Santa Clara, CA) as a solid phase and 0.15M NH₄Ac, pH 4.0 as a mobile phase. In this r-TLC condition, unconjugated (Free)²⁰³Pb moves to the solvent front of the r-TLC and radiolabeled ²⁰³Pb-Conjugate 1 stays at the origin of the r-TLC plate. Based on the tested radiolabeling condition, the reaction achieved >99% yield radiolabeling in 30 minutes at pH 6.0 using 0.15M ammonium acetate as a reaction buffer via stirring at 37° C. The radiolabeling yields, as function of reaction time, temperature, and mass of the starting Conjugate-1 are summarized in Table 39. Formation of radiolabeled product ²⁰³Pb-Conjugate-1 was also confirmed by radio HPLC. The same HPLC method used for ⁶⁴Cu was also used for the ²⁰³Pb-Conjugate 1. FIG. 38 shows the HPLC traces of the ²⁰³Pb-Conjugate 1. The molar activity (Aₘ) of the ²⁰³Pb-Conjugate 1 was 0.107 GBq/μmol.

TABLE 39

| | Volume of | | | | | Reaction | % |
|---|---|---|---|---|---|---|---|
| | Solution | Mass | | Temp | | time | Radiolabeling |
| Rxn | (μL) | (μg) | Buffer and pH | (C.) | pH | (minute) | Yield |
| 1 | 100 | 100 | 0.15M | 37 | 6.0 | 30.0 | 99.88 |
| | | | ammonium acetate | | | | |
| | | | (pH 6.5-7.0) | | | | |
| | | | Reaction pH 6.0 | | | | |

### Example 8—²⁰³Pb-Labeling of Conjugate 2

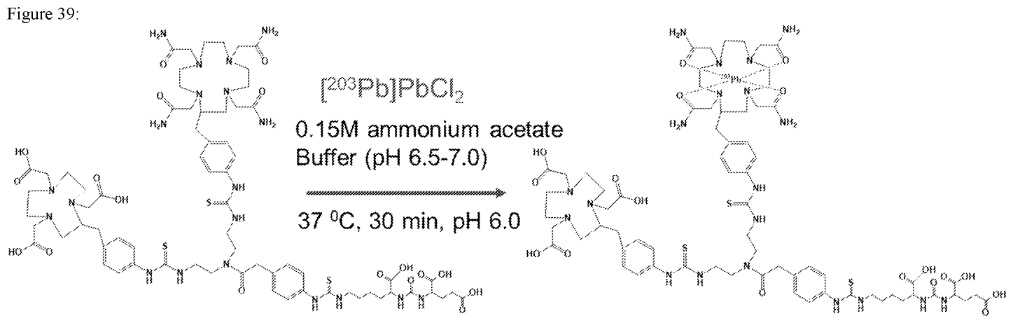

As shown in FIG. 39, Conjugate 2 was labeled with ²⁰³Pb to form the ²⁰³Pb-Conjugate 2. To radiolabel Conjugate 2 with ²⁰³Pb, radioactive Pb-203 was used as [²⁰³Pb]PbCl₂, a surrogate radioisotope for Pb-212 to test the feasibility of radiolabeling. Approximately, 200 μg of Conjugate-2 was used and the pH was adjusted to 6.0 using 0.15M ammonium acetate (pH 6.5-7.0) after addition of [²⁰³Pb]PbCl₂. The resultant reaction mixture was stirred at 37° C. for 30 minutes. The progress and yield of the reaction was monitored by radioactive thin layer chromatography (r-TLC) using iTLC (silica gel coated on paper, Agilent Technologies Inc., Santa Clara, CA) as a solid phase and 0.15M NH₄Ac, pH 4.0 as a mobile phase. In this r-TLC condition, unconjugated (Free)²⁰³Pb moves to the solvent front of the r-TLC and radiolabeled ²⁰³Pb-Conjugate 2 stays at the origin of the r-TLC plate. Based on our tested radiolabeling condition, the reaction achieved >99% yield radiolabeling in 30 minutes at pH 6.0 using 0.15M ammonium acetate as a reaction buffer via stirring at 37° C. The radiolabeling yields, as function of reaction time, temperature, and mass of the starting Conjugate 2 are summarized in Table 43. Formation of radiolabeled product ²⁰³Pb-Conjugate 2 was also confirmed by radio HPLC.

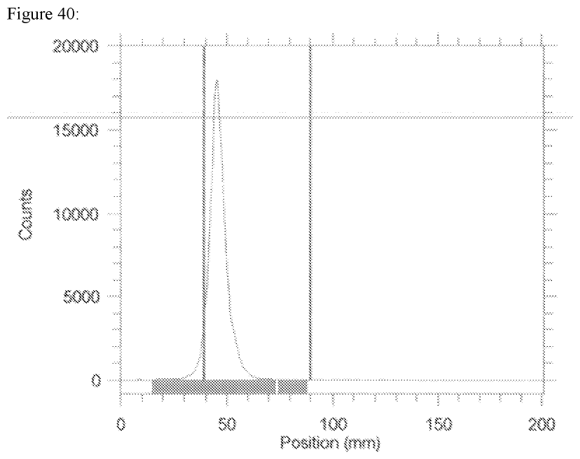

The same TLC method that was used to analyze the ²⁰³Pb-Conjugate 1 was used to analyze the ²⁰³Pb-Conjugate 2. The TLC trace is shown in FIG. 40 and Table 40 below.

TABLE 40

| | Start | Stop | Centroid | | Region | Region | % of | % of |
|---|---|---|---|---|---|---|---|---|
| Peak | (mm) | (mm) | (mm) | RF | Counts | CPM | Total | ROI |
| 1 | 15.1 | 73.1 | 45.9 | 0.135 | 186401.0 | 657885.9 | 99.35 | 99.82 |
| 2 | 74.8 | 88.4 | 80.9 | 0.821 | 338.0 | 1192.9 | 0.18 | 0.18 |
| | | | | | 186739.0 | 659078.8 | 99.53 | 100.00 |

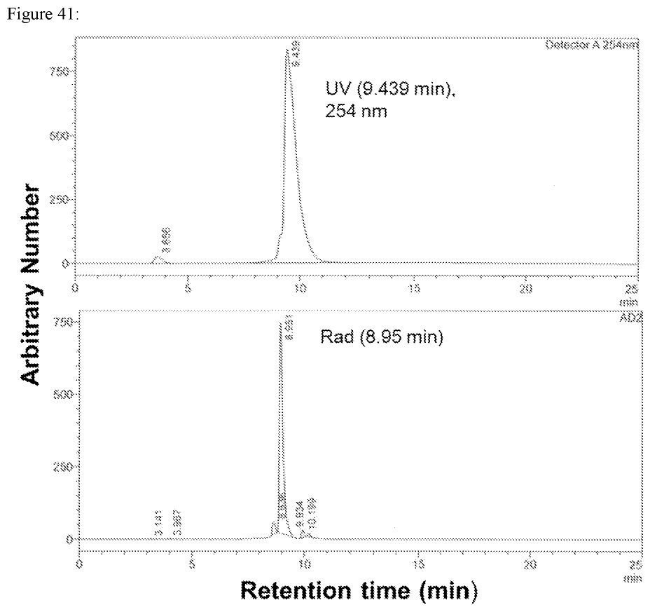

The same HPLC method that was used to analyze the ²⁰³Pb-Conjugate 1 was used to analyze the ²⁰³Pb-Conjugate 2. The HPLC trace is shown in FIG. 41 and Tables 31 and 32 below.

TABLE 41

| Peak # | Ret. Time | Area | % Conc. |
|---|---|---|---|
| 1 | 3.656 | 705084 | 2.126 |
| 2 | 9.439 | 834553 | 97.874 |
| Total | | 862608 | 100.00 |

TABLE 42

| Peak # | Ret. Time | Area | % Conc. |
|---|---|---|---|
| 1 | 3.141 | 12826 | 0.149 |
| 2 | 3.967 | 21936 | 0.255 |
| 3 | 8.636 | 352962 | 4.096 |
| 4 | 8.951 | 7931818 | 92.040 |
| 5 | 9.934 | 210714 | 2.445 |
| 6 | 10.199 | 87509 | 1.015 |
| Total | | 8617765 | 100.00 |

The TLC method developed for unlabeled ^{203/212}Pb was used to measure reaction yield of the ²⁰³Pb-Conjugate 2 and is shown in Table 43 below. The molar activity (Aₘ) of the ²⁰³Pb-Conjugate 2 was 0.299 GBq/μmol.

TABLE 43

| | Volume of | | | | Reaction | % |
|---|---|---|---|---|---|---|
| | Solution | Mass | Buffer | Temp | time | Radiolabeling |
| Rxn | (μL) | (μg) | and pH | (C.) | (minute) | Yield |
| 2 | 100 | 200 | 0.15M | 37 | 30 | 99.82 |
| | | | ammonium acetate | | | |
| | | | (pH 6.5-7.0) | | | |
| | | | Reaction pH 6.0 | | | |

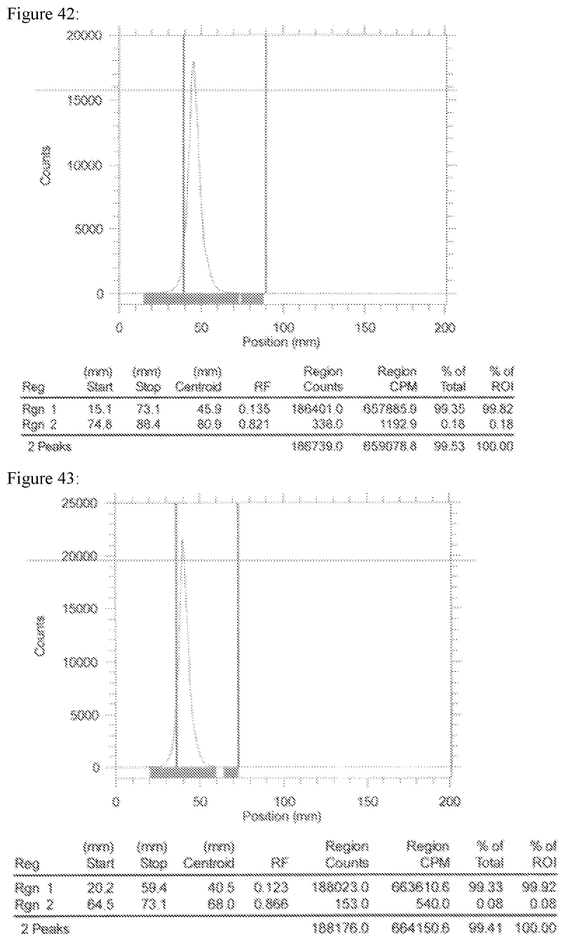

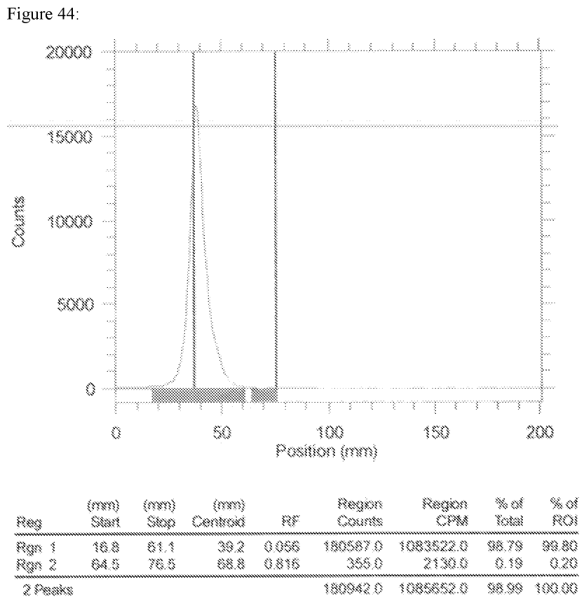

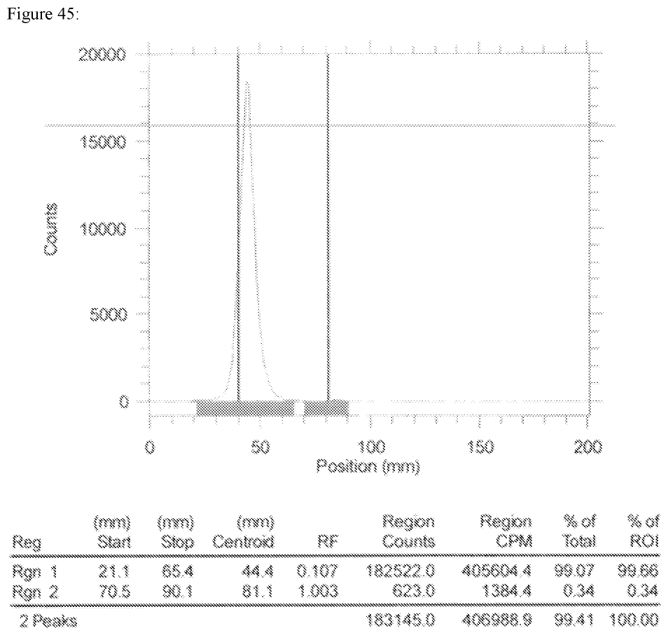

The stability of the ²⁰³Pb-Conjugate 2 was also analyzed by TLC at various time points using the same TLC method as unlabeled ²⁰³Pb. The time points included: 40 minutes (FIG. 42), 2 hours (FIG. 43), 4 hours (FIG. 44), 21 hours (FIG. 45). The results showed that the ²⁰³Pb-Conjugate 2 was stable up to 21 hours.

### Example 9—Mixed Labeling of Conjugate 2 with ⁶⁴Cu and ²⁰³Pb

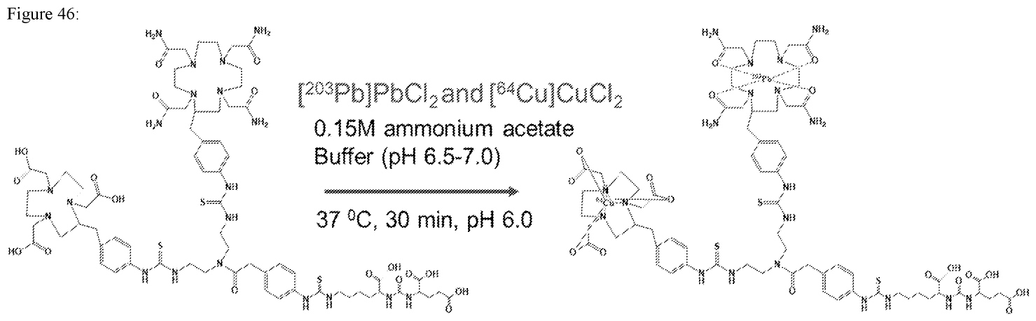

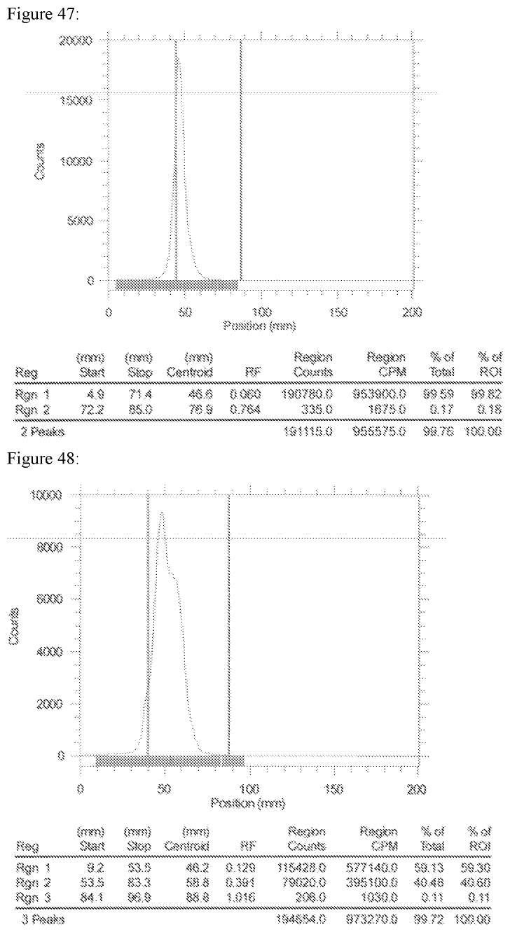

As shown in FIG. 46, Conjugate 2 was labeled with both ²⁰³Pb and ⁶⁴Cu to form the mixed labeled conjugate, ⁶⁴Cu/²⁰³Pb-Conjugate 2. Since Conjugate 2 is designed as a theranostic molecule to serve both as an imaging and radiotherapy molecule, Conjugate2 was radiolabeled with both ²⁰³Pb and ⁶⁴Cu radioisotopes. However, in this experiment, ²⁰³Pb was used as a surrogate isotope for ²¹²Pb. Firstly, Conjugate 2 was radiolabeled with ²⁰³Pb, for which radioactive Pb-203 was used as [²⁰³Pb]PbCl₂. Approximately, 200 μg of Conjugate 2 was used, and the pH was adjusted to 6.0 using 0.15M ammonium acetate (pH 6.5-7.0) after addition of [²⁰³Pb]PbCl₂. The resultant reaction mixture was stirred at 37° C. for 20-30 minutes. The progress and yield of the reaction was monitored by radioactive thin layer chromatography (r-TLC) using iTLC (silica gel coated on paper, Agilent Technologies Inc., Santa Clara, CA) as a solid phase and 0.15M NH₄Ac, pH 4.0 as a mobile phase. In this r-TLC condition, unconjugated (Free)²⁰³Pb moves to the solvent front of the r-TLC, and radiolabeled ²⁰³Pb-Conjugate 2 stays at the origin of the r-TLC plate. Based on the tested radiolabeling condition, the reaction achieved >99% yield radiolabeling in 20-30 minutes at pH 6.0 using 0.15M ammonium acetate as a reaction buffer via stirring at 37° C. The TLC results are shown in FIG. 47. After confirming radiolabeling with ²⁰³Pb, the temperature of the reaction was adjusted to room temperature, and Cu-64 as [⁶⁴Cu]CuCl₂ produced from cyclotron was added. The pH was adjusted to 5.0 using 0.1M sodium acetate, and the resultant reaction mixture was stirred for additional 10 minutes at room temperature. The progress and yield of the reaction was monitored by radioactive thin layer chromatography (r-TLC) using iTLC (silica gel coated on paper, Agilent Technologies Inc., Santa Clara, CA) and 0.1M sodium citrate as a mobile phase. In this r-TLC condition, unconjugated (Free)⁶⁴Cu moves to the solvent front of the r-TLC and radiolabeled ⁶⁴Cu-Conjugate-1 stays at the origin of the r-TLC plate. Based on the tested radiolabeling condition, the reaction achieved >99% yield radiolabeling of ⁶⁴Cu in 10 minutes. The TLC results are shown in FIG. 48.

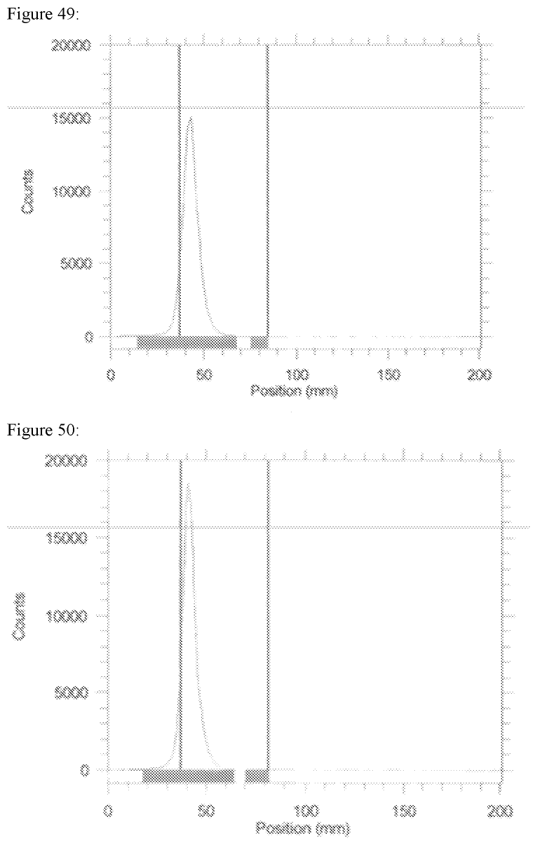

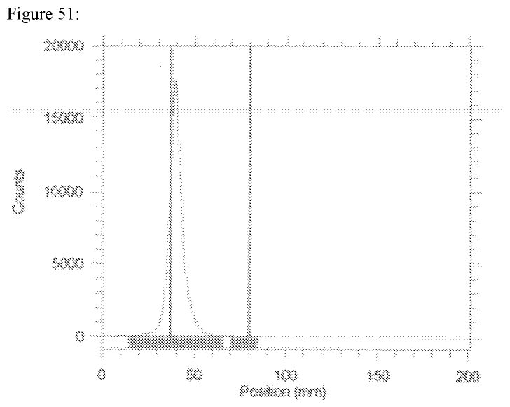

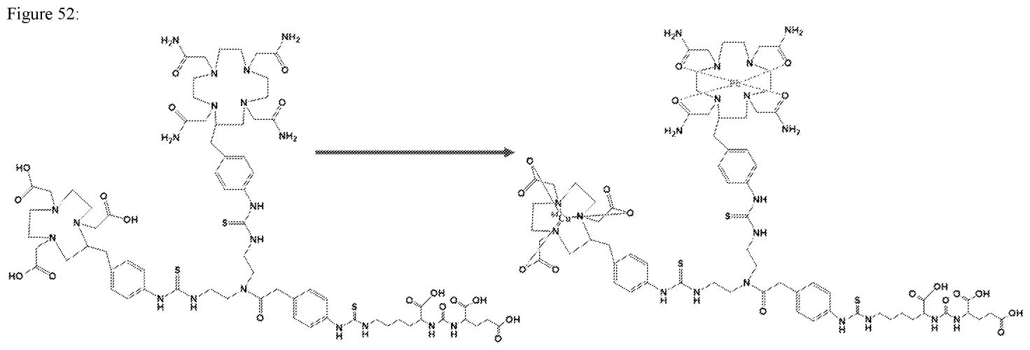

The ⁶⁴Cu/²⁰³Pb-Conjugate 2 was stability tested using TLC to measure stability. The TLC analysis was done using two different solvent systems. The first solvent system was 0.1M sodium citrate, and the second solvent system was 0.15M NH₄Ac, pH 4.0. The stability was measured at various time points including: 1 hour (FIG. 49), 4 hours (FIG. 50), and 21 hours (FIG. 51). The ⁶⁴Cu/²⁰³Pb-Conjugate 2 was found to be stable up to 21 hours at room temperature.

### Example 10—Mixed Labeling of Conjugate 2 with ⁶⁴Cu and Non-Radioactive Pb

As shown in FIG. 46, Conjugate 2 was labeled with both non-radioactive Pb and ⁶⁴Cu to form the mixed labeled conjugate, ⁶⁴Cu/Pb-Conjugate 2. The ⁶⁴Cu/Pb-Conjugate 2 was formed in two steps. First, PbCl₂ was added to 0.15M ammonium acetate buffer (pH 6.5-7), and the mixture was stirred at 37° C. for about 20 minutes at pH of 6 to form a complexed Pb-Conjugate 2. Second, the Pb-Conjugate 2 was added to a 0.1M sodium acetate buffer (pH 5.0), and ⁶⁴CuCl₂ was then added to the mixture. The mixture was stirred at room temperature for about 20 minutes at pH of 5.

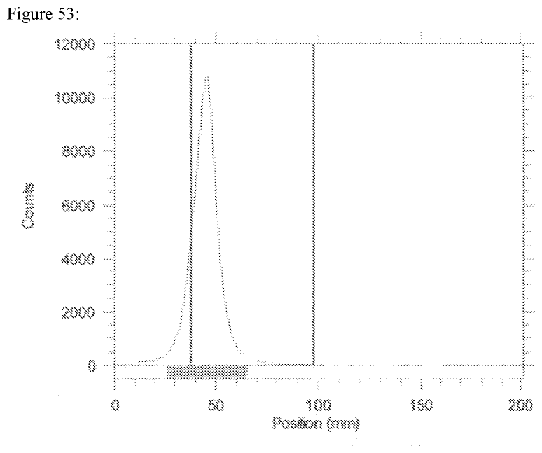

The ⁶⁴Cu/Pb-Conjugate 2 was analyzed by TLC using a silica gel solid phase and a 0.1M sodium citrate mobile phase. The TLC results are shown in FIG. 53 and Table 44 below.

TABLE 44

| | Start | Stop | Centroid | | Region | Region | % of | % of |
|---|---|---|---|---|---|---|---|---|
| Peak | (mm) | (mm) | (mm) | RF | Counts | CPM | Total | ROI |
| 1 | 26.2 | 65.4 | 44.7 | 0.111 | 181476.0 | 1088856.0 | 95.52 | 100.0 |

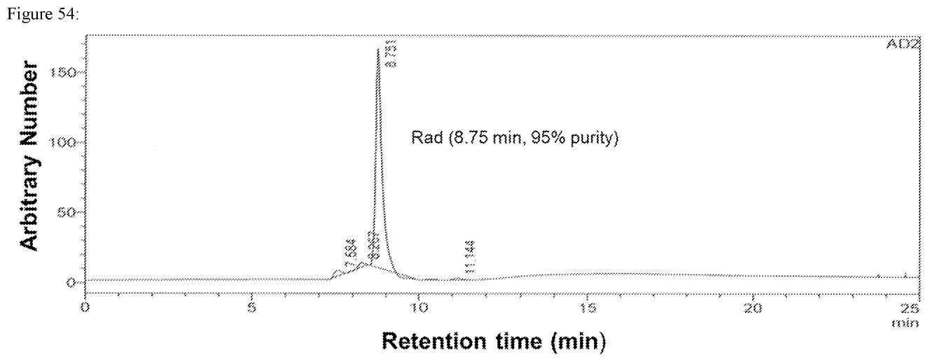

The ⁶⁴Cu/Pb-Conjugate 2 was analyzed by HPLC using the same HPLC method as used in the unlabeled ⁶⁴Cu HPLC method. The HPLC trace is shown in FIG. 54. The molar activity (Aₘ) was 52 GBq/μM.

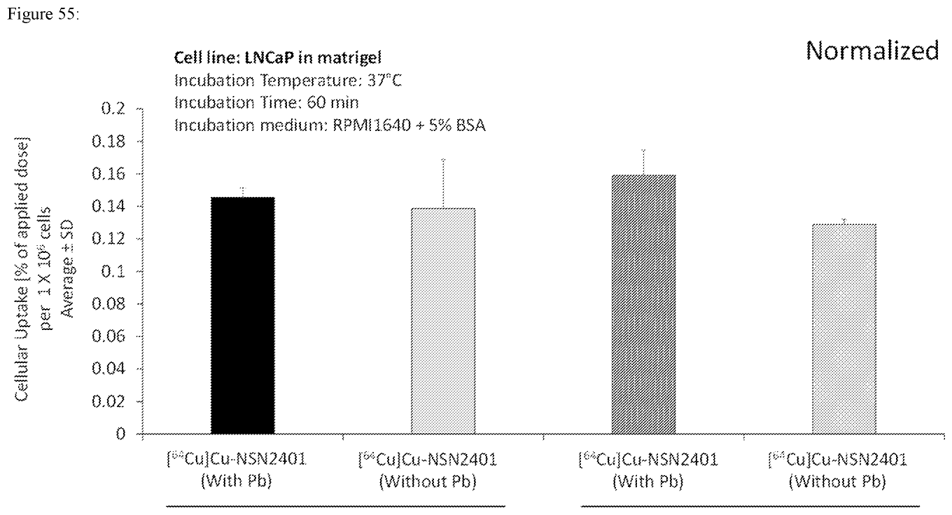

The in vitro uptake of the ⁶⁴Cu/Pb-Conjugate 2 was tested. The cell line used was LNCaP in matrigel with an incubation temperature of 37° C., an incubation time of 1 hour, and an incubation medium of RPMI1640+5% bovine serum albumin. The results of the in vitro uptake of the ⁶⁴Cu/Pb-Conjugate 2 compared to the ⁶⁴Cu-Conjugate 2 without lead showed an increase in cellular uptake of the ⁶⁴Cu/Pb-Conjugate 2 when normalized (FIG. 55).

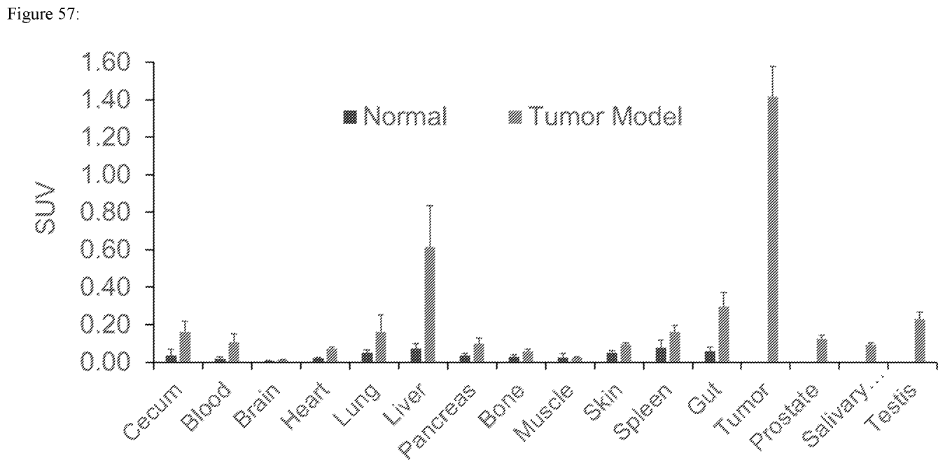

The in vivo uptake of the ⁶⁴Cu/Pb-Conjugate 2 was tested. PET images were taken of a LNCaP tumor model. The results showing the in vivo uptake of the ⁶⁴Cu/Pb-Conjugate 2 are shown in FIGS. 56 and 57 as well as Table 45.

TABLE 45

| | Uptake (SUV) in | Uptake in tumor |
|---|---|---|
| | Normal mice | Bearing mice |
| | (n = 3) | (n = 3) |
| Cecum | 0.037 ± 0.031 | 0.164 ± 0.053 |
| Blood | 0.019 ± 0.013 | 0.108 ± 0.044 |
| Brain | 0.007 ± 0.002 | 0.014 ± 0.001 |
| Heart | 0.020 ± 0.005 | 0.071 ± 0.009 |
| Lung | 0.052 ± 0.015 | 0.162 ± 0.089 |
| Liver | 0.075 ± 0.022 | 0.615 ± 0.221 |
| Pancreas | 0.034 ± 0.014 | 0.100 ± 0.028 |
| Bone | 0.028 ± 0.01 | 0.059 ± 0.008 |
| Muscle | 0.024 ± 0.022 | 0.023 ± 0.007 |
| Skin | 0.051 ± 0.012 | 0.096 ± 0.005 |
| Spleen | 0.076 ± 0.043 | 0.161 ± 0.035 |
| Gut | 0.057 ± 0.02 | 0.298 ± 0.073 |
| Tumor | — | 1.419 ± 0.160 |
| Prostate | — | 0.124 ± 0.020 |
| Salivary Gland | — | 0.091 ± 0.011 |
| Testis | — | 0.229 ± 0.39 |

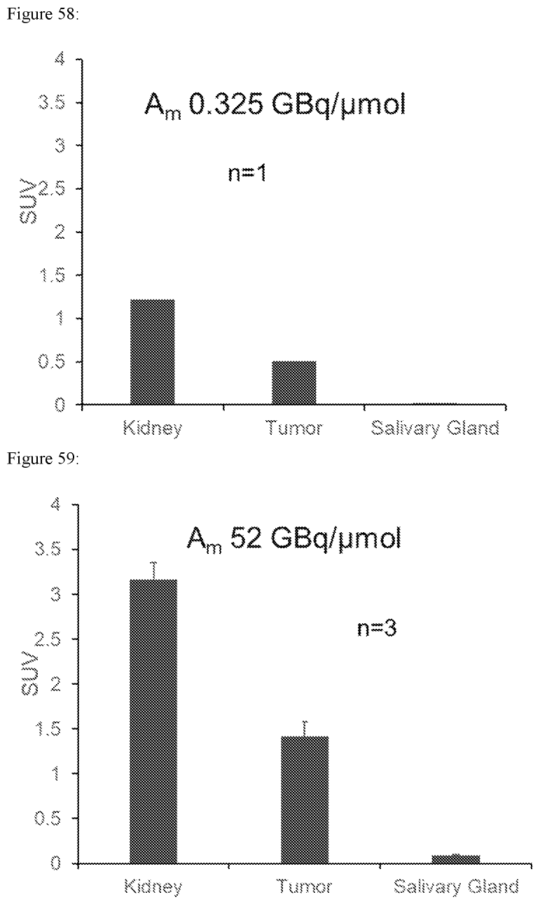

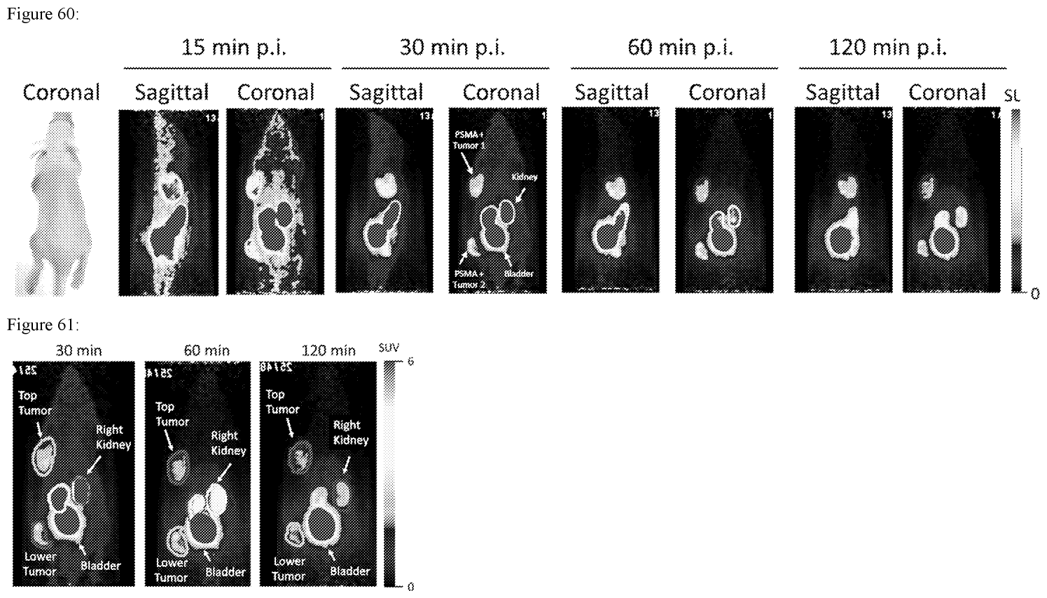

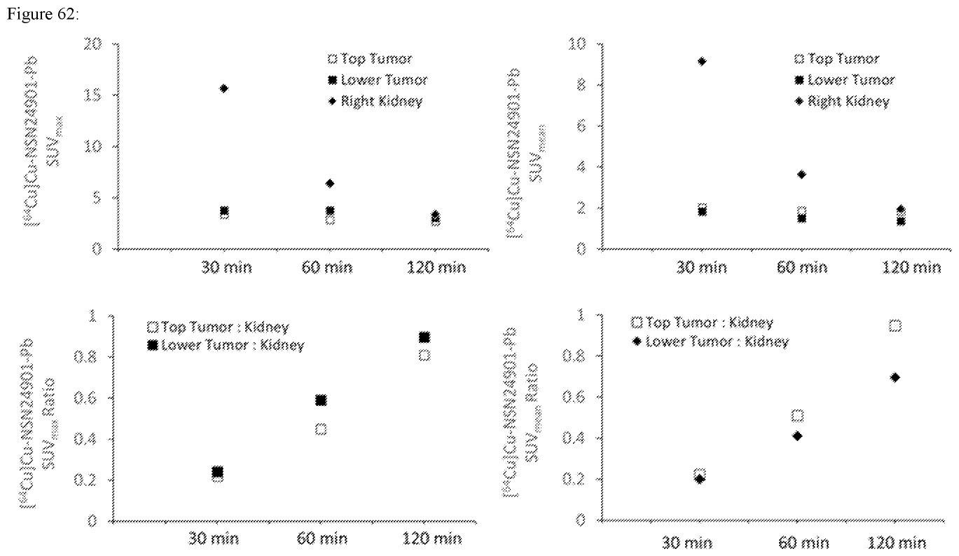

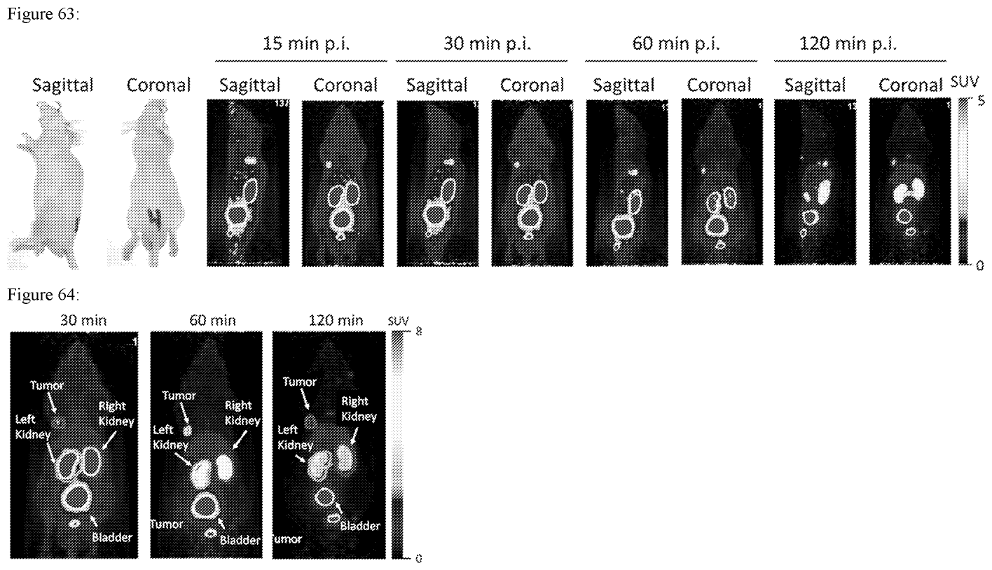

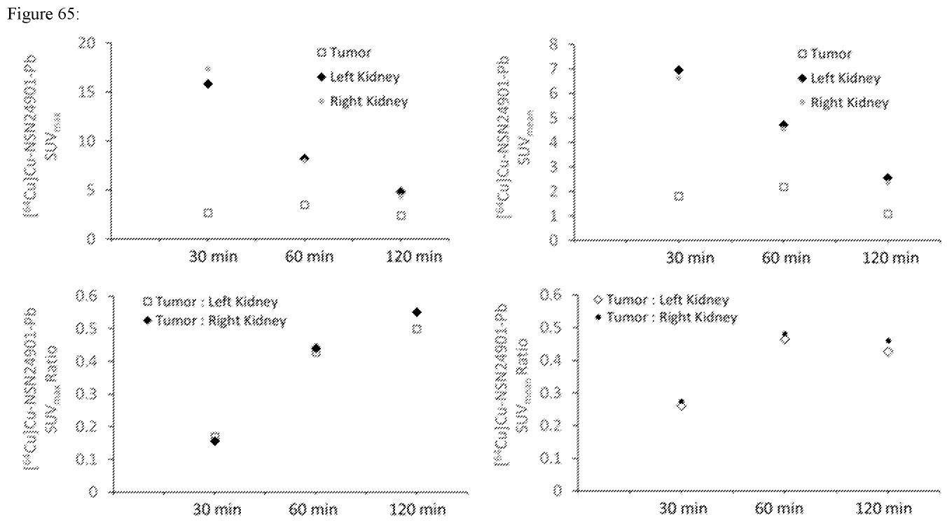

The effect of molar activity (Aₘ) on the in vivo uptake was tested in LNCaP tumor model at 120 minutes post intravenous injection. The results showed that the higher the molar activity, the higher the in vivo uptake (FIGS. 58 and 59). Further, a comparison of the uptake of the ⁶⁴Cu/Pb-Conjugate 2 between normal and tumor bearing mice 120 minutes post injection (n=3 each group) was done. The results showed that the uptake was much higher in the tumor bearing mice than in normal mice (FIG. 57). The specific molar activity was measured as radioactivity/micromoles of the ligand. The tumor specific SUV max and mean were studied in the in vivo uptake of the ⁶⁴Cu/Pb-Conjugate 2. The in vivo uptake was done in a LNCaP tumor model with ⁶⁴Cu/Pb-Conjugate 2 having an Aₘ of 52 GBq/μmol. The results are shown in FIGS. 60, 61 and 62 and in Table 45 below as well as FIGS. 63, 64, and 65 and in Table 46 below. The results shown in FIGS. 60-62 and Table 46 were from an experiment using a different animal having with different tumor locations than was used in the experiment that generated the results shown in FIGS. 63-65 and Table 47. The results showed the strength of the imaging probe and highlighted tumor heterogeneity.

TABLE 46

| | SUVₘₐₓ | | | | | SUVₘₑₐₙ | |
|---|---|---|---|---|---|---|---|
| | 30 | 60 | 120 | | 30 | 60 | 120 |
| | minute | minute | minute | | minute | minute | minute |
| Top Tumor | 3.4 | 2.88 | 2.76 | Top Tumor | 2.04 | 1.86 | 1.85 |
| Lower Tumor | 3.78 | 3.79 | 3.06 | Lower Tumor | 1.83 | 1.5 | 1.36 |
| Right Kidney | 15.65 | 6.41 | 3.41 | Right Kidney | 9.16 | 3.64 | 1.95 |
| Top Tumor: | 0.22 | 0.45 | 0.81 | Top Tumor: | 0.22 | 0.51 | 0.95 |
| Kidney | | | | Kidney | | | |
| Lower Tumor: | 0.24 | 0.59 | 0.90 | Lower Tumor: | 0.20 | 0.41 | 0.70 |
| Kidney | | | | Kidney | | | |

TABLE 47

| | SUVₘₐₓ | | | | | SUVₘₑₐₙ | |
|---|---|---|---|---|---|---|---|
| | 30 | 60 | 120 | | 30 | 60 | 120 |
| | minute | minute | minute | | minute | minute | minute |
| Tumor | 2.72 | 3.52 | 2.41 | Tumor | 1.82 | 2.19 | 1.09 |
| Left Kidney | 15.88 | 8.21 | 4.81 | Left Kidney | 6.97 | 4.71 | 2.56 |
| Right Kidney | 17.39 | 7.99 | 4.37 | Right Kidney | 6.62 | 4.55 | 2.37 |
| Tumor: | 0.17 | 0.43 | 0.50 | Tumor: | 0.26 | 0.46 | 0.43 |
| Left Kidney | | | | Left Kidney | | | |
| Tumor: | 0.16 | 0.44 | 0.55 | Tumor: | 0.27 | 0.48 | 0.46 |
| Right Kidney | | | | Right Kidney | | | |

### Example 11—In Vitro Uptake of the ⁶⁴Cu-Conjugate 2

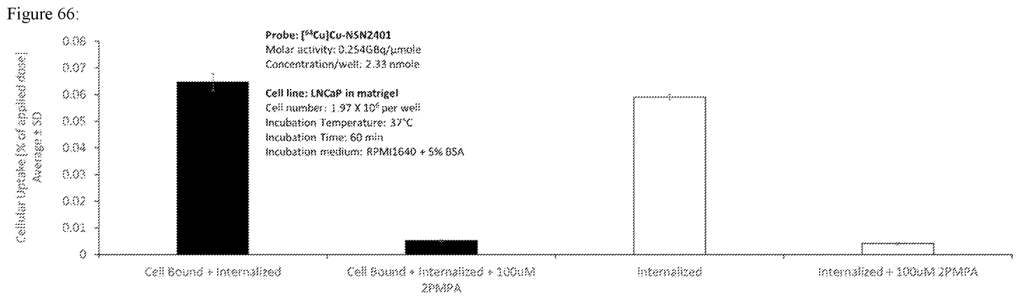

The in vitro uptake of the ⁶⁴Cu-Conjugate 2 was tested. The cell line used was LNCaP in matrigel with an incubation temperature of 37° C., an Aₘ of 0.254 GBq/μmol, a concentration/well of 2.33 nmol, a cell number per well of 1.97×10⁶, an incubation time of 1 hour, and an incubation medium of RPMI1640+5% bovine serum albumin. The results of the in vitro uptake of the ⁶⁴Cu/Conjugate 2 conjugate is shown in FIG. 66

A comparison of ex vivo biodistribution uptake of the ⁶⁴Cu-Conjugate 2 in normal and tumor bearing mice was performed. The results are shown in FIG. 57.

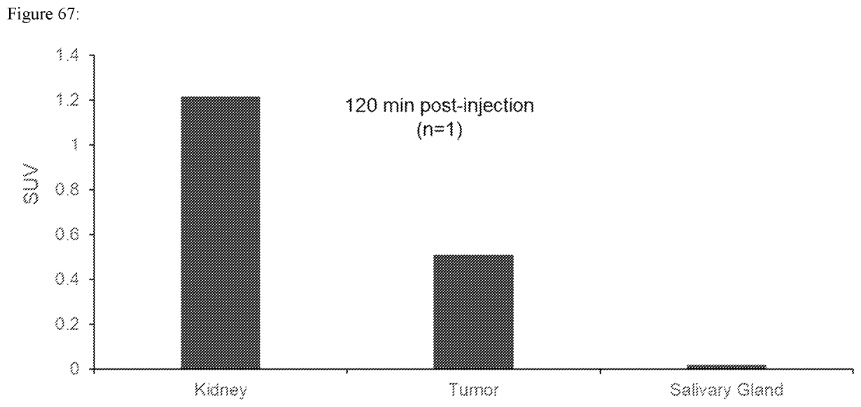

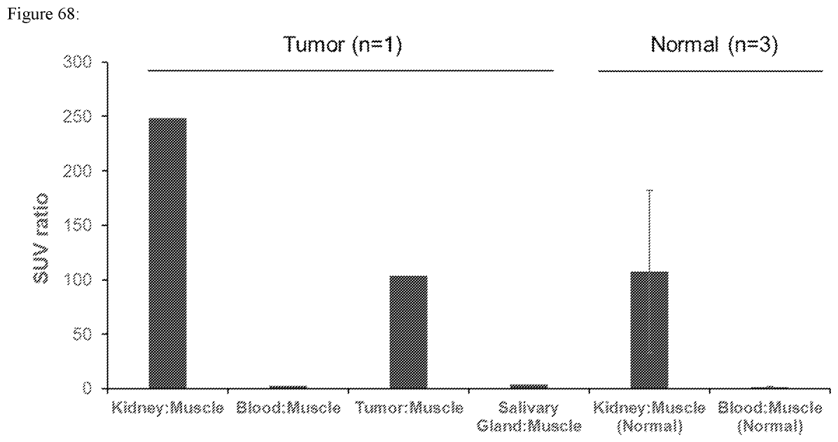

The organ specific uptake of the ⁶⁴Cu-Conjugate 2 in a LNCaP tumor model was evaluated. The results are shown in FIG. 67. The SUV ratio of the organ/tissue to muscle uptake was determined. The results are shown in FIG. 68.

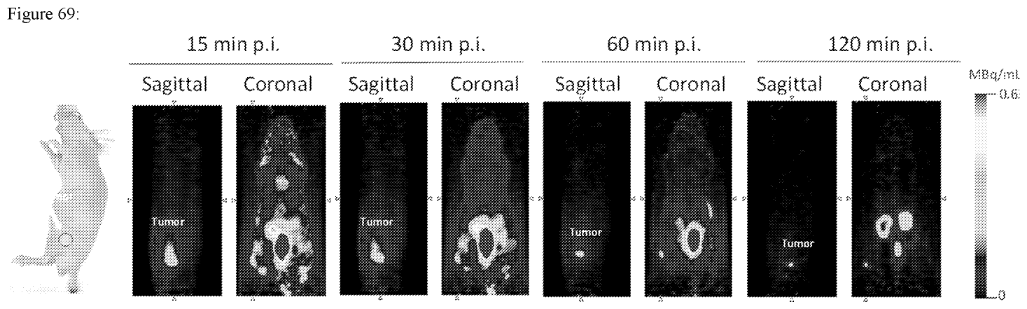

Further, micro PET images of tumor bearing mice were taken after injecting the mice with the ⁶⁴Cu-Conjugate 2. The micro PET images of the mice are shown in FIG. 69.

### Example 12—Syntheses of Alpha-PET Conjugates for Enhanced Tumor Uptake, Retention and Redundancy

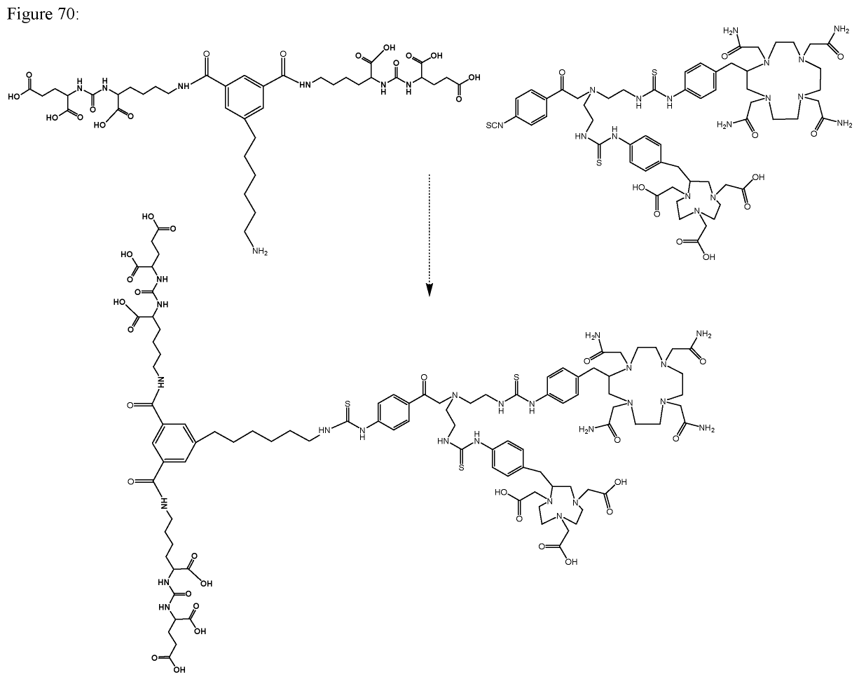

As shown in FIG. 70, two PSMA targeting vectors (e.g., lysine and glutamic acid covalently bonded together via urea bond) or analogues thereof are mixed together to form a dual targeting conjugate. The dual targeting conjugate is synthesized using a phthalic acid based aromatic moiety having three functional groups; two for tethering the PSMA vector and the third for the attachment of a dual chelator for imaging and radiotherapy applications. The dual targeting conjugate includes a six-carbon alkyl chain as a spacer between chelators and the PSMA binding vector to avoid steric hindrance in target binding and synthesis.

As shown in FIG. 71, two PSMA targeting vectors are mixed together to form a dual targeting conjugate. The dual targeting conjugate is synthesized using a diethylenetriamine based aliphatic moiety having three functional groups; two for tethering the PSMA vector and the third for the attachment of a dual chelator for imaging and radiotherapy applications. The dual targeting conjugate includes a six-carbon alkyl chain as a spacer between chelators and the PSMA binding vector to avoid steric hindrance in target binding and synthesis.

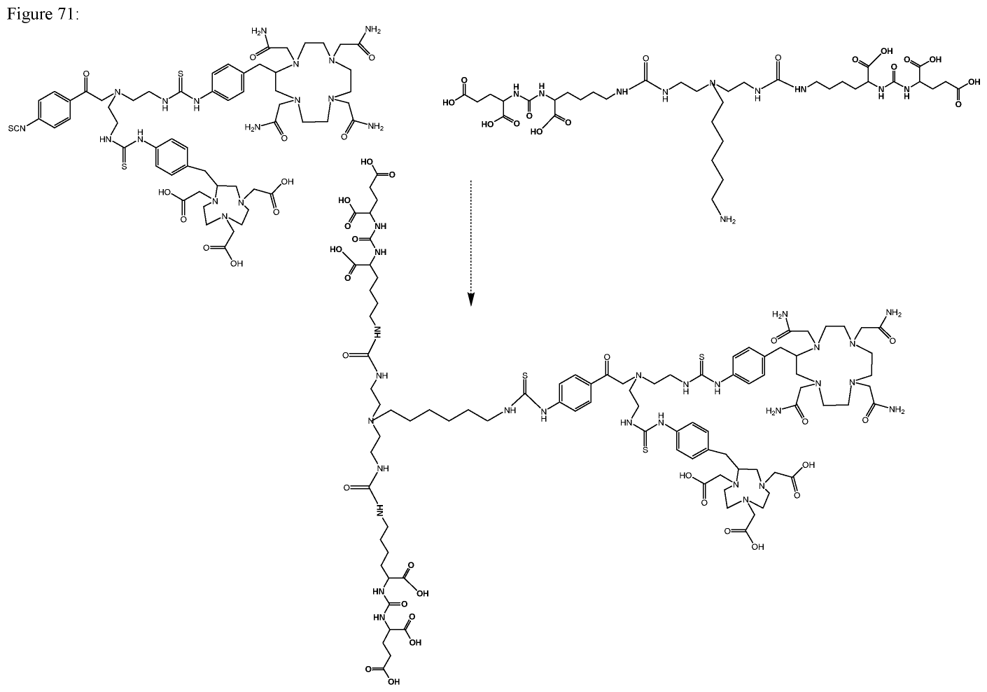

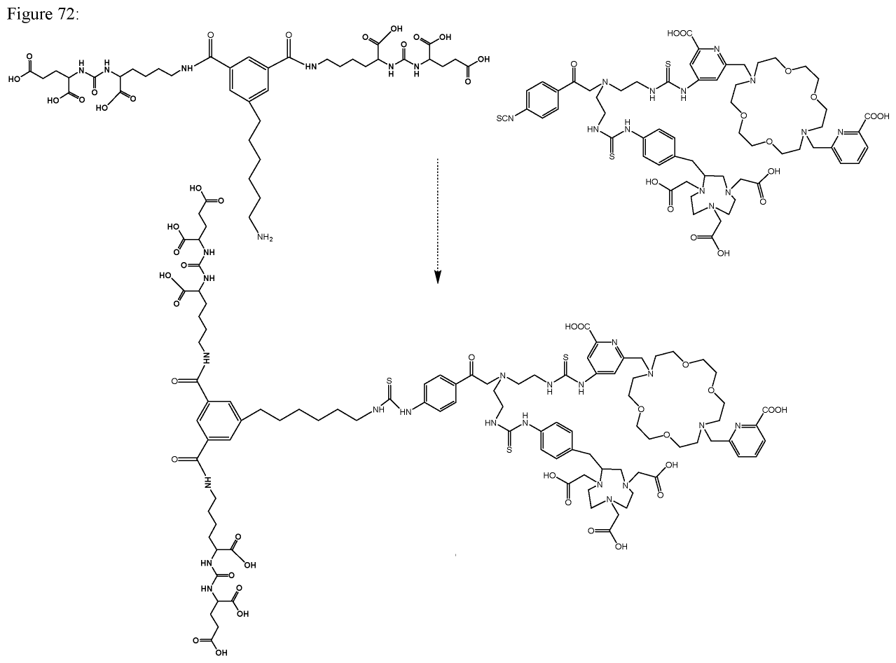

As shown in FIG. 72, two PSMA targeting vectors are mixed together to form a dual targeting conjugate. The dual targeting conjugate is synthesized using a phthalic acid based aromatic moiety having three functional groups; two for tethering the PSMA vector and the third for the attachment of a dual chelator for imaging and radiotherapy applications. The dual targeting conjugate includes a MACROPA chelator that allows chelation of additional alpha emitting radioisotopes such as ²²³Ra, ²²⁵Ac, and ²¹³Bi. The dual targeting vector includes a six-carbon alkyl chain as a spacer between chelators and the PSMA binding vector to avoid steric hindrance in target binding and synthesis.

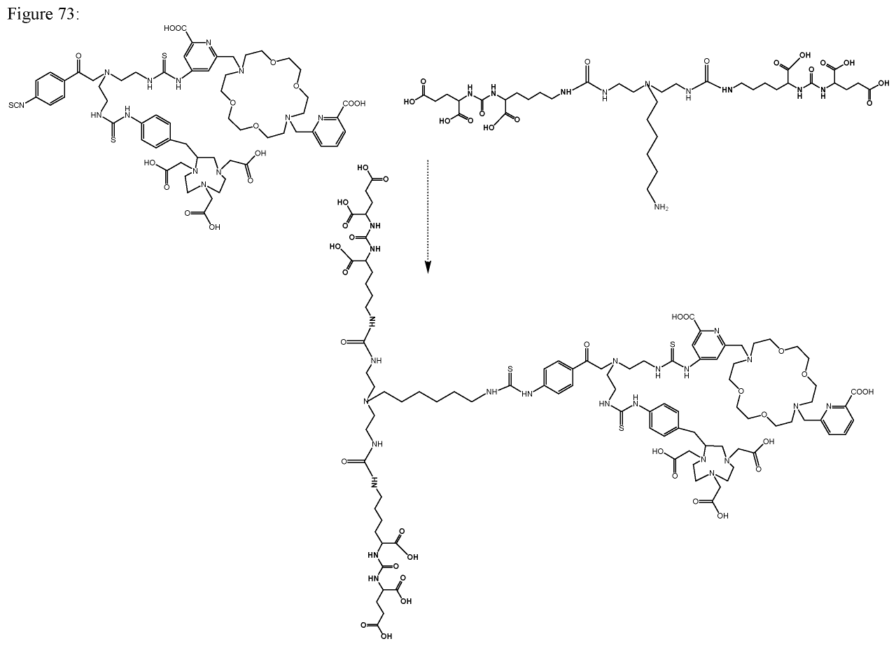

As shown in FIG. 73, two PSMA targeting vectors are mixed together to form a dual targeting conjugate. The dual targeting conjugate is synthesized using a diethylenetriamine based aliphatic moiety having three functional groups, two for tethering the PSMA vector and the third for the attachment of a dual chelator for imaging and radiotherapy applications. The dual targeting conjugate includes a MACROPA chelator that allows chelation of additional alpha emitting radioisotopes such as ²²³Ra, ²²⁵Ac, and ²¹³Bi. The dual targeting conjugate includes a six-carbon alkyl chain as a spacer between chelators and the PSMA binding vector to avoid steric hindrance in target binding and synthesis.

As shown in FIG. 74, two PSMA targeting vectors are mixed together to form a dual targeting conjugate. The dual targeting conjugate is synthesized using a phthalic acid based aromatic moiety having three functional groups; two for tethering the PSMA vector and the third for the attachment of a dual chelator for imaging and radiotherapy applications. The dual targeting vector includes a MACROPA chelator that allows chelation of additional alpha emitting radioisotopes such as ²²³Ra, ²²⁵Ac, and ²¹³Bi. The dual targeting conjugate additionally includes a DFO chelator to conjugate with a longer-lived PET isotope, such as ⁸⁹Zr. The dual targeting conjugate includes a six-carbon alkyl chain as a spacer between chelators and the PSMA binding vector to avoid steric hindrance in target binding and synthesis.

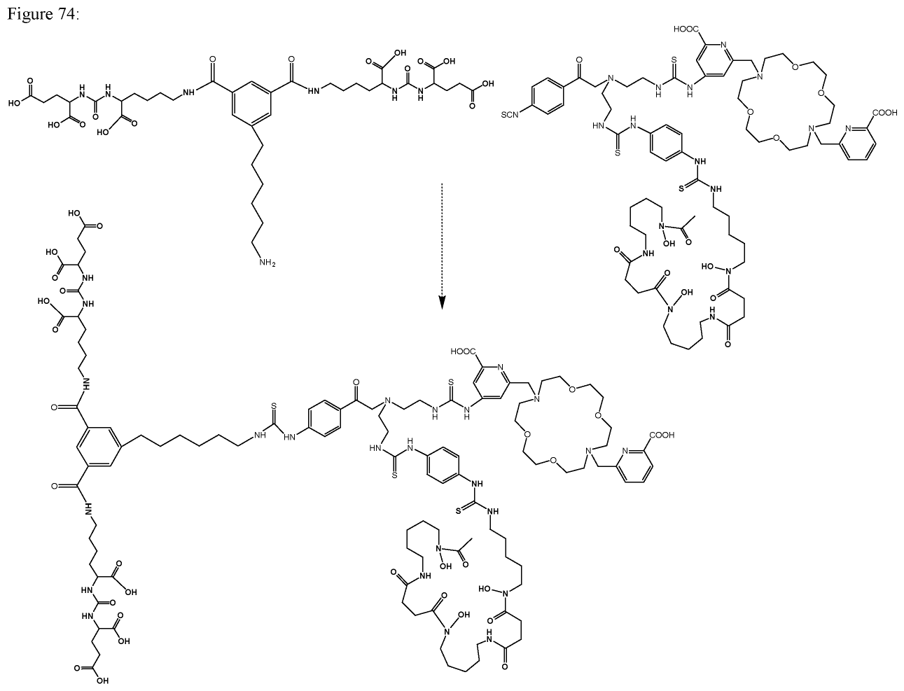

As shown in FIG. 75, two PSMA targeting vectors are mixed together to form a dual targeting conjugate. The dual targeting conjugate is synthesized using a diethylenetriamine based aliphatic moiety having three functional groups; two for tethering the PSMA vector and the third for the attachment of a dual chelator for imaging and radiotherapy applications. The dual targeting conjugate includes a MACROPA chelator that allows chelation of additional alpha emitting radioisotopes such as ²²³Ra, ²²⁵Ac, and ²¹³Bi. The dual targeting conjugate additionally includes a DFO chelator to conjugate with a longer-lived PET isotope, such as ⁸⁹Zr. The dual targeting conjugate includes a six-carbon alkyl chain as a spacer between chelators and the PSMA binding vector to avoid steric hindrance in target binding and synthesis.

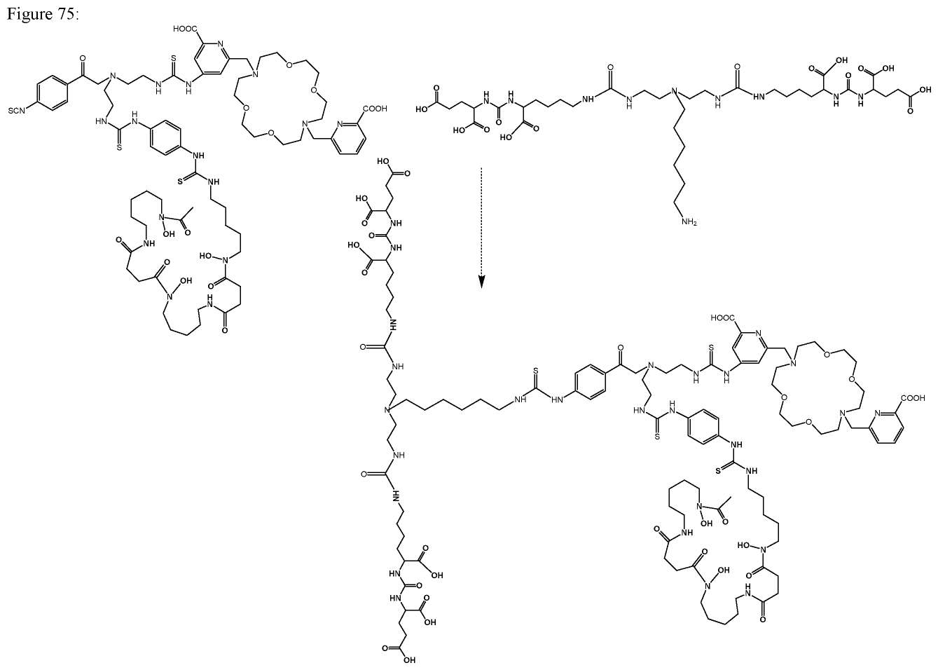

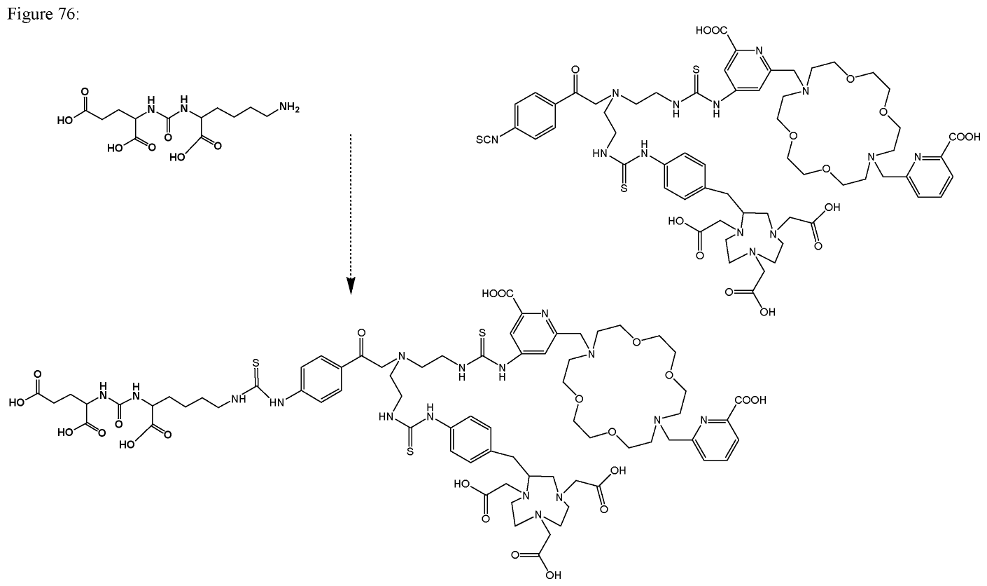

As shown in FIG. 76, a single PSMA targeting vector is mixed with a chelator to form a single targeting conjugate. The single targeting conjugate includes a MACROPA chelator that allows chelation of additional alpha emitting radioisotopes such as ²²³Ra, ²²⁵Ac, and ²¹³Bi. The single targeting conjugate with the MACROPA chelator is thought to enhance the uptake, and retention of the designed compound in the tumor and/or a longer exposure may not be needed for the effective radiotherapy.

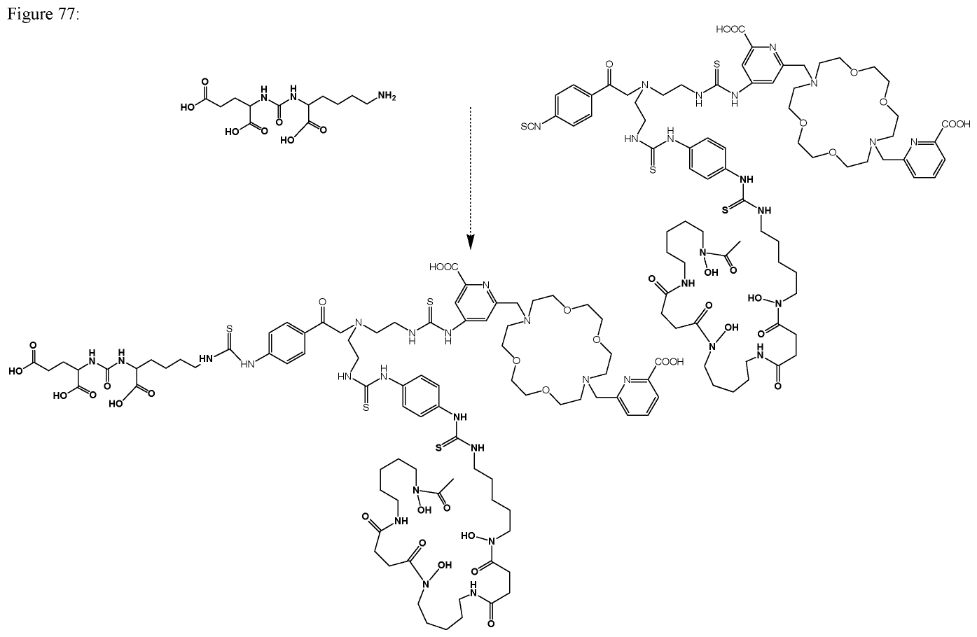

As shown in FIG. 77, a single PSMA targeting vector is mixed with a chelator to form a single targeting conjugate. The single targeting conjugate includes a MACROPA chelator that allows chelation of additional alpha emitting radioisotopes such as ²²³Ra, ²²⁵Ac, and ²¹³Bi. The single targeting conjugate with the MACROPA chelator and an additional chelator is thought to enhance the uptake and retention of the designed compound in the tumor and/or a longer exposure may not be needed for the effective radiotherapy.

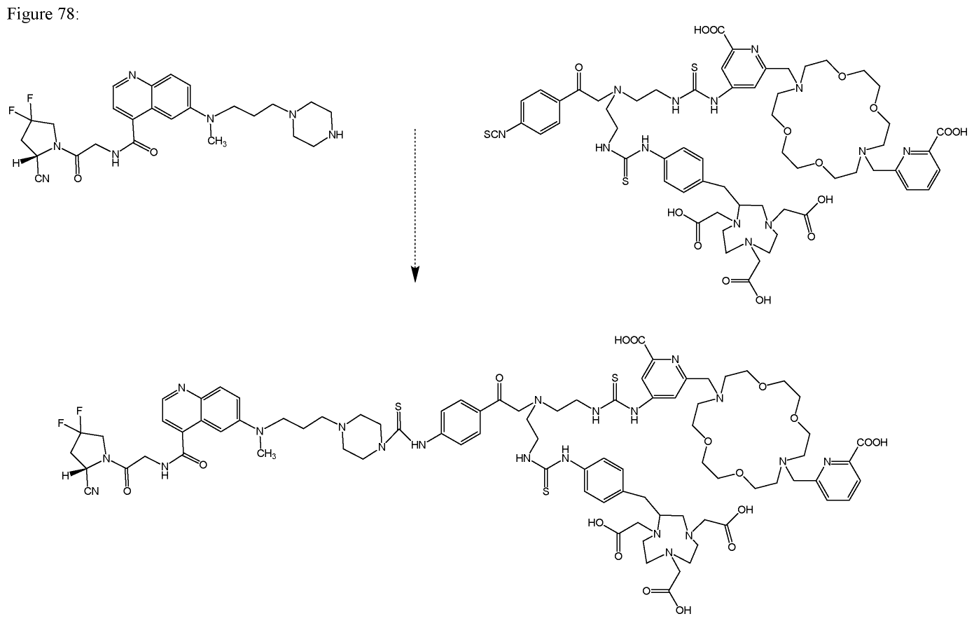

As shown in FIG. 78, a single FAPI targeting vector is mixed with a chelator to form a single targeting conjugate. The single targeting conjugate includes a MACROPA chelator that allows chelation of additional alpha emitting radioisotopes such as ²²³Ra, ²²⁵Ac, and ²¹³Bi. The single targeting conjugate with the MACROPA chelator is thought to enhance the uptake, and retention of the designed compound in the tumor, and/or a longer exposure may not be needed for the effective radiotherapy.

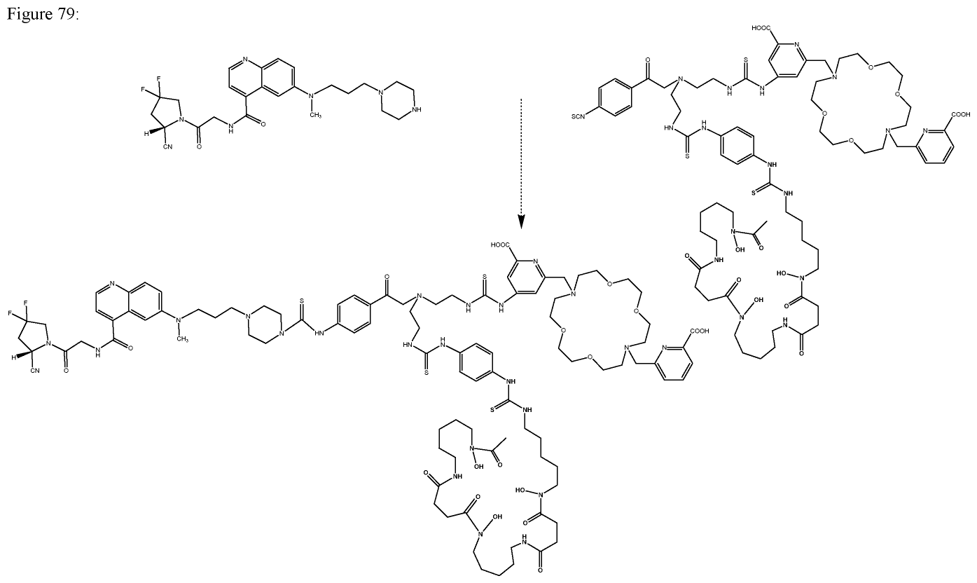

As shown in FIG. 79, a single FAPI targeting vector is mixed with a chelator to form a single targeting conjugate. The single targeting conjugate includes a MACROPA chelator that allows chelation of additional alpha emitting radioisotopes such as ²²³Ra, ²²⁵Ac, and ²¹³Bi. The single targeting conjugate with the MACROPA chelator and an additional DFO chelator is thought to enhance the uptake and retention of the designed compound in the tumor, and/or a longer exposure may not be needed for the effective radiotherapy.

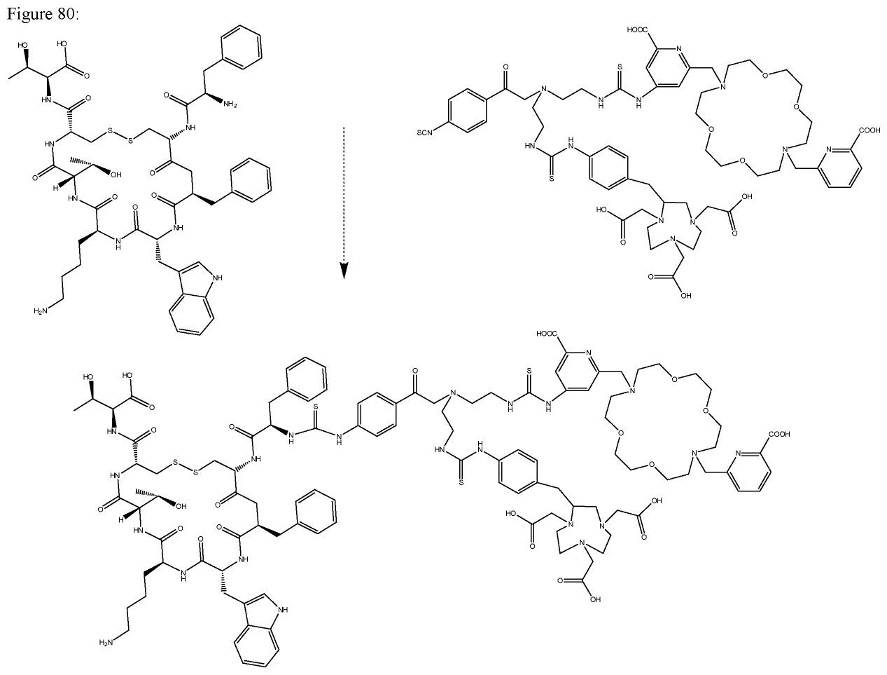

As shown in FIG. 80, a single octreotide targeting vector is mixed with a chelator to form a single targeting conjugate. The single targeting conjugate includes a MACROPA chelator that allows chelation of additional alpha emitting radioisotopes such as ²²³Ra, ²²⁵Ac, and ²¹³Bi. The single targeting conjugate with the MACROPA chelator is thought to enhance the uptake and retention of the designed compound in the tumor, and/or a longer exposure may not be needed for the effective radiotherapy.

As shown in FIG. 81, a single octreotide targeting vector is mixed with a chelator to form a single targeting conjugate. The single targeting conjugate includes a MACROPA chelator that allows chelation of additional alpha emitting radioisotopes such as ²²³Ra, ²²⁵Ac, and ²¹³Bi. The single targeting conjugate with the MACROPA chelator and an additional DFO chelator is thought to enhance the uptake and retention of the designed compound in the tumor, and/or a longer exposure may not be needed for the effective radiotherapy.

### Example 13—Dual Labeling of a FAP-Targeting Multifunctional Chelate with Both ⁶⁴Cu and Nonradioactive Pb

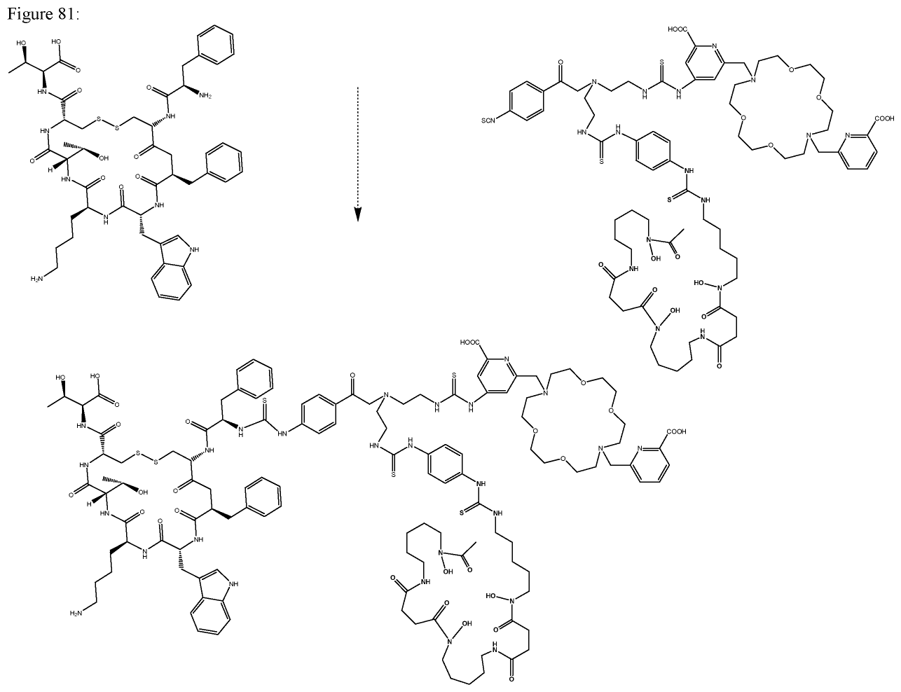

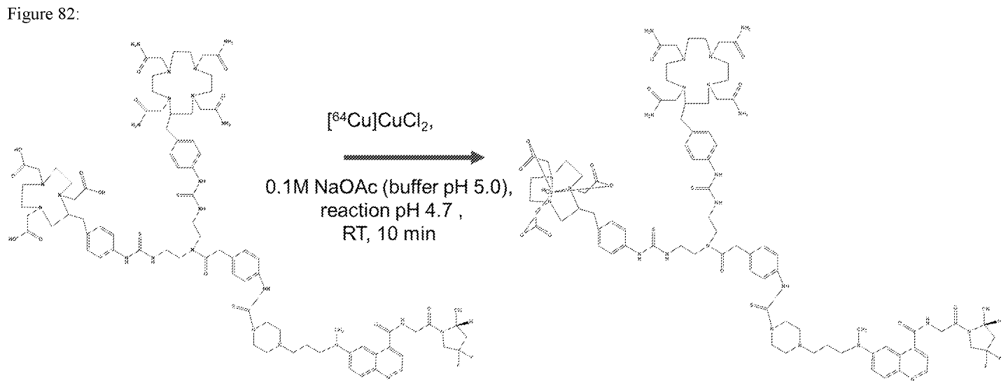

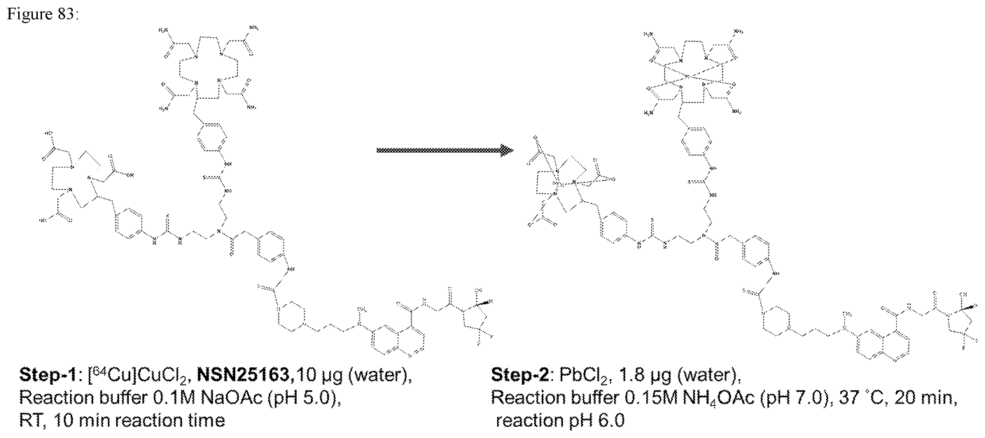

As shown in FIG. 82, Conjugate 3 was labeled with ⁶⁴Cu to form the ⁶⁴Cu-FAPI conjugate (⁶⁴Cu-Conjugate 3). A stock solution of Conjugate 3 (FAPI-NOTA-TCMC) with a concentration of 1.0 mg/mL was prepared by using 300 μg Conjugate 3 in 300 μL of 0.1 M NaOAc (pH 5.0) prior to the radiolabeling. Cyclotron produced [⁶⁴Cu]CuCl₂ was reconstituted in 2.0 mL of 0.1M NaOAc (pH 5.0) (FIG. 82). As shown in FIG. 83, Conjugate 3 was labeled with ⁶⁴Cu and nonradioactive Pb to form the ⁶⁴Cu/Pb-FAPI conjugate (⁶⁴Cu/Pb-Conjugate 3). For dual labeling with Cu-64 and nonradioactive Pb, radiolabeling reaction was performed with 10 μg Conjugate 3 (FAPI-NOTA-TCMC) dissolved in 0.1M NaOAc, (pH 5.0), of which a 10 μL of 0.15 M NH₄Ac (pH 7.0), and 1.8 μL PbCl₂ (1.0 mg/mL in 0.15 M NH₄Ac, pH 7.0) was added, and the reaction mixture was stirred at 37° C. for 20 minutes, followed by addition of 200 μL of [⁶⁴Cu]CuCl₂. The resultant reaction mixture was stirred at room temperature having a final reaction pH of ˜5.0 (4.7-5.0) for additional 10 minutes (FIG. 83). Progress of the reaction and reaction yield were measured using Rad-TLC. For rad-TLC, i-TLC (paper TLC coated with silica gel) was used and 0.1M sodium citrate (pH 4.5) as a mobile phase. The Conjugate 3 (FAPI-NOTA-TCMC) was also separately radiolabeled with Cu-64 using 10 μg FAPI with 200 μL of Cu-64 at room temperature for 10 min, having final reaction pH of 4.4-4.7 with almost 100% radiolabeling yield.

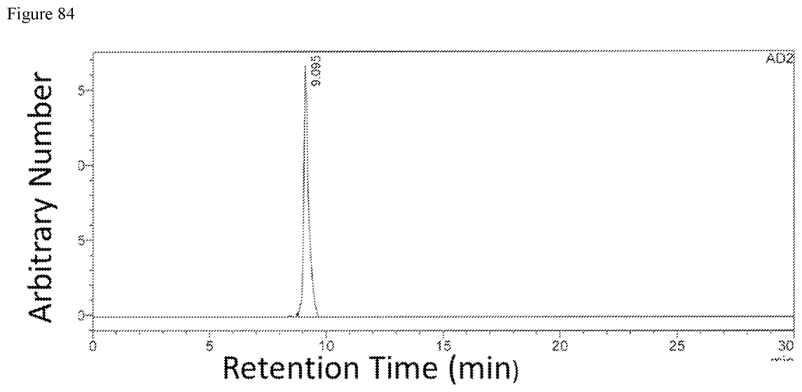

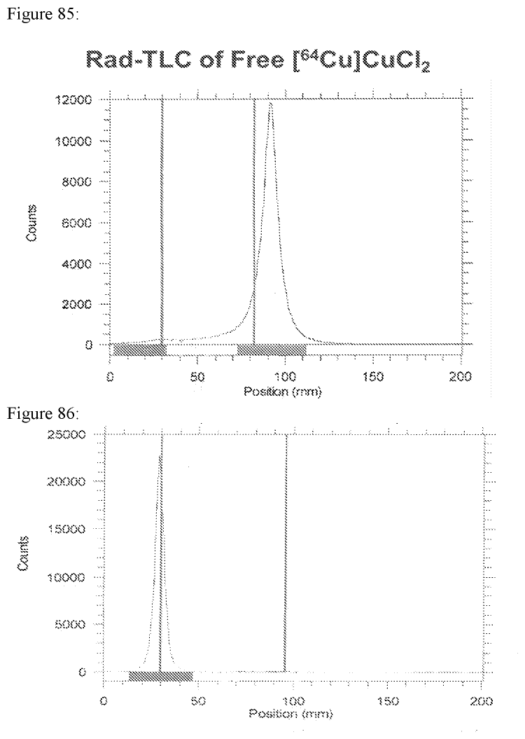

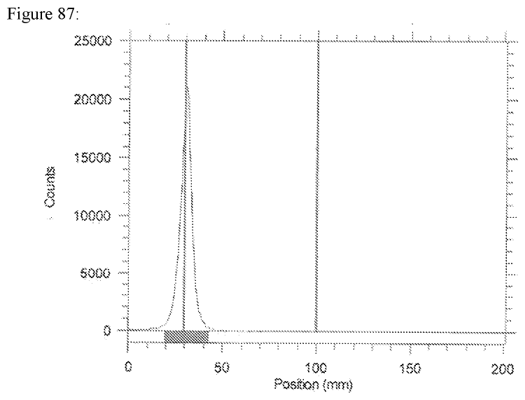

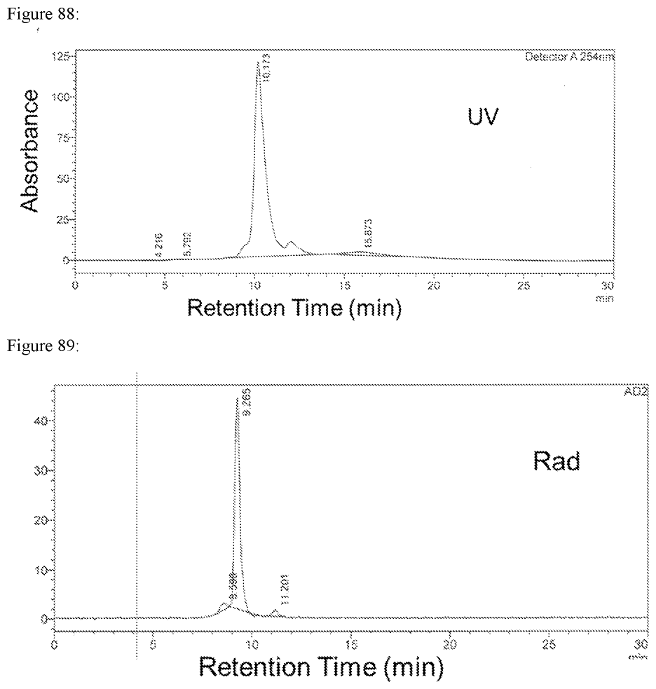

The radiolabeling reactions were also performed successfully by reversing the sequence of labeling meaning labeling with Pb followed by Cu-64 and vice versa with appropriate temperature and pH. Synthesized compounds were successfully characterized with rad-TLC, HPLC and rad-HPLC using reference compounds and control TLC of free [⁶⁴Cu]CuCl₂. As shown in FIG. 84, an UV HPLC trace of the ⁶⁴Cu-Conjugate 3 after complex formation was accomplished using a gradient solvent of 0.0 (95% B)-12:00 (45% B)—23 (95% B)-30(stop) (0.1% TFA water %, solvent B). FIG. 85 showed a rad-TLC trace of free [⁶⁴CuCl₂], FIG. 86 showed a rad-TLC trace of ⁶⁴Cu-Conjugate 3, and FIG. 87 showed a rad-TLC of ⁶⁴Cu/Pb-Conjugate 3. As shown in FIGS. 88 and 89, HPLC traces were taken of both US and radiation analyzing the purity of the dual labeled ⁶⁴Cu/Pb-Conjugate 3 after complex formation.

By HPLC, a single peak was observed, indicating complete complex formation (FIG. 84). A comparison of the rad-TLC traces of free [⁶⁴Cu]CuCl₂ and ⁶⁴Cu-Conjugate 3 revealed a significant shift in the radiation population (FIGS. 85 and 86). As by HPLC, a single peak was observed by rad-TLC for the ⁶⁴Cu-Conjugate 3, suggesting complete complex formation.

By HPLC, analysis with both UV detection and radiation detection identified one predominant peak accounting for approximately 94% of the product (FIGS. 88 and 89), indicating highly efficient complex formation. This reaction efficiency was further confirmed by comparison of the rad-TLC traces for [⁶⁴Cu]CuCl₂ and ⁶⁴Cu/Pb-Conjugate 3 (FIG. 87). Analysis of the complex revealed a single, pure peak.

### Example 14—Dual Labeling of a Somatostatin-Targeting Multifunctional Chelate with Both ⁶⁴Cu and Nonradioactive Pb

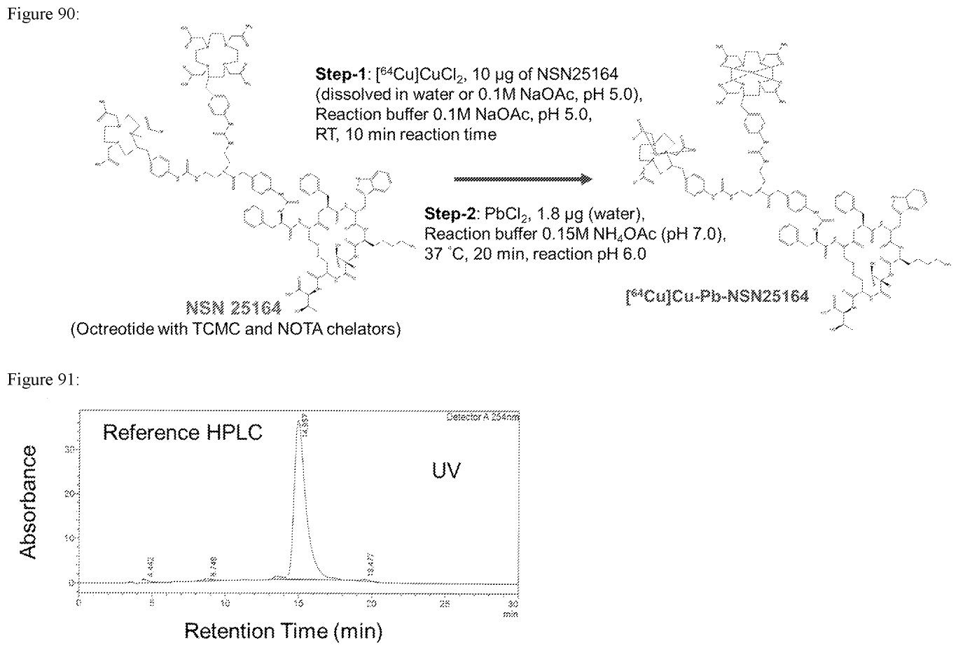

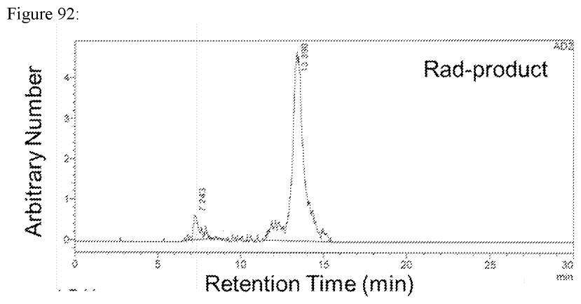

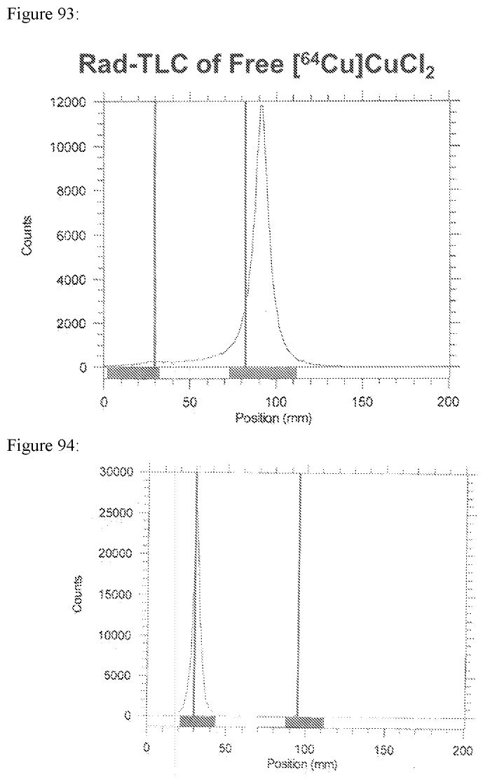

As shown in FIG. 90, Conjugate 4 was labeled with ⁶⁴Cu and nonradioactive Pb to form the ⁶⁴Cu/Pb-FAPI conjugate (⁶⁴Cu/Pb-Conjugate 3). A stock solution of Conjugate 4 (Octreotide-NOTA-TCMC) with a concentration of 1.0 mg/mL was prepared by using 300 μg Conjugate 4 in 300 μL of 0.1 M NaOAc (pH 5.0) or in water prior to the radiolabeling. Cyclotron produced [⁶⁴Cu]CuCl₂ was reconstituted in 2.0 mL of 0.1M NaOAc (pH 5.0). For dual labeling with Cu-64 and nonradioactive Pb, radiolabeling reaction was performed with 10 μg, 20 μg, and 50 μg of Conjugate 4 dissolved in 0.1M NaOAc, (pH 5.0) or in water, of which a 10 μL of 0.15 M NH₄Ac (pH 7.0) and 1.8 μL PbCl₂ (1.0 mg/mL in 0.15 M NH₄Ac, pH 7.0) was added. The reaction mixture was stirred at 37° C. for 20 minutes, followed by addition of 25 μL or 50 μL of [⁶⁴Cu]CuCl₂ (FIG. 90). The resultant reaction mixture was stirred at room temperature having a final reaction pH of ˜5.0 (4.7-5.0) for additional 10 minutes. Progress of the reaction and reaction yield were measured using Rad-TLC. For rad-TLC, i-TLC (paper TLC coated with silica gel) was used and 0.1M sodium citrate (pH 4.5) as a mobile phase. The Conjugate 4 (Octreotide-NOTA-TCMC) was also separately radiolabeled with Cu-64 using 10 μg Conjugate 4 (Octreotide-NOTA-TCMC) with 50 μL of Cu-64 at room temperature for 10 minutes, having final reaction pH of 4.4-4.7 with almost 100% radiolabeling yield. The radiolabeling reactions were also performed successfully by reversing the sequence of labeling meaning labeling with Pb followed by Cu-64 and vice versa with appropriate reaction temperature and pH. Synthesized compounds were successfully characterized with rad-TLC, HPLC and rad-HPLC using reference compounds and control TLC of free [⁶⁴Cu]CuCl₂. As shown in FIGS. 91 and 92, the UV and radiation HPLC traces were analyzed of the ⁶⁴Cu/Pb-Conjugate 4. Rad-TLC traces were taken of both free [⁶⁴Cu]CuCl₂ (FIG. 93) and the ⁶⁴Cu/Pb-Conjugate 4 (FIG. 94).

Following the reaction as shown in FIG. 90, the ⁶⁴Cu/Pb-Conjugate 4 complex was validated by both HPLC and rad-TLC. By HPLC, analysis with both UV detection and radiation detection identified one predominant peak accounting for approximately 94% of the product (FIGS. 91 and 92), indicating highly efficient complex formation. This reaction efficiency was further confirmed by comparison of the rad-TLC traces for [⁶⁴Cu]CuCl₂ and ⁶⁴Cu/Pb-Conjugate 4 (FIGS. 93 and 94). Analysis of the complex reveals a single, pure peak.

### Example 15—²¹²Pb-Conjugate 2 Synthesis and Radionuclide Therapy of Prostate Tumor

### Methods

Tumor Model Generation: Prostate cancer cell line, LNCaP was obtained from American Type Culture Collection (Manassas, VA). LNCaP tumor model was generated using male athymic nude mice obtained from Charles Rivers Laboratories (Wilmington, MA) or The Jackson Laboratory (Bar Harbor, ME) following well established LNCaP subcutaneous tumor protocol (Horoszewicz et al. Prog Clin Biol Res. 1980; 37:115-32; Horoszewicz et al. Cancer Res. 1983 April; 43(4):1809-18). On the day of cell implantation, the LNCaP cells in culture were trypsinized and washed two times in serum free RPMI-1640 medium. The cells were then resuspended in serum free RPMI-1640 medium at a concentration of 5×10⁶ cells/100 μL. A 100 μL LNCaP cell suspension was injected subcutaneously between the shoulder blades of each animal. The presence of subcutaneous tumor was confirmed on physical examination of the animal and PET imaging using ⁶⁴Cu-Conjugate 2 PSMA imaging probe. Approximately 100 ρCi of ⁶⁴Cu-Conjugate 2 was injected intravenously via tail vein injection for PET imaging based confirmation, and a 15 minutes static PET image was acquired at 1 hour post injection using a small animal Micro-PET/X-ray system (Sofie BioSystems Genesys4, Culver City, CA, USA). The PET images were visualized and analyzed using MIM 7 software (MIM Software Inc., Cleveland, OH, USA).

²¹²Pb-Conjugate 2 Radionuclide Therapy: After physical examination and confirmation via PET imaging using ⁶⁴Cu-Conjugate 2, the presence of PSMA+LNCaP tumor in an animals were established. On the day of radionuclide therapy, 4.2 mCi [²¹²Pb]PbCl₂ was received in 2.1 mL sodium acetate (1 M, pH 6.0) solution from the vendor. In order to prepare ²¹²Pb-Conjugate 2, the reaction mixture was prepared by aliquoting 1.0 mL of [²¹²Pb]PbCl₂ (2.1mCi) in a 5.0 mL of V-shaped vial followed by addition of 25 μg of Conjugate 2. The reaction mixture was then stirred for 20 minutes at 37° C. A chelation efficiency of 100% was confirmed using rad-TLC with ammonium acetate (0.15 M, pH 4.0) as a mobile phase.

Following 100% chelation, 4.0 mL of deionized water was added to the reaction mixture of ²¹²Pb-Conjugate 2 to get 2.1mCi/5.0 mL or 50 μCi/100 μL formulation of pH 6.0. A rad-TLC was again analyzed for chelation efficiency of 100% after the dilution. A single bolus dose of [²¹²Pb]Pb-NSN-24901 (0.096±0.002 mCi, n=4 mice) was injected intravenously via tail vein injection into each athymic nude mouse bearing the PSMA+LNCaP tumor. The animals were then observed and tumor size was measured at 3, 5, 9, 14, and 18 days post ²¹²Pb-Conjugate 2 injection. The total reduction in tumor size or tumor shrinkage percentage was calculated based on changes in tumor size (cm²) at 3, 5, 9, 14, and 18 days post ²¹²Pb-Conjugate 2 injection relative to tumor size observed before ²¹²Pb-Conjugate 2 therapy. At 18 days post-therapy, the absence of tumor was also confirmed using ⁶⁴Cu-Conjugate 2 PSMA imaging along with physical examination showing no tumor.

### Results

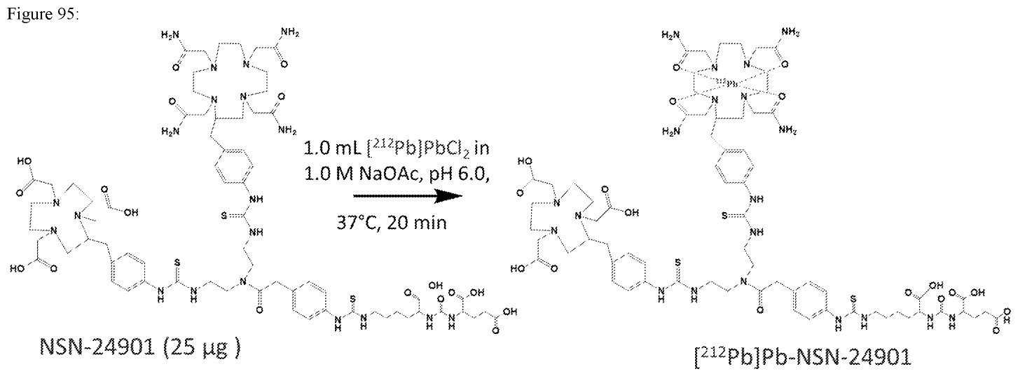

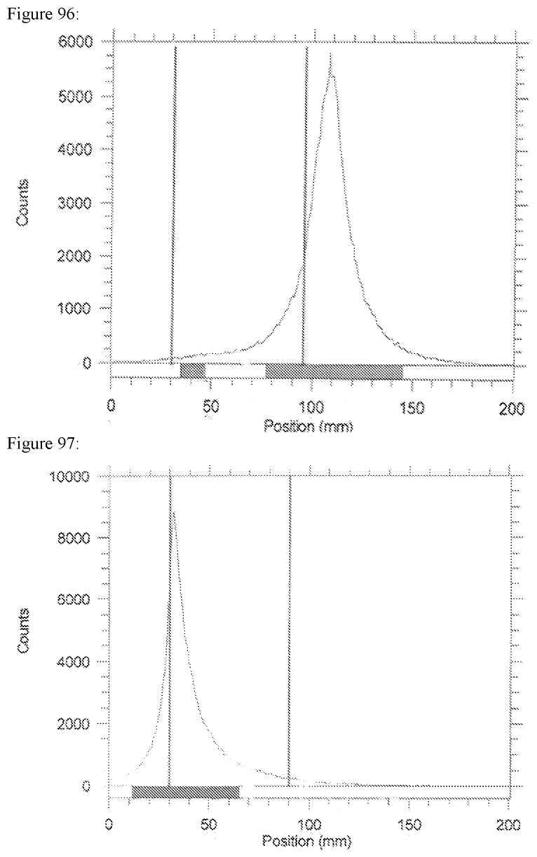

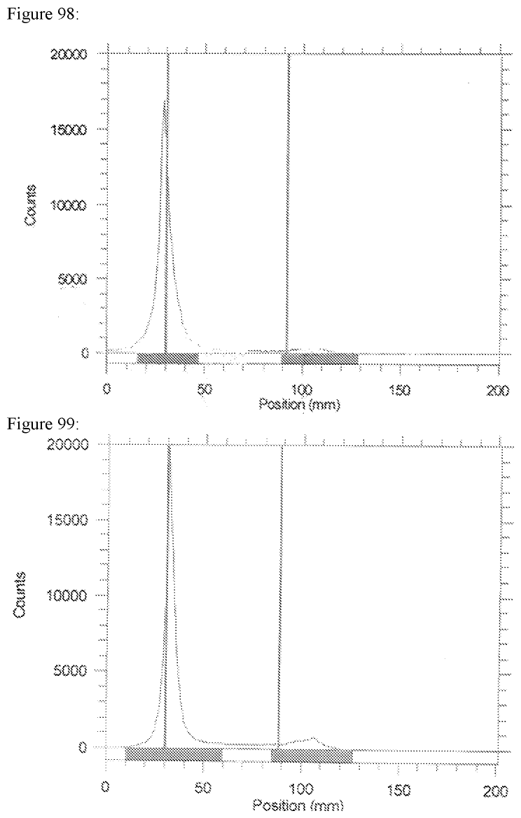

Production and Stability of ²¹²Pb-Conjugate 2: ²¹²Pb-Conjugate 2 was prepared according to the scheme shown in FIG. 95. Formation of the complex was confirmed with rad-TLC shown in FIG. 97 and free [²¹²Pb]PbCl₂ for comparison is shown in FIG. 96. Comparison of the rad-TLC for free [²¹²Pb]PbCl₂ to that of [²¹²Pb]Pb-NSN-24901 demonstrates 100% complex formation. The stability of the complex over time was also monitored by rad-TLC. The complex remained 95.0% intact after 2 hours of incubation (FIG. 98) and 89.7% intact after 22 hours of incubation (FIG. 99). This suggested that the complex was sufficiently stable over the time needed to be used therapeutically.

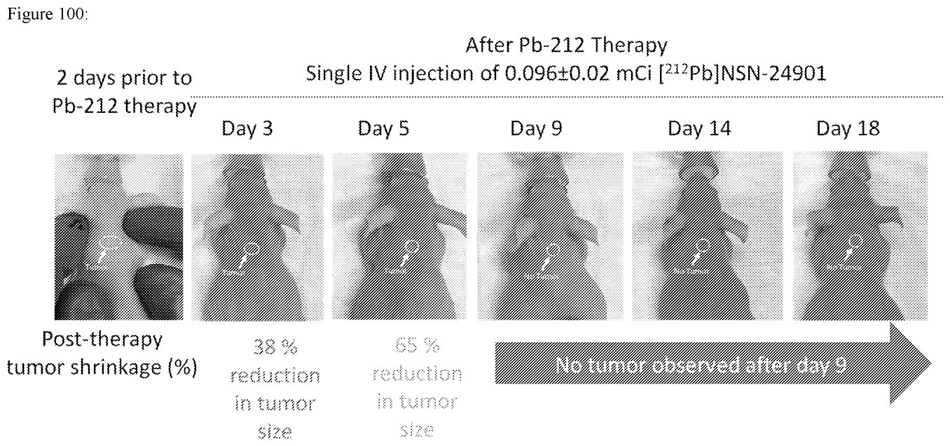

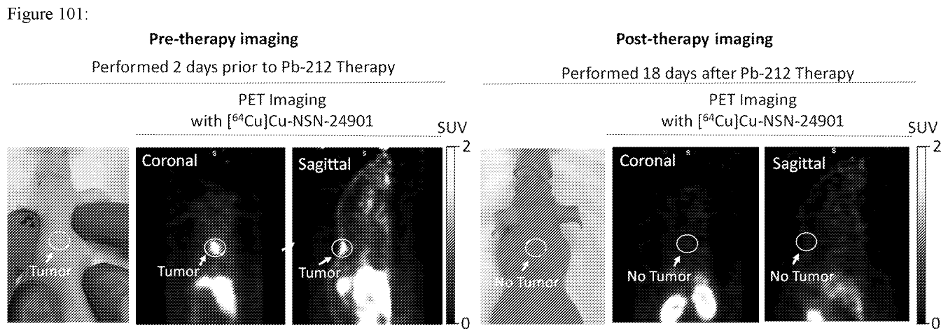

Radionuclide therapy of prostate tumor with alpha emitting ²¹²Pb-Conjugate 2: Following the establishment of LNCaP tumors in nude mice, they were treated with ²¹²Pb-Conjugate 2 (0.096±0.002 mCi, n=4 mice), and tumor size was monitored over time by both physical examination (FIG. 100). The tumor of one mouse was also monitored via PET imaging using ⁶⁴Cu-Conjugate 2, the same molecule that was used therapeutically, loaded with a PET imaging radionuclide (FIG. 101). Both physical examination and PET imaging demonstrate that the radionuclide therapy successfully reduced tumor size over time. Results for individual mice are summarized in Table 48.

TABLE 48

| | | Tumor size Post ²¹²Pb-therapy via single | | | | |
|---|---|---|---|---|---|---|
| | | intravenous injection of ²¹²Pb-Conjugate 2 | | | | |
| | Injected | 3 days | 5 days | 9 days | 14 days | 18 days |
| Mouse | Dose | post- | post- | post- | post- | post- |
| # | (mCi) | therapy | therapy | therapy | therapy | therapy |
| 1 | 0.098 | NS | NS | NT | NT | NT |
| 2 | 0.096 | 38% | 64% | NT | NT | NT |
| | | reduction | reduction | | | |
| 3 | 0.095 | NS | NS | NT | NT | NT |
| 4 | 0.094 | NT | NT | NT | NT | NT |

NS = No shrinkage observed;
NT = No tumor observed

### Example 16—Exemplary Conjugates

This Example provides the structures of exemplary conjugates described herein.

### Exemplary Conjugates for Targeting Prostate Cancers

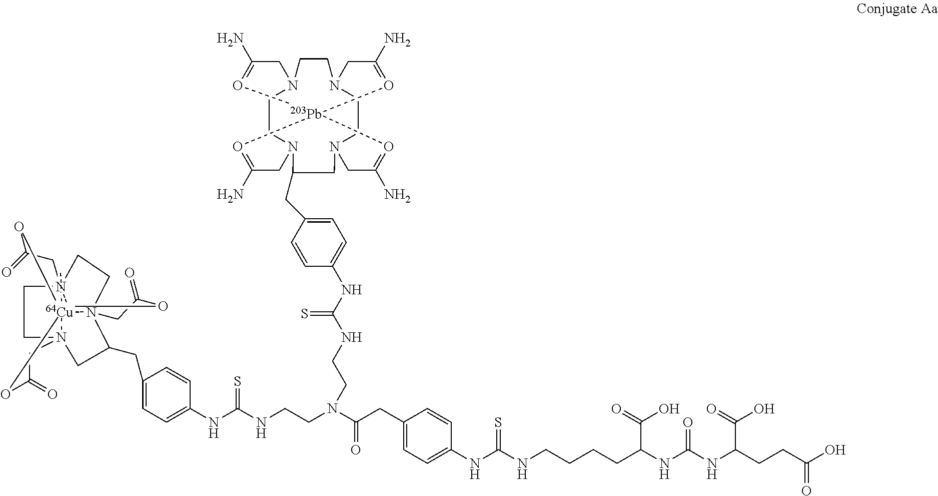

In some cases, this structure can have various combinations with Cu-64, Cu-61, Cu-67, nonradioactive Cu, and Pb-212/Pb-203/Pb-nonradioactive.

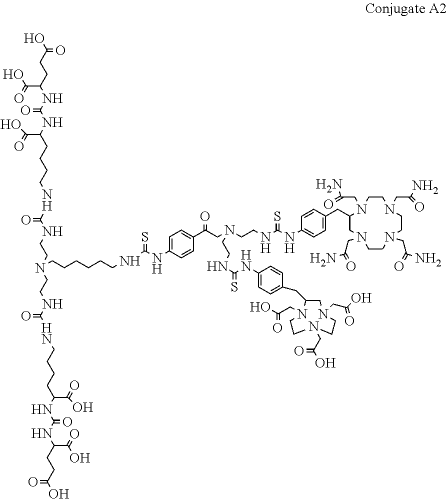 [SMILES: NC(=O)CN1CCN(CC(N)=O)CCN(CC(N)=O)C(Cc2ccc(NC(=S)NCCN(CCNC(=S)Nc3ccc(CC4CN(CC(=O)O)CCN(CC(=O)O)CCN4CC(=O)O)cc3)CC(=O)c3ccc(NC(=S)NCCCCCCN(CCNC(=O)NCCCCC(NC(=O)NC(CCC(=O)O)C(=O)O)C(=O)O)CCNC(=O)NCCCCC(NC(=O)NC(CCC(=O)O)C(=O)O)C(=O)O)cc3)cc2)CN(CC(N)=O)CC1]

In some cases, this structure can have various combinations with Cu-64, Cu-61, Cu-67, nonradioactive Cu and Pb-212/Pb-203/Pb-nonradioactive.

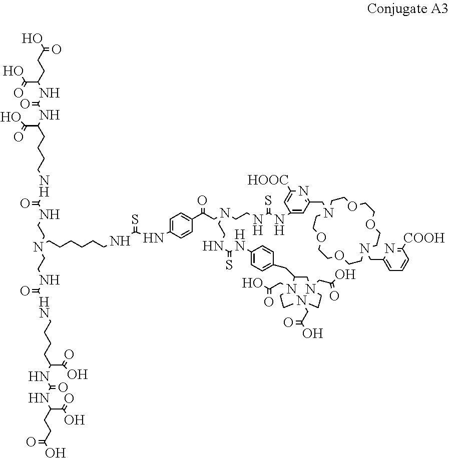 [SMILES: O=C(O)CCC(NC(=O)NC(CCCCNC(=O)NCCN(CCCCCCNC(=S)Nc1ccc(C(=O)CN(CCNC(=S)Nc2ccc(CC3CN(CC(=O)O)CCN(CC(=O)O)CCN3CC(=O)O)cc2)CCNC(=S)Nc2cc(CN3CCOCCOCCN(Cc4cccc(C(=O)O)n4)CCOCCOCC3)nc(C(=O)O)c2)cc1)CCNC(=O)NCCCCC(NC(=O)NC(CCC(=O)O)C(=O)O)C(=O)O)C(=O)O)C(=O)O]

In some cases, this structure can have various combinations with Cu-64, Cu-61, Cu-67, nonradioactive Cu and Ac-225/Ac-226/Ra-223 both radioactive and

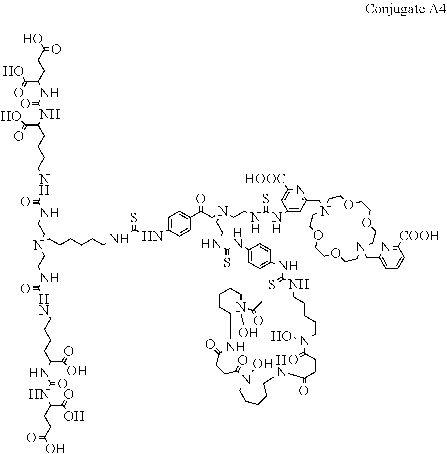 [SMILES: CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)NCCCCCCN(CCNC(=O)NCCCCC(NC(=O)NC(CCC(=O)O)C(=O)O)C(=O)O)CCNC(=O)NCCCCC(NC(=O)NC(CCC(=O)O)C(=O)O)C(=O)O)cc2)cc1]

In some cases, this structure can have various combinations with Zr-89 radioactive and nonradioactive along with Ac-225/Ac-226/Ra-223 both radioactive and nonradioactive isotopes.

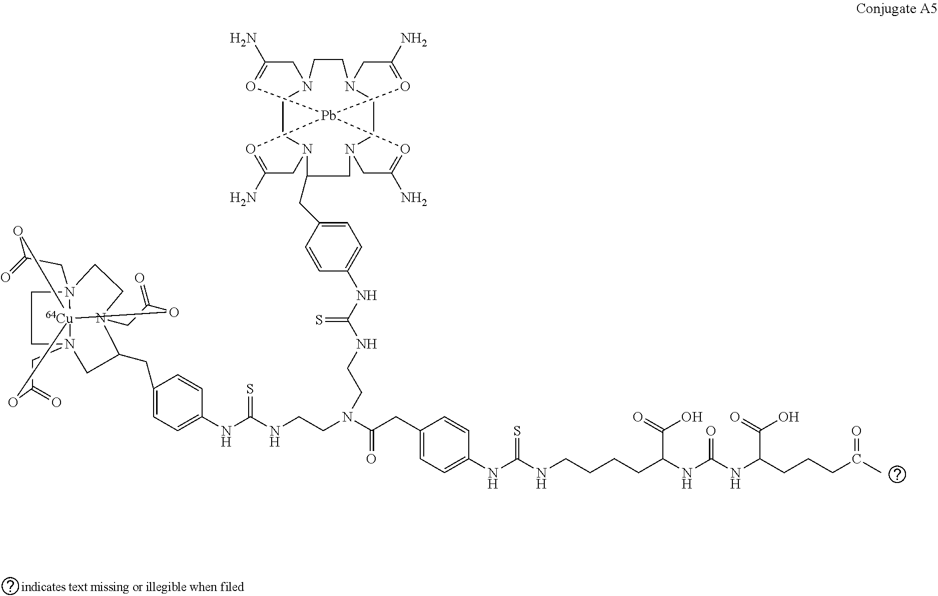

In some cases, this structure can have various combinations with Cu-64, Cu-61, Cu-67, nonradioactive Cu, and Pb-212/Pb-203/Pb-nonradioactive.

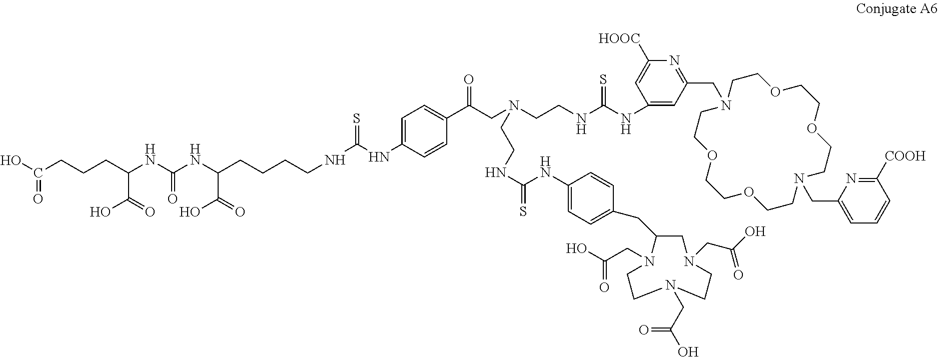 [SMILES: O=C(O)CCCC(NC(=O)NC(CCCCNC(=S)Nc1ccc(C(=O)CN(CCNC(=S)Nc2ccc(CC3CN(CC(=O)O)CCN(CC(=O)O)CCN3CC(=O)O)cc2)CCNC(=S)Nc2cc(CN3CCOCCOCCN(Cc4cccc(C(=O)O)n4)CCOCCOCC3)nc(C(=O)O)c2)cc1)C(=O)O)C(=O)O]

In some cases, this structure can have various combinations with Cu-64, Cu-61, Cu-67, nonradioactive Cu, and Ac-225/Ac-226/Ra-223 both radioactive and nonradioactive isotopes.

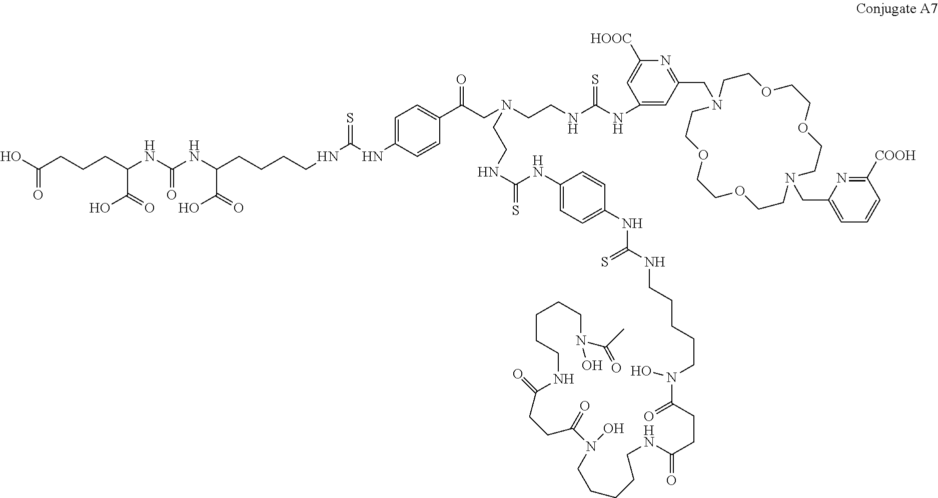 [SMILES: CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)NCCCCC(NC(=O)NC(CCCC(=O)O)C(=O)O)C(=O)O)cc2)cc1]

In some cases, this structure can have various combinations with Zr-89 radioactive and nonradioactive, along with Ac-225/Ac-226/Ra-223 both radioactive and nonradioactive isotopes.

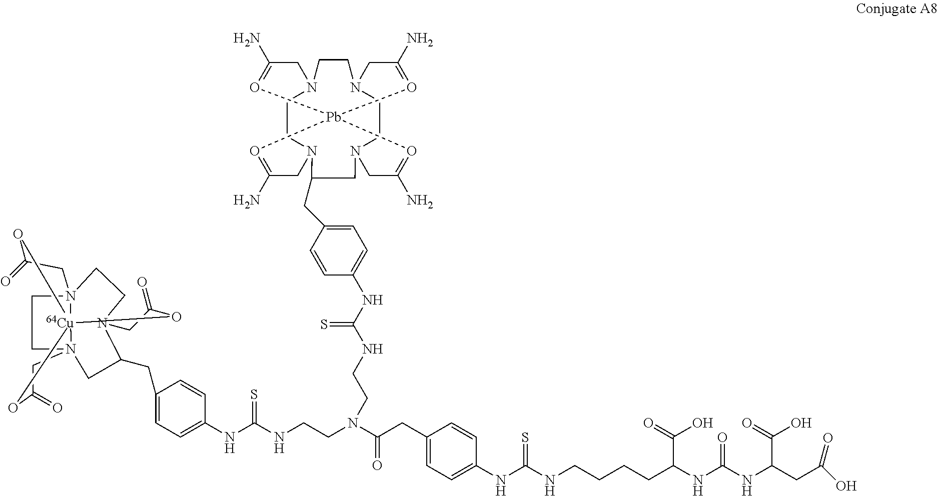

In some cases, this structure can have various combinations with Cu-64, Cu-61, Cu-67, nonradioactive Cu and Pb-212/Pb-203/Pb-nonradioactive.

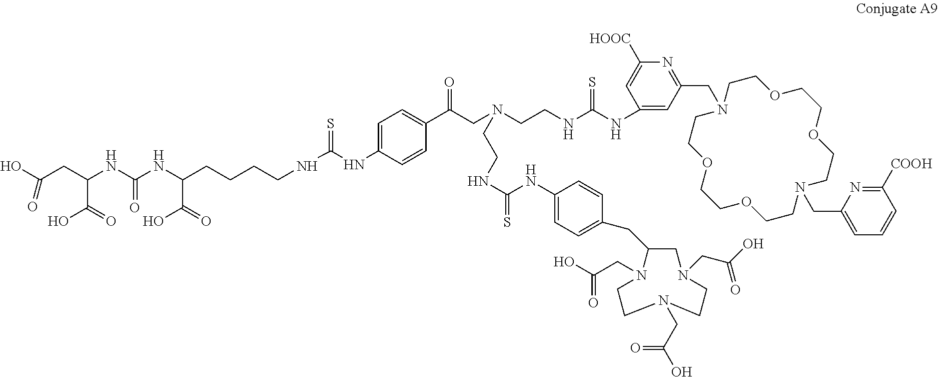 [SMILES: O=C(O)CC(NC(=O)NC(CCCCNC(=S)Nc1ccc(C(=O)CN(CCNC(=S)Nc2ccc(CC3CN(CC(=O)O)CCN(CC(=O)O)CCN3CC(=O)O)cc2)CCNC(=S)Nc2cc(CN3CCOCCOCCN(Cc4cccc(C(=O)O)n4)CCOCCOCC3)nc(C(=O)O)c2)cc1)C(=O)O)C(=O)O]

In some cases, this structure can have various combinations with Cu-64, Cu-61, Cu-67, nonradioactive Cu and Ac-225/Ac-226/Ra-223 both radioactive and nonradioactive isotopes.

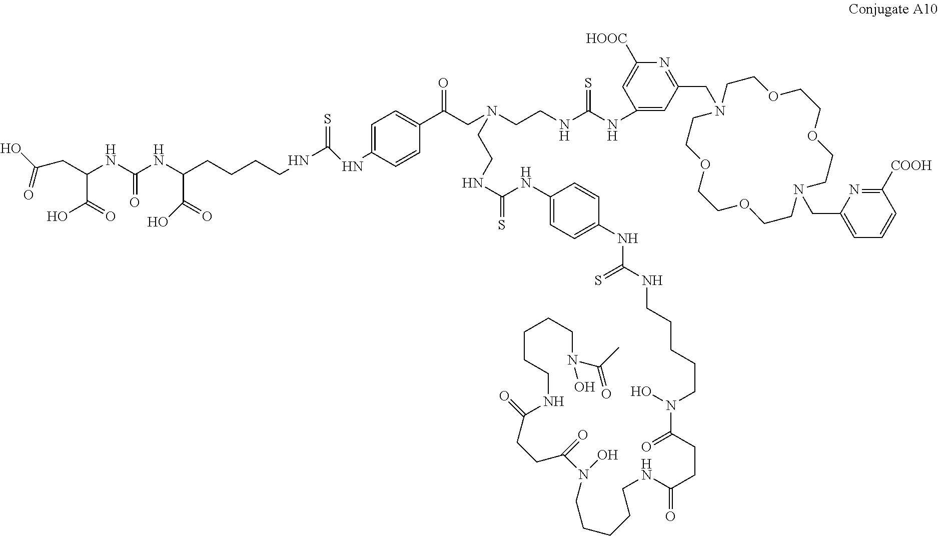 [SMILES: CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)NCCCCC(NC(=O)NC(CC(=O)O)C(=O)O)C(=O)O)cc2)cc1]

In some cases, this structure can have various combinations with Zr-89 radioactive and nonradioactive along with Ac-225/Ac-226/Ra-223 both radioactive and nonradioactive isotopes.

### Exemplary Conjugates for Targeting Neuroendocrine Tumors

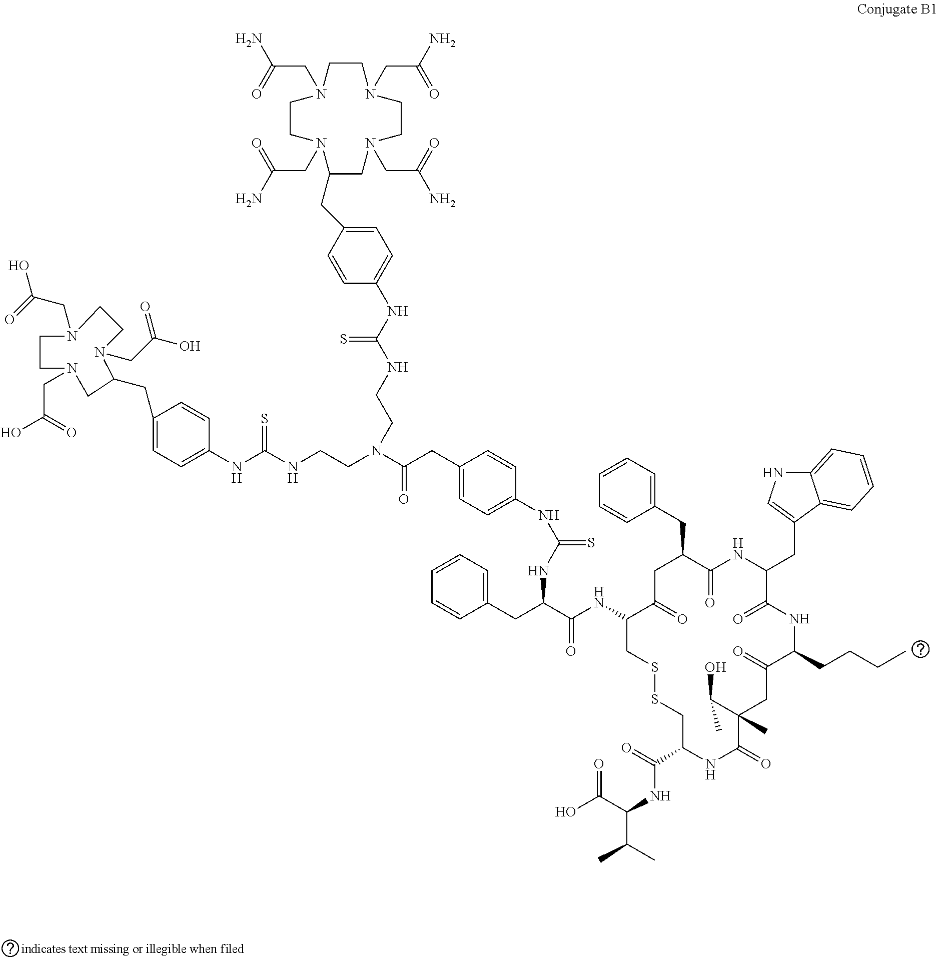

In some cases, this structure can have various combinations with Cu-64, Cu-61, Cu-67, nonradioactive Cu and Pb-212/Pb-203/Pb-nonradioactive.

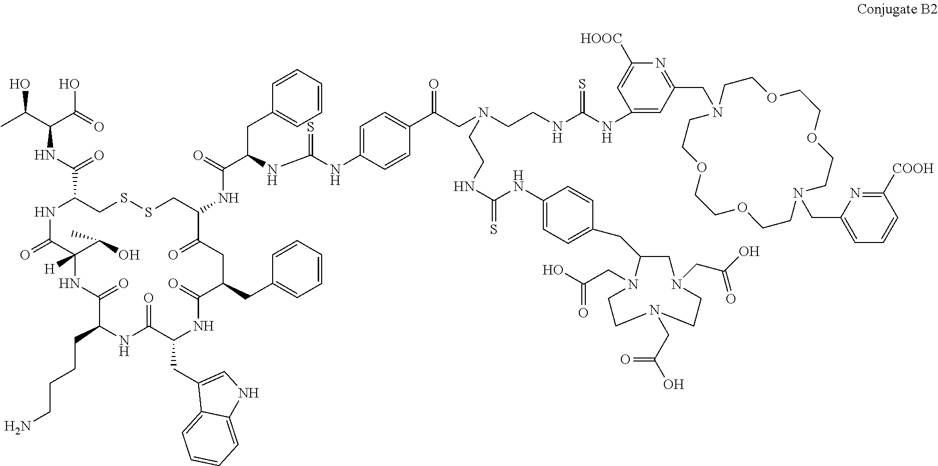

In some cases, this structure can have various combinations with Cu-64, Cu-61, Cu-67, nonradioactive Cu and Ac-225/Ac-226/Ra-223 both radioactive and nonradioactive isotopes.

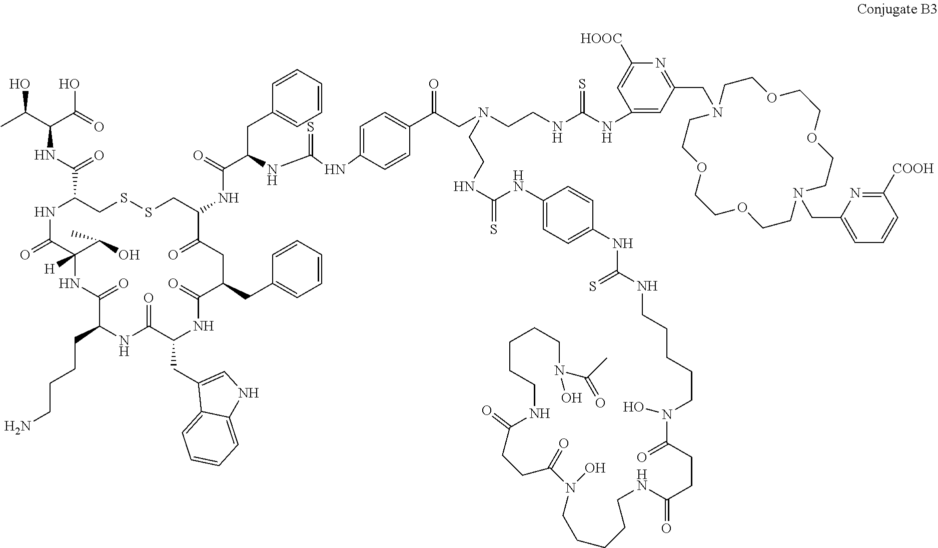

In some cases, this structure can have various combinations with Zr-89 radioactive and nonradioactive along with Ac-225/Ac-226/Ra-223 both radioactive and nonradioactive isotopes.

### Exemplary Conjugates for Targeting Fibroblast Activating Protein (FAP)

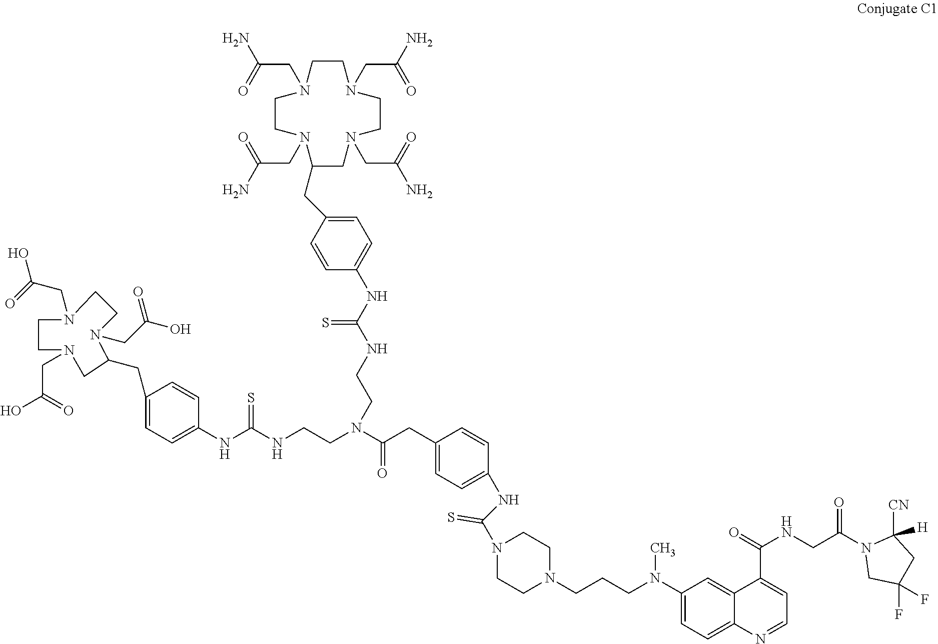 [SMILES: CN(CCCN1CCN(C(=S)Nc2ccc(CC(=O)N(CCNC(=S)Nc3ccc(CC4CN(CC(N)=O)CCN(CC(N)=O)CCN(CC(N)=O)CCN4CC(N)=O)cc3)CCNC(=S)Nc3ccc(CC4CN(CC(=O)O)CCN(CC(=O)O)CCN4CC(=O)O)cc3)cc2)CC1)c1ccc2nccc(C(=O)NCC(=O)N3CC(F)(F)C[C@H]3C#N)c2c1]

In some cases, this structure can have various combinations with Cu-64, Cu-61, Cu-67, nonradioactive Cu and Pb-212/Pb-203/Pb-nonradioactive.

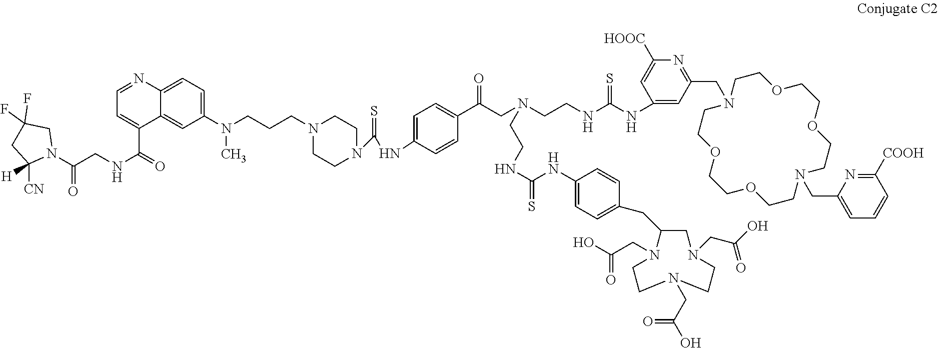 [SMILES: CN(CCCN1CCN(C(=S)Nc2ccc(C(=O)CN(CCNC(=S)Nc3ccc(CC4CN(CC(=O)O)CCN(CC(=O)O)CCN4CC(=O)O)cc3)CCNC(=S)Nc3cc(CN4CCOCCOCCN(Cc5cccc(C(=O)O)n5)CCOCCOCC4)nc(C(=O)O)c3)cc2)CC1)c1ccc2nccc(C(=O)NCC(=O)N3CC(F)(F)C[C@H]3C#N)c2c1]

In some cases, this structure can have various combinations with Cu-64, Cu-61, Cu-67, nonradioactive Cu and Ac-225/Ac-226/Ra-223 both radioactive and nonradioactive isotopes.

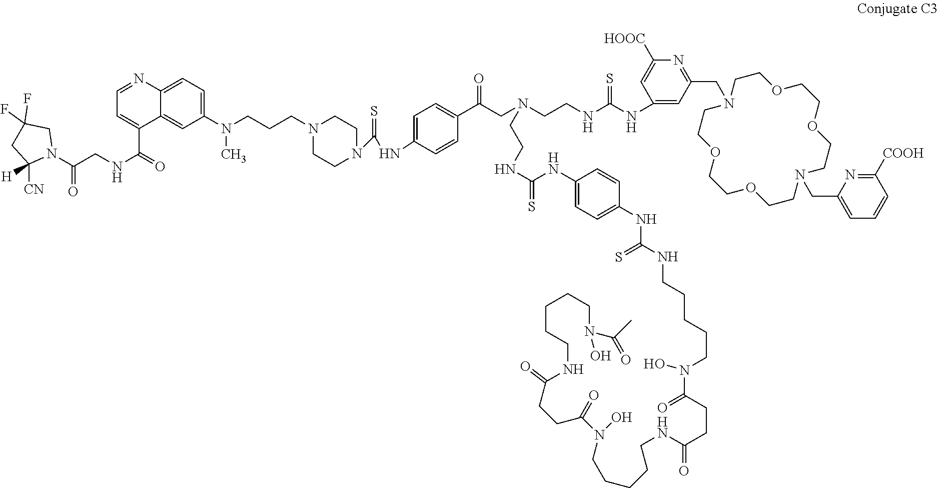 [SMILES: CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)N3CCN(CCCN(C)c4ccc5nccc(C(=O)NCC(=O)N6CC(F)(F)C[C@H]6C#N)c5c4)CC3)cc2)cc1]

In some cases, this structure can have various combinations with Zr-89 radioactive and nonradioactive along with Ac-225/Ac-226/Ra-223 both radioactive and nonradioactive isotopes.

### Exemplary Conjugates for Targeting Folate

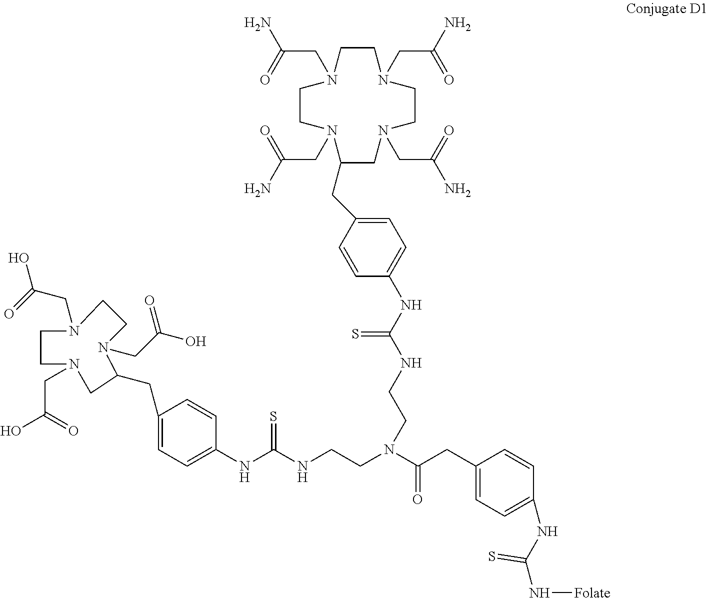

In some cases, this structure can have various combination with Cu-64, Cu-61, Cu-67, nonradioactive Cu and Pb-212/Pb-203/Pb-nonradioactive.

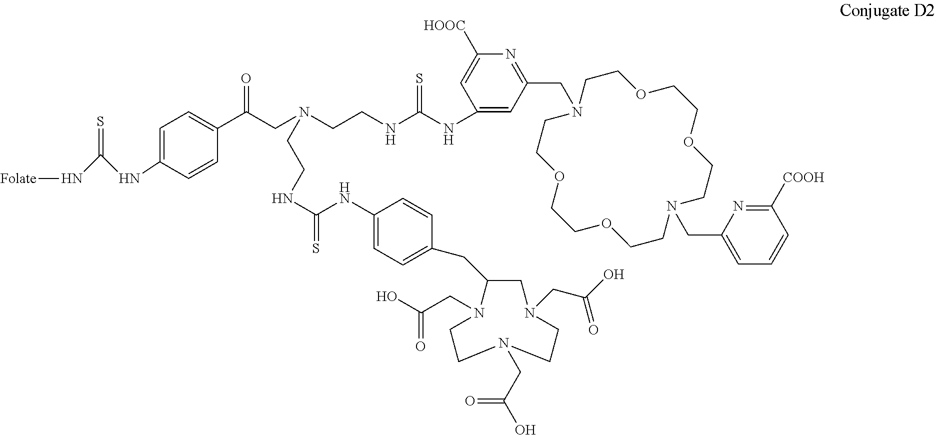

In some cases, this structure can have various combination with Cu-64, Cu-61, Cu-67, nonradioactive Cu and Ac-225/Ac-226/Ra-223 both radioactive and nonradioactive isotopes.

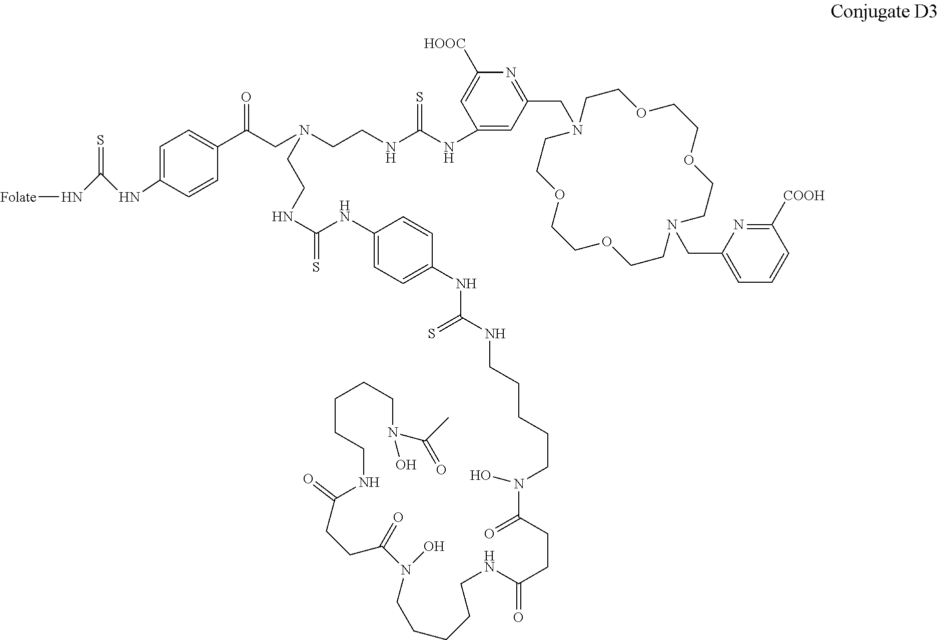

In some cases, this structure can have various combinations with Zr-89 radioactive and nonradioactive along with Ac-225/Ac-226/Ra-223 both radioactive and nonradioactive isotopes.

### Example 17—Exemplary Binding Moieties

This Example provides the amino acid sequences of exemplary binding moieties that can be used the conjugates described herein.

### Exemplary Anti-PSMA Antibody Sequences

SEQ ID No:

ANTIBODY 1

| | | |
|---|---|---|
| 393 | VL | DIQMTQSPSSLSASVGDSVTITCRASQSINNYLNWYQQKP |
| | | GKAPKLLIYTASSLLSGVPSRFSGSGSGTDFTLTISGLHPE |
| | | DFATYFCQQSFSTPPITFGQGTRLDIK |
| 394 | VH | EVQLVESGGALVQTGGSLRLSCVASGFTFSNYWMSWVR |
| | | QSPGKGLQWVASIKKDGSDEDY |
| | | VDSVKGRFTISRDNAENSLYLQMTSLRIEDTAVYYCARFI |
| | | SAVGVDWGQGALVTVSS |
| 1 | LC CDR1 | QSINNY |
| 2 | LC CDR2 | TAS |
| 3 | LC CDR3 | QQSFSTPPIT |
| 395 | HC CDR1 | GFTFSNYW |
| 396 | HC CDR2 | IKKDGSDE |
| 397 | HC CDR3 | ARFISAVGVD |

ANTIBODY 2

| | | |
|---|---|---|
| 398 | VL | DIQMTQSPSSLSASVGDRVTITCRASQGIRNDLGWYQQK |
| | | PGRAPKRLIYGASNLQSGVPSRFSGSGSGTEFTLTISSLQP |
| | | EDFATYYCLQHNSHPYTFGQGTKLEIK |
| 399 | VH | QVQLVESGGGVVQPGRSLRLSCAASGFTFITYGMHWVR |
| | | QAPGKGLEWVAIIYYDESNKYY |
| | | ADSVKGRFTISRDISKNTLYLQMNGLRAEDTAVYYCARA |
| | | PRVAVEEDYSYYYGMDVWGQG TTVTVSS |
| 49 | LC CDR1 | QGIRND |
| 59 | LC CDR2 | GAS |
| 68 | LC CDR3 | LQHNSHPYT |
| 77 | HC CDR1 | GFTFITYG |
| 91 | HC CDR2 | IYYDESNK |
| 400 | HC CDR3 | ARAPRVAVEEDYSYYYGMDV |

ANTIBODY 3

| | | |
|---|---|---|
| 401 | VL | DIVMTQSHKFMSTSVGDRVSIICKASQDVGTAVDWYQQ |
| | | KPGQSPKLLIYWASTRHTGVPDRFTGSGSGTDFTLAITNV |
| | | QSEDLADYFCQQYNSYPLTFGAGTKLEIKR |
| 402 | VH | EVQLQQSGPELVKPGTSVRISCKTSGYTFTEYTIHWVKQS |
| | | NQKFEDKATLTVDKSSSTAYMELRSLTSEDSAVYYCAA |
| | | GWNFDYWGQGTTLT |
| 54 | LC CDR1 | KASQDVGTAVD |
| 63 | LC CDR2 | WASTRHT |
| 75 | LC CDR3 | QQYNSYPLT |
| 403 | HC CDR1 | EYTIH |
| 101 | HC CDR2 | NINPNNGGTTYNQKFED |
| 118 | HC CDR3 | GWNFDY |

ANTIBODY 4

| | | |
|---|---|---|
| 404 | VL | EIVLTQSPITMAAFLGERITITCSASSSISSNYLHWYQQKP |
| | | GFSPKLLIYRTSNLASGVPIRFSGSGSGTSYSLTIGTMEAE |
| | | DVATYYCQQGSYIPFTFGSGTKLEIKR |
| 405 | VH | QVQLQESGGGLVKPGGSLKLSCAASGFTFSDFYMYWVR |
| | | QTPEKRLEWVATISDGGGYTSYPDSVKGRFTISRDIAKNN |
| | | LYLQMNSLKSEDTAKYYCARGLWLRDALDYWGQGTSV |
| | | TVSS |
| 50 | LC CDR1 | SASSSISSNYLH |
| 60 | LC CDR2 | RTSNLAS |
| 69 | LC CDR3 | QQGSYIPFT |
| 81 | HC CDR1 | GFTFSDFYMY |
| 406 | HC CDR2 | TISDGGGYTSYPDSVK |
| 113 | HC CDR3 | GLWLRDALDY |

ANTIBODY 5

| | | |
|---|---|---|
| 407 | VL | NIVMTQFPKSMSISVGERVTLTCKASENVGTYVSWYQQ |
| | | KPEQSPKMLIYGASNRFTGVPDRFTGSGSATDFILTISSVQ |
| | | TEDLVDYYCGQSYTFPYTFGGGTKLEMK |
| 408 | VH | EVKLEESGGGLVQPGGSMKLSCVASGFTFSNYWMNWV |
| | | RQSPEKGLEWVAEIRSQSNNFATHYAESVKGRVIISRDDS |
| | | KSSVYLQMNNLRAEDTGIYYCTRRWNNFWGQGTTLTVS |
| | | S |
| 55 | LC CDR1 | KASENVGTYVS |
| 64 | LC CDR2 | GASNRFT |
| 73 | LC CDR3 | GQSYTFPYT |
| 87 | HC CDR1 | GFTFSNYWMN |
| 102 | HC CDR2 | EIRSQSNNFATHYAESVKG |
| 119 | HC CDR3 | RWNNF |

ANTIBODY 6

| | | |
|---|---|---|
| 409 | VL | DIVLTQSPASLAVSLGQRATISCRASESIDSYDNTFMHWY |
| | | QQKPGQPPNLLIFRASILESGIPARFSGSGSGTDFTLTIYPV |
| | | EADDVATYYCHQSIEDPYTFGGGTKLEIK |
| 410 | VH | EVQLQQSGPELVKPGASVKMSCKASGYTFTGYVMHWV |
| | | KQKPGQVLEWIGYINPYNDVTRYNGKFKGKATLTSDKY |
| | | SSTAYMELSGLTSEDSAVYYCARGENWYYFDSWGRGAT |
| | | LTVSS |
| 56 | LC CDR1 | RASESIDSYDNTFMH |
| 65 | LC CDR2 | RASILES |
| 74 | LC CDR3 | HQSIEDPYT |
| 411 | HC CDR1 | GYTFTGYVMH |
| 103 | HC CDR2 | YINPYNDVTRYNGKFKG |
| 120 | HC CDR3 | GENWYYFDS |

ANTIBODY 7

| | | |
|---|---|---|
| 412 | VL | NIVMTQSQKFMSTSPGDRVRVTCKASQNVGSDVAWYQ |
| | | AKPGQSPRILIYSTSYRYSGVPDRFTAYGSGTDFTLTITNV |
| | | QSEDLTEYFCQQYNSYPLTFGAGTKLELK |
| 413 | VH | QVQLKESGPGLVASSQSLSITCTVSGFSLTAYGINWVRQP |
| | | PGKGLEWLGVIWPDGNTDYNSTLKSRLNIFKDNSKNQVF |
| | | LKMSSFQTDDTARYFCARDSYGNFKRGWFDFWGQGTTL |
| | | TVSS |
| 57 | LC CDR1 | KASQNVGSDVA |
| 66 | LC CDR2 | STSYRYS |
| 75 | LC CDR3 | QQYNSYPLT |
| 89 | HC CDR1 | GFSLTAYGIN |
| 414 | HC CDR2 | VIWPDGNTDYNSTLKS |
| 121 | HC CDR3 | DSYGNFKRGWFDF |

### Exemplary Anti-Somatostatin Antibody Sequences

SEQ ID No:

ANTIBODY 1

| | | |
|---|---|---|
| 415 | VL | DIVMTQSPDSLAVSLGERATINCKSSQSLLNSRNRKNYLA |
| | | WYQQKPDQSPKLLIYWASTRESGVPDRFSGSGSGTDFTL |
| | | TISSLQAEDVAVYYCKQSYYLWTFGGGTKVEIK |
| 416 | VH | EVQLVESGGGLVQPGGSLRLSCAASGFTFSDYGMAWFR |
| | | QAPGKGLEWVSFISNLGYSIYYADSVKGRFTISRDNAKN |
| | | SLYLQMNSLRAEDTAVYYCARAPYDYDSFDPMDYWGQ |
| | | GTLVTVS |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |

ANTIBODY 2

| | | |
|---|---|---|
| 417 | VL | DIVMTQSPDSLAVSLGERATINCKSSQSLLNSRTRKNYLA |
| | | WYQQKPDQSPKLLIYWASTRESGVPDRFSGSGSGTDFTL |
| | | TISSLQAEDVAVYYCKQSYYLWTFGGGTKVEIK |
| 418 | VH | EVQLVESGGGLVQPGGSLRLSCAASGFTFSDYGMAWFR |
| | | QAPGKGPEWVSFISNLAYSIYYADSVKGRFTISRDNAKNS |
| | | LYLQMNSLRAEDTAVYYCARAPYDYDSFYPMDYWGQG |
| | | TLVTVSS |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |

ANTIBODY 3

| | | |
|---|---|---|
| 419 | VL | DIVMTQSPDSLAVSLGERATINCKSSQSLLNSRTRKNYLA |
| | | WYQQKPDQSPKLLIYWASTRASGVPDRFSGSGSGTDFTL |
| | | TISSLQAEDVAVYYCKQSYYLWTFGGGTKVEIK |
| 420 | VH | EVQLVESGGGLVQPGGSLRLSCAASGFTFSDYGMAWFR |
| | | QAPGKGPEWVSFISNLAYSIYYADSVKGRFTISRDNAKNS |
| | | LYLQMNSLRAEDTAVYYCARAPYDYDSFDPMDYWGQG |
| | | TLVTVSS |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |

ANTIBODY 4

| | | |
|---|---|---|
| 421 | VL | EVQLVESGGGLVQPGGSLRLSCAASGFTFSDVGMAWFR |
| | | QAPGKGPEWVSFISNLAVSIVVADSVKGRFTISRDNAKNS |
| | | LVLQMNSLRAEDTAVVVCARAPVDYDSFDPMDYWGQG |
| | | TLVTVSS |
| 422 | VH | DIVMTQSPDSLAVSLGERATINCKSSQSLLNSRNRKSYLA |
| | | WYQQKPDQSPKLLIYYASTRASGVPDRFSGSGSGTDFTL |
| | | TISSLQAEDVAVYYCKQSYYLWTFGGGTKVEIK |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |

ANTIBODY 5

| | | |
|---|---|---|
| 423 | VL | EVQLVESGGGLVQPGGSLRLSCAASGFTFSDYGMAWFR |
| | | QAPGKGPEWVSFISNLAYSIYYADSVKGRFTISRDNAKNS |
| | | LYLQMNSLRAEDTAVYYCARAPYDVDSFDPMDYWGQG |
| | | TLVTVSS |
| 424 | VH | DIVMTQSPDSLAVSLGERATINCKSSQSLLNSRNRKSYLA |
| | | WYQQKPDQSPKLLIYVASTRASGVPDRFSGSGSGTDFTL |
| | | TISSLQAEDVAVYYCKQSYYLWTFGGGTKVEIK |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |

ANTIBODY 6

| | | |
|---|---|---|
| 425 | VL | DVVMTQTPLSLPVSLGDQASISCRSSQSLVHSNGNTYLH |
| | | WYLQRPGQSPKLLIYKVSNRFSGVPDRFSGSGSGTDFTL |
| | | KISRVEAEDLGVYFCSQSTHVPFTFGSGTKLEIK |
| 426 | VH | EVKLVESGGGLVQPGGSLRLSCATSGFTFTDYHLNWVR |
| | | QPPGKALEWLALIRNKRYGYRTEYSASVKGRFTISRDNS |
| | | QSILYLQMNTLRAEDSATYYCARDFYDPFAYWGQGTLV |
| | | TVSA |
| 7 | LC CDR1 | RSSQSLVHSNGNTYLH |
| 8 | LC CDR2 | KVSNRFS |
| 9 | LC CDR3 | SQSTHVPFT |
| 10 | HC CDR1 | DYHLN |
| 427 | HC CDR2 | IRNKRYGYRTEYSASV |
| 12 | HC CDR3 | DFYDPFAY |

ANTIBODY 7

| | | |
|---|---|---|
| 428 | VL | DVVMTQTPLSLPVSLGDQASISCRSSQSLVHSNGNTYLH |
| | | WYLQRPGQSPKLLIYKVSNRFSGVPDRFSGSGSGTDFTL |
| | | KISRVEAEDLGVYFCSQSTHVPFTFGSGTKLEIK |
| 429 | VH | EVKLVESGGGLVQPGGSLRLSCATSGFTFTDYHLNWVR |
| | | QPPGKALEWLALIRNKRYGYRTEYSASVKGRFTISRDNS |
| | | QSILYLQMNTLRAEDSATYYCARDFYDPFAYWGQGTLV |
| | | TVSA |
| 7 | LC CDR1 | RSSQSLVHSNGNTYLH |
| 8 | LC CDR2 | KVSNRFS |
| 9 | LC CDR3 | SQSTHVPFT |
| 10 | HC CDR1 | DYHLN |
| 11 | HC CDR2 | IRNKRYGYRTEYSASVKG |
| 12 | HC CDR3 | DFYDPFAY |

ANTIBODY 8

| | | |
|---|---|---|
| 430 | VL | DVVMTQTPLSLPVSLGDQASISCRSSQSLVHSNGNTYLH |
| | | WYLQRPGQSPNLLIYKVSNRFSGVPDRFSGSGSGTDFTL |
| | | KISRVEAEDLGLYFCSQSTRVPFTFGSGTKLEIK |
| 431 | VH | EVKLVESGGGLVQPGGSLRLSCATSGFTFTDYHMNWVR |
| | | QPPGKALEWLALIRNKANGYRTEYSASVKGRFTISRDNS |
| | | QNILYLQMNTLRAEDSATYYCARDFYDPFAYWGQGTLV |
| | | TVSA |
| 7 | LC CDR1 | RSSQSLVHSNGNTYLH |
| 8 | LC CDR2 | KVSNRFS |
| 127 | LC CDR3 | SQSTRVPFT |
| 129 | HC CDR1 | DYHMN |
| 131 | HC CDR2 | LIRNKANGYRTEYSASVKG |
| 12 | HC CDR3 | DFYDPFAY |

### Exemplary Anti-FAP Antibody Sequences

SEQ ID No:

ANTIBODY 1

| | | |
|---|---|---|
| 432 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYKFPYTFGQGTKLEI |
| 433 | VH | EVQIIESGGGIVQPGGSLRLSCAASGFTFSSYAMSWVRQA |
| | | PGKGIEWVSAISGSGGTTYYADSVKGRFTISRDNSKNTL |
| | | YIQMNSLRAEDTAVYYCAKDAGRPYFDYWGQGTLVTV |
| | | SS |
| 13 | LC CDR1 | RASQSISSYLN |
| 14 | LC CDR2 | AASSLQS |
| 434 | LC CDR3 | QQSYKFPYT |
| 16 | HC CDR1 | SYAMS |
| 17 | HC CDR2 | AISGSGGTTYYADSVKG |
| 435 | HC CDR3 | DAGRPYFD |

ANTIBODY 2

| | | |
|---|---|---|
| 436 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATY YCQQSYSTPRTFGQGTKLEIK |
| 437 | VH | EVQLLESGGGLVQPGGSLRLSCAASGFTFSSYAMSWVRQ |
| | | APGKGLEWVSAISGSGGGTRYADSVKGRFTISRDNSKNT |
| | | LYLQMNSLRAEDTAVYYCAKHSSGFHWYFDYWGQGTL |
| | | VTVSS |
| 438 | LC CDR1 | RASQSISSYL |
| 14 | LC CDR2 | AASSLQS |
| 143 | LC CDR3 | QQSYSTPRT |
| 16 | HC CDR1 | SYAMS |
| 152 | HC CDR2 | AISGSGGGTRYADSVKG |
| 157 | HC CDR3 | HSSGFHWYFDY |

ANTIBODY 3

| | | |
|---|---|---|
| 439 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATY YCQQSYSSPYTFGQGTKLEIK |
| 440 | VH | EVQLLESGGGLVQPGGSLRLSCAASGFTFSSYAMSWVRQ |
| | | APGKGLEWVSGISGSGGSTYYADSVKGRFTISRDNSKNT |
| | | LYLQMNSLRAEDTAVYYCAKISFYPGGTYFDYWGQGTL |
| | | VTVSS |
| 13 | LC CDR1 | RASQSISSYLN |
| 14 | LC CDR2 | AASSLQS |
| 441 | LC CDR3 | QQSYSSPYT |
| 16 | HC CDR1 | SYAMS |
| 442 | HC CDR2 | GISGSGGSTYYADSVKG |
| 158 | HC CDR3 | ISFYPGGTYFDY |

ANTIBODY 4

| | | |
|---|---|---|
| 443 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPYTFGQGTKLEI K |
| 444 | VH | EVQLLESGGGLVQPGGSLRLSCAASGFTFSSYAMSWVRQ |
| | | APGKGLEWVSGISGSGGSTYYADSVKGRFTISRDNSKNT |
| | | LYLQMNSLRAEDTAVYYCAKISFYPGGTYFDYWGQGTL |
| | | VTVSS |
| 13 | LC CDR1 | RASQSISSYLN |
| 14 | LC CDR2 | AASSLQS |
| 445 | LC CDR3 | QQSYSTPYT |
| 16 | HC CDR1 | SYAMS |
| 442 | HC CDR2 | GISGSGGSTYYADSVKG |
| 158 | HC CDR3 | ISFYPGGTYFDY |

ANTIBODY 5

| | | |
|---|---|---|
| 446 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYVCQQSYSTPYTFGQGTKLEI K |
| 447 | VH | EVQLLESGGGLVQPGGSLRLSCAASGFTFSSYAMNWVR |
| | | QAPGKGLEWVSTISSSGSRTYYADSVKGRFTISRDNSKNT |
| | | LYLQMNSLRAEDTAVYVCAKGLVASAPFDYWGQGTLV |
| | | TVSS |
| 13 | LC CDR1 | RASQSISSYLN |
| 14 | LC CDR2 | AASSLQS |
| 445 | LC CDR3 | QQSYSTPYT |
| 149 | HC CDR1 | SYAMN |
| 448 | HC CDR2 | TISSSGSRTYYADSVKG |
| 159 | HC CDR3 | GLVASAPFD |

ANTIBODY 6

| | | |
|---|---|---|
| 449 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPYTFGQGTKLEIK |
| 450 | VH | EVQLLESGGGLVQPGGSLRLSCAASGFTFSSYAMSWVRQ |
| | | APGKGLEWVSGISGGGGSTYYADSVKGRFTISRDNSKNT |
| | | LYLQMNSLRAEDTAVYYCAKIAHSRIGWHFDYWGQGTL |
| | | VTVSS |
| 13 | LC CDR1 | RASQSISSYLN |
| 14 | LC CDR2 | AASSLQS |
| 445 | LC CDR3 | QQSYSTPYT |
| 16 | HC CDR1 | SYAMS |
| 155 | HC CDR2 | GISGGGGSTYYADSVKG |
| 160 | HC CDR3 | IAHSRIGWHFDY |

ANTIBODY 7

| | | |
|---|---|---|
| 451 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPYTFGQGTKLEI K |
| 452 | VH | EVQLLESGGGLVQPGGSLRLSCAASGFTFSRYAMTWVR |
| | | QAPGKGLEWVSSISASGGSTYYADSVKGRFTISRDNSKN |
| | | TLYLQMNSLRAEDTAVYVCAKTFSGYAHYDFDYWGQG |
| | | TLVTVSS |
| 13 | LC CDR1 | RASQSISSYLN |
| 14 | LC CDR2 | AASSLQS |
| 445 | LC CDR3 | QQSYSTPYT |
| 151 | HC CDR1 | RYAM |
| 453 | HC CDR2 | SISASGGSTYYADSVKG |
| 161 | HC CDR3 | TFSGYAHYDFDY |

ANTIBODY 8

| | | |
|---|---|---|
| 454 | VL | EIVLTQSPGTLSLSPGERATLSCRASQSVSRNYLAWYQQ |
| | | KPGQAPRLLIYGASSRATGIPDRFSGSGSGTDFTLTISRLE |
| | | PEDFAVYYCQQSLGYPPTFGQGTKVEIK |
| 455 | VH | EVQLLESGGGLVQPGGSLRLSCAASGFTFSPAYMSWVRQ |
| | | APGKGLEWVSAISGSGGSTYYADSVKGRFTISRDNSKNT |
| | | LYLQMNSLRAEDTAVYYCAKGWRAFDYWGQGTLVTVS |
| | | S |
| 137 | LC CDR1 | RASQSVSRNYLA |
| 140 | LC CDR2 | GASSRAT |
| 145 | LC CDR3 | QQSLGYPPT |
| 456 | HC CDR1 | GFTFSPAYMS |
| 457 | HC CDR2 | AISGSGGSTYYADSVK |
| 458 | HC CDR3 | GWRAFDY |

### Exemplary Anti-CD3 Antibody Sequences

SEQ ID No:

ANTIBODY 1

| | | |
|---|---|---|
| 459 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPPLTFGGGTKVEIK |
| 460 | VH | QVQLVESGGGVVQPGRSLRLSCAASGFKFSGYGMHWVR |
| | | QAPGKGLEWVAVIWYDGSKKYYVDSVKGRFTISRDNSK |
| | | NTLYLQMNSLRAEDTAVYYCARQMGYWHFDLWGRGT |
| | | LVTVSS |
| 19 | LC CDR1 | RASQSVSSYLA |
| 20 | LC CDR2 | DASNRAT |
| 21 | LC CDR3 | QQRSNWPPLT |
| 22 | HC CDR1 | GYGMH |
| 461 | HC CDR2 | VIWYDGSKKYYVDSVKGR |
| 24 | HC CDR3 | QMGYWHFDL |

ANTIBODY 2

| | | |
|---|---|---|
| 461 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPPLTFGGGTKVEIK |
| 462 | VH | QVQLVQSGGGVVQSGRSLRLSCAASGFKFSGYGMHWV |
| | | RQAPGKGLEWVAVIWYDGSKKYYVDSVKGRFTISRDNS |
| | | KNTLYLQMNSLRGEDTAVYYCARQMGYWHFDLWGRG |
| | | TLVTVSS |
| 19 | LC CDR1 | RASQSVSSYLA |
| 20 | LC CDR2 | DASNRAT |
| 21 | LC CDR3 | QQRSNWPPLT |
| 22 | HC CDR1 | GYGMH |
| 462 | HC CDR2 | IWYDGSKKYYVDSVKG |
| 24 | HC CDR3 | QMGYWHFDL |

ANTIBODY 3

| | | |
|---|---|---|
| 463 | VL | EIVLTQSPATLSLSPGERATLSCRASQSVSSYLAWYQQKP |
| | | GQAPRLLIYDASNRATGIPA |
| | | RFSGSGSGTDFTLTISSLEPEDFAVYYCQQRSNWPWTFG |
| | | QGTKVEIK |
| 464 | VH | QVQLVESGGGVVQPGRSLRLSCAASGFTFRSYGMHWVR |
| | | QAPGKGLEWVAIIWYDGSKKNYADSVKGRFTISRDNSK |
| | | NTLYLQMNSLRAEDTAVYYCARGTGYNWFDPWGQGTL |
| | | VTVSS |
| 19 | LC CDR1 | RASQSVSSYLA |
| 20 | LC CDR2 | DASNRAT |
| 170 | LC CDR3 | QQRSNWPWT |
| 172 | HC CDR1 | SYGMH |
| 174 | HC CDR2 | AIWYNGRKQDYADSVKG |
| 175 | HC CDR3 | GTGYNWFDP |

ANTIBODY 4

| | | |
|---|---|---|
| 465 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 466 | VH | EVQLVESGGGLVQPGGSLKLSCAASGFTFNIYAMNWVR |
| | | QAPGKGLEWVARIRSKYNNYATYYADSVKSRFTISRDDS |
| | | KNTAYLQMNNLKTEDTAVYYCVRHGNFGNSYVSFFAY |
| | | WGQGTLVTVSS |
| 467 | LC CDR1 | GSSTGAVTSGYYPN |
| 468 | LC CDR2 | GTKFLAP |
| 469 | LC CDR3 | ALWYSNRWV |
| 470 | HC CDR1 | IYAMN |
| 471 | HC CDR2 | RIRSKYNNYATYYADSVKS |
| 472 | HC CDR3 | HGNFGNSYVSFFAY |

ANTIBODY 5

| | | |
|---|---|---|
| 473 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 474 | VH | EVQLVESGGGLVQPGGSLKLSCAASGFTFNKYAMNWVR |
| | | QAPGKGLEWVARIRSKYNNYATYYADSVKDRFTISRDD |
| | | SKNTAYLQMNNLKTEDTAVYYCVRHGNFGNSYISYWA |
| | | YWGQGTLVTVSS |
| 467 | LC CDR1 | GSSTGAVTSGYYPN |
| 468 | LC CDR2 | GTKFLAP |
| 469 | LC CDR3 | ALWYSNRWV |
| 475 | HC CDR1 | KYAMN |
| 471 | HC CDR2 | RIRSKYNNYATYYADSVKS |
| 472 | HC CDR3 | HGNFGNSYISYWAY |

ANTIBODY 6

| | | |
|---|---|---|
| 473 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 474 | VH | EVQLVESGGGLEQPGGSLKLSCAASGFTFNSYAMNWVR |
| | | QAPGKGLEWVARIRSKYNNYATYYADSVKGRFTISRDD |
| | | SKNTAYLQMNNLKTEDTAVYYCVRHGNFGNSYLSFWA |
| | | YWGQGTLVTVSS |
| 467 | LC CDR1 | GSSTGAVTSGYYPN |
| 468 | LC CDR2 | GTKFLAP |
| 469 | LC CDR3 | ALWYSNRWV |
| 149 | HC CDR1 | SYAMN |
| 475 | HC CDR2 | RIRSKYNNYATYYADSVKG |
| 476 | HC CDR3 | HGNFGNSYLSFWAY |

ANTIBODY 7

| | | |
|---|---|---|
| 477 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 478 | VH | EVQLVESGGGLVQPGGSLKLSCAASGFTFNRYAMNWVR |
| | | QAPGKGLEWVARIRSKYNNYATYYADSVKGRFTISRDD |
| | | SKNTAYLQMNNLKTEDTAVYYCVRHGNFGNSYLSYFA |
| | | YWGQGTLVTVSS |
| 467 | LC CDR1 | GSSTGAVTSGYYPN |
| 468 | LC CDR2 | GTKFLAP |
| 469 | LC CDR3 | ALWYSNRWV |
| 479 | HC CDR1 | RYAMN |
| 480 | HC CDR2 | RIRSKYNNYATYYADS |
| 481 | HC CDR3 | HGNFGNSYLSYFAY |

### Exemplary Anti-CD20 Antibody Sequences

SEQ ID No:

ANTIBODY 1

| | | |
|---|---|---|
| 482 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPLTFGGGTKVEIK |
| 483 | VH | AVQLVESGGGLVQPGRSLRLSCAASGFTFGDYTMHWVR |
| | | QAPGKGLEWVSGISWNSGSIGYADSVKGRFTISRDNAKN |
| | | SLYLQMNSLRAEDTALYYCTKDNQYGSGSTYGLGVWG |
| | | QGTLVTVSS |
| 19 | LC CDR1 | RASQSVSSYLA |
| 20 | LC CDR2 | DASNRAT |
| 484 | LC CDR3 | QQRSNWPLT |
| 485 | HC CDR1 | DYTMH |
| 486 | HC CDR2 | GISWNSGSILGYADSVKG |
| 487 | HC CDR3 | DNQYGSGSTYGLGV |

ANTIBODY 2

| | | |
|---|---|---|
| 488 | VL | DIQMTQSPSSLSASVGDRVTITCRASQGISSWLAWYQQK |
| | | PEKAPKSLIYAASSLQSGVPSRFSGSGSGTDFTLTISSLQPE |
| | | DFATYYCQQYNSVFTFGPGTKVDIK |
| 489 | VH | AVQLVESGGGLVQPGRSLRLSCAASGFTFGDYTMHWVR |
| | | QAPGKGLEWVSGISWNSGSIGYADSVKGRFTISRDNAKN |
| | | SLYLQMNSLRAEDTALYYCTKDNQYGSGSTYGLGVWG |
| | | QGTLVTVSS |
| 490 | LC CDR1 | RASQGISSWLA |
| 14 | LC CDR2 | AASSLQS |
| 491 | LC CDR3 | QQYNSVFT |
| 485 | HC CDR1 | DYTMH |
| 486 | HC CDR2 | GISWNSGSILGYADSVKG |
| 487 | HC CDR3 | DNQYGSGSTYGLGV |

ANTIBODY 3

| | | |
|---|---|---|
| 492 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSDWPLTFGGGTKVEIK |
| 493 | VH | AVQLVESGGGLVQPGRSLRLSCAASGFTFGDYTMHWVR |
| | | QAPGKGLEWVSGISWNSGSIGYADSVKGRFTISRDNAKN |
| | | SLYLQMNSLRAEDTALYYCTKDNQYGSGSTYGLGVWG |
| | | QGTLVTVSS |
| 19 | LC CDR1 | RASQSVSSYLA |
| 20 | LC CDR2 | DASNRAT |
| 494 | LC CDR3 | QQRSDWPLT |
| 485 | HC CDR1 | DYTMH |
| 486 | HC CDR2 | GISWNSGSILGYADSVKG |
| 487 | HC CDR3 | DNQYGSGSTYGLGV |

ANTIBODY 4

| | | |
|---|---|---|
| 495 | VL | QIVLSQSPAILSASPYEKVTMTCRASSSVSYIHWFQQKPG |
| | | SSPKPWIYATSNLASGVPVRFSGSGSGTSYSLTISRVEAED |
| | | AATYYCQQWTSNPPTFGGGTKLEIK |
| 496 | VH | QVQLQQPGAELVKPGASVKMSCKASGYTFTSYNMHWV |
| | | KQTPGRGLEWIGAIYPGNGDTSYNQKFKGKATLTADKSS |
| | | STAYMQLSSLTSEDSAVYYCARSTYYGGDWYFNVWGT |
| | | TVTVSA |
| 25 | LC CDR1 | RASSSVSYIH |
| 26 | LC CDR2 | ATSNLAS |
| 27 | LC CDR3 | QQWTSNPPT |
| 28 | HC CDR1 | SYNMH |
| 29 | HC CDR2 | AIYPGNGDTSYNQKFKG |
| 192 | HC CDR3 | STYYGGDWYFNV |

### Exemplary Anti-CXCR4 Antibody Sequences

SEQ ID No:

ANTIBODY 1

| | | |
|---|---|---|
| 497 | VL | DIVMTQSPDSLAVSLGERATINCKSSQSLFNSRTRKNYLA |
| | | WYQQKPGQPPKLLIYWASTRESGVPDRFSGSGSGTDFTL |
| | | TISSLQAEDVAVYYCMQSFNLRTFGQGTKVEIK |
| 498 | VH | EVQLVESGGGLVQPGRSLRLSCTASGFTFTDNYMSWVR |
| | | QAPGKGLEWVGFIRNKANGYTT |
| | | EYAASVKGRFTISRDDSKSIAYLQMNSLKTEDTAVYYCA |
| | | RDVGSNYFDYWGQGTLVTVSS |
| 499 | LC CDR1 | NSLFNSRTRKNY |
| 32 | LC CDR2 | WAS |
| 211 | LC CDR3 | MQSFNLRT |
| 221 | HC CDR1 | GFTFTDNY |
| 35 | HC CDR2 | IRNKANGYTT |
| 268 | HC CDR3 | ARDVGSNYFDY |

ANTIBODY 2

| | | |
|---|---|---|
| 500 | VL | DIVMTQSPSSLAVSLGERATMSCKSSQSLFNSRTRKNYL |
| | | AWYQQKPGQSPKLLIYWASARDSGVPARFTGSGSETYFT |
| | | LTISRVQAEDLAVYYCMQSFNLRTFGQGTKVEIK |
| 501 | VH | EVNLVESGGGLVQPGRSLRLSCTASGFTFTDNYMSWVR |
| | | QAPGKGLEWLGFIRNKANGYTTDYAASVRGRFTISRDNS |
| | | KSILYLQMNALRTEDTAVYYCARDVGSNYFDYWGQGT |
| | | LVTVSS |
| 499 | LC CDR1 | NSLFNSRTRKNY |
| 32 | LC CDR2 | WAS |
| 211 | LC CDR3 | MQSFNLRT |
| 221 | HC CDR1 | GFTFTDNY |
| 268 | HC CDR3 | ARDVGSNYFDY |

ANTIBODY 3

| | | |
|---|---|---|
| 502 | VL | DIVMSQSPSSLAVSAGEKVTMSCKSSQSLFNSRTRKNYL |
| | | AWYQQKPGQSPKLLIYWASARDSGVPARFTGSGSETYFT |
| | | LTISRVQAEDLAVYYCMQSFNLRTFGGGTKLEIK |
| 503 | VH | EVNLVESGGGLVQPGGSLRLSCATSGFTFTDNYMSWVR |
| | | QPPGKALEWLGFIRNKANGYTTDYSASVRGRFTISRDNS |
| | | QSILYLQMNALRAEDSATYYCARDVGSNYFDYWGQGTT |
| | | LTVSS |
| 504 | LC CDR1 | QSLFNSRTRKNY |
| 32 | LC CDR2 | WAS |
| 211 | LC CDR3 | MQSFNLRT |
| 221 | HC CDR1 | GFTFTDNY |
| 35 | HC CDR2 | IRNKANGYTT |
| 268 | HC CDR3 | ARDVGSNYFDY |

ANTIBODY 4

| | | |
|---|---|---|
| 505 | VL | SSELTQDPAVSVALGQTVRITCQGDSLRKFFASWYQQKP |
| | | GQAPVLVIYGKNSRPSGIPDRFSGSNSRNTASLTITGAQA |
| | | EDEGDYYCNSRDSRDNHQVFGAGTKVTVLS |
| 506 | VH | QVQLVQSGAEVKKPGASVKVSCKASGYTFTSYGISWVR |
| | | QAPGQGLEWMGWISAYNGNTNYAQKLQGRVTMTTDTS |
| | | TSTAYMELRSLRSDDTAVYYCARDTPGIAARRYYYYGM |
| | | DVWGQGT TVTVSS |
| 197 | LC CDR1 | QGDSLRKFFAS |
| 206 | LC CDR2 | GKNSRPS |
| 215 | LC CDR3 | NSRDSRDNHQV |
| 227 | HC CDR1 | SYGIS |
| 249 | HC CDR2 | WISAYNGNTNYAQKLQG |
| 274 | HC CDR3 | DTPGIAARRYYYYGMDV |

ANTIBODY 5

| | | |
|---|---|---|
| 507 | VL | QPVLTQPRSVSGSPGQSVTISCTGTISDVGGHNFVSWYQQ |
| | | NPGKAPKLIIFEVTKRPAGVPDRFSGSKSGNTASLTVSGL |
| | | QAEDEGEYYCSSYGGSNDVIFGGGTKLSVLG |
| 508 | VH | EVQLVESGGGVVQPGRSLRLSCAASGFTFSSYGMHWVR |
| | | QAPGKGLEWVAVISYDGSNKYYADSVKGRFTISRDNSK |
| | | NTLYLQVSSLRAEDTAVYYCVRDLVAAAGTAFDIWGQG |
| | | TTVTVSS |
| 195 | LC CDR1 | TGTISDVGGHNFVS |
| 204 | LC CDR2 | EVTKRPA |
| 213 | LC CDR3 | SSYGGSNDVI |
| 225 | HC CDR1 | SYGMH |
| 247 | HC CDR2 | VISYDGSNKYYADSVKG |
| 272 | HC CDR3 | DLVAAAGTAFDI |

ANTIBODY 6

| | | |
|---|---|---|
| 509 | VL | QSVLTQPPSVSKGLRQTATLTCTGNSNNVGNQGAAWLQ |
| | | QHQGHPPKLLSYRNNNRPSGIS |
| | | ERFSASRSGNTASLTITGLQPEDEADYYCSAWDNRLKTY |
| | | VFGTGGKVTVLG |
| 510 | VH | QVQLQQSGPGLVKPSQTLSLTCAVSGDSVSSNFVAWNWI |
| | | RQSPSRGLEWLGRTYYRSRWY |
| | | NDYAVSVQSRIRVTPDTSKNQFSLHLDSVTPEDTAVYYC |
| | | ARGQHSGFDFWGQGTLVTVSS |
| 196 | LC CDR1 | TGNSNNVGNQGAA |
| 205 | LC CDR2 | RNNNRPS |
| 214 | LC CDR3 | SAWDNRLKTYV |
| 226 | HC CDR1 | SNFVAWN |
| 248 | HC CDR2 | RTYYRSRWYNDYAVSVQS |
| 273 | HC CDR3 | GQHSGFDF |

ANTIBODY 7

| | | |
|---|---|---|
| 511 | VL | EIVLTQSPATLSVSPGERATLSCRASQSVNTNLAWYQQK |
| | | PGQAPRLLIYGASSRATGIPDRFSGSGSGTDFTLTISRLEPE |
| | | DFAVYYCQHYGSSPLTFGGGTKLEIKR |
| 512 | VH | QVQLVQSGGGVVQPGRSLRLSCAASGFTFSSYPMHWVR |
| | | QAPGKGLEWMTVISSDGRNKYYPDSVKGRFTISRDNSKN |
| | | TLYLQMNSLRPEDTAVYYCARGGYHDFWSGPDYWGQG |
| | | TLVTVSS |
| 198 | LC CDR1 | RASQSVNTNLA |
| 140 | LC CDR2 | GASSRAT |
| 216 | LC CDR3 | QHYGSSPLT |
| 228 | HC CDR1 | SYPMH |
| 250 | HC CDR2 | VISSDGRNKYYPDSVKG |
| 275 | HC CDR3 | GGYHDFWSGPDY |

ANTIBODY 8

| | | |
|---|---|---|
| 513 | VL | SSELTQDPAVSVALGQTVRITCQGDSLRSYYASWYQQKP |
| | | GQAPVLVIYGKNNRPSGIPDRFSGSKSHNTAYLTITGAQA |
| | | EDEADYFCNSRSGSQRVFGGGTKLTVLG |
| 524 | VH | EVQLVQSGGGLVQPGGSLRLSCAASGFTFSSYAMSWVR |
| | | QAPGKGLEWVANIKQDGSEKYYVDSVKGRFTISRDNAK |
| | | NSLYLQMNSLRAEDTAVYYCARDQVSGITIFGGKWRSP |
| | | DVWGQGTTVTVSS |
| 199 | LC CDR1 | QGDSLRSYYAS |
| 208 | LC CDR2 | GKNNRPS |
| 217 | LC CDR3 | NSRSGSQRV |
| 231 | HC CDR1 | SYAMS |
| 251 | HC CDR2 | NIKQDGSEKYYVDSVKG |
| 276 | HC CDR3 | DQVSGITIFGGKWRSPDV |

ANTIBODY 9

| | | |
|---|---|---|
| 525 | VL | QSVLTQPPSASGTPGQRVTISCSGSRSNIGSNTVNWYQQL |
| | | PGTAPKLLIYTNNQRPSGVPDRFSGSKSGTSASLAITGLQ |
| | | AEDEADYYCLSFDSSLTSYVFGTGTKVTVLG |
| 526 | VH | QVTLKESGGGVVQPGRSLRLSCAASGFTFTNYGLHWVR |
| | | QAPGKGLEWVAVISHDGTKKYYADSVKGRFTISRDSSEN |
| | | TLYLQMNSLRPEDSALYYCARDGGYCSGGRCYSYGMD |
| | | VWGQGTTVTVSS |
| 200 | LC CDR1 | SGSRSNIGSNTVN |
| 209 | LC CDR2 | TNNQRPS |
| 218 | LC CDR3 | LSFDSSLTSYV |
| 229 | HC CDR1 | NYGLH |
| 252 | HC CDR2 | VISHDGTKKYYADSVKG |
| 277 | HC CDR3 | DGGYCSGGRCYSYGMDV |

ANTIBODY 10

| | | |
|---|---|---|
| 527 | VL | QSVLTQPPSASGTPGQRVTISCSGSRSNIGGNTVNWYQQL |
| | | PGTAPKLLIYANNQRPSGVP |
| | | DRFSGSKSGTSASLAISGLRSEDEADYYCAAWDDNLSGH |
| | | VVFGGGTKLTVLR |
| 528 | VH | QVQLVQSGGGVVLPGRSLRLSCVASGFTFRRYGMHWVR |
| | | QAPGKGLEWVSLISYDGSKTFYGESVKGRFTISRDNSKN |
| | | TLYLQMNSLRAEDTAVYYCARATVTTDGYYYMDVWG |
| | | KGTTVTV SS |
| 201 | LC CDR1 | SGSRSNIGGNTVN |
| 210 | LC CDR2 | ANNQRPS |
| 529 | LC CDR3 | AAWDDNLSGHVV |
| 230 | HC CDR1 | RYGMH |
| 253 | HC CDR2 | LISYDGSKTFYGESVKG |
| 278 | HC CDR3 | ATVTTDGYYYMDV |

### Exemplary Anti-HER2 Antibody Sequences

SEQ ID No:

ANTIBODY 1

| | | |
|---|---|---|
| 530 | VL | DIQMTQSPSSLSASVGDRVTITCRASQDVNTAVAYQQKP |
| | | GKAPKLLIYSASFLYSGVPSRFSGSRSGTDFTLTISSLQPE |
| | | DFATYYCQQHYTTPPTFGQGTKVEIKRT |
| 531 | VH | EVQLVESGGGLVQPGGSLRLSCAASGFNIKDTYIHWVRQ |
| | | APGKGLEWVARIYPTNGYTRYADSVKGRFTISADTSKNT |
| | | AYLQMNSLRAEDTAVYYCSRWGGDGFYAMDYWGQGT |
| | | LVTVSSA |
| 295 | LC CDR1 | RASQDVNTAVA |
| 304 | LC CDR2 | SASFLYS |
| 532 | LC CDR3 | QQHYTTPPT |
| 309 | HC CDR1 | GFNIKDTYIH |
| 533 | HC CDR2 | RIYPTNGYTRYADSVKG |
| 316 | HC CDR3 | WGGDGFYAMDY |

ANTIBODY 2

| | | |
|---|---|---|
| 534 | VL | DIQMTQSPSSLSASVGDRVTITCKASQDVSIGVAWYQQK |
| | | PGKAPKLLIYSASYRYTGVPSRFSGSGSGTDFTLTISSLQP |
| | | EDFATYYCQQYYIYPYTFGQGTKVEIK |
| 535 | VH | EVQLVESGGGLVQPGGSLRLSCAASGFTFTDYTMDWVR |
| | | QAPGKGLEWVADVNPNSGGSIYNQRFKGRFTLSVDRSK |
| | | NTLYLQMNSLRAEDTAVYYCARNLGPSFYFDYWGQGTL |
| | | VTVSS |
| 37 | LC CDR1 | KASQDVSIGVA |
| 536 | LC CDR2 | SASYRY |
| 537 | LC CDR3 | QQYYIYPY |
| 40 | HC CDR1 | GFTFTDYTMD |
| 538 | HC CDR2 | ADVNPNSGGSIYNQRFK |
| 539 | HC CDR3 | ARNLGPSFYFDY |

ANTIBODY 3

| | | |
|---|---|---|
| 540 | VL | DTVMTQSHKIMSTSVGDRVSITCKASQDVSIGVAWYQQ |
| | | RPGQSPKLLIYSASYRYTGVPDRFTGSGSGTDFTFTISSVQ |
| | | AEDLAVYYCQQYYIYPYTFGGGTKLEIK |
| 541 | VH | EVQLQQSGPELVKPGTSVKISCKASGFTFTDYTMDWVK |
| | | QSHGKSLEWIGDVNPNSGGSIYNQRFKGKASLTVDRSSRI |
| | | VYMELRSLTFEDTAVYYCARNLGPSFYFDYWGQGTTLT |
| | | VSS |
| 37 | LC CDR1 | KASQDVSIGVA |
| 384 | LC CDR2 | SASYRYT |
| 39 | LC CDR3 | QQYYIYPYT |
| 40 | HC CDR1 | GFTFTDYTMD |
| 41 | HC CDR2 | DVNPNSGGSIYNQRFKG |
| 42 | HC CDR3 | NLGPSFYFDY |

ANTIBODY 4

| | | |
|---|---|---|
| 542 | VL | DIQMTQSPSSLSASVGDRVTITCKASQDVSIGVAWYQQK |
| | | PGKAPKLLIYSASYRYTGVPSRFSGSGSGTDFTLTISSLQP |
| | | EDFATYYCQQYYIYPYTFGQGTKVEIK |
| 543 | VH | EVQLVESGGGLVQPGGSLRLSCAASGFTFTDYTMDWVR |
| | | QAPGKGLEWVADVNPNSGGSIYNQRFKGRFTLSVDRSK |
| | | NTLYLQMNSLRAEDTAVYYCARNLGPSFYFDYWGQGTL |
| | | VTVSS |
| 37 | LC CDR1 | KASQDVSIGVA |
| 384 | LC CDR2 | SASYRYT |
| 39 | LC CDR3 | QQYYIYPYT |
| 40 | HC CDR1 | GFTFTDYTMD |
| 41 | HC CDR2 | DVNPNSGGSIYNQRFKG |
| 42 | HC CDR3 | NLGPSFYFDY |

ANTIBODY 5

| | | |
|---|---|---|
| 544 | VL | DIQMTQSPSSLSASVGDRVTITCRASQSISNYLAWYQQKP |
| | | GKAPKLLIYAASSLESGVPSRFSGSGSGTDFTLTISSLQPE |
| | | DFATYYCQQYNSLPWTFGQGTKVEIK |
| 545 | VH | EVQLVESGGGLVQPGGSLRLSCAASGFTFSSYAMSWVR |
| | | QAPGKGLEWVAVISGDGGSTYYADSVKGRFTISRDNSK |
| | | NTLYLQMNSLRAEDTAVYYCARGRVGYSLYDYWGQGT |
| | | LVTVSS |
| 546 | LC CDR1 | RASQSISNYLA |
| 547 | LC CDR2 | AASSLES |
| 548 | LC CDR3 | QQYNSLPWT |
| 549 | HC CDR1 | GFTFSSYAMS |
| 550 | HC CDR2 | VISGDGGSTYYADSVKG |
| 551 | HC CDR3 | GRVGYSLYDY |

Exemplary Anti-VEGF Antibody Sequences

SEQ ID No:

ANTIBODY 1

| | | |
|---|---|---|
| 552 | VL | DIQMTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIK |
| 553 | VH | EVQLVESGGGLVQPGGSLRLSCAASGYTFTNYGMNWVR |
| | | QAPGKGLEWVGWINTYTGEPTYAADFKRRFTSLDTSKST |
| | | AYLQMNSLRAEDTAVYYCAKYPHYYGSSHWYFDVWG |
| | | QGTLVTVSS |
| 554 | LC CDR1 | QDISNY |
| 555 | LC CDR2 | FTS |
| 45 | LC CDR3 | QQYSTVPWT |
| 556 | HC CDR1 | GYTFTNYG |
| 557 | HC CDR2 | INTYTGEP |
| 558 | HC CDR3 | AKYPHYYGSSHWYFDV |

ANTIBODY 2

| | | |
|---|---|---|
| 559 | VL | DIQLTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIK |
| 560 | VH | EVQLVESGGGLVQPGGSLRLSCAASGYDFTHYGMNWV |
| | | RQAPGKGLEWVGWINTYTGEPTYAADFKRRFTSLDTSK |
| | | STAYLQMNSLRAEDTAVYYCAKYPYYYGTSHWYFDVW |
| | | GQGTLVTVSS |
| 554 | LC CDR1 | QDISNY |
| 555 | LC CDR2 | FTS |
| 45 | LC CDR3 | QQYSTVPWT |
| 561 | HC CDR1 | GYDFTHYG |
| 557 | HC CDR2 | INTYTGEP |
| 562 | HC CDR3 | AKYPYYYGTSHWYFDV |

ANTIBODY 3

| | | |
|---|---|---|
| 563 | VL | EIVMTQSPSTLSASVGDRVIITCQASEIIHSWLAWYQQKP |
| | | GKAPKLLIYLASTLASGVPSRFSGSGSGAEFTLTISSLQPD |
| | | DFATYYCQNVYLASTNGANFGQGTKLTVLG |
| 564 | VH | SLRLSCTASGFSLTDYYYMTWVRQAPGKGLEWVGFIDP |
| | | DDDPYYATWAKGRFTISRDNSKNTLYLQMNSLRAEDTA |
| | | VYYCAGGDHNSGWGLDIWGQGTLVTVSS |
| 565 | LC CDR1 | QASEIIHSWLA |
| 566 | LC CDR2 | LASTLAS |
| 567 | LC CDR3 | QNVYLASTNGAN |
| 568 | HC CDR1 | GFSLTDYYYMT |
| 569 | HC CDR2 | FIDPDDDPYYATWAKG |
| 570 | HC CDR3 | GDHNSGWGLDI |

ANTIBODY 4

| | | |
|---|---|---|
| 571 | VL | EIVMTQSPSTLSASVGDRVIITCQSSQSVYGNIWMAWYQ |
| | | QKPGKAPKLLIYQASKLASGVPSRFSGSGSGAEFTLTISSL |
| | | QPDDFATYYCQGNFNTGDRYAFGQGTKLTVLG |
| 572 | VH | EVQLVESGGGLVQPGGSLRLSCAASGFTISRSYWICWVR |
| | | QAPGKGLEWVSCIYGDNDITPLYANWAKGRFTISRDNSK |
| | | NTLYLQMNSLRAEDTAVYYCAKLGYADYAYDLWGQGT |
| | | LVTVSS |
| 573 | LC CDR1 | QSSQSVYGNIWMA |
| 574 | LC CDR2 | QASKLAS |
| 575 | LC CDR3 | QGNFNTGDRYA |
| 576 | HC CDR1 | GFTISRSYWIC |
| 577 | HC CDR2 | CIYGDNDITPLYANWA |
| 578 | HC CDR3 | LGYADYAYDL |

ANTIBODY 5

| | | |
|---|---|---|
| 579 | VL | DIQMTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIKRTV |
| 580 | VH | EVQLVESGGGLVQPGGSLRLSCAASGYTFTNYGMNWVR |
| | | QAPGKGLEWVGWINTYTGEPTYAADFKRRFTFSLDTSKS |
| | | TAYLQMNSLRAEDTAVYYCAKYPHYYGSSHWYFDVWG |
| | | QGTL |
| 43 | LC CDR1 | SASQDISNYLN |
| 44 | LC CDR2 | FTSSLHS |
| 45 | LC CDR3 | QQYSTVPWT |
| 46 | HC CDR1 | GYTFTNYGMN |
| 337 | HC CDR2 | WINTYTGEPTYAADFKR |
| 581 | HC CDR3 | YPHYYGSSHWYFDV |

ANTIBODY 6

| | | |
|---|---|---|
| 582 | VL | DIQLTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIKRTV |
| 583 | VH | EVQLVESGGGLVQPGGSLRLSCAASGYDFTHYGMNWV |
| | | RQAPGKGLEWVGWINTYTGEPTYAADFKRRFTFSLDTS |
| | | KSTAYLQMNSLRAEDTAVYYCAKYPYYYGTSHWYFDV |
| | | WGQGTL |
| 43 | LC CDR1 | SASQDISNYLN |
| 44 | LC CDR2 | FTSSLHS |
| 45 | LC CDR3 | QQYSTVPWT |
| 332 | HC CDR1 | GYDFTHYGMN |
| 337 | HC CDR2 | WINTYTGEPTYAADFKR |
| 584 | HC CDR3 | YPYYYGTSHWYFDV |

Exemplary anti-PD-L1 antibody sequences

SEQ ID No:

ANTIBODY 1

| | | |
|---|---|---|
| 585 | VL | DIQMTQSPSSLSASVGDRVTITCRASQDVSTAVAWYQQK |
| | | PGKAPKLLIYSASFLYSGVPSRFSGSGSGTDFTLTISSLQPE |
| | | DFATYYCQQYLYHPATFGQGTKVEIK |
| 586 | VH | EVQLVESGGGLVQPGGSLRLSCAASGFTFSDSWIHWVRQ |
| | | APGKGLEWVAWISPYGGSTYYADSVKGRFTISADTSKNT |
| | | AYLQMNSLRAEDTAVYYCARRHWPGGFDYWGQGTLVT |
| 345 | LC CDR1 | DVSTAVA |
| 346 | LC CDR2 | SASFLY |
| 347 | LC CDR3 | QQYLYHPAT |
| 348 | HC CDR1 | DSWIH |
| 349 | HC CDR2 | WISPYGGSTY |
| 350 | HC CDR3 | RHWPGGF |

ANTIBODY 2

| | | |
|---|---|---|
| 587 | VL | EIVLTQSPGTLSLSPGERATLSCRASQRVSSSYLAWYQQK |
| | | PGQAPRLLIYDASSRATGIPDRFSGSGSGTDFTLTISRLEPE |
| | | DFAVYYCQQYGSLPWTFGQGTKVEIK |
| 588 | VH | EVQLVESGGGLVQPGGSLRLSCAASGFTFSRYWMSWVR |
| | | QAPGKGLEWVANIKQDGSEKYYVDSVKGRFTISRDNAK |
| | | NSLYLQMNSLRAEDTAVYYCAREGGWFGELAFDYWGQ |
| | | GTLVT |
| 357 | LC CDR1 | RVSSSYLA |
| 361 | LC CDR2 | DASSRA |
| 365 | LC CDR3 | QQYGSLPWT |
| 369 | HC CDR1 | RYWMS |
| 373 | HC CDR2 | NIKQDGSEKY |
| 377 | HC CDR3 | EGGWFGELAF |

ANTIBODY 3

| | | |
|---|---|---|
| 589 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPTFGQGTKVEIK |
| 590 | VH | QVQLVQSGAEVKKPGSSVKVSCKTSGDTFSTYAISWVRQ |
| | | APGQGLEWMGGIIPIFGKAHYAQKFQGRVTITADESTST |
| | | AYMELSSLRSEDTAVYFCARKFHFVSGSPFGMDVWGQG |
| | | TTVT |
| 358 | LC CDR1 | SVSSYLA |
| 362 | LC CDR2 | DASNRA |
| 366 | LC CDR3 | QQRSNWPT |
| 370 | HC CDR1 | TYAIS |
| 374 | HC CDR2 | GIIPIFGKAH |
| 378 | HC CDR3 | KFHFVSGSPFGM |

ANTIBODY 4

| | | |
|---|---|---|
| 591 | VL | QSALTQPASVSGSPGQSITISCTGTSSDVGGYNYVSWYQ |
| | | QHPGKAPKLMIYDVSNRPSGVSNRFSGSKSGNTASLTISG |
| | | LQAEDEADYYCSSYTSSSTRVFGTGTKVTVL |
| 592 | VH | EVQLLESGGGLVQPGGSLRLSCAASGFTFSSYIMMWVRQ |
| | | APGKGLEWVSSIYPSGGITFYADTVKGRFTISRDNSKNTL |
| | | YLQMNSLRAEDTAVYYCARIKLGTVTTVDYWGQGTLV |
| | | T |
| 359 | LC CDR1 | VGGYNYVS |
| 363 | LC CDR2 | DVSNRP |
| 367 | LC CDR3 | SSYTSSSTRV |
| 371 | HC CDR1 | SYIMM |
| 375 | HC CDR2 | SIYPSGGITF |
| 379 | HC CDR3 | IKLGTVTTV |

ANTIBODY 5

| | | |
|---|---|---|
| 593 | VL | QVQLVQSGAEVKKPGSSVKVSCKASGGTFSRSAISWVRQ |
| | | APGQGLEWMGVIIPAFGEANYAQKFQGRVTITADESTST |
| | | AYMELSSLRSEDTAVYYCARGRQMFGAGIDFWGQGTLV |
| | | TVSS |
| 594 | VH | EVQLVESGGGLVQPGGSLRLSCAASGFTFSDSWIHWVRQ |
| | | APGKGLEWVAWISPYGGSTYYADSVKGRFTISADTSKNT |
| | | AYLQMNSLRAEDTAVYYCARRHWPGGFDYWGQGTLVT |
| 360 | LC CDR1 | TRSSGSIDSNYVQ |
| 364 | LC CDR2 | EDNQRPS |
| 368 | LC CDR3 | QSYDSNNRHVI |
| 372 | HC CDR1 | GTFSRSAIS |
| 376 | HC CDR2 | VIIPAFGEANYAQKFQG |
| 380 | HC CDR3 | ARGRQMFGAGIDF |

### Exemplary Anti-TROP2 Antibody Sequences

SEQ ID No:

ANTIBODY 1

| | | |
|---|---|---|
| 595 | VL | DIQLTQSHKFMSTSVGDRVSITCKASQDVSIAVAWYQQK |
| | | PGQSPKLLIYSASYRYTGVPDRFTGSGSGTDFTFTISSVQA |
| | | EDLAVYYCQQHYITPLTFGAGTKLELKR |
| 596 | VH | VKLQESGPELKKPGETVKISCKASGYTFTNYGMNWVKQ |
| | | APGKGLKWMGWINTYTGEPTYTDDFKGRFAFSLETSAT |
| | | TAYLQINNLKSEDMATYFCARGGFGSSYWYFDVWGQG |
| | | TTVTVSS |
| 351 | LC CDR1 | KASQDVSIAVA |
| 352 | LC CDR2 | SASYRTT |
| 353 | LC CDR3 | QQHYITPLT |
| 354 | HC CDR1 | YNYGM |
| 355 | HC CDR2 | WINTYTGEPTYTDDFKG |
| 356 | HC CDR3 | GGFGSSYWYFDV |

ANTIBODY 2

| | | |
|---|---|---|
| 597 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPLTFGGGTKVEI |
| 598 | VH | VQLVQSGSELKKPGASVKVSCKASGYTFTSYAMNWVRQ |
| | | APGQGLEWMGWINTNTGNPTYAQGFTGRFVFSLDTSVS |
| | | TAYLQISSLKADDTAVYYCAREDSNGYKIFDY |
| 13 | LC CDR1 | RASQSISSYLN |
| 14 | LC CDR2 | AASSLQS |
| 385 | LC CDR3 | QQSYSTPLT |
| 387 | HC CDR1 | TSYAM |
| 389 | HC CDR2 | WINTNTGNPTYAQGFTG |
| 391 | HC CDR3 | EDSNGYKIFDY |

ANTIBODY 3

| | | |
|---|---|---|
| 599 | VL | DIQLTQSPSSLSASVGDRVSITCKASQDVSIAVAWYQQKP |
| | | GKAPKLLIYSASYRYTGVPDRFSGSGSGTDFTLTISSLQPE |
| | | DFAVYYCQQHYITPLTFGAGTKVEIKR |
| 600 | VH | QVQLQQSGSELKKPGASVKVSCKASGYTFTNYGMNWV |
| | | KQAPGQGLKWMGWINTYTGEPTYTDDFKGRFAFSLDTS |
| | | VSTAYLQISSLKADDTAVYFCARGGFGSSYWYFV |
| 351 | LC CDR1 | KASQDVSIAVA |
| 384 | LC CDR2 | SASYRYT |
| 353 | LC CDR3 | QQHYITPLT |
| 388 | HC CDR1 | TNYGM |
| 355 | HC CDR2 | WINTYTGEPTYTDDFKG |
| 356 | HC CDR3 | GGFGSSYWYFDV |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A conjugate comprising two or more chelators and a binding moiety, wherein one of said chelators is a chelator of an imaging isotope and one of said chelators is a chelator of a radiotherapy isotope, and wherein said chelators and said binding moiety are linked via a moiety of Formula (I): 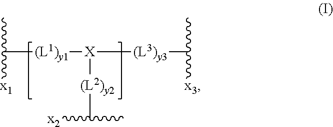
wherein:
each X is independently selected from N, P, P(═O), CR^{N}, and a moiety of formula (i): 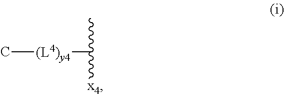
each of x₁, x₂, x₃, and x₄ independently indicates a point of attachment of the moiety of Formula (I) to said chelators or said binding moiety;
each of L¹, L², L³, and L⁴ is independently selected from C(═O), C(═S), N(R^{N}), O, S, S(═O), S(═O)₂, —CR^{N}═NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

2. The conjugate of claim 1, wherein said isotope used for radiotherapy is an α-emitter.

3. The conjugate of claim 1, wherein said radiotherapy isotope ²²⁵Ac, ²¹²Pb, ²¹¹At, ²¹³Bi, ²¹²Bi, ²¹¹Bi, ^{152/160/161}Tb, ²²⁷Th, ²²³Ra, ²¹¹Po, ²²¹Fr, ²¹⁷At, ²¹³Po, ²¹²Po, ²¹⁵Po, or ¹⁷⁷Lu.

4. The conjugate of claim 1, wherein said imaging isotope is ⁶⁸Ga, ⁴⁴Sc, ^{60/61/62/64}Cu, ^{84/86/87/89}Zr, ⁶³Zn, ^{43/44}Sc, ^{192/193/194/196}Au, ^{52m}Mn, ^{90/92ml}Nb, ^{51/52}Mn, ^{148/151/151m/152}Tb, ⁴⁵Ti, ^{65/66/67}Ga, ^{94m}Tc, ⁵⁵Co, ^{80/81/83}Sr, ³⁸K, ^{70/71/72/74}As, ^{81/82m}Rb, ⁵²Fe, or ⁸⁶Y.

5. The conjugate of claim 1, wherein said imaging isotope is ⁶⁴Cu and wherein said radiotherapy isotope is ²¹²Pb.

6. (canceled)

7. (canceled)

8. The conjugate of claim 1, wherein each of said chelators independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), and diethylenetraminepentacetic acid (DTPA).

9. The conjugate of claim 1, wherein said binding moiety is a polypeptide.

10-13. (canceled)

14. The conjugate of claim 1, wherein said chelators are covalently attached to said binding moiety.

15. The conjugate of claim 1, wherein said chelators and said binding moiety are covalently attached via a linker.

16. The conjugate of claim 15, wherein said compound of Formula (I) has formula:

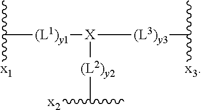

17. The conjugate of claim 16, herein the moiety of Formula (I) has any one of the following formulae:

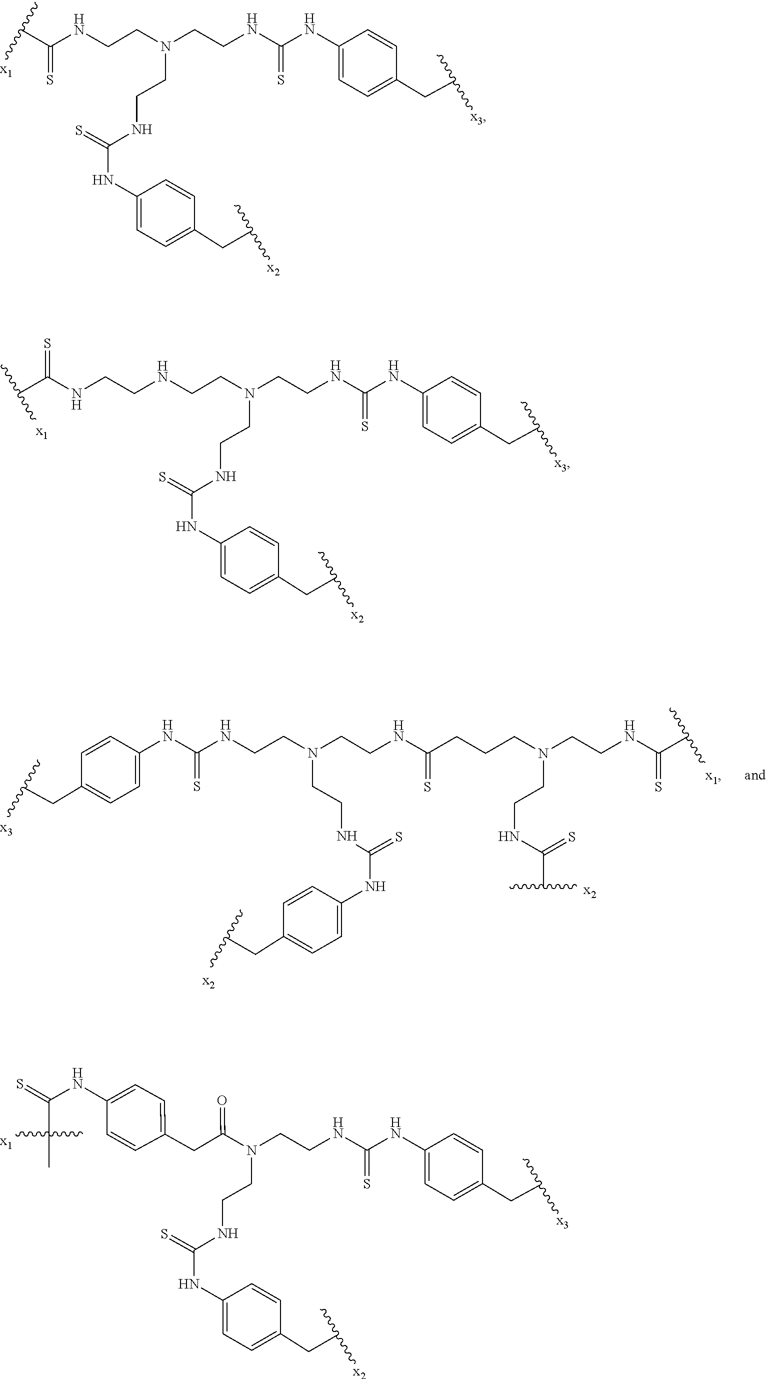

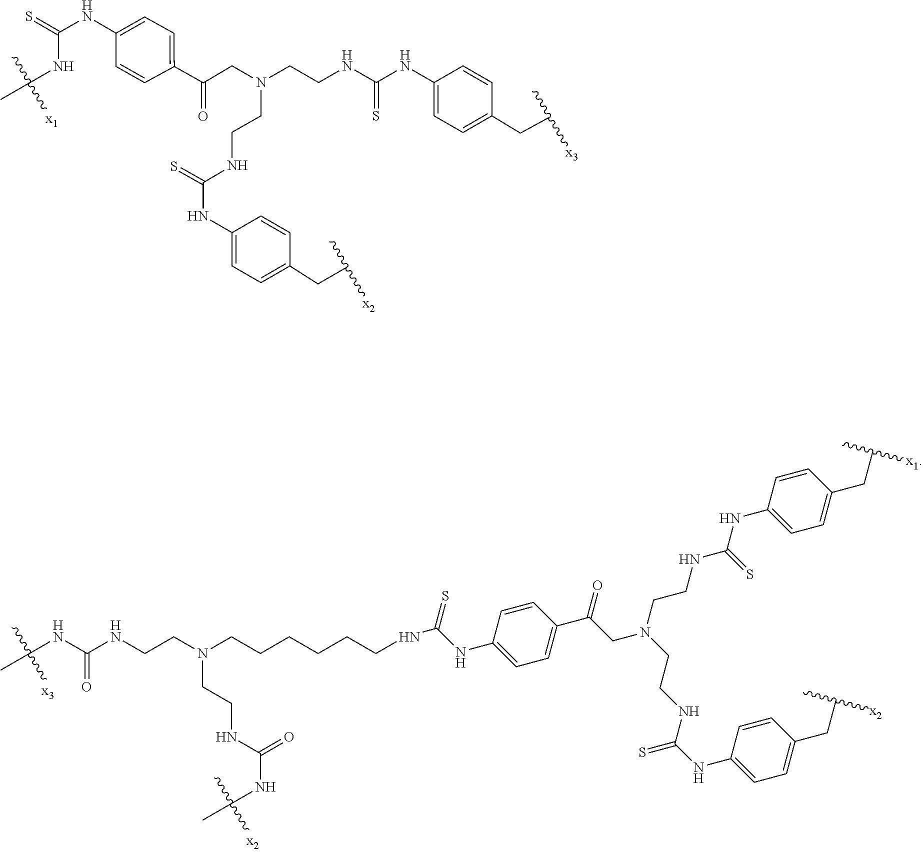

18. The conjugate of any one of claim 1, wherein said chelators and said binding moiety are linked via a moiety of Formula (II): 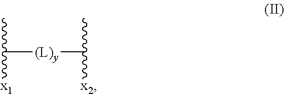
wherein:
x₁ indicates a point of attachment of the Formula (II) to the chelator;
x₂ indicates a point of attachment of the Formula (II) to the chelator or the binding moiety;
each L is independently selected from C(═O), C(═S), N(R^{N}), O, S, S(═O), S(═O)₂, —CR^{N}═NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
y is an integer from 1 to 30; and
each R^{N} is independently selected from H, C₁₋₃ alkyl, and C₁₋₃ haloalkyl.

19. The conjugate of claim 18, wherein the moiety of Formula (II) has any one of the following formulae:

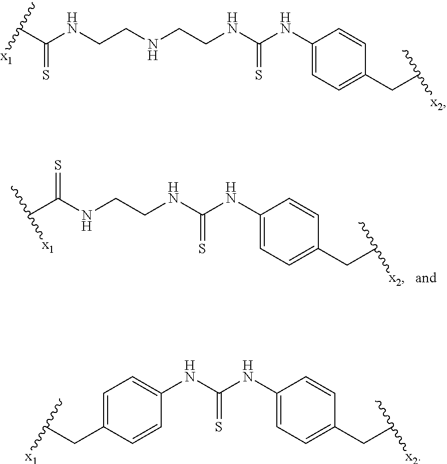

20. A method of treating cancer in a mammal in need thereof, wherein said method comprises administering a conjugate of claim 1 to said mammal, wherein said conjugate comprises said imaging isotope complexed to said chelator of said imaging isotope and wherein said conjugate comprises said radiotherapy isotope complexed to said chelator of said radiotherapy isotope.

21. A method of treating cancer in a mammal, wherein said method comprises:
a) administering, to said mammal, a first conjugate comprising two or more chelators and a binding moiety, wherein one of said chelators is a chelator of an imaging isotope and one of said chelators is a chelator of a radiotherapy isotope, wherein said first conjugate comprises said imaging isotope complexed to said chelator of said imaging isotope;
b) determining, in said mammal, the biodistribution of said first conjugate; and
c) administering, to said mammal, an amount of a second conjugate that is identical to said first conjugate except that said second conjugate comprises said radiotherapy isotope complexed to said chelator of said radiotherapy isotope.

22. The method of claim 21, said method further comprising determining, in said mammal, the biodistribution of said second conjugate comprising said imaging isotope complexed to said chelator of said imaging isotope and said radiotherapy isotope complexed to said chelator of said radiotherapy isotope.

23. The method of claim 20, wherein said cancer is selected from the group consisting of prostate cancer, a neuroendocrine cancer, colon cancer, lung cancer, pancreatic cancer, melanoma, and a lymphoid cancer.

24. (canceled)

25. A method of treating cancer in a mammal in need thereof, wherein said method comprises administering, to said mammal, two or more conjugates,
wherein each conjugate comprises two or more chelators and a binding moiety, wherein one of said chelators is a chelator of an imaging isotope and one of said chelators is a chelator of a radiotherapy isotope,
wherein one of said conjugates administered to said mammal comprises an imaging isotope complexed to said chelator of said imaging isotope, and
wherein one of said conjugates administered to said mammal comprises a radiotherapy isotope complexed to said chelator of said radiotherapy isotype.

26. (canceled)

27. The conjugate of claim 9, wherein said polypeptide binds prostate specific membrane antigen, a somatostatin receptor, a fibroblast activation protein, or a melanocortin-1 receptor.

28. The conjugate of claim 1, wherein said conjugate comprises two or more binding moieties.

29. (canceled)

30. (canceled)

31. The conjugate of claim 1, wherein said conjugate comprises three or more chelators.

32. The conjugate of claim 1, wherein each of said chelators independently comprises a compound selected from the group consisting ofNOTA, DOTA, TCMC, DiAmSar, HBED, DFO, DTPA, NTA, BisTris, EGTA, EDTA, BAPTA, DO2A, DTPA, DO3 A, and MACROPA.

33. The conjugate of claim 1, wherein said conjugate has any one of the following structures:

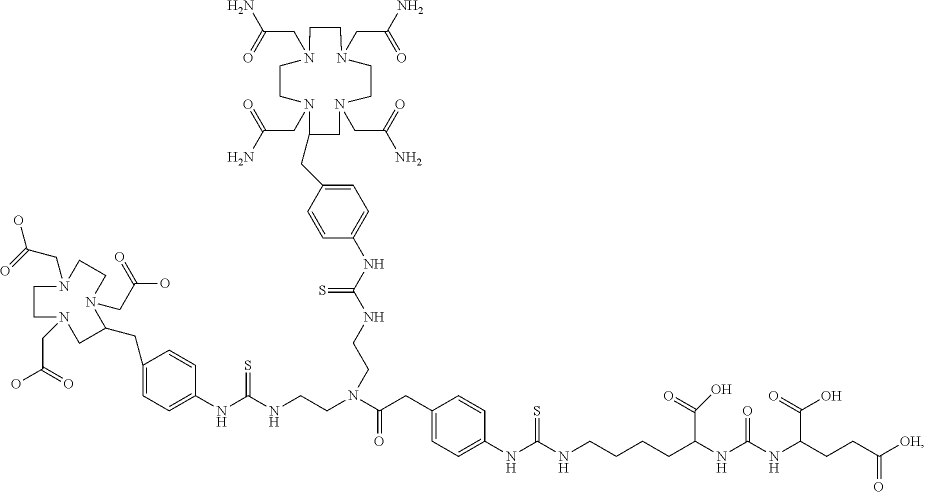 [SMILES: NC(=O)CN1CCN(CC(N)=O)CCN(CC(N)=O)C(Cc2ccc(NC(=S)NCCN(CCNC(=S)Nc3ccc(CC4CN(CC(=O)O)CCN(CC(=O)O)CCN4CC(=O)O)cc3)C(=O)Cc3ccc(NC(=S)NCCCCC(NC(=O)NC(CCC(=O)O)C(=O)O)C(=O)O)cc3)cc2)CN(CC(N)=O)CC1]

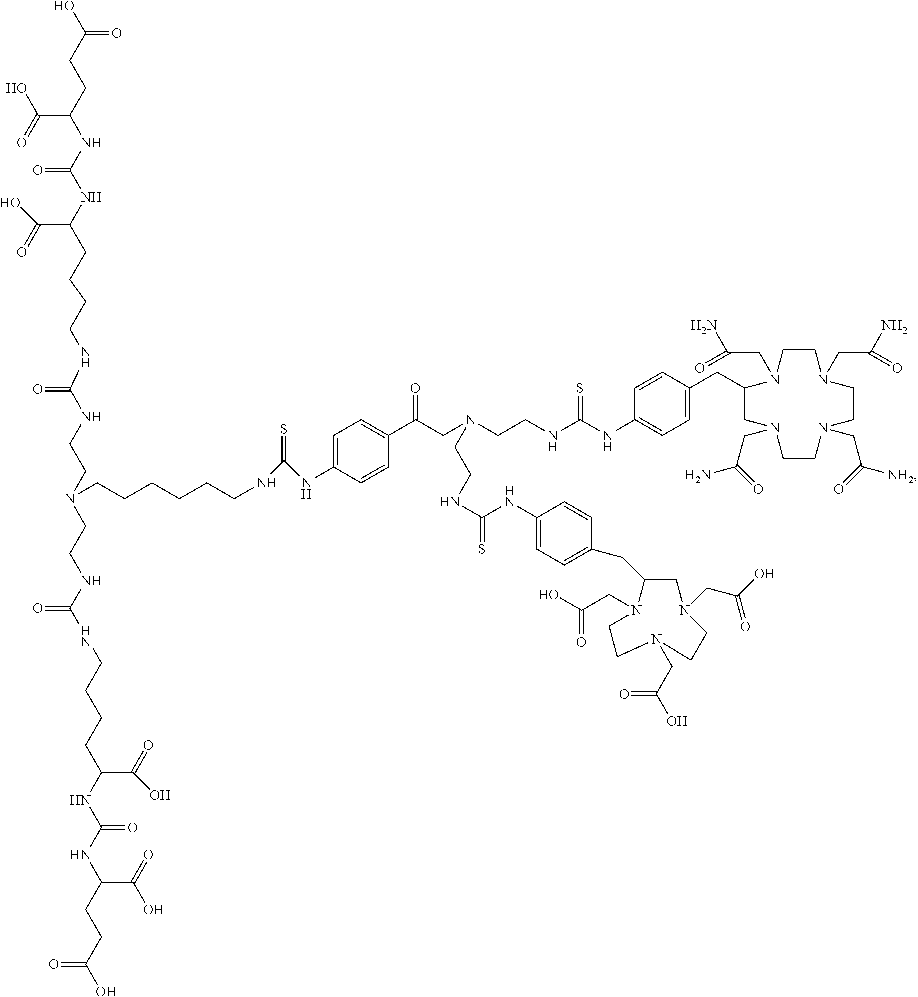 [SMILES: NC(=O)CN1CCN(CC(N)=O)CCN(CC(N)=O)C(Cc2ccc(NC(=S)NCCN(CCNC(=S)Nc3ccc(CC4CN(CC(=O)O)CCN(CC(=O)O)CCN4CC(=O)O)cc3)CC(=O)c3ccc(NC(=S)NCCCCCCN(CCNC(=O)NCCCCC(NC(=O)NC(CCC(=O)O)C(=O)O)C(=O)O)CCNC(=O)NCCCCC(NC(=O)NC(CCC(=O)O)C(=O)O)C(=O)O)cc3)cc2)CN(CC(N)=O)CC1]

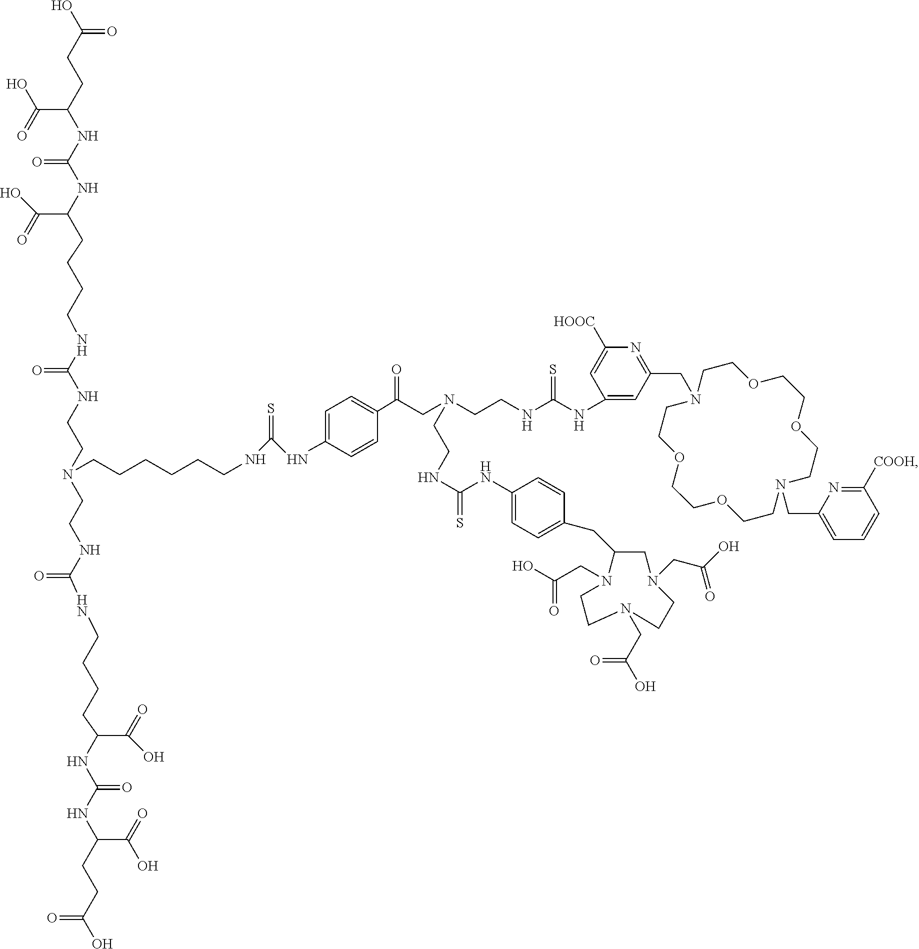 [SMILES: O=C(O)CCC(NC(=O)NC(CCCCNC(=O)NCCN(CCCCCCNC(=S)Nc1ccc(C(=O)CN(CCNC(=S)Nc2ccc(CC3CN(CC(=O)O)CCN(CC(=O)O)CCN3CC(=O)O)cc2)CCNC(=S)Nc2cc(CN3CCOCCOCCN(Cc4cccc(C(=O)O)n4)CCOCCOCC3)nc(C(=O)O)c2)cc1)CCNC(=O)NCCCCC(NC(=O)NC(CCC(=O)O)C(=O)O)C(=O)O)C(=O)O)C(=O)O]

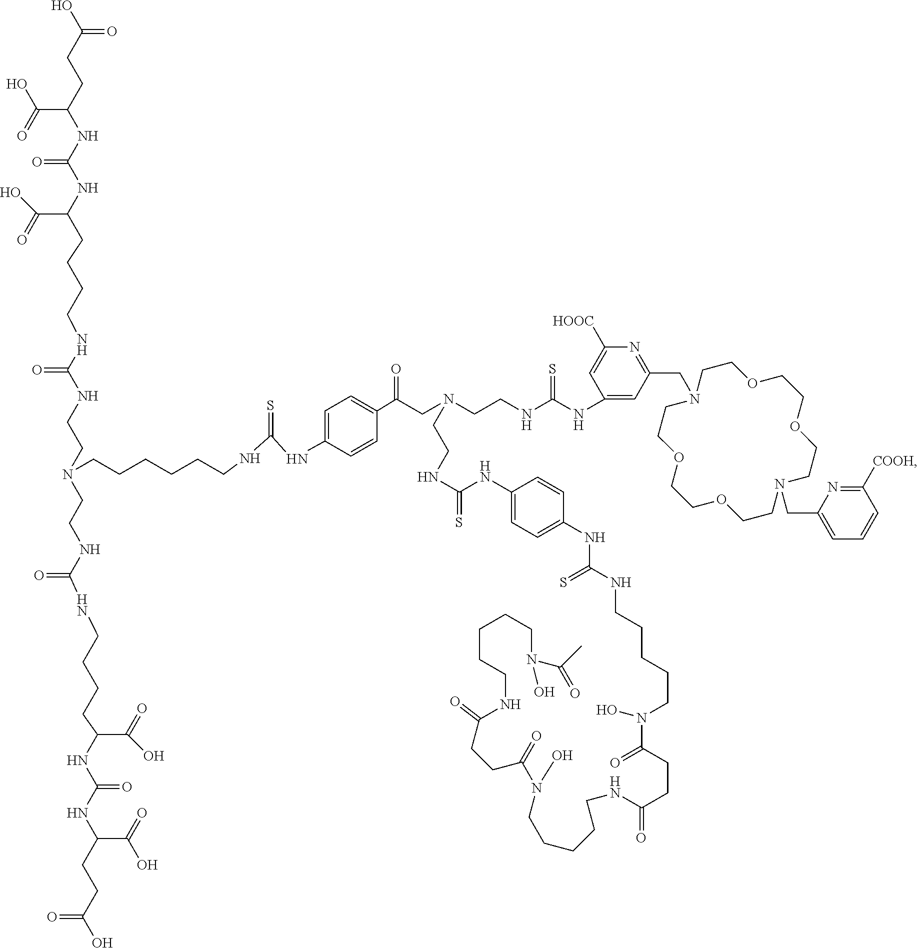 [SMILES: CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)NCCCCCCN(CCNC(=O)NCCCCC(NC(=O)NC(CCC(=O)O)C(=O)O)C(=O)O)CCNC(=O)NCCCCC(NC(=O)NC(CCC(=O)O)C(=O)O)C(=O)O)cc2)cc1]

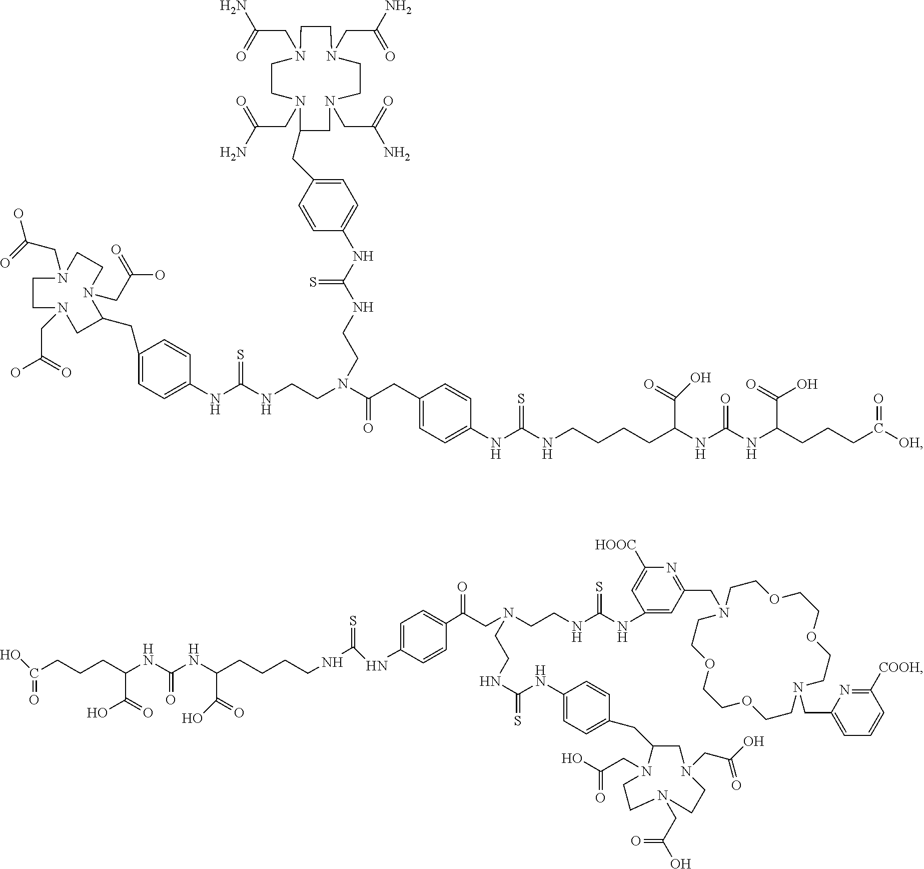 [SMILES: NC(=O)CN1CCN(CC(N)=O)CCN(CC(N)=O)C(Cc2ccc(NC(=S)NCCN(CCNC(=S)Nc3ccc(CC4CN(CC(=O)O)CCN(CC(=O)O)CCN4CC(=O)O)cc3)C(=O)Cc3ccc(NC(=S)NCCCCC(NC(=O)NC(CCCC(=O)O)C(=O)O)C(=O)O)cc3)cc2)CN(CC(N)=O)CC1 ; O=C(O)CCCC(NC(=O)NC(CCCCNC(=S)Nc1ccc(C(=O)CN(CCNC(=S)Nc2ccc(CC3CN(CC(=O)O)CCN(CC(=O)O)CCN3CC(=O)O)cc2)CCNC(=S)Nc2cc(CN3CCOCCOCCN(Cc4cccc(C(=O)O)n4)CCOCCOCC3)nc(C(=O)O)c2)cc1)C(=O)O)C(=O)O]

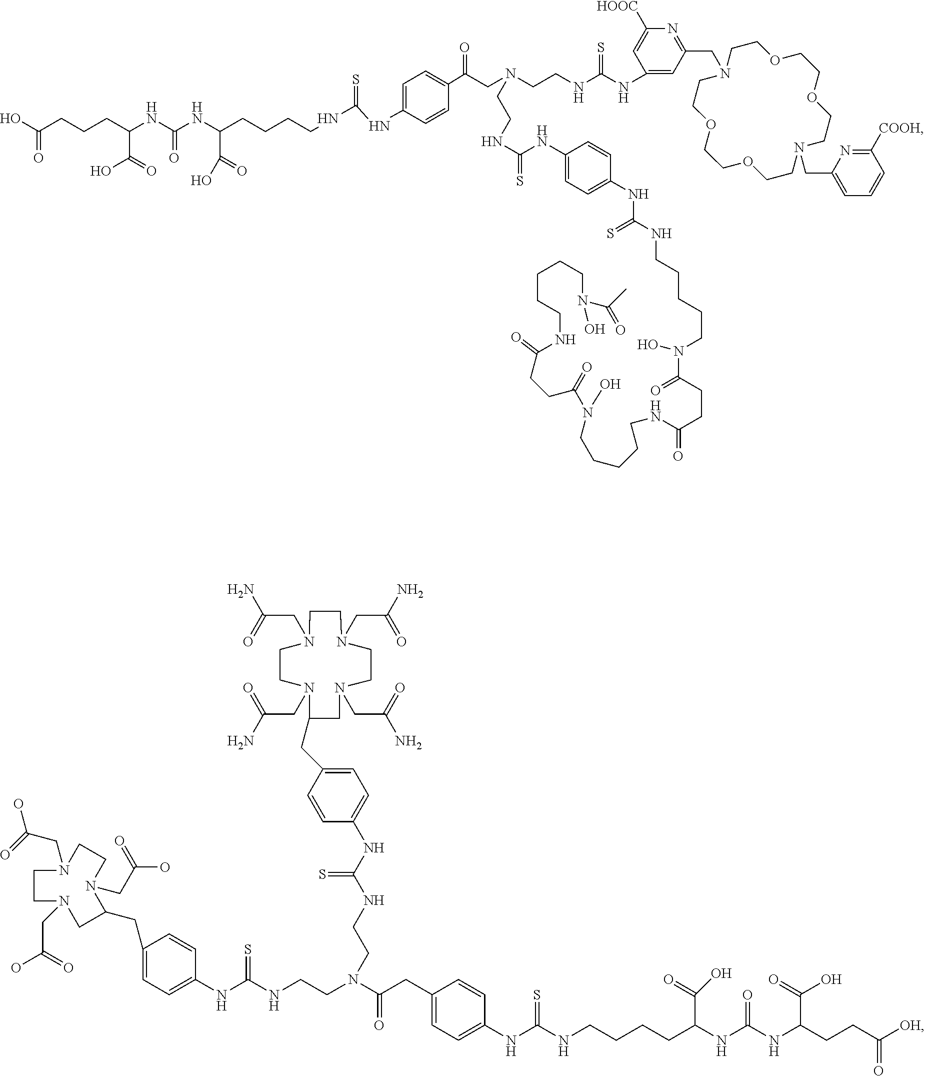 [SMILES: CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)NCCCCC(NC(=O)NC(CCCC(=O)O)C(=O)O)C(=O)O)cc2)cc1 ; NC(=O)CN1CCN(CC(N)=O)CCN(CC(N)=O)C(Cc2ccc(NC(=S)NCCN(CCNC(=S)Nc3ccc(CC4CN(CC(=O)O)CCN(CC(=O)O)CCN4CC(=O)O)cc3)C(=O)Cc3ccc(NC(=S)NCCCCC(NC(=O)NC(CCC(=O)O)C(=O)O)C(=O)O)cc3)cc2)CN(CC(N)=O)CC1]

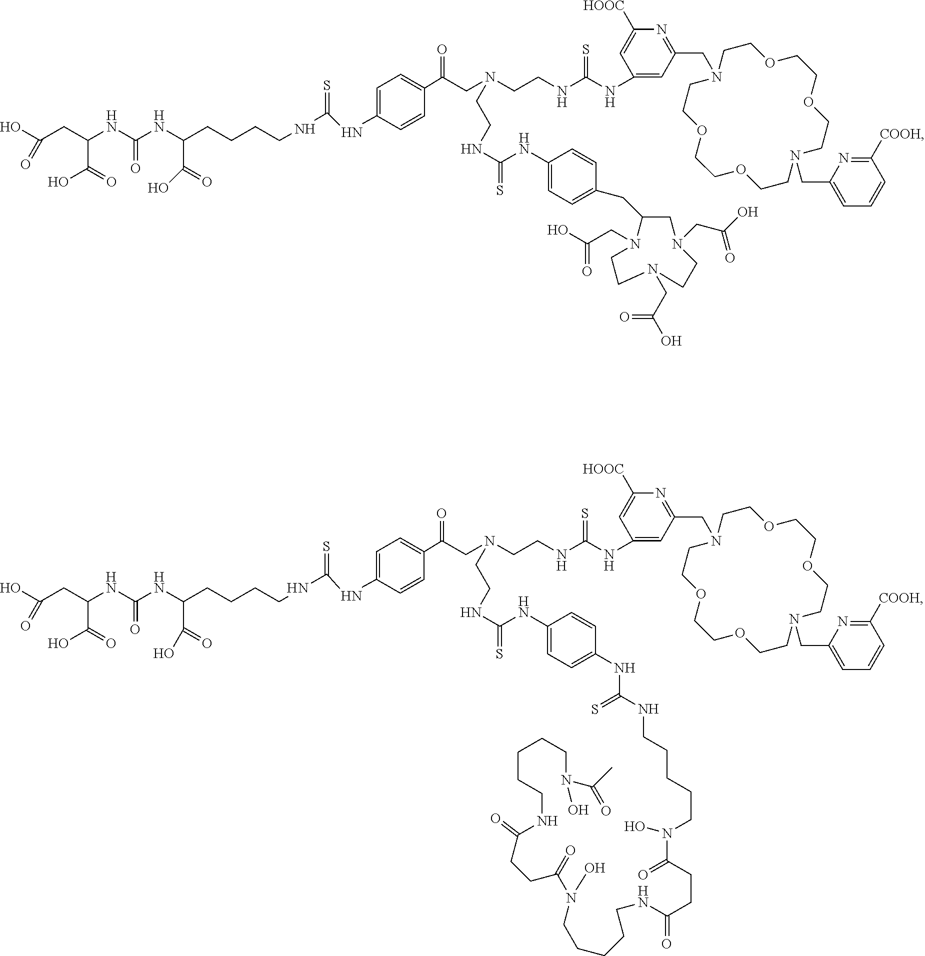 [SMILES: O=C(O)CC(NC(=O)NC(CCCCNC(=S)Nc1ccc(C(=O)CN(CCNC(=S)Nc2ccc(CC3CN(CC(=O)O)CCN(CC(=O)O)CCN3CC(=O)O)cc2)CCNC(=S)Nc2cc(CN3CCOCCOCCN(Cc4cccc(C(=O)O)n4)CCOCCOCC3)nc(C(=O)O)c2)cc1)C(=O)O)C(=O)O ; CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)NCCCCC(NC(=O)NC(CC(=O)O)C(=O)O)C(=O)O)cc2)cc1]

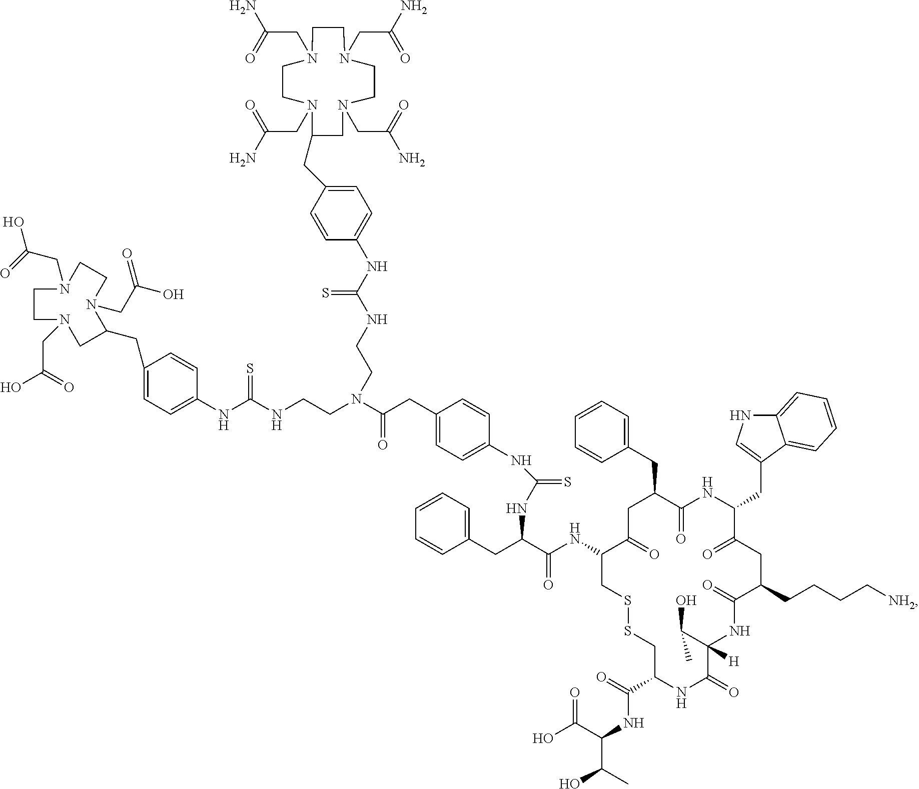

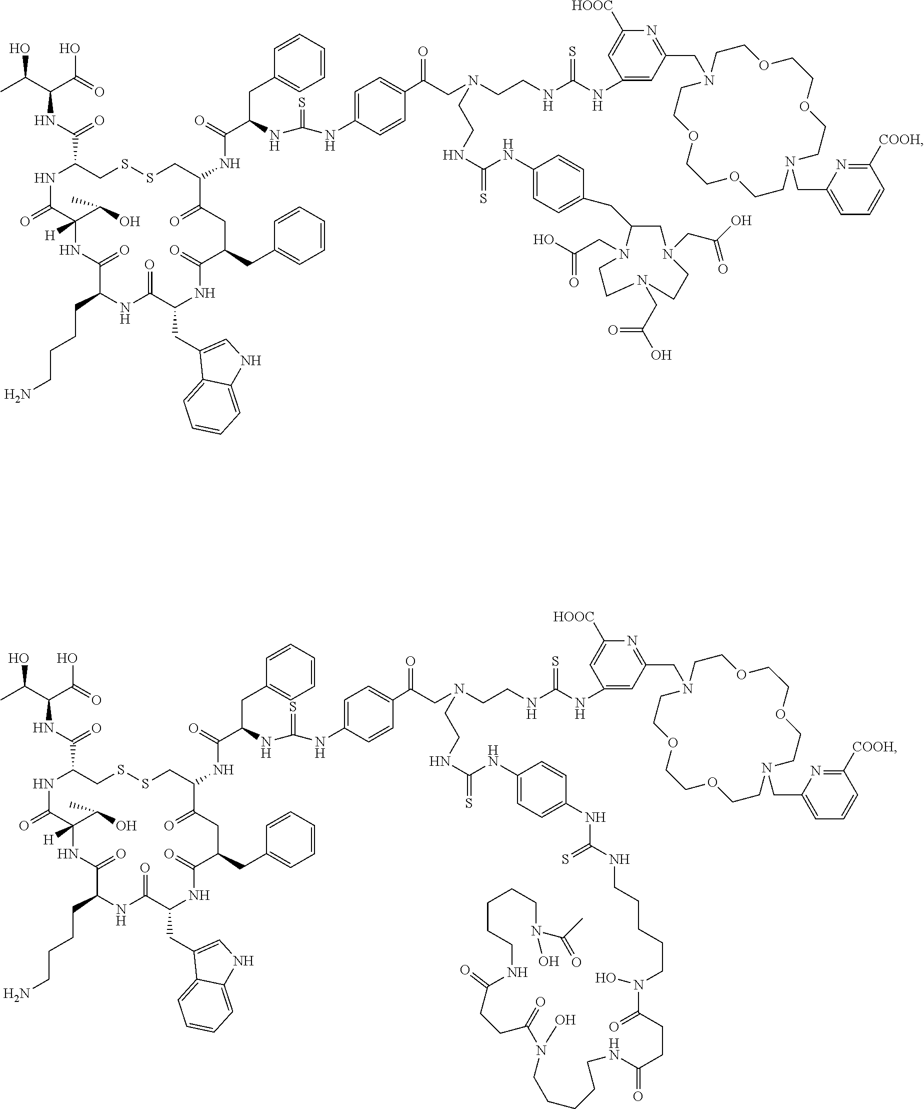

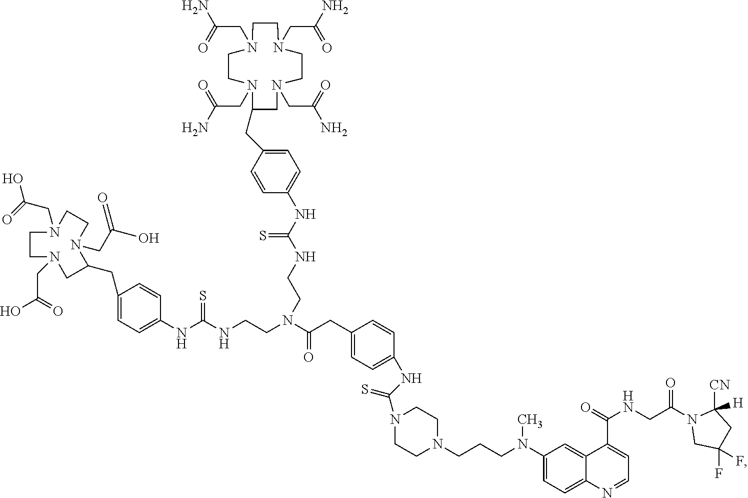 [SMILES: CN(CCCN1CCN(C(=S)Nc2ccc(CC(=O)N(CCNC(=S)Nc3ccc(CC4CN(CC(N)=O)CCN(CC(N)=O)CCN(CC(N)=O)CCN4CC(N)=O)cc3)CCNC(=S)Nc3ccc(CC4CN(CC(=O)O)CCN(CC(=O)O)CCN4CC(=O)O)cc3)cc2)CC1)c1ccc2nccc(C(=O)NCC(=O)N3CC(F)(F)C[C@H]3C#N)c2c1]

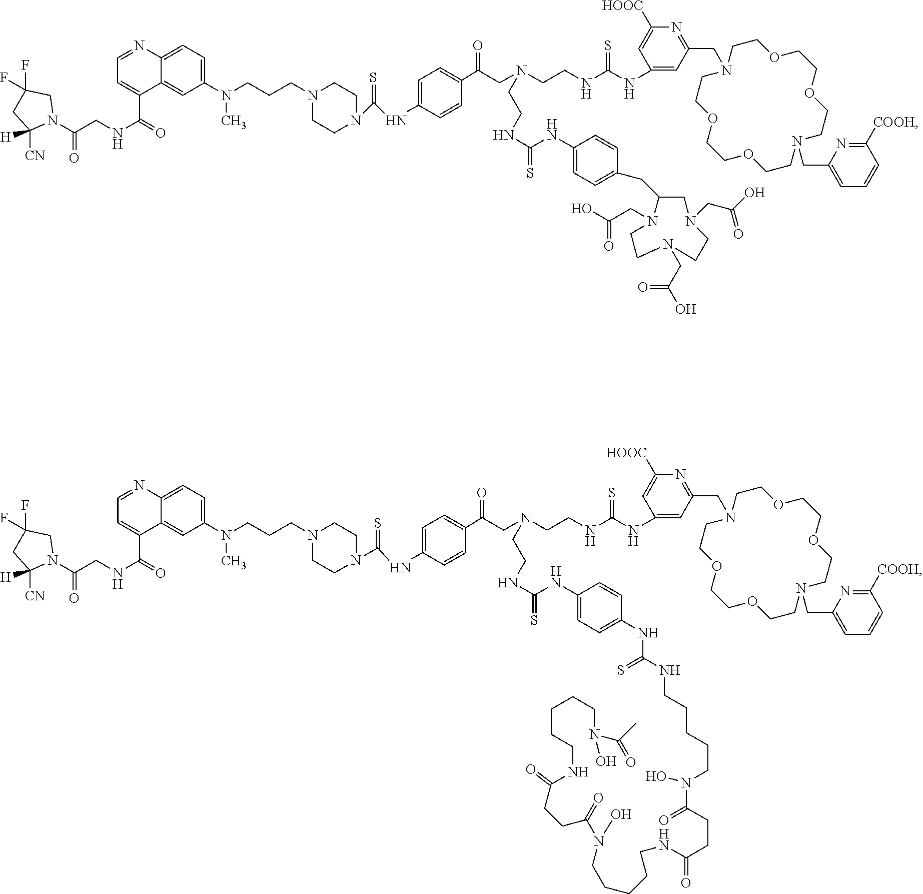 [SMILES: CN(CCCN1CCN(C(=S)Nc2ccc(C(=O)CN(CCNC(=S)Nc3ccc(CC4CN(CC(=O)O)CCN(CC(=O)O)CCN4CC(=O)O)cc3)CCNC(=S)Nc3cc(CN4CCOCCOCCN(Cc5cccc(C(=O)O)n5)CCOCCOCC4)nc(C(=O)O)c3)cc2)CC1)c1ccc2nccc(C(=O)NCC(=O)N3CC(F)(F)C[C@H]3C#N)c2c1 ; CC(=O)N(O)CCCCCNC(=O)CCC(=O)N(O)CCCCCNC(=O)CCC(=O)N(O)CCCCCNC(=S)Nc1ccc(NC(=S)NCCN(CCNC(=S)Nc2cc(CN3CCOCCOCCN(Cc4cccc(C(=O)O)n4)CCOCCOCC3)nc(C(=O)O)c2)CC(=O)c2ccc(NC(=S)N3CCN(CCCN(C)c4ccc5nccc(C(=O)NCC(=O)N6CC(F)(F)C[C@H]6C#N)c5c4)CC3)cc2)cc1]

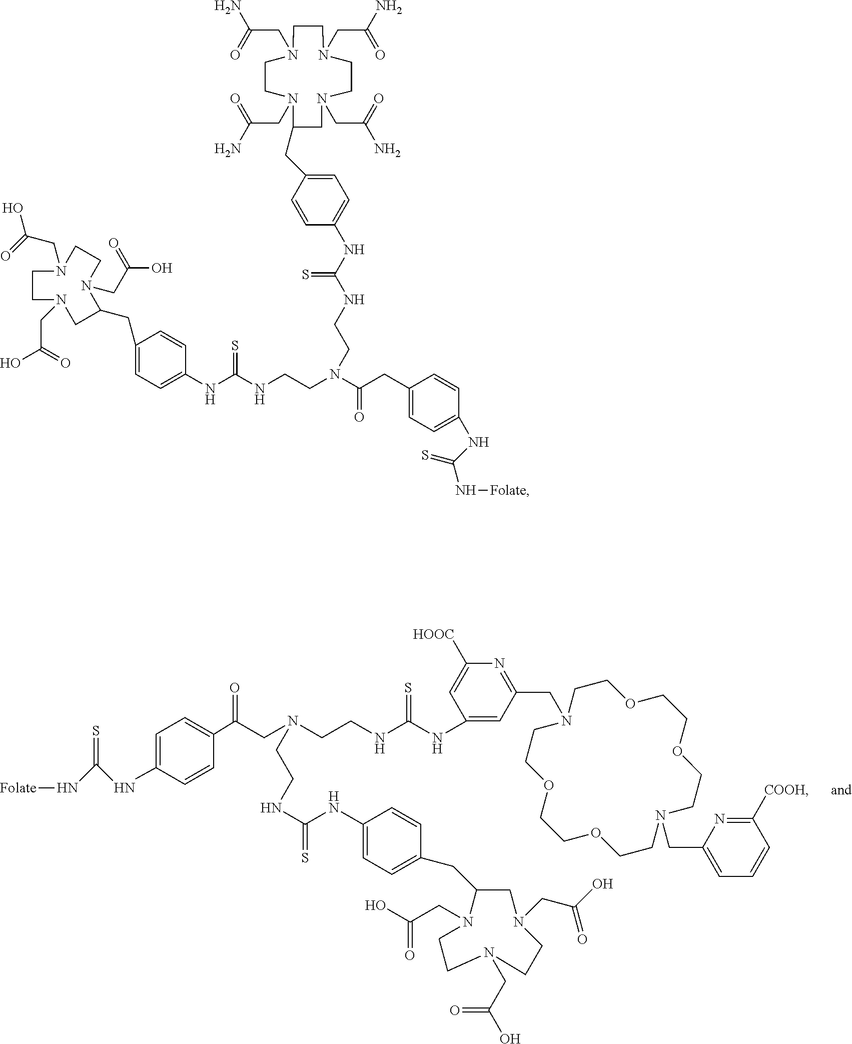

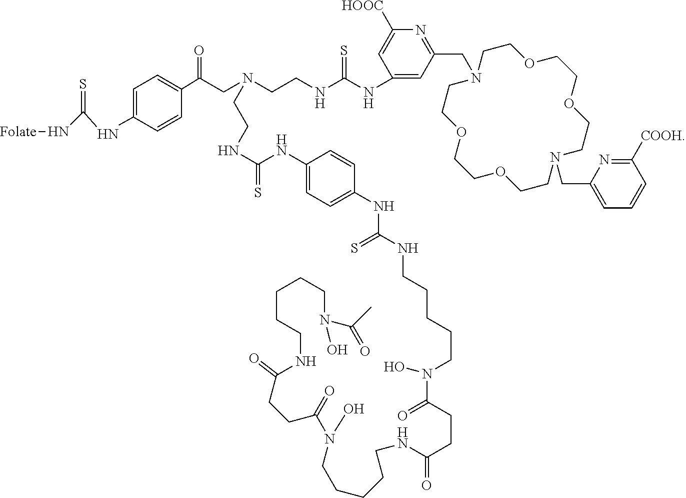

34-51. (canceled)
